(19) United States
(12) Patent Application Publication
(10) Pub. No.: US 2009/0176786 A1
(43) Pub. Date: Jul. 9, 2009

(54) Benzoylurea Compounds and Use Thereof

(76) Inventors: Masato Konobe, Toyonaka-shi (JP); Shigeyuki Itoh, Oita-shi (JP); Norihisa Sakamoto, Sanda-shi (JP); Araki Tomohiro, Toyonaka-shi (JP); Yoshitomo Tohyama, Hirakata-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/083,781

(22) PCT Filed: Oct. 16, 2006
(86) PCT No.: PCT/JP2006/320984

§ 371 Date: Aug. 8, 2008

(30) Foreign Application Priority Data
Oct. 20, 2005 (JP) 2005-305418
Mar. 31, 2006 (JP) 2006-097942
Jul. 13, 2006 (JP) 2006-192557

(51) Int. Cl. A01N 47/28; A01N 43/84; A01N 43/40; A01N 43/08; C07D 265/30; C07D 213/36; C07D 317/10; C07D 307/04; C07C 275/12; A01P 7/00

(52) U.S. Cl. 514/237.8; 514/357; 514/463; 514/471; 514/594; 544/159; 546/332; 549/452; 549/493; 564/44

(57) ABSTRACT

The present invention relates to a benzoylurea compound represented by formula (I):

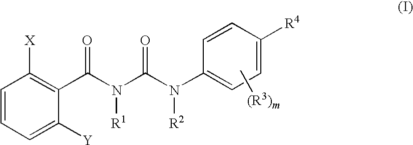

wherein, X and Y represent a fluorine atom or a chlorine atom, R¹ represents a lower alkyl group or the like, R² represents a lower alkyl group, R³ represents a halogen atom or the like, R⁴ represents an alkylthio group optionally substituted with one or more of halogen atoms, or a salt thereof, and use thereof for controlling pests and the like.

### TECHNICAL FIELD

The present invention relates to a benzoylurea compound and use thereof for pest control.

### BACKGROUND ART

EP 0263438A2, EP 0165903A2, U.S. Pat. No. 4,468,405, U.S. Pat. No. 4,170,657, U.S. Pat. No. 4,234,600, US 2005-0159599A1 and the like disclose benzoylurea compounds and derivatives thereof having a pesticidal activity.

### DISCLOSURE OF INVENTION

However, sometimes these compounds may not necessarily show a sufficient controlling efficacy for pests.

The problems of the present invention are to provide a compound having an excellent controlling efficacy for pests.

As a result of intensive studies to solve the above-mentioned problem, the present inventors found out that the benzoylurea compound represented by the following formula (I) has an excellent controlling efficacy for pests, and have completed the present invention.

That is, the present invention provides:

[1] A benzoylurea compound represented by formula (I) (hereinafter, referred to as compound (I))

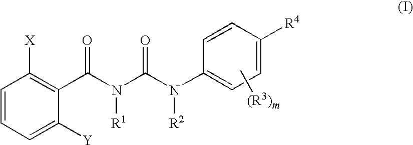

wherein, X and Y independently represent a fluorine atom or chlorine atom, respectively,
R¹ represents a hydrogen atom, a lower alkyl group optionally substituted with one or more of halogen atoms, a lower alkenyl group optionally substituted with one or more of halogen atoms, a lower alkynyl group, an aryl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of, halogen atoms, a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, a lower alkoxycarbonyl group, an aryl lower alkyloxycarbonyl group, a N,N-di(lower alkyl)carbamoyl group, a lower alkanoyl group optionally substituted with one or more of halogen atoms, formyl group, a lower alkylsulfonyl group optionally substituted with one or more of halogen atoms, an arylsulfonyl group, an aryloxycarbonyl group, a lower cycloalkyl group, a lower cycloalkyl lower alkyl group, a di(lower alkyl)amino group, a lower alkoxy group, a lower alkanoyloxy lower alkyl group, an aryl lower alkoxy lower alkyl group, 6-membered saturated heterocyclic group, or a group represented by —(CH₂)ₗ-A wherein l represents an integer of 1 to 4 and A represents a di(lower alkoxy)methyl group, a lower alkoxycarbonyl group, or a 5- or 6-membered heterocyclic group optionally substituted with a halogen atom,
R² represents a lower alkyl group,
R³ represents a halogen atom or a lower alkyl group optionally substituted with one or more of halogen atoms,
R⁴ represents a lower alkoxycarbonyl group, or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, and
m represents an integer of 0 to 4; or a salt thereof.
[2] The compound according to the above-mentioned [1], wherein X and Y independently represents a fluorine atom or a chlorine atom, respectively,
R¹ represents a hydrogen atom, a lower alkyl group optionally substituted with one or more of halogen atoms, a lower alkenyl group optionally substituted with one or more of halogen atoms, a lower alkynyl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a N,N-di(lower alkyl)amino lower alkyl group, a lower alkylthio lower alkyl group, a lower alkylsulfinyl lower alkyl group, a lower alkylsulfonyl lower alkyl group, a lower alkoxycarbonyl group, an aryl lower alkyloxycarbonyl group, a N,N-di(lower alkyl)carbamoyl group, a lower alkanoyl group optionally substituted with one or more of halogen atoms, a lower alkylsulfonyl group optionally substituted with one or more of halogen atoms, an arylsulfonyl group, an aryloxycarbonyl group, a lower cycloalkyl group, a lower cycloalkyl lower alkyl group, a di(lower alkyl)amino group, a lower alkoxy group, an aryl lower alkoxy lower alkyl group, a 6-membered saturated heterocyclic group, or a group represented by —(CH₂)ₗ-A wherein l represents an integer of 1 or 2, and A represents a di(lower alkoxy)methyl group, a lower alkoxycarbonyl group, or a 5- or 6-membered heterocyclic group optionally substituted with a halogen atom,
R² represents a lower alkyl group,
R³ represents a halogen atom or a lower alkyl group optionally substituted with one or more of halogen atoms,
R⁴ represents a lower alkoxycarbonyl group, or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2,
m represents an integer of 0 to 2.
[3] The compound according to the above-mentioned [1] or
[2], wherein R¹ represents a hydrogen atom, a lower alkyl group optionally substituted with one or more of halogen atoms, a lower alkenyl group, a lower alkynyl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a lower alkylthio lower alkyl group, a lower alkylsulfinyl lower alkyl group, a lower alkylsulfonyl lower alkyl group, a lower alkoxycarbonyl group, an aryl lower alkyloxycarbonyl group, a N,N-di(lower alkyl)carbamoyl group, a lower alkanoyl group, a lower alkylsulfonyl group, an arylsulfonyl group, a lower cycloalkyl group, a lower cycloalkyl lower alkyl group, a di(lower alkyl)amino group, a lower alkoxy group, a 6-membered saturated heterocyclic group, or a group represented by —(CH₂)ₗ-A wherein l represents an integer of 1 or 2, and A represents a lower alkoxycarbonyl group, or a 5- or 6-membered heterocyclic group optionally substituted with a halogen atom,
R³ represents a halogen atom or a lower alkyl group,
R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms.
[4] The compound according to any one of the above-mentioned [1] to [3], wherein n represents an integer of 1 or 2.
[5] The compound according to any one of the above-mentioned [1] to [3], wherein R⁴ represents a lower alkoxycarbonyl group.
[6] The compound according to any one of the above-mentioned [1] to [3], wherein R¹ represents a lower alkyl group substituted with one or more of halogen atoms.
[7] A benzoylurea compound represented by formula (I-a)

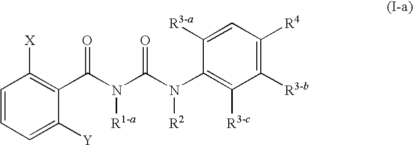

wherein, X and Y independently represent a fluorine atom or chlorine atom, respectively,
R^{1-a} represents a hydrogen atom or a lower alkyl group,
R² represents a lower alkyl group, and

(1) when R^{3-a} and R^{3-b} represent a halogen atom, R^{3-c} represents a hydrogen atom, or

(2) when R^{3-a} and R^{3-c} represent a halogen atom, R^{3-b} represents a hydrogen atom, or

(3) when R^{3-a} represents a halogen atom or a lower alkyl group, R^{3-b} and R^{3-c} represent a hydrogen atom, and

R⁴ represents a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, or a salt thereof.
[8] The compound according to the above-mentioned [7], wherein (1) when R^{3-a} and R^{3-b} represent a halogen atom, R^{3-c} represents a hydrogen atom, or

(2) when R^{3-a} and R^{3-c} represent a halogen atom, R^{3-b} represents a hydrogen atom, and

R⁵ represents a lower alkyl group optionally substituted with one or more of halogen atoms.
[9] The compound according to the above-mentioned [8], wherein R^{3-a} represents a halogen atom or a lower alkyl group,
R^{3-b} and R^{3-c} represent a hydrogen atom,
R⁵ represents a lower alkyl group optionally substituted with one or more of halogen atoms.
[10] The compound according to any one of the above-mentioned [1] to [3], wherein R³ represents a lower alkyl group substituted with a halogen atom.
[11] A process for producing a compound represented by formula (I-7)

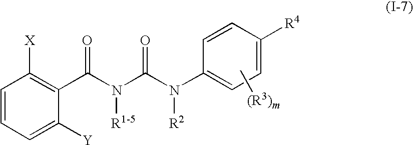

wherein, X and Y independently represent a fluorine atom or a chlorine atom, respectively,
R¹⁻⁵ represents a lower alkyl group optionally substituted with one or more of halogen atoms, a lower alkenyl group optionally substituted with one or more of halogen atoms, a lower alkynyl group, an aryl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, a lower alkoxycarbonyl group, an aryl lower alkyloxycarbonyl group, a N,N-di(lower alkyl)carbamoyl group, a lower alkanoyl group optionally substituted with one or more of halogen atoms, formyl group, a lower alkylsulfonyl group optionally substituted with one or more of halogen atoms, an arylsulfonyl group, an aryloxycarbonyl group, a lower cycloalkyl group, a lower cycloalkyl lower alkyl group, a di(lower alkyl)amino group, a lower alkoxy group, a lower alkanoyloxy lower alkyl group, an aryl lower alkoxy lower alkyl group, 6-membered saturated heterocyclic group, or a group represented by —(CH₂)ₗ-A wherein l represents an integer of 1 to 4, and A represents a di(lower alkoxy)methyl group, a lower alkoxycarbonyl group, or a 5- or 6-membered heterocyclic group optionally substituted with a halogen atom,
R² represents a lower alkyl group,
R³ represents a halogen atom, or a lower alkyl group optionally substituted with one or more of halogen atoms,
R⁴ represents a lower alkoxycarbonyl group, or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, and
m represents an integer of 0 to 4,
which comprises reacting a compound represented by formula (II)

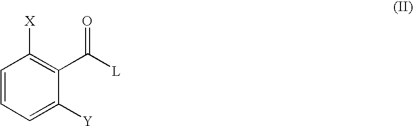

wherein X and Y are as defined above, and L represents a halogen atom, with a compound represented by formula (III)

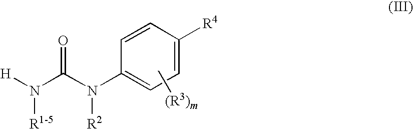

wherein each symbol is as defined above, in an organic solvent in the presence of an organic base or a metal carbonate, and isolating.
[12] The process according to the above-mentioned [11], wherein R¹⁻⁵ represents a lower alkyl group,
R³ represents a halogen atom, or a lower alkyl group,
R⁴ represents a group represented by S(O)ₙR⁵ wherein
R⁵ represents a lower alkyl group optionally substituted with one or more of halogen atoms, and n represents an integer of 0, and
m represents an integer of 1.
[13] A pesticide comprising the compound or a salt thereof according to any one of the above-mentioned [1] to [10] as an active ingredient.
[14] Use of the compound or a salt thereof according to any one of the above-mentioned [1] to [10] for pest control.
[15] Use of the compound according to any one of the above-mentioned [1] to [10] for manufacturing a pesticide for controlling pests.
[16] A method for controlling pests which comprises applying the compound or a salt thereof according to any one of the above-mentioned [1] to [10] to pests directly or habitat of pests.
[17] A compound represented by formula (III)

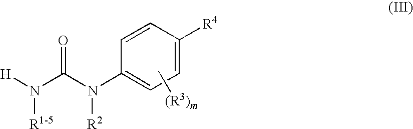

wherein, R¹⁻⁵ represents a lower alkyl group optionally substituted with one or more of halogen atoms, a lower alkenyl group optionally substituted with one or more of halogen atoms, a lower alkynyl group, an aryl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a lower alkanoyloxy lower alkyl group, an aryl lower alkoxy lower alkyl group, a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, a lower alkoxycarbonyl group, an aryl lower alkyloxycarbonyl group, a N,N-di(lower alkyl)carbamoyl group, a lower alkanoyl group optionally substituted with one or more of halogen atoms, formyl group, a lower alkylsulfonyl group optionally substituted with one or more of halogen atom, an aryl sulfonyl group, an aryloxycarbonyl group, a lower cycloalkyl group, a lower cycloalkyl lower alkyl group, a di(lower alkyl)amino group, a lower alkoxy group, 6-membered saturated heterocyclic group, or a group represented by —(CH₂)ₗ-A wherein l represents an integer of 1 to 4, and A represents a di(lower alkoxy)methyl group, a lower alkoxycarbonyl group, or a 5- or 6-membered heterocyclic group optionally substituted with a halogen atom,
R² represents a lower alkyl group,
R³ represents a halogen atom, or a lower alkyl group optionally substituted with one or more of halogen atoms,
R⁴ represents a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, and
m represents an integer of 0 to 4.
[18] The compound according to [17], wherein R¹⁻⁵ represents a lower alkyl group optionally substituted with one or more of halogen atoms, a lower alkenyl group optionally substituted with one or more of halogen atoms, a lower alkynyl group, an aryl lower alkyl group optionally substituted with one or more of lower alkyl groups, a lower alkoxy lower alkyl group optionally substituted with one or more of halogen atoms, an aryloxy lower alkyl group optionally substituted with one or more of halogen atoms, a N,N-di(lower alkyl)amino lower alkyl group, a lower alkylthio lower alkyl group, a lower alkylsulfinyl lower alkyl group, a lower alkylsulfonyl lower alkyl group, a lower cycloalkyl group, a lower cycloalkyl lower alkyl group, a di(lower alkyl)amino group, a lower alkoxy group, 6-membered saturated heterocyclic group, or a group represented by —(CH₂)ₗ-A wherein l represents an integer of 1 to 4, and A represents a di(lower alkoxy)methyl group, a lower alkoxycarbonyl group, or a 5- or 6-membered heterocyclic group optionally substituted with a halogen atom.

Suitable examples relevant to a variety of definitions and examples included in the scope of the present invention used in the above-described and below-described descriptions of the present specification will be described in detail below.

The term “lower” indicates a group having 6 or less carbon atoms unless otherwise mentioned herein, and preferably, it may be a group having 4 or less carbon atoms.

A suitable example of the “one or more” includes 1 to 6, preferably 1 to 4.

Suitable examples of the “lower alkyl group” and “lower alkyl” include a straight-chain or branched C1-C6 alkyl group, for example, methyl, ethyl, n-propyl, isopropyl, n-butyl, isobutyl, sec-butyl, tert-butyl, n-pentyl, sec-pentyl, isopentyl, neopentyl, n-hexyl, isohexyl and the like are exemplified.

The “lower cycloalkyl” is referred to cycloalkyl, and indicates a group having 6 or less carbon atoms which constitute the ring.

Suitable examples of the “lower cycloalkyl group” and “lower cycloalkyl” include a cyclic C3-C6 alkyl group, for example, cyclopropyl, cyclobutyl, cyclopentyl, cyclohexyl and the like are exemplified.

Suitable examples of the “lower alkenyl group” include a straight-chain or branched C2-C6 alkenyl group, for example, vinyl, allyl, isopropenyl, isobutenyl, 1-methylallyl, 2-pentenyl, 2-hexenyl and the like are exemplified.

Suitable examples of the “lower alkynyl group” include a C2-C6 alkynyl group, for example, ethynyl, 2-propynyl, 1-propynyl, 2-butynyl, 3-butynyl, 3-pentynyl, 3-hexynyl and the like are exemplified.

Suitable examples of the “aryl group” and “aryl” include a C6-C14 aromatic hydrocarbon group such as phenyl optionally substituted with lower alkyl (e.g., phenyl, mesityl, xylyl, tolyl and the like), naphtyl, anthryl, indanyl and the like, preferably phenyl and naphtyl, and these “aryl group” and “aryl” may have a suitable substituent such as a lower alkyl group, a halogen, an aryl group and the like.

As the halogen, fluorine, chlorine, bromine and iodine are exemplified.

Suitable examples of the “lower alkoxy group” and “lower alkoxy” include a straight-chain or branched C1-C6 alkoxy group, for example, methoxy, ethoxy, propoxy, isopropoxy, butoxy, isobutoxy, tert-butoxy, pentyloxy, tert-pentyloxy, neo-pentyloxy, hexyloxy, isohexyloxy and the like are exemplified, and preferably methoxy, ethoxy, propoxy, butoxy, pentyloxy, hexyloxy, isohexyloxy are exemplified.

Suitable examples of the “lower alkanoyl group” include a straight-chain or branched C2-C6 alkanoyl groups, for example, acetyl, 2-methyl acetyl, 2,2-dimethylacetyl, propionyl, butylyl, isobutylyl, pentanoyl, 2,2-dimethylpropionyl, hexanoyl and the like are exemplified.

In R¹, examples of the “lower alkyl group optionally substituted with one or more of halogen atoms” include methyl, ethyl, 2-bromoethyl, 2,2,2-trifluoroethyl, propyl, 3,3,3-trifluoropropyl, isopropyl, butyl, isobutyl, sec-butyl, tert-butyl, 4,4,4-trifluorobutyl, pentyl, isopentyl, neopentyl, 5,5,5-trifluoropentyl, hexyl and 6,6,6-trifluorohexyl.

Examples of the “lower alkenyl group optionally substituted with one or more of halogen atoms” include vinyl, 1-propenyl, 2-propenyl, isopropenyl, 2-butenyl, isobutenyl and 3,3-dichloro-2-propenyl.

Examples of the “lower alkynyl group” include ethynyl, 2-propynyl and 1-propynyl.

Examples of the “aryl group” include phenyl, 1-naphthyl, 2-naphthyl and biphenylyl.

Examples of the “aryl lower alkyl group optionally substituted with one or more of lower alkoxy groups” include benzyl, phenethyl, 2-methoxybenzyl, 3-methoxybenzyl and 4-methoxybenzyl.

Examples of the “lower alkoxy lower alkyl group optionally substituted with one or more of halogen atoms” include methoxymethyl, ethoxymethyl, 1-propoxymethyl, 2-methoxyethyl, 2-ethoxyethyl, 3-methoxypropyl, 3-ethoxypropyl and 2-chloroethoxymethyl.

Examples of the “aryloxy lower alkyl group optionally substituted with one or more of halogen atoms” include phenoxymethyl, 2-phenoxyethyl and 4-chlorophenoxymethyl.

Examples of the “N,N-di(lower alkyl)amino lower alkyl group” include dimethylaminomethyl, 2-(dimethylamino)ethyl, diethylaminomethyl and 2-(diethylamino)ethyl.

Examples of the “lower alkylthio lower alkyl group” include methylthiomethyl, ethylthiomethyl, 2-(methylthio)ethyl and 2-(ethylthio)ethyl.

Examples of the “lower alkylsulfinyl lower alkyl group” include methylsulfinylmethyl, ethylsulfinylmethyl, 2-(methylsulfinyl)ethyl and 2-(ethylsulfinyl)ethyl.

Examples of the “lower alkylsulfonyl lower alkyl group” include methylsulfonylmethyl, ethylsulfonylmethyl, 2-(methylsulfonyl)ethyl and 2-(ethylsulfonyl)ethyl.

Examples of the “lower alkoxy lower alkoxy lower alkyl group” include (2-methoxyethoxy)methyl.

Examples of the “lower alkoxycarbonyl group” include methoxycarbonyl, ethoxycarbonyl, propyloxycarbonyl, isopropyloxycarbonyl, butoxycarbonyl and tert-butoxycarbonyl.

Examples of the “aryl lower alkyloxycarbonyl group” include benzyloxycarbonyl.

Examples of the “N,N-di(lower alkyl)carbamoyl group” include dimethylcarbamoyl and diethylcarbamoyl.

Examples of the “lower alkanoyl group optionally substituted with one or more of halogen atoms” include acetyl, propionyl, trifluoroacetyl and chloroacetyl.

Examples of the “lower alkylsulfonyl group optionally substituted with one or more of halogen atoms” include methane sulfonyl, ethane sulfonyl and trifluoromethane sulfonyl.

Examples of the “aryl sulfonyl group” include benzenesulfonyl and toluenesulfonyl.

Examples of the “aryloxy carbonyl group” include phenoxycarbonyl.

Suitable examples of the “lower cycloalkyl group” and “lower cycloalkyl” include a cyclic C3-C6 alkyl group, for example, cyclopropyl, cyclobutyl, cyclopentyl, cyclohexyl and the like are exemplified.

Examples of the “lower cycloalkyl lower alkyl group” include cyclopropylmethyl, cyclopropylethyl, cyclobutylmethyl, cyclobutylethyl, cyclopentylmethyl, cyclopentylethyl, cyclohexylmethyl, and cyclohexylethyl.

Examples of the “di(lower alkyl)amino group” include dimethylamino, and diethylamino.

Examples of the “lower alkoxy group” include methoxy and ethoxy.

Examples of the “lower alkanoyloxy lower alkyl group” include acetoxymethyl and acetoxyethyl.

Examples of the “aryl lower alkoxy lower alkyl group” include benzyloxymethyl and benzyloxyethyl.

Examples of the “6-membered saturated heterocyclic ring” include morpholino and 4-tetrahydropyranyl.

Examples of the “5- or 6-membered heterocyclic ring which may be substituted with a halogen atom” in A include 2-furyl, 3-furyl, morpholino, 2-tetrahydrofuryl, 3-tetrahydrofuryl, 1,3-dioxolan-2-yl, 2-thiazolyl, 4-thiazolyl, 5-thiazolyl, 2-chlorothiazol-5-yl, 2-pyridyl, 3-pyridyl and 4-pyridyl.

Examples of the “di(lower alkoxy)methyl group” include dimethoxymethyl.

Examples of the “lower alkoxycarbonyl group” include methoxycarbonyl.

In R², examples of the “lower alkyl group” include methyl and ethyl.

In R³, examples of the “halogen atom” include fluorine, chlorine, bromine and iodine.

Examples of the “lower alkyl group optionally substituted with one or more of halogen atoms” include methyl, chloromethyl, difluoromethyl, trichloromethyl, trifluoromethyl, ethyl, 2-bromoethyl, 2,2,2-trifluoroethyl, 1,1,2,2,2-pentafluoroethyl, propyl, 3,3,3-trifluoropropyl, isopropyl, butyl, isobutyl, sec-butyl, tert-butyl, and 4,4,4-trifluorobutyl.

In R⁴, examples of the “lower alkoxycarbonyl group” include methoxycarbonyl, ethoxycarbonyl, propyloxycarbonyl, isopropyloxycarbonyl, butoxycarbonyl, and tert-butoxy carbonyl.

In R⁵, examples of the “lower alkyl group optionally substituted with one or more of halogen atoms” include methyl, ethyl, isopropyl, tert-butyl, difluoromethyl, trifluoromethyl, trichloromethyl, dichlorofluoromethyl, chlorodifluoromethyl, 2,2,2-trifluoroethyl, 1,1,2,2-tetrafluoroethyl, 1,1,2,2,2-pentafluoroethyl, 1,1,2,2,3,3,3-heptafluoro-1-propyl, 1,1,2,3,3,3-hexafluoro-1-propyl, 1,1,1,2,3,3,3-heptafluoro-2-propyl, and trichloromethyl.

Examples of the “lower alkenyl group optionally substituted with one or more of halogen atoms” include 2-propenyl and 3,3-dichloro-2-propenyl.

Examples of the “lower alkynyl group” include 2-propinyl.

Examples of the “lower alkoxy lower alkyl group optionally substituted with one or more of halogen atoms” include 2-trifluoromethoxy-1,1,2-trifluoroethyl.

In addition, it should be noted that in the present specification, methyl group may be referred to as Me, and ethyl group may be referred to as Et.

As examples of the embodiment of compound (I), the followings are exemplified:

### EMBODIMENT 1

In the formula (I),

a benzoylurea compound, wherein
X and Y independently represent a fluorine atom or a chlorine atom, respectively,
R¹ represents a hydrogen atom, a lower alkyl group optionally substituted with one or more of halogen atoms, a lower alkenyl group optionally substituted with one or more of halogen atoms, a lower alkynyl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, N,N-di(lower alkyl)amino lower alkyl group, a lower alkylthio lower alkyl group, a lower alkylsulfinyl lower alkyl group, a lower alkylsulfonyl lower alkyl group, a lower alkoxycarbonyl group, an aryl lower alkyloxycarbonyl group, N,N-di(lower alkyl)carbamoyl group, a lower alkanoyl group optionally substituted with one or more of halogen atoms, a lower alkylsulfonyl group optionally substituted with one or more of halogen atoms, an arylsulfonyl group, an aryloxycarbonyl group, a lower cycloalkyl group, a lower cycloalkyl lower alkyl group, di(lower alkyl)amino group, a lower alkoxy group, an aryl lower alkoxy lower alkyl group, a 6-membered saturated heterocyclic group, or a group represented by —(CH₂)ₗ-A, wherein l represents an integer of 1 or 2, and A represents a di(lower alkoxy)methyl group, a lower alkoxycarbonyl group, or a 5- or 6-membered heterocyclic group optionally substituted with a halogen atom,
R² represents a lower alkyl group,
R³ represents a halogen atom, or a lower alkyl group optionally substituted with one or more of halogen atoms,
R⁴ represents a lower alkoxycarbonyl group or a group represented by S(O)ₙR⁵, wherein R⁵ represents a lower alkyl group optionally substituted with one or more of halogen atoms, a lower alkenyl group optionally substituted with one or more of halogen atoms, a lower alkynyl group or a lower alkoxy lower alkyl group optionally substituted with one or more of halogen atoms, and n represents an integer of 0 to 2, and
m represents an integer of 0 to 2, or a salt thereof.

### EMBODIMENT 2

In the formula (I),

a benzoylurea compound, wherein
X and Y independently represent a fluorine atom or a chlorine atom, respectively,
R¹ represents a hydrogen atom, a lower alkyl group optionally substituted with one or more of halogen atoms, a lower alkenyl group, a lower alkynyl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a lower alkylthio lower alkyl group, a lower alkylsulfinyl lower alkyl group, a lower alkylsulfonyl lower alkyl group, a lower alkoxycarbonyl group, an aryl lower alkyloxycarbonyl group, N,N-di(lower alkyl)carbamoyl group, a lower alkanoyl group, a lower alkylsulfonyl group, an arylsulfonyl group, a lower cycloalkyl group, a lower cycloalkyl lower alkyl group, di(lower alkyl)amino group, a lower alkoxy group, an aryl lower alkoxy lower alkyl group, a 6-membered saturated heterocyclic group, or a group represented by —(CH₂)ₗ-A, wherein l represents an integer of 1 or 2, and A represents a lower alkoxycarbonyl group, or a 5- or 6-membered heterocyclic group optionally substituted with a halogen atom,
R² represents a lower alkyl group,
R³ represents a halogen atom, or a lower alkyl group,
R⁴ represents a lower alkoxycarbonyl group or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, and
m represents an integer of 0 to 2, or a salt thereof.

### EMBODIMENT 3

In the formula (I),

a benzoylurea compound wherein
X and Y independently represent a fluorine atom and a chlorine atom, respectively,
R¹ represents a hydrogen atom, methyl, ethyl, 2,2,2-trifluoroethyl, 2-propenyl, 2-propinyl, benzyl, methoxymethyl, 2-methoxyethyl, 2-phenoxyethyl, 2-(dimethylamino)ethyl, 2-(methylthio)ethyl, 2-(methylsulfinyl)ethyl, 2-(methylsulfonyl)ethyl, methoxycarbonyl, benzyloxycarbonyl, dimethylcarbamoyl, acetyl, methanesulfonyl, benzenesulfonyl, phenoxycarbonyl, cyclopropyl, cyclohexyl, cyclopropylmethyl, cyclohexylmethyl, dimethylamino, methoxy, morpholino, 4-tetrahydropyranyl, 2,2-dimethoxyethyl, methoxycarbonylmethyl, 2-tetrahydrofurylmethyl, 2-furylmethyl, (1,3-dioxolan-2-yl)methyl, 2-pyridylmethyl, 3-pyridylmethyl, (2-chlorothiazol-5-yl)methyl, 2-methoxybenzyl, 3-methoxybenzyl, 4-methoxybenzyl, ethoxymethyl, 2-chloroethoxymethyl, benzyloxymethyl, (2-methoxyethoxy)methyl, or 2-morpholinoethyl,
R² represents methyl or ethyl,
R³ represents fluorine atom, chlorine atom, trifluoromethyl, or methyl,
R⁴ represents tert-butoxycarbonyl, trifluoromethylthio, trifluoromethysulfinyl, trifluoromethylsulfonyl, difluoromethylthio, trichloromethylthio, methylthio, ethylthio, 1,1,2,2-tetrafluoroethylthio, 1,1,2,2-tetrafluoroethylsulfinyl, 1,1,2,2-tetrafluoroethylsulfonyl, 2,2,2-trifluoroethylthio, 1,1,2,2,2-pentafluoroethylthio, 1,1,2,2,3,3,3-heptafluoro-1-propylthio, 1,1,2,3,3,3-hexafluoro-1-propylthio, 2-propenylthio, 2-propenylsufinyl, 2-proenylsulfonyl, 3,3-dichloro-2-propenylthio, 2-propynylthio, 2-propynylsulfinyl, 2-propynylsulfonyl, or 1,1,2-trifluoro-2-trifluoromethoxyethylthio, and
m represents an integer of 0 to 2, or a salt thereof.

### EMBODIMENT 4

In the formula (I),

a benzoylurea compound wherein
X and Y independently represent a fluorine atom and a chlorine atom, respectively,
R¹ represents a hydrogen atom, methyl, ethyl, 2,2,2-trifluoroethyl, 2-propenyl, 2-propynyl, benzyl, methoxymethyl, 2-methoxyethyl, 2-(methylthio)ethyl, 2-(methylsulfinyl)ethyl, 2-(methylsulfonyl)ethyl, methoxycarbonyl, benzyloxycarbonyl, dimethylcarbamoyl, acetyl, methanesulfonyl, benzenesulfonyl, cyclopropyl, cyclopropylmethyl, dimethylamino, methoxy, morpholino, 4-tetrahydropyranyl, methoxycarbonylmethyl, 2-tetrahydrofurylmethyl, 2-furylmethyl, 2-pyridylmethyl, 3-pyridylmethyl, (2-chlorothiazol-5-yl)methyl, 2-methoxybenzyl, 3-methoxybenzyl, 4-methoxybenzyl, ethoxymethyl, 2-chloroethoxymethyl, benzyloxymethyl, (2-methoxyethoxy)methyl, or 2-morpholinoethyl,
R² represents methyl or ethyl,
R³ represents fluorine atom, chlorine atom, trifluoromethyl, or methyl,
R⁴ represents tert-butoxycarbonyl, trifluoromethylthio, trifluoromethysulfinyl, trifluoromethylsulfonyl, difluoromethylthio, trichloromethylthio, methylthio, ethylthio, 1,1,2,2-tetrafluoroethylthio, 1,1,2,2-tetrafluoroethylsulfinyl, 1,1,2,2-tetrafluoroethylsulfonyl, 2,2,2-trifluoroethylthio, 1,1,2,2,2-pentafluoroethylthio, 1,1,2,2,3,3,3-heptafluoro-1-propylthio, 1,1,2,3,3,3-hexafluoro-1-propylthio, 2-propenylthio, 2-propynylthio, or 1,1,2-trifluoro-2-trifluoromethoxyethylthio, and
m represents an integer of 0 to 2, or a salt thereof.

### EMBODIMENT 5

Any one of compounds represented by the followings:
3-(2,6-difluorobenzoyl)-1-[2-fluoro-4-(trifluoromethylthio)phenyl]-1-methylurea,
3-(2,6-difluorobenzoyl)-1-[2-fluoro-4-(1,1,2,2-tetrafluoroethylthio)phenyl]-1-methylurea,
3-(2-chloro-6-fluorobenzoyl)-1-[2-fluoro-4-(trifluoromethylthio)phenyl]-1-methylurea,
3-(2,6-difluorobenzoyl)-1-ethyl-1-[2-fluoro-4-(trifluoromethylthio)phenyl]urea,
3-(2,6-difluorobenzoyl)-1-(2-fluoro-4-methylthiophenyl)-1-methylurea,
3-(2-chloro-6-fluorobenzoyl)-1-(2-fluoro-4-methylthiophenyl)-1-methylurea,
3-(2,6-difluorobenzoyl)-1-[4-(trifluoromethylthio)phenyl]-1-methylurea,
3-(2,6-difluorobenzoyl)-1-(2-fluoro-4-ethylthiophenyl)]-1-methylurea,
3-(2-chloro-6-fluorobenzoyl)-1-(2-fluoro-4-ethylthiophenyl)-1-methylurea,
1-(2,6-difluorobenzoyl)-3-[2-fluoro-4-(trifluoromethylthio)phenyl]-1,3-dimethylurea,
1-(2,6-difluorobenzoyl)-3-[2-fluoro-4-(trifluoromethylthio)phenyl]-1-(methoxymethyl)-3-methylurea,
1-(2,6-difluorobenzoyl)-3-[2-fluoro-4-(1,1,2,2-tetrafluoroethylthio)phenyl]-1,3-dimethylurea,
1-(2-chloro-6-fluorobenzoyl)-3-[2-fluoro-4-(trifluoromethylthio)phenyl]-1,3-dimethylurea,
1-(2,6-difluorobenzoyl)-3-(2-fluoro-4-methylthiophenyl)-1,3-dimethylurea,
1-(2-chloro-6-fluorobenzoyl)-3-(2-fluoro-4-methylthiophenyl)-1,3-dimethylurea,
1-(2,6-difluorobenzoyl)-3-(2-fluoro-4-ethylthiophenyl)-1,3-dimethylurea,
1-(2-chloro-6-fluorobenzoyl)-3-(2-fluoro-4-ethylthiophenyl)-1,3-dimethylurea,
3-(2,6-difluorobenzoyl)-1-[2-fluoro-4-(trifluoromethylsulfinyl)phenyl)-1-methylurea,
1-(2,6-difluorobenzoyl)-3-[2-fluoro-4-(trifluoromethylsulfinyl)phenyl]-1,3-dimethylurea,
3-(2,6-difluorobenzoyl)-1-[2-fluoro-4-(trifluoromethylsulfonyl)phenyl]-1-methylurea,
1-(2,6-difluorobenzoyl)-3-[2-fluoro-4-(trifluoromethylsulfonyl)phenyl]-1,3-dimethylurea,
1-(2,6-difluorobenzoyl)-3-[2-fluoro-4-(1,1,2,2,3,3,3-heptafluoro-1-propylthio)phenyl]-1,3-dimethylurea,
3-(2-chloro-6-fluorobenzoyl)-1-[2-fluoro-4-(1,1,2,2-tetrafluoroethylthio)phenyl]-1-methylurea,
3-(2,6-difluorobenzoyl)-1-[2-fluoro-4-(1,1,2,2-tetrafluoroethanesulfinyl)phenyl]-1-methylurea,
3-(2,6-difluorobenzoyl)-1-[2-fluoro-4-(1,1,2,2-tetrafluoroethanesulfonyl)phenyl]-1-methylurea,
3-(2,6-difluorobenzoyl)-1-[2-fluoro-4-(2,2,2-trifluoroethylthio)phenyl]-1-methylurea,
1-[2-chloro-4-(trifluoromethylthio)phenyl]-3-(2,6-difluorobenzoyl)-1-methylurea,
3-(2,6-difluorobenzoyl)-1-[2-fluoro-4-(1,1,2,2,3,3,3-heptafluoro-1-propylthio)phenyl]-1-methylurea,
3-(2-chloro-6-fluorobenzoyl)-1-[2-fluoro-4-(1,1,2,2,3,3,3-heptafluoro-1-propylthio)phenyl]-1-methylurea,
3-(2,6-difluorobenzoyl)-1-[2-fluoro-4-(1,1,2,2,2-pentafluoroethylthio)phenyl]-1-methylurea,
3-(2,6-difluorobenzoyl)-1-[2-fluoro-4-(1,1,2,3,3,3-hexafluoro-1-propylthio)phenyl]-1-methylurea,
3-(2,6-difluorobenzoyl)-1-[2,3-dimethyl-4-(trifluoromethylthio)phenyl]-1-methylurea,
3-(2,6-difluorobenzoyl)-1-[2,3-dimethyl-4-(1,1,2,2,2-pentafluoroethylthio)phenyl]-1-methylurea,
1-[2-chloro-4-(difluoromethylthio)phenyl]-3-(2,6-difluorobenzoyl)-1-methylurea,
3-(2,6-difluorobenzoyl)-1-[4-(difluoromethylthio)-2-methylphenyl]-1-methylurea,
3-(2,6-difluorobenzoyl)-1-methyl-1-[2-methyl-4-(trifluoromethylthio)phenyl]urea,
3-(2,6-difluorobenzoyl)-1-[2-fluoro-4-(t-butoxycarbonyl)phenyl]-1-methylurea,
1-(2-chloro-6-fluorobenzoyl)-3-[2-fluoro-4-(1,1,2,2-tetrafluoroethylthio)phenyl]-1,3-dimethylurea,
1-(2,6-difluorobenzoyl)-3-[2-fluoro-4-(1,1,2,2-tetrafluoroethanesulfinyl)phenyl]-1,3-dimethylurea,
1-(2,6-difluorobenzoyl)-3-[2-fluoro-4-(1,1,2,2-tetrafluoroethanesulfonyl)phenyl]-1,3-dimethylurea,
1-(2,6-difluorobenzoyl)-3-ethyl-3-[2-fluoro-4-(trifluoromethylthio)phenyl]-1-methylurea,
1-(2,6-difluorobenzoyl)-1,3-dimethyl-3-[2-methyl-4-(trifluoromethylthio)phenyl]urea,
1-(2,6-difluorobenzoyl)-3-[2-fluoro-4-(t-butoxycarbonyl)phenyl]-1,3-dimethylurea,
1-(2,6-difluorobenzoyl)-3-[2-fluoro-4-(2,2,2-trifluoroethylthio)phenyl]-1,3-dimethylurea,
1-(2-chloro-6-fluorobenzoyl)-3-[2-fluoro-4-(1,1,2,2,3,3,3-heptafluoro-1-propylthio)phenyl]-1,3-dimethylurea,
1-(2,6-difluorobenzoyl)-3-[2-fluoro-4-(1,1,2,2,2-pentafluoroethylthio)phenyl]-1,3-dimethylurea,
1-(2,6-difluorobenzoyl)-3-[2-fluoro-4-(1,1,2,3,3,3-hexafluoro-1-propylthio)phenyl]-1,3-dimethylurea,
1-(2,6-difluorobenzoyl)-3-[2,3-dimethyl-4-(trifluoromethylthio)phenyl]-1,3-dimethylurea,
1-(2,6-difluorobenzoyl)-3-[2,3-dimethyl-4-(1,1,2,2,2-pentafluoroethylthio)phenyl]-1,3-dimethylurea,
1-[2-chloro-4-(difluoromethylthio)phenyl]-3-(2,6-difluorobenzoyl)-1,3-dimethylurea,
1-(2,6-difluorobenzoyl)-3-[4-(difluoromethylthio)-2-methylphenyl]-1,3-dimethylurea,
1-acetyl-1-(2,6-difluorobenzoyl)-3-[2-fluoro-4-(trifluoromethylthio)phenyl]-3-methylurea,
1-(2,6-difluorobenzoyl)-3-[2-fluoro-4-(trifluoromethylthio)phenyl]-1-methoxycarbonyl-3-methylurea,
1-(2,6-difluorobenzoyl)-3-[2-fluoro-4-(trifluoromethylthio)phenyl]-1-methanesulfonyl-3-methylurea,
1-(2,6-difluorobenzoyl)-1-(N,N-dimethylcarbamoyl)-3-[2-fluoro-4-(trifluoromethylthio)phenyl]-3-methylurea,
1-(2,6-difluorobenzoyl)-1-ethyl-3-[2-fluoro-4-(trifluoromethylthio)phenyl]-3-methylurea,
3-(2,6-difluorobenzoyl)-1-[4-(difluoromethylthio)-2-fluorophenyl]-1-methylurea,
1-(2,6-difluorobenzoyl)-3-[4-(difluoromethylthio)-2-fluorophenyl]-1,3-dimethylurea,
1-allyl-1-(2,6-difluorobenzoyl)-3-[2-fluoro-4-(trifluoromethylthio)phenyl]-3-methylurea,
1-(2,6-difluorobenzoyl)-3-[2-fluoro-4-(trifluoromethylthio)phenyl]-3-methyl-1-propargylurea,
1-benzyl-1-(2,6-difluorobenzoyl)-3-[2-fluoro-4-(trifluoromethylthio)phenyl]-3-methylurea,
1-(2,6-difluorobenzoyl)-3-[2-fluoro-4-(trifluoromethylthio)phenyl]-3-methyl-1-(2-phenoxyethyl)urea,
1-(2,6-difluorobenzoyl)-3-[2-fluoro-4-(trifluoromethylthio)phenyl]-3-methyl-1-(2-tetrahydrofurylmethyl)urea,
1-(2,6-difluorobenzoyl)-3-[2-fluoro-4-(trifluoromethylthio)phenyl]-1-(2-furylmethyl)-3-methylurea,
1-(2,6-difluorobenzoyl)-3-[2-fluoro-4-(trifluoromethylthio)phenyl]-1-(2-methoxyethyl)-3-methylurea,
1-cyclopropyl-1-(2,6-difluorobenzoyl)-3-[2-fluoro-4-(trifluoromethylthio)phenyl]-3-methylurea,
1-(2,6-difluorobenzoyl)-3-[2-fluoro-4-(trifluoromethylthio)phenyl]-3-methyl-1-(2,2,2-trifluoroethyl)urea,
1-cyclopropylmethyl-1-(2,6-difluorobenzoyl)-3-[2-fluoro-4-(trifluoromethylthio)phenyl]-3-methylurea,
1-(2,6-difluorobenzoyl)-3-[2-fluoro-4-(trifluoromethylthio)phenyl]-3-methyl-3-(2-methylthioethyl)urea,
1-(2,6-difluorobenzoyl)-3-[2-fluoro-4-(trifluoromethylthio)phenyl]-3-methyl-3-(2-methylsulfinylethyl)urea,
1-(2,6-difluorobenzoyl)-3-[2-fluoro-4-(trifluoromethylthio)phenyl]-3-methyl-3-(2-methylsulfonylethyl)urea,
1-(2,6-difluorobenzoyl)-3-[2-fluoro-4-(trifluoromethylthio)phenyl]-3-methyl-1-(2-pyridylmethyl)urea,
1-(2,6-difluorobenzoyl)-3-[2-fluoro-4-(trifluoromethylthio)phenyl]-3-methyl-1-(3-pyridylmethyl)urea,
1-[(2-chlorothiazol-5-yl)methyl]-1-(2,6-difluorobenzoyl)-3-[2-fluoro-4-(trifluoromethylthio)phenyl]-3-methylurea,
1-(2,6-difluorobenzoyl)-3-[2-fluoro-4-(trifluoromethylthio)phenyl]-3-methyl-1-morpholinourea,
1-(2,6-difluorobenzoyl)-3-[2-fluoro-4-(trifluoromethylthio)phenyl]-3-methyl-1-(2-morpholinoethyl)urea,
1-(2,6-difluorobenzoyl)-3-[2-fluoro-4-(trifluoromethylthio)phenyl]-1-methoxycarbonylmethyl-3-methylurea,
1-[4-(t-butoxycarbonyl)phenyl]-3-(2,6-difluorobenzoyl)-1-methylurea,
3-(2,6-difluorobenzoyl)-1-[2-fluoro-4-(2-propenylthio)phenyl]-1-methylurea,
3-(2,6-difluorobenzoyl)-1-[2-fluoro-4-(2-propynylthio)phenyl]-1-methylurea,
1-[3,5-dichloro-4-(1,1,2,2-tetrafluoroethylthio)phenyl]-3-(2,6-difluorobenzoyl)-1-methylurea,
3-(2,6-difluorobenzoyl)-1-methyl-1-[4-(1,1,2,2-tetrafluoroethylthio)phenyl]urea,
3-(2,6-dichlorobenzoyl)-1-[2-fluoro-4-(trifluoromethylthio)phenyl]-1-methylurea,
1-(2,6-dichlorobenzoyl)-3-[2-fluoro-4-(trifluoromethylthio)phenyl]-1,3-dimethylurea,
1-benzyloxycarbonyl-1-(2,6-difluorobenzoyl)-3-[2-fluoro-4-(trifluoromethylthio)phenyl]-3-methylurea,
1-(2,6-difluorobenzoyl)-3-[2-fluoro-4-(trifluoromethylthio)phenyl]-3-methyl-1-phenylsulfonylurea,
3-(2,6-difluorobenzoyl)-1-[2,5-difluoro-4-(trifluoromethylthio)phenyl]-1-methylurea,
1-(2,6-difluorobenzoyl)-3-[2,5-difluoro-4-(trifluoromethylthio)phenyl]-1,3-dimethylurea,
3-(2,6-difluorobenzoyl)-1-[2,6-difluoro-4-(trifluoromethylthio)phenyl]-1-methylurea,
1-(2,6-difluorobenzoyl)-3-[2,6-difluoro-4-(trifluoromethylthio)phenyl]-1,3-dimethylurea,
1-[2-chloro-4-(trifluoromethylthio)phenyl]-3-(2,6-difluorobenzoyl)-1,3-dimethylurea,
3-(2,6-difluorobenzoyl)-1-methyl-1-[2-methyl-4-(pentafluoroethylthio)phenyl]urea,
1-(2,6-difluorobenzoyl)-1,3-dimethyl-3-[2-methyl-4-(pentafluoroethylthio)phenyl]urea,
1-[2-chloro-4-(pentafluoroethylthio)phenyl]-3-(2,6-difluorobenzoyl)-1-methylurea,
1-[2-chloro-4-(pentafluoroethylthio)phenyl]-3-(2,6-difluorobenzoyl)-1,3-dimethylurea,
3-(2,6-difluorobenzoyl)-1-[2-fluoro-4-(1,1,2-trifluoro-2-trifluoromethoxyethylthio)phenyl]-1-methylurea,
1-(2,6-difluorobenzoyl)-3-[2-fluoro-4-(1,1,2-trifluoro-2-trifluoromethoxyethylthio)phenyl]-1,3-dimethylurea,
3-(2,6-difluorobenzoyl)-1-[4-(difluoromethylthio)-2,3-dimethylphenyl]-1-methylurea,
1-(2,6-difluorobenzoyl)-3-[4-(difluoromethylthio)-2,3-dimethylphenyl]-1,3-dimethylurea,
1-dimethylamino-1-(2,6-difluorobenzoyl)-3-[2-fluoro-4-(trifluoromethylthio)phenyl]-3-methylurea, or
1-(2,6-difluorobenzoyl)-3-[2-fluoro-4-(trifluoromethylthio)phenyl]-1-methoxy-3-methylurea.

### EMBODIMENT 6

A benzoylurea compound (I-a) represented by Formula (I-a)

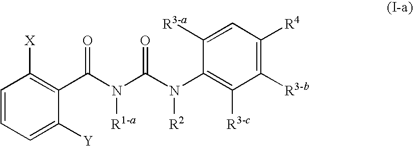

wherein, X and Y independently represent fluorine atom or chlorine atom, respectively,
R^{1-a} represents hydrogen atom or a lower alkyl group,
R² represents a lower alkyl group, and

(1) when R^{3-a} and R^{3-b} represent a halogen atom, R^{3-c} represents a hydrogen atom,

(2) when R^{3-a} and R^{3-c} represent a halogen atom, R^{3-b} represents a hydrogen atom, or,

(3) when R^{3-a} represents a halogen atom or a lower alkyl group, R^{3-b} and R^{3-c} represent a hydrogen atom, and

R⁴ represents a group represented by S(O)ₙR⁵, wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, or a salt thereof.

### EMBODIMENT 7

A benzoylurea compound wherein in the formula (I-a), X and Y independently represent fluorine atom or chlorine atom, respectively,

R^{1-a} represents a hydrogen atom, or a lower alkyl group,
R² represents a lower alkyl group, and

(1) when R^{3-a} and R^{3-b} represent a halogen atom, R^{3-c} represents a hydrogen atom,

(2) when R^{3-a} and R^{3-c} represent a halogen atom, R^{3-b} represents a hydrogen atom, and

R⁴ represents a group represented by S(O)ₙR⁵ wherein R⁵ represents a lower alkyl group optionally substituted with one or more of halogen atoms, and n represents an integer of 0 to 2, or a salt thereof.

### EMBODIMENT 8

A benzoylurea compound wherein in the formula (I-a), X and Y independently represent fluorine atom or chlorine atom, respectively,

R^{1-a} represents a hydrogen atom or a lower alkyl group,
R² represents a lower alkyl group,
R^{3-a} represents a halogen atom or a lower alkyl group,
R^{3-b} and R^{3-c} represent a hydrogen atom, and
R⁴ represents a group represented by S(O)ₙR⁵ wherein R⁵ represents a lower alkyl group optionally substituted with one or more of halogen atoms, and n represents an integer of 0 to 2, or a salt thereof.

### EMBODIMENT 9

A benzoylurea compound wherein in the formula (I), X and Y represent a fluorine atom or a chlorine atom, respectively,

R¹ represents a hydrogen atom, a C1-C6 alkyl group optionally substituted with one or more of halogen atoms, a C2-C6 alkenyl group optionally substituted with one or more of halogen atoms, a C2-C6 alkynyl group, a C6-C14 aryl group, a C7-C11 aralkyl group, a C2-C6 alkoxyalkyl group, a C7-C14 aryloxyalkyl group, a C3-C6 N,N-di(alkyl)aminoalkyl group, a C2-C6 alkylthioalkyl group, a C2-C6 alkylsulfinylalkyl group, a C2-C6 alkylsulfonylalkyl group, a C3-C9 alkoxyalkoxyalkyl group, a C2-C6 alkoxycarbonyl group, a C8-C12 aralkyloxycarbonyl group, a N,N-di(C1-C6 alkyl)carbamoyl group, a C2-C6 alkylcarbonyl group optionally substituted with one or more of halogen atoms, a formyl group, a C1-C5 alkylsulfonyl group optionally substituted with one or more of halogen atoms or a C6-C10 arylsulfonyl group,
R² represents a C1-C2 alkyl group,
R³ represents a halogen atom or a C1-C4 alkyl group optionally substituted with one or more of halogen atoms,
R⁹ represents a C2-C6 alkoxycarbonyl group or a group represented by S(O)ₙR⁵,
R⁵ represents a C1-C4 alkyl group optionally substituted with one or more of halogen atoms, a C2-C4 alkenyl group optionally substituted with one or more of halogen atoms, a C2-C4 alkynyl group, or a C2-C4 alkoxyalkyl group optionally substituted with one or more of halogen atoms,
m represents any one of an integer of 0 to 4, and
n represents any one of an integer of 0 to 2, or a salt thereof.

### EMBODIMENT 10

A benzoylurea compound wherein in the formula (I), X and Y represent a fluorine atom or a chlorine atom, respectively,

R¹ represents a hydrogen atom or a C1-C6 alkyl group optionally substituted with one or more of halogen atoms or a C2-C6 alkoxyalkyl group,
R² represents a C1-C2 alkyl group,
R³ represents a halogen atom or a C1-C4 alkyl group optionally substituted with one or more of halogen atoms,
R⁴ represents a C2-C6 alkoxycarbonyl group or a group represented by S(O)ₙR⁵,
R⁵ represents a C1-C4 alkyl group optionally substituted with one or more of halogen atoms, a C2-C4 alkenyl group optionally substituted with one or more of halogen atoms, a C2-C4 alkynyl group, or a C2-C4 alkoxyalkyl group optionally substituted with one or more of halogen atoms,
m represents any one of an integer of 0 to 4, and
n represents any one of an integer of 0 to 2, or a salt thereof.

### EMBODIMENT 11

A benzoylurea compound wherein in the formula (I), X and Y represent a fluorine atom or a chlorine atom, respectively,

R¹ represents a C1-C6 alkyl group optionally substituted with one or more of halogen atoms or a C2-C6 alkoxyalkyl group,
R² represents a C1-C2 alkyl group,
R³ represents a halogen atom or a C1-C4 alkyl group optionally substituted with one or more of halogen atoms,
R⁴ represents a C2-C6 alkoxycarbonyl group or a group represented by S(O)ₙR⁵,
R⁵ represents a C1-C4 alkyl group optionally substituted with one or more of halogen atoms, a C2-C4 alkenyl group optionally substituted with one or more of halogen atoms, a C2-C4 alkynyl group, or a C2-C4 alkoxyalkyl group optionally substituted with one or more of halogen atoms,
m represents any one of an integer of 0 to 4, and
n represents any one of an integer of 0 to 2, or a salt thereof.

### EMBODIMENT 12

A benzoylurea compound wherein in the formula (I), X and Y represent a fluorine atom or a chlorine atom, respectively,

R¹ represents a hydrogen atom, a C1-C6 alkyl group optionally substituted with one or more of halogen atoms, a C2-C6 alkenyl group optionally substituted with one or more of halogen atoms, a C2-C6 alkynyl group, a C6-C14 aryl group, a C7-C11 aralkyl group, a C2-C6 alkoxyalkyl group, a C7-C14 aryloxyalkyl group, a C3-C6 N,N-di(alkyl)aminoalkyl group, a C2-C6 alkylthioalkyl group, a C2-C6 alkylsulfinylalkyl group, a C2-C6 alkylsulfonylalkyl group, C3-C9 alkoxyalkoxyalkyl group, a C2-C6 alkoxycarbonyl group, C8-12 aralkyloxycarbonyl group, or N,N-di(C1-C6 alkyl)carbamoyl group, a C2-C6 alkylcarbonyl group optionally substituted with one or more of halogen atoms, a formyl group, a C1-C5 alkylsulfonyl group optionally substituted with one or more of halogen atoms or a C6-C10 arylsulfonyl group,
R² represents a C1-C2 alkyl group,
R³ represents a halogen atom,
R⁴ represents a C2-C6 alkoxycarbonyl group or a group represented by S(O)ₙR⁵,
R⁵ represents a C1-C4 alkyl group optionally substituted with one or more of halogen atoms, a C2-C4 alkenyl group optionally substituted with one or more of halogen atoms, a C2-C4 alkynyl group, or a C2-C4 alkoxyalkyl group optionally substituted with one or more of halogen atoms,
m represents an integer of 1 or 2, and in the case where m represents 2, R³ may be the same or different, and
n represents any one of an integer of 0 to 2, or a salt thereof.

### EMBODIMENT 13

A benzoylurea compound wherein in the formula (I), X and Y represent a fluorine atom or a chlorine atom, respectively,

R¹ represents a hydrogen atom, a C1-C6 alkyl group optionally substituted with one or more of halogen atoms, a C2-C6 alkenyl group optionally substituted with one or more of halogen atoms, C2-C6 alkynyl group, a C6-C14 aryl group, a C7-C11 aralkyl group, a C2-C6 alkoxyalkyl group, a C7-C14 aryloxyalkyl group, a C3-C6 N,N-di(alkyl)aminoalkyl group, a C2-C6 alkylthioalkyl group, a C2-C6 alkylsulfinylalkyl group, C2-C6 alkylsulfonylalkyl group, a C3-C9 alkoxyalkoxyalkyl group, a C2-C6 alkoxycarbonyl group, a C8-C12 aralkyloxycarbonyl group, a N,N-di(C1-C6 alkyl)carbamoyl group, a C2-C6 alkylcarbonyl group optionally substituted with one or more of halogen atoms, a formyl group, a C1-C5 alkylsulfonyl group optionally substituted with one or more of halogen atoms or a C6-C10 arylsulfonyl group,
R² represents a C1-C2 alkyl group,
R³ represents a halogen atom,
R⁴ represents a C2-C6 alkoxycarbonyl group or a group represented by S(O)ₙR⁵,
R⁵ represents a C1-C4 alkyl group optionally substituted with one or more of halogen atoms, a C2-C4 alkenyl group optionally substituted with one or more of halogen atoms, a C2-C4 alkynyl group, or a C2-C4 alkoxyalkyl group optionally substituted with one or more of halogen atoms,
m represents an integer of 1, and
n represents any one of an integer of 0 to 2, or a salt thereof.

### EMBODIMENT 14

A benzoylurea compound wherein in the formula (I), X and Y represent a fluorine atom or a chlorine atom, respectively,

R¹ represents a hydrogen atom, a C1-C6 alkyl group optionally substituted with one or more of halogen atoms or a C2-C6 alkoxyalkyl group,
R² represents a C1-C2 alkyl group,
R³ represents a halogen atom,
R⁴ represents a C2-C6 alkoxycarbonyl group or a group represented by S(O)ₙR⁵,
R⁵ represents a C1-C4 alkyl group optionally substituted with one or more of halogen atoms, a C2-C4 alkenyl group optionally substituted with one or more of halogen atoms, a C2-C4 alkynyl group, or a C2-C4 alkoxyalkyl group optionally substituted with one or more of halogen atoms,
m represents an integer of 1, and
n represents any one of an integer of 0 to 2, or a salt thereof.

### EMBODIMENT 15

A benzoylurea compound wherein in the formula (I), X and Y represent a fluorine atom or a chlorine atom, respectively,

R¹ represents a hydrogen atom, a C1-C6 alkyl group optionally substituted with one or more of halogen atoms, a C2-C6 alkenyl group optionally substituted with one or more of halogen atoms, C2-C6 alkynyl group, a C6-C14 aryl group, a C7-C11 aralkyl group, a C2-C6 alkoxyalkyl group, a C7-C14 aryloxyalkyl group, a C3-C6 N,N-di(alkyl)aminoalkyl group, a C2-C6 alkylthioalkyl group, a C2-C6 alkylsulfinylalkyl group, a C2-C6 alkylsulfonylalkyl group, a C3-C9 alkoxyalkoxyalkyl group, a C2-C6 alkoxycarbonyl group, a C8-C12 aralkyloxycarbonyl group, a N,N-di(C1-C6 alkyl)carbamoyl group, a C2-C6 alkylcarbonyl group optionally substituted with one or more of halogen atoms, a formyl group, a C1-C5 alkylsulfonyl group optionally substituted with one or more of halogen atoms or a C6-C10 arylsulfonyl group,
R² represents a C1-C2 alkyl group,
R³ represents a halogen atom, or a C1-C4 alkyl group optionally substituted with one or more of halogen atoms,
R⁴ represents a group represented by S(O)ₙR⁵,
R⁵ represents a C1-C4 alkyl group optionally substituted with one or more of halogen atoms, a C2-C4 alkenyl group optionally substituted with one or more of halogen atoms, a C2-C4 alkynyl group, or a C2-C4 alkoxyalkyl group optionally substituted with one or more of halogen atoms,
m represents an integer of 0 to 4, and
n represents any one of an integer of 0 to 2, or a salt thereof.

### EMBODIMENT 16

A benzoylurea compound wherein in the formula (I), X and Y represent a fluorine atom or a chlorine atom, respectively,

R¹ represents a hydrogen atom, a C1-C6 alkyl group optionally substituted with one or more of halogen atoms, a C2-C6 alkenyl group optionally substituted with one or more of halogen atoms, C2-C6 alkynyl group, a C6-C14 aryl group, a C7-C11 aralkyl group, a C2-C6 alkoxyalkyl group, a C7-C14 aryloxyalkyl group, a C3-C6 N,N-di(alkyl)aminoalkyl group, a C2-C6 alkylthioalkyl group, a C2-C6 alkylsulfinylalkyl group, a C2-C6 alkylsulfonylalkyl group, a C3-C9 alkoxyalkoxyalkyl group, a C2-C6 alkoxycarbonyl group, a C8-C12 aralkyloxycarbonyl group, a N,N-di(C1-C6 alkyl)carbamoyl group, a C2-C6 alkylcarbonyl group optionally substituted with one or more of halogen atoms, a formyl group, a C1-C5 alkylsulfonyl group optionally substituted with one or more of halogen atoms or a C6-C10 arylsulfonyl group,
R² represents a C1-C2 alkyl group,
R³ represents a halogen atom or a C1-C4 alkyl group optionally substituted with one or more of halogen atoms,
R⁴ represents a group represented by S(O)ₙR⁵,
R⁵ represents a C1-C4 alkyl group optionally substituted with one or more of halogen atoms,
m represents an integer of 0 to 4, and
n represents any one of an integer of 0 to 2, or a salt thereof.

### EMBODIMENT 17

A benzoylurea compound wherein in the formula (I), X and Y represent a fluorine atom or a chlorine atom, respectively,

R¹ represents a hydrogen atom or a C1-C6 alkyl group optionally substituted with one or more of halogen atoms, a C2-C6 alkenyl group optionally substituted with one or more of halogen atoms, a C2-C6 alkynyl group, a C6-C14 aryl group, a C7-C11 aralkyl group, a C2-C6 alkoxyalkyl group, a C7-C14 aryloxyalkyl group, a C3-C6 N,N-di(alkyl)aminoalkyl group, a C2-C6 alkylthioalkyl group, a C2-C6 alkylsulfinylalkyl group, a C2-C6 alkylsulfonylalkyl group, a C3-C9 alkoxyalkoxyalkyl group, a C2-C6 alkoxycarbonyl group, a C8-C12 aralkyloxycarbonyl group, a N,N-di(C1-C6 alkyl)carbamoyl group, a C2-C6 alkylcarbonyl group optionally substituted with one or more of halogen atoms, a formyl group, a C1-C5 alkylsulfonyl group optionally substituted with one or more of halogen atoms or a C6-C10 arylsulfonyl group,
R² represents a C1-C2 alkyl group,
R³ represents a halogen atom,
R⁴ represents a group represented by S(O)ₙR⁵,
R⁵ represents a C1-C4 alkyl group optionally substituted with one or more of halogen atoms, a C2-C4 alkenyl group optionally substituted with one or more of halogen atoms, a C2-C4 alkynyl group, or a C2-C4 alkoxyalkyl group optionally substituted with one or more of halogen atoms,
m represents an integer of 1, and
n represents any one of an integer of 0 to 2, or a salt thereof.

### EMBODIMENT 18

A benzoylurea compound wherein in the formula (I), X and Y represent a fluorine atom or a chlorine atom, respectively,

R¹ represents a hydrogen atom, a C1-C6 alkyl group optionally substituted with one or more of halogen atoms, a C2-C6 alkenyl group optionally substituted with one or more of halogen atoms, C2-C6 alkynyl group, a C6-C14 aryl group, a C7-C11 aralkyl group, a C2-C6 alkoxyalkyl group, a C7-C14 aryloxyalkyl group, a C3-C6 N,N-di(alkyl)aminoalkyl group, a C2-C6 alkylthioalkyl group, a C2-C6 alkylsulfinylalkyl group, a C2-C6 alkylsulfonylalkyl group, a C3-C9 alkoxyalkoxyalkyl group, a C2-C6 alkoxycarbonyl group, a C8-C12 aralkyloxycarbonyl group, a N,N-di(C1-C6 alkyl)carbamoyl group, a C2-C6 alkylcarbonyl group optionally substituted with one or more of halogen atoms, a formyl group, a C1-C5 alkylsulfonyl group optionally substituted with one or more of halogen atoms or a C6-C10 arylsulfonyl group,
R² represents a C1-C2 alkyl group,
R³ represents a halogen atom,
R⁴ represents a group represented by S(O)ₙR⁵,
R⁵ represents a C1-C4 alkyl group optionally substituted with one or more of halogen atoms,
m represents an integer of 1, and
n represents any one of an integer of 0 to 2, or a salt thereof.

### EMBODIMENT 19

A benzoylurea compound wherein in the formula (I), X and Y represent a fluorine atom or a chlorine atom, respectively,

R¹ represents a hydrogen atom, a C1-C6 alkyl group optionally substituted with one or more of halogen atoms or a C2-C6 alkoxyalkyl group,
R² represents a C1-C2 alkyl group,
R³ represents a halogen atom or a C1-C4 alkyl group optionally substituted with one or more of halogen atoms,
R⁴ represents a group represented by S(O)ₙR⁵,
R⁵ represents a C1-C4 alkyl group optionally substituted with one or more of halogen atoms,
m represents any one of integers of 0 to 4, and
n represents any one of integers of 0 to 2, or a salt thereof.

### EMBODIMENT 20

A benzoylurea compound wherein in the formula (I), X and Y represent a fluorine atom or a chlorine atom, respectively,

R¹ represents a C1-C6 alkyl group optionally substituted with one or more of halogen atoms or a C2-C6 alkoxyalkyl group,
R² represents a C1-C2 alkyl group,
R³ represents a halogen atom or a C1-C4 alkyl group optionally substituted with one or more of halogen atoms,
R⁴ represents a group represented by S(O)ₙR⁵,
R⁵ represents a C1-C4 alkyl group optionally substituted with one or more of halogen atoms,
m represents any one of integers of 0 to 4, and
n represents any one of integers of 0 to 2, or a salt thereof.

### EMBODIMENT 21

A benzoylurea compound wherein in the formula (I), X and Y represent a fluorine atom, respectively,

R¹ represents a C1-C6 alkyl group,
R² represents a C1-C2 alkyl group,
R³ represents a halogen atom,
R⁴ represents a group represented by SR⁵,
R⁵ represents a C1-C4 alkyl group optionally substituted with one or more of halogen atoms, and
m represents any one of integers of 0 to 2, or a salt thereof.

### EMBODIMENT 22

A benzoylurea compound wherein in the formula (I), X and Y independently represent a fluorine atom or a chlorine atom, respectively,

R¹ represents a hydrogen atom, a lower alkyl group optionally substituted with one or more of halogen atoms, a lower alkenyl group optionally substituted with one or more of halogen atoms, a lower alkynyl group, an aryl group, an aryl lower alkyl group, a lower alkoxy lower alkyl group, an aryloxy lower alkyl group, a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, a lower alkoxycarbonyl group, an aryl lower alkyloxycarbonyl group, a N,N-di(lower alkyl)carbamoyl group, a lower alkanoyl group optionally substituted with one or more of halogen atoms, a formyl group, a lower alkylsulfonyl group optionally substituted with one or more of halogen atoms, an arylsulfonyl group, an aryloxycarbonyl group, a lower cycloalkyl group, a lower cycloalkyl lower alkyl group, a di(lower alkyl)amino group, a lower alkoxy group, a 6-membered saturated heterocyclic group, or a group represented by —(CH₂)ₗ-A, wherein l represents an integer of 1 to 4 and A represents a di(lower alkoxy)methyl group, a lower alkoxycarbonyl group, or a 5- or 6-membered heterocyclic group optionally substituted with a halogen atom,
R² represents a lower alkyl group,
R³ represents a halogen atom, or a lower alkyl group optionally substituted with one or more of halogen atoms,
R⁴ represents a lower alkoxycarbonyl group, or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, and
m represents an integer of 0 to 4, or a salt thereof.

### EMBODIMENT 23

A benzoylurea compound wherein in the formula (I), X and Y independently represent a fluorine atom or a chlorine atom, respectively,

R¹ represents a hydrogen atom, a lower alkyl group optionally substituted with one or more of halogen atoms, a lower alkenyl group optionally substituted with one or more of halogen atoms, a lower alkynyl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a N,N-di(lower alkyl)amino lower alkyl group, a lower alkylthio lower alkyl group, a lower alkylsulfinyl lower alkyl group, a lower alkylsulfonyl lower alkyl group, a lower alkoxycarbonyl group, an aryl lower alkyloxycarbonyl group, a N,N-di(lower alkyl)carbamoyl group, a lower alkanoyl group optionally substituted with one or more of halogen atoms, a lower alkylsulfonyl group optionally substituted with one or more of halogen atoms, an arylsulfonyl group, an aryloxycarbonyl group, a lower cycloalkyl group, a lower cycloalkyl lower alkyl group, a di(lower alkyl)amino group, a lower alkoxy group, an aryl lower alkoxy lower alkyl group, a 6-membered saturated heterocyclic group, or a group represented by —(CH₂)ₗ-A wherein l represents an integer of 1 or 2 and A represents a di(lower alkoxy)methyl group, a lower alkoxycarbonyl group or a 5- or 6-membered heterocyclic group optionally substituted with a halogen atom,
R² represents a lower alkyl group,
R³ represents a halogen atom, or a lower alkyl group optionally substituted with one or more of halogen atoms,
R⁴ represents a lower alkoxycarbonyl group, or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, and
m represents an integer of 0 to 2, or a salt thereof.

### EMBODIMENT 24

A benzoylurea compound wherein in the formula (I), X and Y independently represent a fluorine atom or a chlorine atom, respectively,

R¹ represents a hydrogen atom, a lower alkyl group optionally substituted with one or more of halogen atoms, a lower alkenyl group, a lower alkynyl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a lower alkylthio lower alkyl group, a lower alkylsulfinyl lower alkyl group, a lower alkylsulfonyl lower alkyl group, a lower alkoxycarbonyl group, an aryl lower alkyloxycarbonyl group, a N,N-di(lower alkyl)carbamoyl group, a lower alkanoyl group, a lower alkylsulfonyl group, an arylsulfonyl group, a lower cycloalkyl group, a lower cycloalkyl lower alkyl group, a di(lower alkyl)amino group, a lower alkoxy group, a 6-membered saturated heterocyclic group, or a group represented by —(CH₂)ₗ-A wherein l represents an integer of 1 or 2, and A represents a lower alkoxycarbonyl group, or a 5- or 6-membered heterocyclic group optionally substituted with a halogen atom,
R² represents a lower alkyl group,
R³ represents a halogen atom, or a lower alkyl group,
R⁴ represents a lower alkoxycarbonyl group, or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group or a lower alkoxy lower alkyl group optionally substituted with one or more of halogen atoms, and n represents an integer of 0 to 2, and
m represents an integer of 0 to 2, or a salt thereof.

### EMBODIMENT 25

A benzoylurea compound wherein in the formula (I), X and Y independently represent a fluorine atom and a chlorine atom, respectively,

R¹ represents a hydrogen atom, methyl, ethyl, 2,2,2-trifluoroethyl, 2-propenyl, 2-propinyl, benzyl, methoxymethyl, 2-methoxyethyl, 2-phenoxyethyl, 2-(dimethylamino)ethyl, 2-(methylthio)ethyl, 2-(methylsulfinyl)ethyl, 2-(methylsulfonyl)ethyl, methoxycarbonyl, benzyloxycarbonyl, dimethylcarbamoyl, acetyl, methanesulfonyl, benzenesulfonyl, phenoxycarbonyl, cyclopropyl, cyclohexyl, cyclopropylmethyl, cyclohexylmethyl, dimethylamino, methoxy, morpholino, 2,2-dimethoxyethyl, methoxycarbonylmethyl, 2-tetrahydrofurylmethyl, 2-furylmethyl, (1,3-dioxolan-2-yl)methyl, 2-pyridylmethyl, 3-pyridylmethyl, (2-chlorothiazol-5-yl)methyl, 2-methoxybenzyl, 3-methoxybenzyl, 4-methoxybenzyl, ethoxymethyl, 2-chloroethoxymethyl, benzyloxymethyl, trifluoromethyl, or 2-morpholinoethyl,
R² represents methyl or ethyl,
R³ represents a fluorine atom, a chlorine atom or methyl,
R⁴ represents tert-butoxycarbonyl, trifluoromethylthio, trifluoromethysulfinyl, trifluoromethylsulfonyl, difluoromethylthio, methylthio, ethylthio, 1,1,2,2-tetrafluoroethylthio, 1,1,2,2-tetrafluoroethylsulfinyl, 1,1,2,2-tetrafluoroethylsulfonyl, 2,2,2-trifluoroethylthio, 1,1,2,2,2-pentafluoroethylthio, 1,1,2,2,3,3,3-heptafluoro-1-propylthio, 1,1,2,3,3,3-hexafluoro-1-propylthio, 2-propenylthio, 2-propenylsufinyl, 2-propenylsulfonyl, 3,3-dichloro-2-propenylthio, 2-propynylthio, 2-propynylsulfinyl, 2-propynylsulfonyl, or 1,1,2-trifluoro-2-trifluoromethoxyethylthio, and
m represents an integer of 0 to 2, or a salt thereof.

Hereinafter, a method for producing compound (I) will be explained.

The compound (I) can be produced according to the following (Production Process 1) to (Production Process 8).

### (Production Process 1)

Among compound (I), a method for producing a benzoylurea compound represented by formula (I-1) wherein R¹ and R² are the same lower alkyl group.

Among compound (I), the compound represented by the formula (I-1):

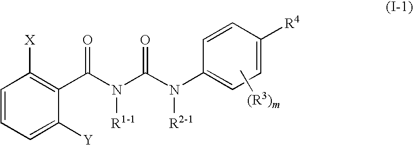

wherein, X and Y independently represent a fluorine atom or a chlorine atom, respectively,
R¹⁻¹ and R²⁻¹ represent the same lower alkyl group,
R³ represents a halogen atom, or a lower alkyl group optionally substituted with one or more of halogen atoms,
R⁴ represents a lower alkoxycarbonyl group, or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, and
m represents an integer of 0 to 4, can be produced by reacting a compound represented by formula (IV):

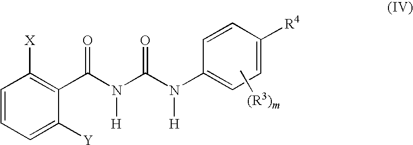

wherein, X, Y, R³, R⁴ and m are as defined above, with a compound represented by formula (V):

L¹-R¹⁻¹ (V)

wherein, R¹⁻¹ is as defined above, and L¹ represents a halogen atom, methanesulfonyloxy group, benzenesulfonyloxy group, toluenesulfonyloxy group, methoxysulfonyloxy group, or ethoxysulfonyloxy group.

The reaction is usually carried out in a solvent under the presence of a base.

Examples of the solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitrites such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water, and a mixture thereof.

Examples of the base used for the reaction include hydroxides of alkali metal or alkali earth metal such as sodium hydroxide, potassium hydroxide, calcium hydroxide and the like, hydrides of alkali metal or alkali earth metal such as sodium hydride, potassium hydride, calcium hydride and the like, carbonates of alkali metal or alkali earth metal such as sodium carbonate, potassium carbonate and the like, alcoholates of alkali metal such as sodium ethylate, sodium methylate and the like, an organic lithium reagents such as n-butyl lithium, lithium diisopropylamide and the like, and organic bases such as triethylamine, pyridine, 1,8-diazabicyclo[5.4.0]undec-7-ene and the like.

When a reagent which is liquid under the reaction condition is used, the excess amount of each reagent can be used in terms of the amount of the reagents used for the reaction, but usually, the compound represented by the formula (V) is used with a rate of 2 to 4 mole and the base is used with a rate of 2 to 4 mole relative to one mole of the compound represented by the formula (IV).

The reaction temperature of the reaction is usually in a range of −78 to 150° C., and the reaction time is usually in a range of 0.1 to 100 hours.

After completion of the reaction, the compound represented by formula (I-1) can be isolated by subjecting the reaction mixture to post-treatment operations such as adding the reaction mixture into water, extracting with an organic solvent, drying the organic layer, concentrating the extract and the like. The isolated compound represented by formula (I-1) can be further purified by recrystallization, column chromatography and the like.

### (Production Process 2)

Compounds (I) can be produced by reacting a compound represented by formula (VI):

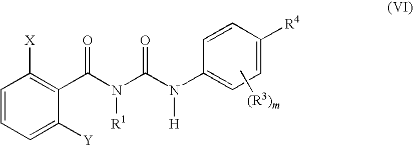

wherein, X, Y, R³, R⁴ and m are as defined above,
R¹ represents a lower alkyl group optionally substituted with one or more of halogen atoms, a lower alkenyl group optionally substituted with one or more of halogen atoms, a lower alkynyl group, an aryl group, an aryl lower alkyl group optionally substituted with one or more of alkoxy groups, a lower alkoxy lower alkyl group optionally substituted with one or more of halogen atoms, an aryloxy lower alkyl group optionally substituted with one or more of halogen atoms, an aryl lower alkoxy lower alkyl group, a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, a lower alkoxycarbonyl group, an aryl lower alkyloxycarbonyl group, a N,N-di(lower alkyl)carbamoyl group, a lower alkanoyl group optionally substituted with one or more of halogen atoms, a formyl group, a lower alkylsulfonyl group optionally substituted with one or more of halogen atoms, an arylsulfonyl group, an aryloxycarbonyl group, a lower cycloalkyl group, a lower cycloalkyl lower alkyl group, a di(lower alkyl)amino group, a lower alkoxy group, a lower alkanoyloxy lower alkyl group, an aryl lower alkoxy lower alkyl group, a 6-membered saturated heterocyclic group, or a group represented by —(CH₂)ₗ-A wherein l represents an integer of 1 to 4, and A represents a di(lower alkoxy)methyl group, a lower alkoxycarbonyl group or a 5- or 6-membered heterocyclic group, with a compound represented by formula (VII):

L²-R² (VII)

wherein, R² represents a lower alkyl group, and
L² represents a halogen atom, methanesulfonyloxy group, benzenesulfonyloxy group, toluenesulfonyloxy group, methoxysulfonyloxy group, or ethoxysulfonyloxy group.

The reaction is usually carried out in a solvent under the presence of a base.

Examples of the solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitrites such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water and a mixture thereof.

Examples of the base used for the reaction include hydroxides of alkali metal or alkali earth metal such as sodium hydroxide, potassium hydroxide, calcium hydroxide and the like, hydrides of alkali metal or alkali earth metal such as sodium hydride, potassium hydride, calcium hydride and the like, carbonates of alkali metal or alkali earth metal such as sodium carbonate, potassium carbonate and the like, alcoholates of alkali metal such as sodium ethylate, sodium methylate and the like, organic lithium reagent such as n-butyl lithium, lithium diisopropylamide and the like, organic bases such as triethylamine, pyridine, 1,8-diazabicyclo[5.4.0]undec-7-ene and the like.

When a reagent which is liquid under the reaction condition is used, the excess amount of each reagent can be used in terms of the amount of the reagents used for the reaction, but usually, the compound represented by formula (VII) is used with a rate of 1 to 3 mole and the base is used with a rate of 1 to 3 mole relative to one mole of the compound represented by formula (VI).

The reaction temperature of the reaction is usually in a range of −78 to 150° C., and the reaction time is usually in a range of 0.1 to 100 hours.

After completion of the reaction, the compound (I) can be isolated by subjecting the reaction mixture to post-treatment operations such as adding the reaction mixture into water, extracting with an organic solvent, drying the organic layer, concentrating the extract and the like. The isolated compound (I) can be further purified by recrystallization, column chromatography and the like.

### (Production Process 3)

Among the compound (I), a compound represented by formula (I-2):

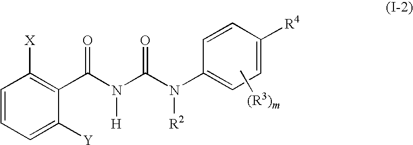

wherein, X, Y, R², R³, R⁴ and m are as defined above, can be produced by reacting a compound represented by formula (VIII):

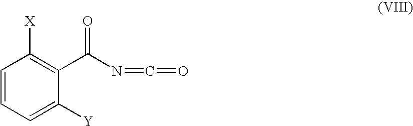

wherein, X and Y are as defined above, with a compound represented by formula (IX):

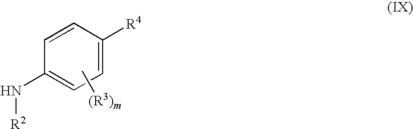

wherein, R², R³, R⁴ and m are as defined above.

The reaction is usually carried out in a solvent.

Examples of the solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitrites such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water and a mixture thereof.

The amount of the compound represented by formula (IX) used for the reaction is usually at a rate of 0.5 to 2 mole relative to one mole of a compound represented by formula (VIII).

The reaction temperature of the reaction is usually in a range of −78 to 150° C., and the reaction time is usually in a range of 0.1 to 100 hours.

After completion of the reaction, the compound represented by formula (I-2) can be isolated by subjecting the reaction mixture to post-treatment operations such as adding the reaction mixture into water, extracting with an organic solvent, drying the organic layer, concentrating the extract and the like. The isolated compound represented by formula (I-2) can be further purified by recrystallization, column chromatography and the like.

### (Production Process 4)

Among the compound (I), the compound represented by formula (I-3):

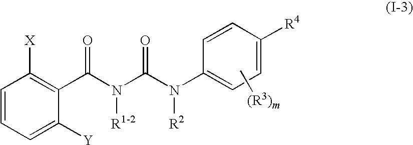

wherein, X, Y, R², R³, R⁴ and m are as defined above,
R¹⁻² represents a lower alkyl group optionally substituted with one or more of halogen atoms, a lower alkenyl group optionally substituted with one or more of halogen atoms, a lower alkynyl group, an aryl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, a lower cycloalkyl group, a lower cycloalkyl lower alkyl group, or a group represented by —(CH₂)ₗ-A wherein l represents an integer of 1 to 4, and A represents a 5- or 6-membered heterocyclic group optionally substituted with a halogen atom, can be produced by reacting a compound represented by formula (X):

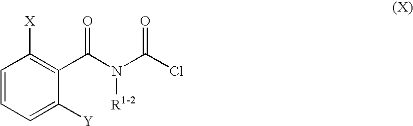

wherein, X, Y and R¹⁻² are as defined above, with a compound represented by formula (IX):

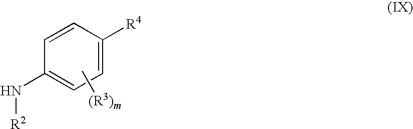

wherein, R², R³, R⁴ and m are as defined above.

The reaction is usually carried out in a solvent under the presence of a base.

Examples of the solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitrites such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water and a mixture thereof.

Examples of the base used for the reaction include hydroxides of alkali metal or alkali earth metal such as sodium hydroxide, potassium hydroxide, calcium hydroxide and the like, hydrides of alkali metal or alkali earth metal such as sodium hydride, potassium hydride, calcium hydride and the like, carbonates of alkali metal or alkali earth metal such as sodium carbonate, potassium carbonate and the like, alcoholates of alkali metal such as sodium ethylate, sodium methylate and the like, organic lithium reagents such as n-butyl lithium, lithium diisopropylamide and the like, organic bases such as triethylamine, pyridine, 1,8-diazabicyclo[5.4.0]undec-7-ene and the like.

When a reagent which is liquid under the reaction condition is used, the excess amount of each reagent can be used in terms of the amount of the reagents used for the reaction, but usually, the compound represented by formula (IX) is used with a rate of 1 to 4 moles and the base is used with a rate of 1 to 4 moles relative to one mole of the compound represented by formula (X).

The reaction temperature of the reaction is usually in a range of −78 to 150° C., and the reaction time is usually in a range of 0.1 to 200 hours.

After completion of the reaction, the compound represented by formula (I-3) can be isolated by subjecting the reaction mixture to post-treatment operations such as adding the reaction mixture into water, extracting with an organic solvent, drying the organic layer, concentrating the extract and the like. The isolated compound represented by formula (I-3) can be further purified by recrystallization, column chromatography and the like.

### (Production Process 5)

Among the compound (I), the compound represented by formula (I-4):

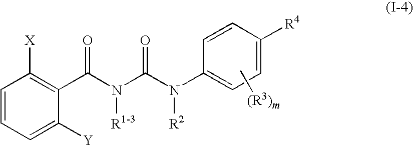

wherein, X, Y, R², R³, R⁴ and m are as defined above, R¹⁻³ represents a lower alkyl group optionally substituted with one or more of halogen atoms, a lower alkenyl group optionally substituted with one or more of halogen atoms, a lower alkynyl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, a lower alkoxycarbonyl group, an aryl lower alkyloxycarbonyl group, a N,N-di(lower alkyl)carbamoyl group, a lower alkanoyl group optionally substituted with one or more of halogen atoms, a formyl group, a lower alkylsulfonyl group optionally substituted with one or more of halogen atoms, an arylsulfonyl group, or an aryloxycarbonyl group, a lower cycloalkyl group, or a lower cycloalkyl lower alkyl group, or a group represented by —(CH₂)ₗ-A wherein l represents an integer of 1 to 4, and A represents a 5- or 6-membered heterocyclic group optionally substituted with a halogen atom), can be produced by reacting a compound represented by formula (I-2):

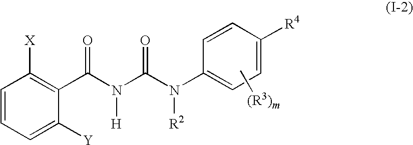

wherein, X, Y, R², R³, R⁴ and m are as defined above, with a compound represented by formula (XII):

L³-R¹⁻³ (XII)

wherein, R¹⁻³ is as defined above,
L³ represents a halogen atom, methanesulfonyloxy group, benzenesulfonyloxy group, toluenesulfonyloxy group, methoxysulfonyloxy group, or ethoxysulfonyloxy group, in the presence of a base.

The reaction is usually carried out in a solvent under the presence of a base.

Examples of the solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitriles such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water and s mixture thereof.

Examples of the base used for the reaction include hydroxides of alkali metal or alkali earth metal such as sodium hydroxide, potassium hydroxide, calcium hydroxide and the like, hydrides of alkali metal or alkali earth metal such as sodium hydride, potassium hydride, calcium hydride and the like, carbonates of alkali metal or alkali earth metal such as sodium carbonate, potassium carbonate and the like, alcoholates of alkali metal such as sodium ethylate, sodium methylate and the like, organic lithium reagents such as n-butyl lithium, lithium diisopropylamide and the like, organic bases such as triethylamine, pyridine, 1,8-diazabicyclo[5.4.0]undec-7-ene and the like.

When a reagent which is liquid under the reaction condition is used, the excess amount of each reagent can be used in terms of the amount of the reagents used for the reaction, but usually, the compound represented by formula (XII) is used with a rate of 1 to 4 moles and the base is used with a rate of 1 to 4 moles relative to one mole of the compound represented by formula (I-2).

The reaction temperature of the reaction is usually in a range of −78 to 150° C., and the reaction time is usually in a range of 0.1 to 100 hours.

After completion of the reaction, the compound represented by formula (I-4) can be isolated by subjecting the reaction mixture to post-treatment operations such as adding the reaction mixture into water, extracting with an organic solvent, drying the organic layer, concentrating the extract and the like. The isolated compound represented by formula (I-4) can be further purified by recrystallization, column chromatography and the like.

### (Production Process 6)

Among the compound (I), the compound represented by formula (I-5):

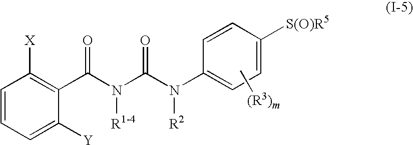

wherein, X, Y, R², R³, R⁵ and m are as defined above,
R¹⁻⁴ represents a hydrogen atom, a lower alkyl group optionally substituted with one or more of halogen atoms, a lower alkenyl group, a lower alkynyl group, an aryl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a N,N-di(lower alkyl)amino lower alkyl group, a lower alkylsulfonyl lower alkyl group, a lower alkoxy lower alkoxy lower alkyl group, an lower alkoxycarbonyl group, an aryl lower alkyloxycarbonyl group, a N,N-di(lower alkyl)carbamoyl group, a lower alkanoyl group optionally substituted with one or more of halogen atoms, a lower alkylsulfonyl group, an arylsulfonyl group, an aryloxycarbonyl group, a lower cycloalkyl group, a lower cycloalkyl lower alkyl group, a di(lower alkyl)amino group, a lower alkoxy group, or a group represented by —(CH₂)ₗ-A wherein l represents an integer of 1 to 4, and A represents a di(lower alkoxy)methyl group or a lower alkoxycarbonyl group, can be produced by subjecting a compound represented by formula (I-5a):

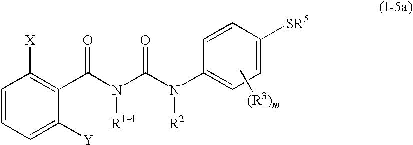

wherein, X, Y, R¹⁻⁴, R², R³, R⁵ and m are as defined above, to an oxidation reaction.

The reaction is usually carried out in a solvent under the presence of an oxidizing agent.

Examples of the solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitrites such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water and a mixture thereof.

Examples of the oxidizing agent used for the reaction include peroxides such as meta-chloroperbenzoic acid, hydrogen peroxide and the like.

The amount of the oxidizing agent used for the reaction is usually at a rate of 1 to 2 moles relative to one mole of the compound represented by the formula (I-5a).

The reaction temperature of the reaction is usually in a range of −78 to 150° C., and the reaction time is usually in a range of 0.1 to 100 hours.

After completion of the reaction, the compound represented by formula (I-5) can be isolated by subjecting the reaction mixture to post-treatment operations such as adding the reaction mixture into water, extracting with an organic solvent, drying the organic layer, concentrating the extract and the like. The isolated compound represented by formula (I-5) can be further purified by recrystallization, column chromatography and the like.

### (Production Process 7)

Among the compound (I), a compound represented by formula (I-6):

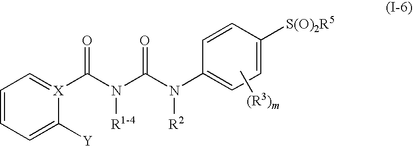

wherein, X, Y, R¹⁻⁴, R², R³, R⁵ and m are as defined above, can be produced by subjecting a compound represented by formula (I-6a):

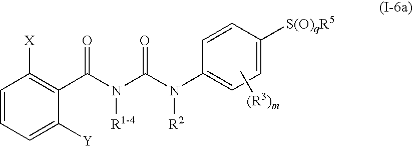

wherein, X, Y, R¹⁻⁴, R², R³, R⁵ and m are as defined above and q represents an integer of 0 or 1, to an oxidation reaction.

The reaction is usually carried out in a solvent under the presence of an oxidizing agent.

Examples of the solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitrites such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water and a mixture thereof.

Examples of the oxidizing agent used for the reaction include peroxides such as meta-chloroperbenzoic acid, hydrogen peroxide and the like.

The amount of the oxidizing agent used for the reaction is usually at a rate of 2 to 10 moles relative to one mole of the compound represented by formula (I-6a).

The reaction temperature of the reaction is usually in a range of −78 to 150° C., and the reaction time is usually in a range of 0.1 to 100 hours.

After completion of the reaction, the compound represented by formula (I-6) can be isolated by subjecting the reaction mixture to post-treatment operations such as adding the reaction mixture into water, extracting with an organic solvent, drying the organic layer, concentrating the extract and the like. The isolated compound represented by formula (I-6) can be further purified by recrystallization, column chromatography and the like.

### (Production Process 8)

Among the compound (I), a compound represented by formula (I-7):

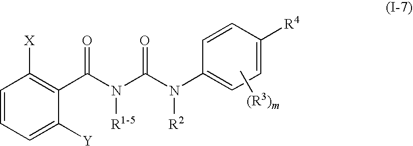

wherein, X, Y, R² and m are as defined above,
R¹⁻⁵ represents a lower alkyl group optionally substituted with one or more of halogen atoms, a lower alkenyl group optionally substituted with one or more of halogen atoms, a lower alkynyl group, an aryl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a lower alkanoyloxy lower alkyl group, an aryl lower alkoxy lower alkyl group,
a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, an lower alkoxycarbonyl group, an aryl lower alkoxycarbonyl group, a N,N-di(lower alkyl)carbamoyl group, a lower alkanoyl group optionally substituted with one or more of halogen atoms, a formyl group, a lower alkylsulfonyl group optionally substituted with one or more of halogen atoms, an arylsulfonyl group, an aryloxycarbonyl group, a lower alkoxy lower alkoxy lower alkyl group, a lower cycloalkyl group, or a lower cycloalkyl lower alkyl group, a di(lower alkyl)amino group, a lower alkoxy group, a 6-membered saturated heterocyclic ring, or a group represented by —(CH₂)ₗ-A wherein l represents an integer of 1 to 4 and A represents a di(lower alkoxy)methyl group, a lower alkoxycarbonyl group or a 5- or 6-membered heterocyclic group optionally substituted with a halogen atom,
R³ represents a halogen atom or a lower alkyl group optionally substituted with one or more of halogen atoms, and
R⁴ represents a lower alkoxycarbonyl group or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group or a lower alkoxy lower alkyl group optionally substituted with one or more of halogen atoms, and n represents an integer of 0 to 2, can be produced by reacting a compound represented by formula (II):

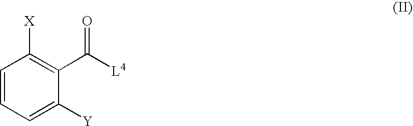

wherein, X and Y are as defined above, and L⁴ represents a halogen atom, with a compound represented by formula (III):

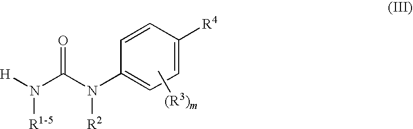

wherein, R¹⁻⁵, R², R³, R⁴ and m are as defined above.

The reaction is carried out in an organic solvent under the presence of a base.

Examples of the organic solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitrites such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water and a mixture thereof, and preferably include aromatic hydrocarbons such as toluene, xylene and the like, halogenated hydrocarbons such as chlorobenzene and the like, or amides such as N,N-dimethylformeamide and the like, and more preferably include toluene, xylene, and chlorobenzene.

Examples of the base used for the reaction include hydroxides of alkali metal or alkali earth metal such as sodium hydroxide, potassium hydroxide, calcium hydroxide and the like, hydrides of alkali metal or alkali earth metal such as sodium hydride, potassium hydride, calcium hydride and the like, carbonates of alkali metal or alkali earth metal such as sodium carbonate, potassium carbonate and the like, alcoholates of alkali metal such as sodium ethylate, sodium methylate and the like, organic lithium reagents such as n-butyl lithium, lithium diisopropylamide and the like, organic bases such as triethylamine, pyridine, 1,8-diazabicyclo[5.4.0]undec-7-ene and the like, and preferably include organic bases such as diisopropylethylamine, triethylamine, pyridine and 1,8-diazabicyclo[5.4.0]undec-7-ene or metal carbonates such as potassium carbonate, and particularly preferably include diisopropylethylamine, triethylamine and the like.

Regarding the amount of the reagent used for the reaction, usually the compound represented by formula (II) is used at a rate of 1 to 4 moles and the base is used at a rate of 1 to 4 moles relative to one mole of the compound represented by formula (III), and preferably the compound represented by formula (II) is used at a rate of 1.0 to 2.0 moles and the base is used at a rate of 1.0 to 2.0 relative to one mole of the compound represented by formula (III).

The reaction temperature of the reaction is usually in a range of −78 to 180° C., and preferably in a range of 80 to 150° C., and particularly preferably in a range of 90 to 120° C. The reaction time is usually in a range of 0.1 to 200 hours, and preferably in a range of 3 to 9 hours.

After completion of the reaction, the compound represented by formula (I-7) can be isolated by subjecting the reaction mixture to post-treatment operations such as adding the reaction mixture into water, extracting with an organic solvent, drying the organic layer, concentrating the extract and the like. The isolated compound represented by formula (I-7) can be further purified by recrystallization, column chromatography and the like.

### (Production Process 9)

Among the compound (I), a compound represented by formula (I-8):

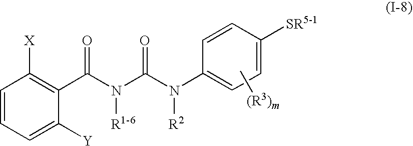

wherein, X, Y, R², R³ and m are as defined above,
R¹⁻⁶ represents a hydrogen atom, a lower alkyl group optionally substituted with one or more of halogen atoms, a lower alkenyl group optionally substituted with one or more of halogen atoms, a lower alkynyl group, an aryl lower alkyl group, a lower alkoxy lower alkyl group, an aryloxy lower alkyl group, a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, an lower alkoxycarbonyl group, an aryl lower alkoxycarbonyl group, a N,N-di(lower alkyl)carbamoyl group, a lower alkanoyl group optionally substituted with one or more of halogen atoms, a formyl group, a lower alkylsulfonyl group optionally substituted with one or more of halogen atoms, an arylsulfonyl group, an aryloxycarbonyl group, a lower cycloalkyl group, or a lower cycloalkyl lower alkyl group, or a group represented by —(CH₂)ₗ-A wherein l represents an integer of 1 to 4 and A represents a 5- or 6-membered heterocyclic group optionally substituted with a halogen atom, and
R⁵⁻¹ represents trifluoromethyl, 1,1,2,2,2-pentafluoroethyl, 1,1,2,2,3,3,3-heptafluoro-1-propyl, or trichloromethyl, can be produced by reacting a compound represented by formula (XVII):

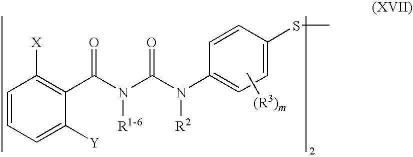

wherein X, Y, R¹⁻⁶, R², R³ and m are as defined above, with a compound represented by formula (XVIII):

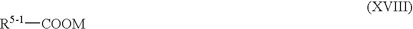

wherein R⁵⁻¹ is as defined above, and M represents sodium or potassium.

The reaction is usually carried out in a solvent.

Examples of the solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitrites such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water and a mixture thereof.

When a reagent which is liquid under the reaction condition is used, the excess amount of each reagent can be used in terms of the amount of the reagents used for the reaction, but usually, the compound represented by formula (XVIII) is used with a rate of 1 to 10 mole relative to one mole of the compound represented by formula (XVII).

The reaction temperature of the reaction is usually in a range of −78 to 150° C., and the reaction time is usually in a range of 0.1 to 100 hours.

After completion of the reaction, the compound represented by formula (I-8) can be isolated by carrying out post-treatment operations such as drying, concentration, and the like after filtering the reaction mixture. The isolated compound represented by formula (I-8) can be further purified by recrystallization, column chromatography and the like.

### (Reference Production Process 1)

The compound represented by formula (X):

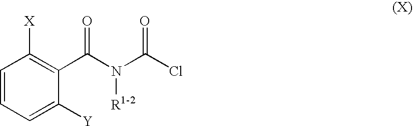

wherein, X, Y, and R¹⁻² are as defined above, can be produced by reacting a compound represented by formula (XV):

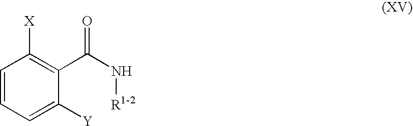

wherein, X, Y and R¹⁻² are as defined above, trialkylchlorosilane compound and chlorocarbonylation reagent.

The reaction is usually carried out in a solvent under the presence of a base.

Examples of the solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitrites such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water and a mixture thereof.

Examples of the base used for the reaction include hydroxides of alkali metal or alkali earth metal such as sodium hydroxide, potassium hydroxide, calcium hydroxide and the like, hydrides of alkali metal or alkali earth metal such as sodium hydride, potassium hydride, calcium hydride and the like, carbonates of alkali metal or alkali earth metal such as sodium carbonate, potassium carbonate and the like, alcoholates of alkali metal such as sodium ethylate, sodium methylate and the like, organic lithium reagents such as n-butyl lithium, lithium diisopropylamide and the like, organic bases such as triethylamine, pyridine, 1,8-diazabicyclo[5.4.0]undec-7-ene and the like.

Examples of the trialkylchlorosilane compound used for the reaction include trimethylchlorosilane and triethylchlorosilane.

Examples of the chlorocarbonylation reagent used for the reaction include phosgene, trichloromethyl chloroformate, bis(trichlormethyl)carbonate and the like.

Regarding the amount of the reagent used in the reaction, trialkylchlorosilane is usually used at a rate of 1 to 4 moles, chlorocarbonylation reagent is usually used at a rate of 1 to 4 moles and the base is usually used at a rate of 1 to 4 moles with respect to one mole of the compound represented by the formula (XV).

The reaction temperature of the reaction is usually in a range of −78 to 150° C., and the reaction time is usually in a range of 0.1 to 200 hours.

After completion of the reaction, the compound represented by formula (X) can be isolated by subjecting the reaction mixture to post-treatment operations such as concentrating the reaction mixture as it is. The isolated compound represented by formula (X) can be used for the next step without purifying.

### (Reference Production Process 2)

The compound represented by formula (III):

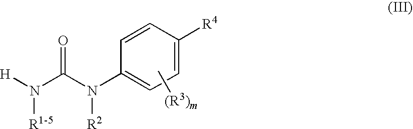

wherein, R¹⁻⁵, R², R³, R⁴ and m are as defined above, can be produced by reacting a compound represented by formula (XVI):

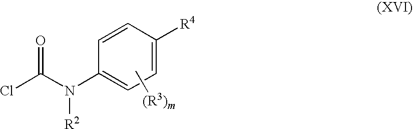

wherein, R², R³, R⁴, and m are as defined above, and a compound represented by the formula (XVII):

H₂N—R¹⁻⁵ (XVII)

wherein, R¹⁻⁵ is as defined above.

The reaction is usually carried out in a solvent under the presence of a base.

Examples of the solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitrites such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water and a mixture thereof.

Examples of the base used for the reaction include hydroxides of alkali metal or alkali earth metal such as sodium hydroxide, potassium hydroxide, calcium hydroxide and the like, hydrides of alkali metal or alkali earth metal such as sodium hydride, potassium hydride, calcium hydride and the like, carbonates of alkali metal or alkali earth metal such as sodium carbonate, potassium carbonate and the like, alcoholates of alkali metal such as sodium ethylate, sodium methylate and the like, organic lithium reagents such as n-butyl lithium, lithium diisopropylamide and the like, organic bases such as triethylamine, pyridine, 1,8-diazabicyclo[5.4.0]undec-7-ene and the like. Alternatively, excess amount of (XVII) can be used as base.

Regarding the amount of the reagent used in the reaction, the compound represented by formula (XVII) is usually at a rate of 1 to 6 moles and the base is usually at a rate of 1 to 6 moles with respect to one mole of the compound represented by formula (XVI).

The reaction temperature of the reaction is usually in a range of −78 to 150° C., and the reaction time is usually in a range of 0.1 to 200 hours.

After completion of the reaction, the compound represented by formula (III-1) can be isolated by subjecting the reaction mixture to post-treatment operations such as adding the reaction mixture into water, extracting with an organic solvent, drying the organic layer, concentrating the extract and the like. The isolated compound represented by formula (III-1) can be further purified by recrystallization, column chromatography and the like. Moreover, the compound represented by formula (III-1) can be used in the next step without purifying.

### (Reference Production Process 3)

The compound represented by formula (XVI):

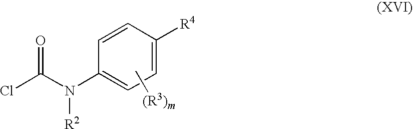

wherein, R², R³, R⁴, and m are as defined above, can be produced by reacting a compound represented by formula (IX):

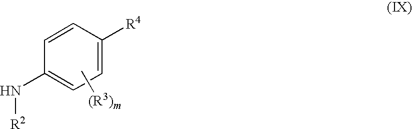

wherein, R², R³, R⁴ and m are as defined above, with a chlorocarbonylation reagent.

The reaction is usually carried out in a solvent under the presence of a base.

Examples of the solvent used for the reaction include ketones such as acetone, methyl ethyl ketone and the like, aromatic hydrocarbons such as benzene, toluene, xylene and the like, aliphatic hydrocarbons such as hexane, heptane and the like, ethers such as diethyl ether, tetrahydrofuran, 1,4-dioxane, 1,2-dimethoxyethane, 1,2-diethoxyethane and the like, halogenated hydrocarbons such as chloroform, chlorobenzene, dichlorobenzene and the like, nitrites such as acetonitrile and the like, aprotic polar solvents such as N,N-dimethylformeamide, N,N-dimethylacetoamide, 1-methyl-2-pyrrolidone, 1,3-dimethylimidazolinone, dimethylsulfoxide and the like, water and a mixture thereof.

Examples of the base used for the reaction include hydroxides of alkali metal or alkali earth metal such as sodium hydroxide, potassium hydroxide, calcium hydroxide and the like, hydrides of alkali metal or alkali earth metal such as sodium hydride, potassium hydride, calcium hydride and the like, carbonates of alkali metal or alkali earth metal such as sodium carbonate, potassium carbonate and the like, alcoholates of alkali metal such as sodium ethylate, sodium methylate and the like, organic lithium reagents such as n-butyl lithium, lithium diisopropylamide and the like, organic base such as triethylamine, pyridine, 1,8-diazabicyclo[5.4.0]undec-7-ene and the like.

Examples of the chlorocarbonylation reagent used for the reaction include phosgene, trichloromethyl chloroformate, bis(trichloromethyl)carbonate and the like.

Regarding the amount of the reagent used in the reaction, chlorocarbonylation reagent is usually used at a rate of 1 to 4 moles and the base is usually used at a rate of 1 to 4 moles with respect to one mole of the compound represented by formula (IX).

The reaction temperature of the reaction is usually in a range of −78 to 150° C., and the reaction time is usually in a range of 0.1 to 200 hours.

After completion of the reaction, the compound represented by formula (XVI) can be isolated by subjecting the reaction mixture to post-treatment operations such as adding the reaction mixture into water, extracting with an organic solvent, drying the organic layer, concentrating the extract and the like. The isolated compound represented by formula (XVI) can be further purified by recrystallization, column chromatography and the like. Further, after completion of the reaction, the compound represented by formula (XVI) can be isolated by operating post-treatments such as concentrating the reaction mixture as it is. The isolated compound represented by formula (XVI) can be used in the next step without purifying.

The compound represented by formulas (IV), (VI) and (VIII) can be produced according to a method for production described in, for example, Journal of Agricultural and Food Chemistry (1973) Vol. 21, (No. 3), P348-354, or an analoguous method thereto.

The compound represented by formula (IX) can be produced according to a method for production described in, for example, Journal of Pesticide Science 23 (3) (1998) P250-254 or Journal of the Chemical Society Chemical Communication (1984) P1334-1335, or an analogous method thereto.

The compound represented by the formula (XV) can be produced according to a method for production described in, for example, Journal of the Chemical Society Perkin Transactions 1 (1985) P1381-1385, or an analogous method thereto.

Further, by subjecting the compound produced by the above-mentioned method for production and the like to a per se known reaction, for example, alkylation, alkenylation, alkynylation, acylation, amination, sulfidation, sulfinylation, sulfonation, oxidation, reduction, halogenation, nitration and the like, its substituent can be converted to other desired substituent.

The compound obtained by the above-mentioned Production Processes 1 to 8 and Reference Production Processes 1 to 3 can be purified by methods such as recrystallization, column chromatography, high performance liquid chromatography, medium pressure preparative high performance liquid chromatography, demineralization resin column chromatography, reprecipitation and the like.

A preferred salt of the compounds (I) is a salt wherein the basic nitrogen atom in the molecule and basic group such as dialkyl amino group and the like in the substituent form an agrochemically acceptable acid addition salt with an inorganic acid, organic acid or the like.

Examples of the inorganic acid addition salt include salt with hydrochloric acid, hydrobromic acid, hydroiodic acid, sulfuric acid, nitric acid, phosphoric acid and perchloric acid, and examples of the organic acid addition salt include a salt with formic acid, acetic acid, propionic acid, oxalic acid, succinic acid, benzoic acid, paratoluenesulfonic acid, methanesulfonic acid and trifluoroacetic acid.

When R¹ of the compound (I) is a hydrogen atom, an anion generated by dissociation of the hydrogen atom and a metal cation can form an agrochemically acceptable salt.

For example, a salt with alkali metal (sodium, potassium and the like) and alkali earth metal (calcium and the like) are exemplified. Moreover, when R¹ of the compound (I) is a hydrogen atom, the compound (I) and an inorganic base or organic base can form an agrochemically acceptable addition salt.

Examples of the inorganic base include a salt with ammonia, and examples of the organic base include a salt with dimethylamine, triethylamine, N,N-dimethylaniline, piperazine, pyrrolidine, piperidine, pyridine, 2-phenylethylamine, benzylamine, ethanolamine, diethanolamine and 1,8-diazabiciclo[5,4,0]undecene and the like.

The salt of compound (I) can be obtained by mixing compound (I) and an acid or a base.

Hereinafter, the compounds of the present invention will be shown specifically.

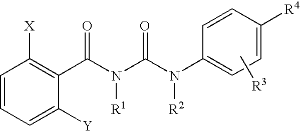

wherein, X, Y, R¹, R², R³ and R⁴ are any one of the combinations of the substituents shown in Table 1.

TABLE 1

| No. | X | Y | R¹ | R² | R³ | R⁴ |
|---|---|---|---|---|---|---|
| I-1 | Cl | F | H | Me | H | SCF₃ |
| I-2 | Cl | Cl | H | Me | H | SCF₃ |
| I-3 | Cl | F | H | Me | 2-Cl | SCF₃ |
| I-4 | Cl | Cl | H | Me | 2-Cl | SCF₃ |
| I-5 | Cl | F | H | Me | 2-Me | SCF₃ |
| I-6 | Cl | Cl | H | Me | 2-Me | SCF₃ |
| I-7 | Cl | F | H | Me | 2,3-Me₂ | SCF₃ |
| I-8 | Cl | Cl | H | Me | 2,3-Me₂ | SCF₃ |
| I-9 | Cl | F | Me | Me | H | SCF₃ |
| I-10 | Cl | Cl | Me | Me | H | SCF₃ |
| I-11 | Cl | F | Me | Me | 2-Cl | SCF₃ |
| I-12 | Cl | Cl | Me | Me | 2-Cl | SCF₃ |
| I-13 | Cl | F | Me | Me | 2-Me | SCF₃ |
| I-14 | Cl | Cl | Me | Me | 2-Me | SCF₃ |
| I-15 | Cl | F | Me | Me | 2,3-Me₂ | SCF₃ |
| I-16 | Cl | Cl | Me | Me | 2,3-Me₂ | SCF₃ |
| I-17 | F | F | H | Et | H | SCF₃ |
| I-18 | Cl | F | H | Et | H | SCF₃ |
| I-19 | Cl | Cl | H | Et | H | SCF₃ |
| I-20 | Cl | F | H | Et | 2-F | SCF₃ |
| I-21 | Cl | Cl | H | Et | 2-F | SCF₃ |
| I-22 | F | F | H | Et | 2-Cl | SCF₃ |
| I-23 | Cl | F | H | Et | 2-Cl | SCF₃ |
| I-24 | Cl | Cl | H | Et | 2-Cl | SCF₃ |
| I-25 | F | F | H | Et | 2-Me | SCF₃ |
| I-26 | Cl | F | H | Et | 2-Me | SCF₃ |
| I-27 | Cl | Cl | H | Et | 2-Me | SCF₃ |
| I-28 | F | F | H | Et | 2,3-Me₂ | SCF₃ |
| I-29 | Cl | F | H | Et | 2,3-Me₂ | SCF₃ |
| I-30 | Cl | Cl | H | Et | 2,3-Me₂ | SCF₃ |
| I-31 | F | F | Me | Et | H | SCF₃ |
| I-32 | Cl | F | Me | Et | H | SCF₃ |
| I-33 | Cl | Cl | Me | Et | H | SCF₃ |
| I-34 | Cl | F | Me | Et | 2-F | SCF₃ |
| I-35 | Cl | Cl | Me | Et | 2-F | SCF₃ |
| I-36 | F | F | Me | Et | 2-Cl | SCF₃ |
| I-37 | Cl | F | Me | Et | 2-Cl | SCF₃ |
| I-38 | Cl | Cl | Me | Et | 2-Cl | SCF₃ |
| I-39 | F | F | Me | Et | 2-Me | SCF₃ |
| I-40 | Cl | F | Me | Et | 2-Me | SCF₃ |
| I-41 | Cl | Cl | Me | Et | 2-Me | SCF₃ |
| I-42 | F | F | Me | Et | 2,3-Me₂ | SCF₃ |
| I-43 | Cl | F | Me | Et | 2,3-Me₂ | SCF₃ |
| I-44 | Cl | Cl | Me | Et | 2,3-Me₂ | SCF₃ |
| I-45 | F | F | H | Me | H | SOCF₃ |
| I-46 | Cl | F | H | Me | H | SOCF₃ |
| I-47 | Cl | Cl | H | Me | H | SOCF₃ |
| I-48 | Cl | F | H | Me | 2-F | SOCF₃ |
| I-49 | Cl | Cl | H | Me | 2-F | SOCF₃ |
| I-50 | F | F | H | Me | 2-Cl | SOCF₃ |
| I-51 | Cl | F | H | Me | 2-Cl | SOCF₃ |
| I-52 | Cl | Cl | H | Me | 2-Cl | SOCF₃ |
| I-53 | F | F | H | Me | 2-Me | SOCF₃ |
| I-54 | Cl | F | H | Me | 2-Me | SOCF₃ |
| I-55 | Cl | Cl | H | Me | 2-Me | SOCF₃ |
| I-56 | F | F | H | Me | 2,3-Me₂ | SOCF₃ |
| I-57 | Cl | F | H | Me | 2,3-Me₂ | SOCF₃ |
| I-58 | Cl | Cl | H | Me | 2,3-Me₂ | SOCF₃ |
| I-59 | F | F | Me | Me | H | SOCF₃ |
| I-60 | Cl | F | Me | Me | H | SOCF₃ |
| I-61 | Cl | Cl | Me | Me | H | SOCF₃ |
| I-62 | F | F | Me | Me | 2-F | SOCF₃ |
| I-63 | Cl | F | Me | Me | 2-F | SOCF₃ |
| I-64 | Cl | Cl | Me | Me | 2-F | SOCF₃ |
| I-65 | F | F | Me | Me | 2-Cl | SOCF₃ |
| I-66 | Cl | F | Me | Me | 2-Cl | SOCF₃ |
| I-67 | Cl | Cl | Me | Me | 2-Cl | SOCF₃ |
| I-68 | F | F | Me | Me | 2-Me | SOCF₃ |
| I-69 | Cl | F | Me | Me | 2-Me | SOCF₃ |
| I-70 | Cl | Cl | Me | Me | 2-Me | SOCF₃ |
| I-71 | F | F | Me | Me | 2,3-Me₂ | SOCF₃ |
| I-72 | Cl | F | Me | Me | 2,3-Me₂ | SOCF₃ |
| I-73 | Cl | Cl | Me | Me | 2,3-Me₂ | SOCF₃ |
| I-74 | F | F | H | Et | H | SOCF₃ |
| I-75 | Cl | F | H | Et | H | SOCF₃ |
| I-76 | Cl | Cl | H | Et | H | SOCF₃ |
| I-77 | F | F | H | Et | 2-F | SOCF₃ |
| I-78 | Cl | F | H | Et | 2-F | SOCF₃ |
| I-79 | Cl | Cl | H | Et | 2-F | SOCF₃ |
| I-80 | F | F | H | Et | 2-Cl | SOCF₃ |
| I-81 | Cl | F | H | Et | 2-Cl | SOCF₃ |
| I-82 | Cl | Cl | H | Et | 2-Cl | SOCF₃ |
| I-83 | F | F | H | Et | 2-Me | SOCF₃ |
| I-84 | Cl | F | H | Et | 2-Me | SOCF₃ |
| I-85 | Cl | Cl | H | Et | 2-Me | SOCF₃ |
| I-86 | F | F | H | Et | 2,3-Me₂ | SOCF₃ |
| I-87 | Cl | F | H | Et | 2,3-Me₂ | SOCF₃ |
| I-88 | Cl | Cl | H | Et | 2,3-Me₂ | SOCF₃ |
| I-89 | F | F | Me | Et | H | SOCF₃ |
| I-90 | Cl | F | Me | Et | H | SOCF₃ |
| I-91 | Cl | Cl | Me | Et | H | SOCF₃ |
| I-92 | F | F | Me | Et | 2-F | SOCF₃ |
| I-93 | Cl | F | Me | Et | 2-F | SOCF₃ |
| I-94 | Cl | Cl | Me | Et | 2-F | SOCF₃ |
| I-95 | F | F | Me | Et | 2-Cl | SOCF₃ |
| I-96 | Cl | F | Me | Et | 2-Cl | SOCF₃ |
| I-97 | Cl | Cl | Me | Et | 2-Cl | SOCF₃ |
| I-98 | F | F | Me | Et | 2-Me | SOCF₃ |
| I-99 | Cl | F | Me | Et | 2-Me | SOCF₃ |
| I-100 | Cl | Cl | Me | Et | 2-Me | SOCF₃ |
| I-101 | F | F | Me | Et | 2,3-Me₂ | SOCF₃ |
| I-102 | Cl | F | Me | Et | 2,3-Me₂ | SOCF₃ |
| I-103 | Cl | Cl | Me | Et | 2,3-Me₂ | SOCF₃ |
| I-104 | F | F | H | Me | H | SO₂CF₃ |
| I-105 | Cl | F | H | Me | H | SO₂CF₃ |
| I-106 | Cl | Cl | H | Me | H | SO₂CF₃ |
| I-107 | Cl | F | H | Me | 2-F | SO₂CF₃ |
| I-108 | Cl | Cl | H | Me | 2-F | SO₂CF₃ |
| I-109 | F | F | H | Me | 2-Cl | SO₂CF₃ |
| I-110 | Cl | F | H | Me | 2-Cl | SO₂CF₃ |
| I-111 | Cl | Cl | H | Me | 2-Cl | SO₂CF₃ |
| I-112 | F | F | H | Me | 2-Me | SO₂CF₃ |
| I-113 | Cl | F | H | Me | 2-Me | SO₂CF₃ |
| I-114 | Cl | Cl | H | Me | 2-Me | SO₂CF₃ |
| I-115 | F | F | H | Me | 2,3-Me₂ | SO₂CF₃ |
| I-116 | Cl | F | H | Me | 2,3-Me₂ | SO₂CF₃ |
| I-117 | Cl | Cl | H | Me | 2,3-Me₂ | SO₂CF₃ |
| I-118 | F | F | Me | Me | H | SO₂CF₃ |
| I-119 | Cl | F | Me | Me | H | SO₂CF₃ |
| I-120 | Cl | Cl | Me | Me | H | SO₂CF₃ |
| I-121 | Cl | F | Me | Me | 2-F | SO₂CF₃ |
| I-122 | Cl | Cl | Me | Me | 2-F | SO₂CF₃ |
| I-123 | F | F | Me | Me | 2-Cl | SO₂CF₃ |
| I-124 | Cl | F | Me | Me | 2-Cl | SO₂CF₃ |
| I-125 | Cl | Cl | Me | Me | 2-Cl | SO₂CF₃ |
| I-126 | F | F | Me | Me | 2-Me | SO₂CF₃ |
| I-127 | Cl | F | Me | Me | 2-Me | SO₂CF₃ |
| I-128 | Cl | Cl | Me | Me | 2-Me | SO₂CF₃ |
| I-129 | F | F | Me | Me | 2,3-Me₂ | SO₂CF₃ |
| I-130 | Cl | F | Me | Me | 2,3-Me₂ | SO₂CF₃ |
| I-131 | Cl | Cl | Me | Me | 2,3-Me₂ | SO₂CF₃ |
| I-132 | F | F | H | Et | H | SO₂CF₃ |
| I-133 | Cl | F | H | Et | H | SO₂CF₃ |
| I-134 | Cl | Cl | H | Et | H | SO₂CF₃ |
| I-135 | F | F | H | Et | 2-F | SO₂CF₃ |
| I-136 | Cl | F | H | Et | 2-F | SO₂CF₃ |
| I-137 | Cl | Cl | H | Et | 2-F | SO₂CF₃ |
| I-138 | F | F | H | Et | 2-Cl | SO₂CF₃ |
| I-139 | Cl | F | H | Et | 2-Cl | SO₂CF₃ |
| I-140 | Cl | Cl | H | Et | 2-Cl | SO₂CF₃ |
| I-141 | F | F | H | Et | 2-Me | SO₂CF₃ |
| I-142 | Cl | F | H | Et | 2-Me | SO₂CF₃ |
| I-143 | Cl | Cl | H | Et | 2-Me | SO₂CF₃ |
| I-144 | F | F | H | Et | 2,3-Me₂ | SO₂CF₃ |
| I-145 | Cl | F | H | Et | 2,3-Me₂ | SO₂CF₃ |
| I-146 | Cl | Cl | H | Et | 2,3-Me₂ | SO₂CF₃ |
| I-147 | F | F | Me | Et | H | SO₂CF₃ |
| I-148 | Cl | F | Me | Et | H | SO₂CF₃ |
| I-149 | Cl | Cl | Me | Et | H | SO₂CF₃ |
| I-150 | F | F | Me | Et | 2-F | SO₂CF₃ |
| I-151 | Cl | F | Me | Et | 2-F | SO₂CF₃ |
| I-152 | Cl | Cl | Me | Et | 2-F | SO₂CF₃ |
| I-153 | F | F | Me | Et | 2-Cl | SO₂CF₃ |
| I-154 | Cl | F | Me | Et | 2-Cl | SO₂CF₃ |
| I-155 | Cl | Cl | Me | Et | 2-Cl | SO₂CF₃ |
| I-156 | F | F | Me | Et | 2-Me | SO₂CF₃ |
| I-157 | Cl | F | Me | Et | 2-Me | SO₂CF₃ |
| I-158 | Cl | Cl | Me | Et | 2-Me | SO₂CF₃ |
| I-159 | F | F | Me | Et | 2,3-Me₂ | SO₂CF₃ |
| I-160 | Cl | F | Me | Et | 2,3-Me₂ | SO₂CF₃ |
| I-161 | Cl | Cl | Me | Et | 2,3-Me₂ | SO₂CF₃ |
| I-162 | F | F | H | Me | H | SCF₂CF₂H |
| I-163 | Cl | F | H | Me | H | SCF₂CF₂H |
| I-164 | Cl | Cl | H | Me | H | SCF₂CF₂H |
| I-165 | Cl | Cl | H | Me | 2-F | SCF₂CF₂H |
| I-166 | Cl | F | H | Me | 2-Cl | SCF₂CF₂H |
| I-167 | Cl | Cl | H | Me | 2-Cl | SCF₂CF₂H |
| I-168 | Cl | F | H | Me | 2-Me | SCF₂CF₂H |
| I-169 | Cl | Cl | H | Me | 2-Me | SCF₂CF₂H |
| I-170 | Cl | F | H | Me | 2,3-Me₂ | SCF₂CF₂H |
| I-171 | Cl | Cl | H | Me | 2,3-Me₂ | SCF₂CF₂H |
| I-172 | F | F | Me | Me | H | SCF₂CF₂H |
| I-173 | Cl | F | Me | Me | H | SCF₂CF₂H |
| I-174 | Cl | Cl | Me | Me | H | SCF₂CF₂H |
| I-175 | Cl | Cl | Me | Me | 2-F | SCF₂CF₂H |
| I-176 | Cl | F | Me | Me | 2-Cl | SCF₂CF₂H |
| I-177 | Cl | Cl | Me | Me | 2-Cl | SCF₂CF₂H |
| I-178 | Cl | F | Me | Me | 2-Me | SCF₂CF₂H |
| I-179 | Cl | Cl | Me | Me | 2-Me | SCF₂CF₂H |
| I-180 | Cl | F | Me | Me | 2,3-Me₂ | SCF₂CF₂H |
| I-181 | Cl | Cl | Me | Me | 2,3-Me₂ | SCF₂CF₂H |
| I-182 | F | F | H | Et | H | SCF₂CF₂H |
| I-183 | Cl | F | H | Et | H | SCF₂CF₂H |
| I-184 | Cl | Cl | H | Et | H | SCF₂CF₂H |
| I-185 | F | F | H | Et | 2-F | SCF₂CF₂H |
| I-186 | Cl | F | H | Et | 2-F | SCF₂CF₂H |
| I-187 | Cl | Cl | H | Et | 2-F | SCF₂CF₂H |
| I-188 | F | F | H | Et | 2-Cl | SCF₂CF₂H |
| I-189 | Cl | F | H | Et | 2-Cl | SCF₂CF₂H |
| I-190 | Cl | Cl | H | Et | 2-Cl | SCF₂CF₂H |
| I-191 | F | F | H | Et | 2-Me | SCF₂CF₂H |
| I-192 | Cl | F | H | Et | 2-Me | SCF₂CF₂H |
| I-193 | Cl | Cl | H | Et | 2-Me | SCF₂CF₂H |
| I-194 | F | F | H | Et | 2,3-Me₂ | SCF₂CF₂H |
| I-195 | Cl | F | H | Et | 2,3-Me₂ | SCF₂CF₂H |
| I-196 | Cl | Cl | H | Et | 2,3-Me₂ | SCF₂CF₂H |
| I-197 | F | F | Me | Et | H | SCF₂CF₂H |
| I-198 | Cl | F | Me | Et | H | SCF₂CF₂H |
| I-199 | Cl | Cl | Me | Et | H | SCF₂CF₂H |
| I-200 | F | F | Me | Et | 2-F | SCF₂CF₂H |
| I-201 | Cl | F | Me | Et | 2-F | SCF₂CF₂H |
| I-202 | Cl | Cl | Me | Et | 2-F | SCF₂CF₂H |
| I-203 | F | F | Me | Et | 2-Cl | SCF₂CF₂H |
| I-204 | Cl | F | Me | Et | 2-Cl | SCF₂CF₂H |
| I-205 | Cl | Cl | Me | Et | 2-Cl | SCF₂CF₂H |
| I-206 | F | F | Me | Et | 2-Me | SCF₂CF₂H |
| I-207 | Cl | F | Me | Et | 2-Me | SCF₂CF₂H |
| I-208 | Cl | Cl | Me | Et | 2-Me | SCF₂CF₂H |
| I-209 | F | F | Me | Et | 2,3-Me₂ | SCF₂CF₂H |
| I-210 | Cl | F | Me | Et | 2,3-Me₂ | SCF₂CF₂H |
| I-211 | Cl | Cl | Me | Et | 2,3-Me₂ | SCF₂CF₂H |
| I-212 | F | F | H | Me | H | SOCF₂CF₂H |
| I-213 | Cl | F | H | Me | H | SOCF₂CF₂H |
| I-214 | Cl | Cl | H | Me | H | SOCF₂CF₂H |
| I-215 | Cl | F | H | Me | 2-F | SOCF₂CF₂H |
| I-216 | Cl | Cl | H | Me | 2-F | SOCF₂CF₂H |
| I-217 | F | F | H | Me | 2-Cl | SOCF₂CF₂H |
| I-218 | Cl | F | H | Me | 2-Cl | SOCF₂CF₂H |
| I-219 | Cl | Cl | H | Me | 2-Cl | SOCF₂CF₂H |
| I-220 | F | F | H | Me | 2-Me | SOCF₂CF₂H |
| I-221 | Cl | F | H | Me | 2-Me | SOCF₂CF₂H |
| I-222 | Cl | Cl | H | Me | 2-Me | SOCF₂CF₂H |
| I-223 | F | F | H | Me | 2,3-Me₂ | SOCF₂CF₂H |
| I-224 | Cl | F | H | Me | 2,3-Me₂ | SOCF₂CF₂H |
| I-225 | Cl | Cl | H | Me | 2,3-Me₂ | SOCF₂CF₂H |
| I-226 | F | F | Me | Me | H | SOCF₂CF₂H |
| I-227 | Cl | F | Me | Me | H | SOCF₂CF₂H |
| I-228 | Cl | Cl | Me | Me | H | SOCF₂CF₂H |
| I-229 | Cl | F | Me | Me | 2-F | SOCF₂CF₂H |
| I-230 | Cl | Cl | Me | Me | 2-F | SOCF₂CF₂H |
| I-231 | F | F | Me | Me | 2-Cl | SOCF₂CF₂H |
| I-232 | Cl | F | Me | Me | 2-Cl | SOCF₂CF₂H |
| I-233 | Cl | Cl | Me | Me | 2-Cl | SOCF₂CF₂H |
| I-234 | F | F | Me | Me | 2-Me | SOCF₂CF₂H |
| I-235 | Cl | F | Me | Me | 2-Me | SOCF₂CF₂H |
| I-236 | Cl | Cl | Me | Me | 2-Me | SOCF₂CF₂H |
| I-237 | F | F | Me | Me | 2,3-Me₂ | SOCF₂CF₂H |
| I-238 | Cl | F | Me | Me | 2,3-Me₂ | SOCF₂CF₂H |
| I-239 | Cl | Cl | Me | Me | 2,3-Me₂ | SOCF₂CF₂H |
| I-240 | F | F | H | Et | H | SOCF₂CF₂H |
| I-241 | Cl | F | H | Et | H | SOCF₂CF₂H |
| I-242 | Cl | Cl | H | Et | H | SOCF₂CF₂H |
| I-243 | F | F | H | Et | 2-F | SOCF₂CF₂H |
| I-244 | Cl | F | H | Et | 2-F | SOCF₂CF₂H |
| I-245 | Cl | Cl | H | Et | 2-F | SOCF₂CF₂H |
| I-246 | F | F | H | Et | 2-Cl | SOCF₂CF₂H |
| I-247 | Cl | F | H | Et | 2-Cl | SOCF₂CF₂H |
| I-248 | Cl | Cl | H | Et | 2-Cl | SOCF₂CF₂H |
| I-249 | F | F | H | Et | 2-Me | SOCF₂CF₂H |
| I-250 | Cl | F | H | Et | 2-Me | SOCF₂CF₂H |
| I-251 | Cl | Cl | H | Et | 2-Me | SOCF₂CF₂H |
| I-252 | F | F | H | Et | 2,3-Me₂ | SOCF₂CF₂H |
| I-253 | Cl | F | H | Et | 2,3-Me₂ | SOCF₂CF₂H |
| I-254 | Cl | Cl | H | Et | 2,3-Me₂ | SOCF₂CF₂H |
| I-255 | F | F | Me | Et | H | SOCF₂CF₂H |
| I-256 | Cl | F | Me | Et | H | SOCF₂CF₂H |
| I-257 | Cl | Cl | Me | Et | H | SOCF₂CF₂H |
| I-258 | F | F | Me | Et | 2-F | SOCF₂CF₂H |
| I-259 | Cl | F | Me | Et | 2-F | SOCF₂CF₂H |
| I-260 | Cl | Cl | Me | Et | 2-F | SOCF₂CF₂H |
| I-261 | F | F | Me | Et | 2-Cl | SOCF₂CF₂H |
| I-262 | Cl | F | Me | Et | 2-Cl | SOCF₂CF₂H |
| I-263 | Cl | Cl | Me | Et | 2-Cl | SOCF₂CF₂H |
| I-264 | F | F | Me | Et | 2-Me | SOCF₂CF₂H |
| I-265 | Cl | F | Me | Et | 2-Me | SOCF₂CF₂H |
| I-266 | Cl | Cl | Me | Et | 2-Me | SOCF₂CF₂H |
| I-267 | F | F | Me | Et | 2,3-Me₂ | SOCF₂CF₂H |
| I-268 | Cl | F | Me | Et | 2,3-Me₂ | SOCF₂CF₂H |
| I-269 | Cl | Cl | Me | Et | 2,3-Me₂ | SOCF₂CF₂H |
| I-270 | F | F | H | Me | H | SO₂CF₂CF₂H |
| I-271 | Cl | F | H | Me | H | SO₂CF₂CF₂H |
| I-272 | Cl | Cl | H | Me | H | SO₂CF₂CF₂H |
| I-273 | Cl | F | H | Me | 2-F | SO₂CF₂CF₂H |
| I-274 | Cl | Cl | H | Me | 2-F | SO₂CF₂CF₂H |
| I-275 | F | F | H | Me | 2-Cl | SO₂CF₂CF₂H |
| I-276 | Cl | F | H | Me | 2-Cl | SO₂CF₂CF₂H |
| I-277 | Cl | Cl | H | Me | 2-Cl | SO₂CF₂CF₂H |
| I-278 | F | F | H | Me | 2-Me | SO₂CF₂CF₂H |
| I-279 | Cl | F | H | Me | 2-Me | SO₂CF₂CF₂H |
| I-280 | Cl | Cl | H | Me | 2-Me | SO₂CF₂CF₂H |
| I-281 | F | F | H | Me | 2,3-Me₂ | SO₂CF₂CF₂H |
| I-282 | Cl | F | H | Me | 2,3-Me₂ | SO₂CF₂CF₂H |
| I-283 | Cl | Cl | H | Me | 2,3-Me₂ | SO₂CF₂CF₂H |
| I-284 | F | F | Me | Me | H | SO₂CF₂CF₂H |
| I-285 | Cl | F | Me | Me | H | SO₂CF₂CF₂H |
| I-286 | Cl | Cl | Me | Me | H | SO₂CF₂CF₂H |
| I-287 | Cl | F | Me | Me | 2-F | SO₂CF₂CF₂H |
| I-288 | Cl | Cl | Me | Me | 2-F | SO₂CF₂CF₂H |
| I-289 | F | F | Me | Me | 2-Cl | SO₂CF₂CF₂H |
| I-290 | Cl | F | Me | Me | 2-Cl | SO₂CF₂CF₂H |
| I-291 | Cl | Cl | Me | Me | 2-Cl | SO₂CF₂CF₂H |
| I-292 | F | F | Me | Me | 2-Me | SO₂CF₂CF₂H |
| I-293 | Cl | F | Me | Me | 2-Me | SO₂CF₂CF₂H |
| I-294 | Cl | Cl | Me | Me | 2-Me | SO₂CF₂CF₂H |
| I-295 | F | F | Me | Me | 2,3-Me₂ | SO₂CF₂CF₂H |
| I-296 | Cl | F | Me | Me | 2,3-Me₂ | SO₂CF₂CF₂H |
| I-297 | Cl | Cl | Me | Me | 2,3-Me₂ | SO₂CF₂CF₂H |
| I-298 | F | F | H | Et | H | SO₂CF₂CF₂H |
| I-299 | Cl | F | H | Et | H | SO₂CF₂CF₂H |
| I-300 | Cl | Cl | H | Et | H | SO₂CF₂CF₂H |
| I-301 | F | F | H | Et | 2-F | SO₂CF₂CF₂H |
| I-302 | Cl | F | H | Et | 2-F | SO₂CF₂CF₂H |
| I-303 | Cl | Cl | H | Et | 2-F | SO₂CF₂CF₂H |
| I-304 | F | F | H | Et | 2-Cl | SO₂CF₂CF₂H |
| I-305 | Cl | F | H | Et | 2-Cl | SO₂CF₂CF₂H |
| I-306 | Cl | Cl | H | Et | 2-Cl | SO₂CF₂CF₂H |
| I-307 | F | F | H | Et | 2-Me | SO₂CF₂CF₂H |
| I-308 | Cl | F | H | Et | 2-Me | SO₂CF₂CF₂H |
| I-309 | Cl | Cl | H | Et | 2-Me | SO₂CF₂CF₂H |
| I-310 | F | F | H | Et | 2,3-Me₂ | SO₂CF₂CF₂H |
| I-311 | Cl | F | H | Et | 2,3-Me₂ | SO₂CF₂CF₂H |
| I-312 | Cl | Cl | H | Et | 2,3-Me₂ | SO₂CF₂CF₂H |
| I-313 | F | F | Me | Et | H | SO₂CF₂CF₂H |
| I-314 | Cl | F | Me | Et | H | SO₂CF₂CF₂H |
| I-315 | Cl | Cl | Me | Et | H | SO₂CF₂CF₂H |
| I-316 | F | F | Me | Et | 2-F | SO₂CF₂CF₂H |
| I-317 | Cl | F | Me | Et | 2-F | SO₂CF₂CF₂H |
| I-318 | Cl | Cl | Me | Et | 2-F | SO₂CF₂CF₂H |
| I-319 | F | F | Me | Et | 2-Cl | SO₂CF₂CF₂H |
| I-320 | Cl | F | Me | Et | 2-Cl | SO₂CF₂CF₂H |
| I-321 | Cl | Cl | Me | Et | 2-Cl | SO₂CF₂CF₂H |
| I-322 | F | F | Me | Et | 2-Me | SO₂CF₂CF₂H |
| I-323 | Cl | F | Me | Et | 2-Me | SO₂CF₂CF₂H |
| I-324 | Cl | Cl | Me | Et | 2-Me | SO₂CF₂CF₂H |
| I-325 | F | F | Me | Et | 2,3-Me₂ | SO₂CF₂CF₂H |
| I-326 | Cl | F | Me | Et | 2,3-Me₂ | SO₂CF₂CF₂H |
| I-327 | Cl | Cl | Me | Et | 2,3-Me₂ | SO₂CF₂CF₂H |
| I-328 | F | F | H | Me | H | SCF₂CF₃ |
| I-329 | Cl | F | H | Me | H | SCF₂CF₃ |
| I-330 | Cl | Cl | H | Me | H | SCF₂CF₃ |
| I-331 | Cl | F | H | Me | 2-F | SCF₂CF₃ |
| I-332 | Cl | Cl | H | Me | 2-F | SCF₂CF₃ |
| I-333 | Cl | F | H | Me | 2-Cl | SCF₂CF₃ |
| I-334 | Cl | Cl | H | Me | 2-Cl | SCF₂CF₃ |
| I-335 | Cl | F | H | Me | 2-Me | SCF₂CF₃ |
| I-336 | Cl | Cl | H | Me | 2-Me | SCF₂CF₃ |
| I-337 | Cl | F | H | Me | 2,3-Me₂ | SCF₂CF₃ |
| I-338 | Cl | Cl | H | Me | 2,3-Me₂ | SCF₂CF₃ |
| I-339 | F | F | Me | Me | H | SCF₂CF₃ |
| I-340 | Cl | F | Me | Me | H | SCF₂CF₃ |
| I-341 | Cl | Cl | Me | Me | H | SCF₂CF₃ |
| I-342 | Cl | F | Me | Me | 2-F | SCF₂CF₃ |
| I-343 | Cl | Cl | Me | Me | 2-F | SCF₂CF₃ |
| I-344 | Cl | F | Me | Me | 2-Cl | SCF₂CF₃ |
| I-345 | Cl | Cl | Me | Me | 2-Cl | SCF₂CF₃ |
| I-346 | Cl | F | Me | Me | 2-Me | SCF₂CF₃ |
| I-347 | Cl | Cl | Me | Me | 2-Me | SCF₂CF₃ |
| I-348 | Cl | F | Me | Me | 2,3-Me₂ | SCF₂CF₃ |
| I-349 | Cl | Cl | Me | Me | 2,3-Me₂ | SCF₂CF₃ |
| I-350 | F | F | H | Et | H | SCF₂CF₃ |
| I-351 | Cl | F | H | Et | H | SCF₂CF₃ |
| I-352 | Cl | Cl | H | Et | H | SCF₂CF₃ |
| I-353 | F | F | H | Et | 2-F | SCF₂CF₃ |
| I-354 | Cl | F | H | Et | 2-F | SCF₂CF₃ |
| I-355 | Cl | Cl | H | Et | 2-F | SCF₂CF₃ |
| I-356 | F | F | H | Et | 2-Cl | SCF₂CF₃ |
| I-357 | Cl | F | H | Et | 2-Cl | SCF₂CF₃ |
| I-358 | Cl | Cl | H | Et | 2-Cl | SCF₂CF₃ |
| I-359 | F | F | H | Et | 2-Me | SCF₂CF₃ |
| I-360 | Cl | F | H | Et | 2-Me | SCF₂CF₃ |
| I-361 | Cl | Cl | H | Et | 2-Me | SCF₂CF₃ |
| I-362 | F | F | H | Et | 2,3-Me₂ | SCF₂CF₃ |
| I-363 | Cl | F | H | Et | 2,3-Me₂ | SCF₂CF₃ |
| I-364 | Cl | Cl | H | Et | 2,3-Me₂ | SCF₂CF₃ |
| I-365 | F | F | Me | Et | H | SCF₂CF₃ |
| I-366 | Cl | F | Me | Et | H | SCF₂CF₃ |
| I-367 | Cl | Cl | Me | Et | H | SCF₂CF₃ |
| I-368 | F | F | Me | Et | 2-F | SCF₂CF₃ |
| I-369 | Cl | F | Me | Et | 2-F | SCF₂CF₃ |
| I-370 | Cl | Cl | Me | Et | 2-F | SCF₂CF₃ |
| I-371 | F | F | Me | Et | 2-Cl | SCF₂CF₃ |
| I-372 | Cl | F | Me | Et | 2-Cl | SCF₂CF₃ |
| I-373 | Cl | Cl | Me | Et | 2-Cl | SCF₂CF₃ |
| I-374 | F | F | Me | Et | 2-Me | SCF₂CF₃ |
| I-375 | Cl | F | Me | Et | 2-Me | SCF₂CF₃ |
| I-376 | Cl | Cl | Me | Et | 2-Me | SCF₂CF₃ |
| I-377 | F | F | Me | Et | 2,3-Me₂ | SCF₂CF₃ |
| I-378 | Cl | F | Me | Et | 2,3-Me₂ | SCF₂CF₃ |
| I-379 | Cl | Cl | Me | Et | 2,3-Me₂ | SCF₂CF₃ |
| I-380 | F | F | H | Me | H | SOCF₂CF₃ |
| I-381 | Cl | F | H | Me | H | SOCF₂CF₃ |
| I-382 | Cl | Cl | H | Me | H | SOCF₂CF₃ |
| I-383 | F | F | H | Me | 2-F | SOCF₂CF₃ |
| I-384 | Cl | F | H | Me | 2-F | SOCF₂CF₃ |
| I-385 | Cl | Cl | H | Me | 2-F | SOCF₂CF₃ |
| I-386 | F | F | H | Me | 2-Cl | SOCF₂CF₃ |
| I-387 | Cl | F | H | Me | 2-Cl | SOCF₂CF₃ |
| I-388 | Cl | Cl | H | Me | 2-Cl | SOCF₂CF₃ |
| I-389 | F | F | H | Me | 2-Me | SOCF₂CF₃ |
| I-390 | Cl | F | H | Me | 2-Me | SOCF₂CF₃ |
| I-391 | Cl | Cl | H | Me | 2-Me | SOCF₂CF₃ |
| I-392 | F | F | H | Me | 2,3-Me₂ | SOCF₂CF₃ |
| I-393 | Cl | F | H | Me | 2,3-Me₂ | SOCF₂CF₃ |
| I-394 | Cl | Cl | H | Me | 2,3-Me₂ | SOCF₂CF₃ |
| I-395 | F | F | Me | Me | H | SOCF₂CF₃ |
| I-396 | Cl | F | Me | Me | H | SOCF₂CF₃ |
| I-397 | Cl | Cl | Me | Me | H | SOCF₂CF₃ |
| I-398 | F | F | Me | Me | 2-F | SOCF₂CF₃ |
| I-399 | Cl | F | Me | Me | 2-F | SOCF₂CF₃ |
| I-400 | Cl | Cl | Me | Me | 2-F | SOCF₂CF₃ |
| I-401 | F | F | Me | Me | 2-Cl | SOCF₂CF₃ |
| I-402 | Cl | F | Me | Me | 2-Cl | SOCF₂CF₃ |
| I-403 | Cl | Cl | Me | Me | 2-Cl | SOCF₂CF₃ |
| I-404 | F | F | Me | Me | 2-Me | SOCF₂CF₃ |
| I-405 | Cl | F | Me | Me | 2-Me | SOCF₂CF₃ |
| I-406 | Cl | Cl | Me | Me | 2-Me | SOCF₂CF₃ |
| I-407 | F | F | Me | Me | 2,3-Me₂ | SOCF₂CF₃ |
| I-408 | Cl | F | Me | Me | 2,3-Me₂ | SOCF₂CF₃ |
| I-409 | Cl | Cl | Me | Me | 2,3-Me₂ | SOCF₂CF₃ |
| I-410 | F | F | H | Et | H | SOCF₂CF₃ |
| I-411 | Cl | F | H | Et | H | SOCF₂CF₃ |
| I-412 | Cl | Cl | H | Et | H | SOCF₂CF₃ |
| I-413 | F | F | H | Et | 2-F | SOCF₂CF₃ |
| I-414 | Cl | F | H | Et | 2-F | SOCF₂CF₃ |
| I-415 | Cl | Cl | H | Et | 2-F | SOCF₂CF₃ |
| I-416 | F | F | H | Et | 2-Cl | SOCF₂CF₃ |
| I-417 | Cl | F | H | Et | 2-Cl | SOCF₂CF₃ |
| I-418 | Cl | Cl | H | Et | 2-Cl | SOCF₂CF₃ |
| I-419 | F | F | H | Et | 2-Me | SOCF₂CF₃ |
| I-420 | Cl | F | H | Et | 2-Me | SOCF₂CF₃ |
| I-421 | Cl | Cl | H | Et | 2-Me | SOCF₂CF₃ |
| I-422 | F | F | H | Et | 2,3-Me₂ | SOCF₂CF₃ |
| I-423 | Cl | F | H | Et | 2,3-Me₂ | SOCF₂CF₃ |
| I-424 | Cl | Cl | H | Et | 2,3-Me₂ | SOCF₂CF₃ |
| I-425 | F | F | Me | Et | H | SOCF₂CF₃ |
| I-426 | Cl | F | Me | Et | H | SOCF₂CF₃ |
| I-427 | Cl | Cl | Me | Et | H | SOCF₂CF₃ |
| I-428 | F | F | Me | Et | 2-F | SOCF₂CF₃ |
| I-429 | Cl | F | Me | Et | 2-F | SOCF₂CF₃ |
| I-430 | Cl | Cl | Me | Et | 2-F | SOCF₂CF₃ |
| I-431 | F | F | Me | Et | 2-Cl | SOCF₂CF₃ |
| I-432 | Cl | F | Me | Et | 2-Cl | SOCF₂CF₃ |
| I-433 | Cl | Cl | Me | Et | 2-Cl | SOCF₂CF₃ |
| I-434 | F | F | Me | Et | 2-Me | SOCF₂CF₃ |
| I-435 | Cl | F | Me | Et | 2-Me | SOCF₂CF₃ |
| I-436 | Cl | Cl | Me | Et | 2-Me | SOCF₂CF₃ |
| I-437 | F | F | Me | Et | 2,3-Me₂ | SOCF₂CF₃ |
| I-438 | Cl | F | Me | Et | 2,3-Me₂ | SOCF₂CF₃ |
| I-439 | Cl | Cl | Me | Et | 2,3-Me₂ | SOCF₂CF₃ |
| I-440 | F | F | H | Me | H | SO₂CF₂CF₃ |
| I-441 | Cl | F | H | Me | H | SO₂CF₂CF₃ |
| I-442 | Cl | Cl | H | Me | H | SO₂CF₂CF₃ |
| I-443 | F | F | H | Me | 2-F | SO₂CF₂CF₃ |
| I-444 | Cl | F | H | Me | 2-F | SO₂CF₂CF₃ |
| I-445 | Cl | Cl | H | Me | 2-F | SO₂CF₂CF₃ |
| I-446 | F | F | H | Me | 2-Cl | SO₂CF₂CF₃ |
| I-447 | Cl | F | H | Me | 2-Cl | SO₂CF₂CF₃ |
| I-448 | Cl | Cl | H | Me | 2-Cl | SO₂CF₂CF₃ |
| I-449 | F | F | H | Me | 2-Me | SO₂CF₂CF₃ |
| I-450 | Cl | F | H | Me | 2-Me | SO₂CF₂CF₃ |
| I-451 | Cl | Cl | H | Me | 2-Me | SO₂CF₂CF₃ |
| I-452 | F | F | H | Me | 2,3-Me₂ | SO₂CF₂CF₃ |
| I-453 | Cl | F | H | Me | 2,3-Me₂ | SO₂CF₂CF₃ |
| I-454 | Cl | Cl | H | Me | 2,3-Me₂ | SO₂CF₂CF₃ |
| I-455 | F | F | Me | Me | H | SO₂CF₂CF₃ |
| I-456 | Cl | F | Me | Me | H | SO₂CF₂CF₃ |
| I-457 | Cl | Cl | Me | Me | H | SO₂CF₂CF₃ |
| I-458 | F | F | Me | Me | 2-F | SO₂CF₂CF₃ |
| I-459 | Cl | F | Me | Me | 2-F | SO₂CF₂CF₃ |
| I-460 | Cl | Cl | Me | Me | 2-F | SO₂CF₂CF₃ |
| I-461 | F | F | Me | Me | 2-Cl | SO₂CF₂CF₃ |
| I-462 | Cl | F | Me | Me | 2-Cl | SO₂CF₂CF₃ |
| I-463 | Cl | Cl | Me | Me | 2-Cl | SO₂CF₂CF₃ |
| I-464 | F | F | Me | Me | 2-Me | SO₂CF₂CF₃ |
| I-465 | Cl | F | Me | Me | 2-Me | SO₂CF₂CF₃ |
| I-466 | Cl | Cl | Me | Me | 2-Me | SO₂CF₂CF₃ |
| I-467 | F | F | Me | Me | 2,3-Me₂ | SO₂CF₂CF₃ |
| I-468 | Cl | F | Me | Me | 2,3-Me₂ | SO₂CF₂CF₃ |
| I-469 | Cl | Cl | Me | Me | 2,3-Me₂ | SO₂CF₂CF₃ |
| I-470 | F | F | H | Et | H | SO₂CF₂CF₃ |
| I-471 | Cl | F | H | Et | H | SO₂CF₂CF₃ |
| I-472 | Cl | Cl | H | Et | H | SO₂CF₂CF₃ |
| I-473 | F | F | H | Et | 2-F | SO₂CF₂CF₃ |
| I-474 | Cl | F | H | Et | 2-F | SO₂CF₂CF₃ |
| I-475 | Cl | Cl | H | Et | 2-F | SO₂CF₂CF₃ |
| I-476 | F | F | H | Et | 2-Cl | SO₂CF₂CF₃ |
| I-477 | Cl | F | H | Et | 2-Cl | SO₂CF₂CF₃ |
| I-478 | Cl | Cl | H | Et | 2-Cl | SO₂CF₂CF₃ |
| I-479 | F | F | H | Et | 2-Me | SO₂CF₂CF₃ |
| I-480 | Cl | F | H | Et | 2-Me | SO₂CF₂CF₃ |
| I-481 | Cl | Cl | H | Et | 2-Me | SO₂CF₂CF₃ |
| I-482 | F | F | H | Et | 2,3-Me₂ | SO₂CF₂CF₃ |
| I-483 | Cl | F | H | Et | 2,3-Me₂ | SO₂CF₂CF₃ |
| I-484 | Cl | Cl | H | Et | 2,3-Me₂ | SO₂CF₂CF₃ |
| I-485 | F | F | Me | Et | H | SO₂CF₂CF₃ |
| I-486 | Cl | F | Me | Et | H | SO₂CF₂CF₃ |
| I-487 | Cl | Cl | Me | Et | H | SO₂CF₂CF₃ |
| I-488 | F | F | Me | Et | 2-F | SO₂CF₂CF₃ |
| I-489 | Cl | F | Me | Et | 2-F | SO₂CF₂CF₃ |
| I-490 | Cl | Cl | Me | Et | 2-F | SO₂CF₂CF₃ |
| I-491 | F | F | Me | Et | 2-Cl | SO₂CF₂CF₃ |
| I-492 | Cl | F | Me | Et | 2-Cl | SO₂CF₂CF₃ |
| I-493 | Cl | Cl | Me | Et | 2-Cl | SO₂CF₂CF₃ |
| I-494 | F | F | Me | Et | 2-Me | SO₂CF₂CF₃ |
| I-495 | Cl | F | Me | Et | 2-Me | SO₂CF₂CF₃ |
| I-496 | Cl | Cl | Me | Et | 2-Me | SO₂CF₂CF₃ |
| I-497 | F | F | Me | Et | 2,3-Me₂ | SO₂CF₂CF₃ |
| I-498 | Cl | F | Me | Et | 2,3-Me₂ | SO₂CF₂CF₃ |
| I-499 | Cl | Cl | Me | Et | 2,3-Me₂ | SO₂CF₂CF₃ |

The pesticide of the present invention may be the compound (I) or a salt thereof itself, but is usually prepared, if necessary, by adding a surfactant or other auxiliary agent for preparation, as an emulsion, a solution, a microemulsion, a flowable formulation, an oil solution, a wettable powder, a water soluble power, a sol formulation, a powder, a granule, a fine granule, a seed coating agent, an immersion coating formulation, a smoking agent, an aerosol, a tablet, a microcapsule, a spray formulation, an EW agent, an ointment, a poison bait, a capsule, a pellet, a film coating formulation, a painting formulation, an injectable, a shampoo preparation or the like, which contains compound (I) or a salt thereof and inert carriers such as a solid carrier, a liquid carrier and a gaseous carrier.

Examples of the liquid carrier used for preparation include water, alcohols (for example, methyl alcohol, ethyl alcohol, n-propyl alcohol, isopropyl alcohol, ethylene glycol and the like), ketones (for example, acetone, methyl ethyl ketone, methyl isobutyl ketone, cyclohexanone and the like), ethers (for example, tetrahydrofuran, ethylene glycol monomethyl ether, diethylene glycol monomethyl ether, propylene glycol monomethyl ether and the like), aliphatic hydrocarbons (for example, kerosine, fuel oil, machine oil and the like), aromatic hydrocarbons (for example, toluene, xylene, solvent naphtha, methyl naphthalene and the like), halogenated hydrocarbons (for example, dichloromethane, chloroform, carbon tetrachloride and the like), acid amides (for example, N,N-dimethylformamide, N,N-dimethylacetoamide, N-methylpyrrolidone and the like), esters (for example, ethyl acetate, butyl acetate, fatty glycerin ester, γ-butylolactone and the like), and nitrites (for example, acetonitrile, propyonitrile and the like).

Examples of the solid carrier include vegetable powder (for example, soybean powder, tobacco powder, wheat powder, woodmeal and the like), mineral powder (for example, clays such as kaolin, bentonite, acid clay and the like, talcs such as talc powder, agalmatolite powder and the like, silicas such as diatomaceous earth, mica powder and the like), alumina, sulfur powder, activated carbon, calcium carbonate, ammonium sulfate, sodium hydrogen carbonate, lactose and urea.

In addition, examples of the ointment base include polyethylene glycol; pectin; polyhydric alcohol ester of higher fatty acid such as monostearic acid glycerin ester and the like; cellulose derivatives such as methylcellulose and the like; sodium alginate; bentonite; higher alcohol; polyhydric alcohol such as glycerin and the like; vaseline; white vaseline; liquid paraffin; lard; various vegetable oils; lanolin; dehydrated lanolin; hardened oil; resins and a mixture of these and a surfactant.

Examples of the surfactant include nonionic and anionic surfactants such as soaps, polyoxyethylene alkyl aryl ethers [e.g. Neugen (trade name), E•A142 (trade name); manufactured by Dai-ichi Kogyo Seiyaku Co., Ltd., Nonal (trade name); manufactured by Toho Chemical Industries Co., Ltd.], alkyl sulfate salts [e.g. Emar 10 (trade name), Emar 40 (trade name); manufactured by Kao Corporation], alkylbenzene sulfonic acid salts [e.g. Neogen (trade name), Neogen T (trade name); manufactured by Dai-ichi Kogyo Seiyaku Co., Ltd., Neoperex; manufactured by Kao Corporation], polyethylene glycol ethers [e.g., Nonipol 85 (trade name), Nonipol 100 (trade name), Nonipol 160 (trade name); manufactured by Sanyo Chemical Industries, Ltd.], polyhydric alcohol esters [e.g. Tween 20 (trade name), Tween 80 (trade name); manufactured by Kao Corporation], alkylsulfosuccinic acid salts [e.g. Sanmolin OT20 (trade name); manufactured by Sanyo Chemical Industries, Ltd.], alkylnaphthalene sulfonic acid salts [e.g. Newcalgen EX70 (trade name); manufactured by Takemoto Oil & Fat Co., Ltd.], alkenyl sulfonic acid salts [e.g. Solpol 5115 (trade name); manufactured by Toho Chemical Industries Co., Ltd.] and the like.

The ratio of Compound (I) or a salt thereof contained in the preparation of the pesticide of the present invention is usually 0.1 to 80% by weight, preferably 1 to 20% by weight relative to the total amount of pesticide of the present invention. Specifically, when the compound is used as an emulsion, a solution, a wettable powder or the like, usually about 1 to 80% by weight, preferably about 1 to 20% by weight is suitable. When used as an oil solution or a powder, usually about 0.1 to 50% by weight, preferably about 0.1 to 20% by weight is suitable. When used in a granule, usually about 5 to 50% by weight, preferably about 1 to 20% by weight is suitable.

The pesticide of the present invention can be used in admixture with other insecticides, acaricides, nematocides, fungicides, herbicides, plant growth regulators, synergists, attractants, repellents, safeners, pigments, fertilizers and the like.

Representative examples of the fungicides, plant growth regulators and herbicides that can be used by mixing with the pesticide of the present invention, and the pesticide and the like such as insecticides, acaricides and nematocides are shown below.

Active ingredients of the insecticide include, for example,

(1) Organic Phosphorous Compounds

Acephate, Aluminium phosphide, butathiofos, cadusafos, chlorethoxyfos, chlorfenvinphos, chlorpyrifos, chlorpyrifos-methyl, cyanophos (CYAP), diazinon, DCIP (dichlorodiisopropyl ether), dichlofenthion (ECP), dichlorvos (DDVP), dimethoate, dimethylvinphos, disulfoton, EPN, ethion, ethoprophos, etrimfos, fenthion (MPP), fenitrothion (MEP), fosthiazate, formothion, Hydrogen phosphide, isofenphos, isoxathion, malathion, mesulfenfos, methidathion (DMTP), monocrotophos, naled (BRP), oxydeprofos (ESP), parathion, phosalone, phosmet (PMP), pirimiphos-methyl, pyridafenthion, quinalphos, phenthoate (PAP), profenofos, propaphos, prothiofos, pyraclorfos, salithion, sulprofos, tebupirimfos, temephos, tetrachlorvinphos, terbufos, thiometon, trichlorphon (DEP), vamidothion and the like;

(2) Carbamate Compounds

Alanycarb, bendiocarb, benfuracarb, BPMC, carbaryl, carbofuran, carbosulfan, cloethocarb, ethiofencarb, fenobucarb, fenothiocarb, fenoxycarb, furathiocarb, isoprocarb (MIPC), metolcarb, methomyl, methiocarb, NAC, oxamyl, pirimicarb, propoxur (PHC), XMC, thiodicarb, xylylcarb and the like;

(3) Synthetic Pyrethroid Compounds

Acrinathrin, allethrin, benfluthrin, beta-cyfluthrin, bifenthrin, cycloprothrin, cyfluthrin, cyhalothrin, cypermethrin, deltamethrin, esfenvalerate, ethofenprox, fenpropathrin, fenvalerate, flucythrinate, flufenoprox, flumethrin, fluvalinate, halfenprox, imiprothrin, permethrin, prallethrin, pyrethrins, resmethrin, sigma-cypermethrin, silafluofen, tefluthrin, tralomethrin, transfluthrin, 2,3,5,6-tetrafluoro-4-(methoxymethyl)benzyl (EZ)-(1RS,3RS;1RS,3SR)-2,2-dimethyl-3-prop-1-enylcyclopropanecarboxylate, 2,3,5,6-tetrafluoro-4-methylbenzyl (EZ)-(1RS,3RS;1RS,3SR)-2,2-dimethyl-3-prop-1-enylcyclopropanecarboxylate, 2,3,5,6-tetrafluoro-4-(methoxymethyl)benzyl (1RS,3RS;1RS,3SR)-2,2-dimethyl-3-(2-methyl-1-propenyl)cyclopropanecarboxylate and the like;

(4) Nereistoxin Compounds

Cartap, bensultap, thiocyclam, monosultap, bisultap and the like;

(5) Neonicotinoid Compounds

Imidacloprid, nitenpyram, acetamiprid, thiamethoxam, thiacloprid, dinotefuran, clothianidin and the like;

(6) Benzoylurea Compounds

Chlorfluazuron, bistrifluoron, diafenthiuron, diflubenzuron, fluazuron, flucycloxuron, flufenoxuron, hexaflumuron, lufenuron, novaluron, noviflumuron, teflubenzuron, triflumuron and the like;

(7) Phenylpyrazole Compounds

Acetoprole, ethiprole, fipronil, vaniliprole, pyriprole, pyrafluprole and the like;

(8) Bt Toxins

Live spores and produced crystal toxin derived from bacillus thuringiensis, and a mixture thereof;

(9) Hydrazine Compounds

Chromafenozide, halofenozide, methoxyfenozide, tebufenozide and the like;

(10) Organic Chlorine Compounds

Aldrin, dieldrin, dienochlor, endosulfan, methoxychlor and the like;

(11) Natural Insecticides

Machine oil, nicotine-sulfate and the like;

(12) Other Insecticides

Avermectin-B, bromopropylate, buprofezin, chlorphenapyr, cyromazine, D-D (1,3-Dichloropropene), emamectin-benzoate, fenazaquin, flupyrazofos, hydroprene, indoxacarb, metoxadiazone, milbemycin-A, pymetrozine, pyridalyl, pyriproxyfen, spinosad, sulfluramid, tolfenpyrad, triazamate, flubendiamide, SI-0009, cyflumetofen, Arsenic acid, benclothiaz, Calcium cyanamide, Calcium polysulfide, chlordane, DDT, DSP, flufenerim, flonicamid, flurimfen, formetanate, metam-ammonium, metam-sodium, Methyl bromide, nidinotefuran, Potassium oleate, protrifenbute, spiromesifen, Sulfur, metaflumizone, spirotetramat, NNI-0101, Chlorantraniliprole

the compound represented by the below formula:

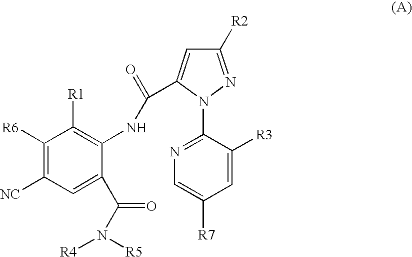

wherein,
R¹ represents a methyl group, a chlorine atom, a bromine atom, or a fluorine atom,
R² represents a fluorine atom, a chlorine atom, a bromine atom, a C1-C4 haloalkyl group or a C1-C4 haloalkoxy group,
R³ represents a fluorine atom, a chlorine atom, or a bromine atom,
R⁴ represents a hydrogen atom, a cyano group, a methylthio group, a methylsulfinyl group, a methylsulfonyl group, or a C1-C4 alkyl group optionally substituted with at least one group selected from the group consisting of methoxy group, C3-C4 alkenyl group, a C3-C4 alkynyl group and C3-C5 cycloalkyl,
R⁵ represents a hydrogen atom, or a methyl group,
R⁶ represents a hydrogen atom, a fluorine atom, or a chlorine atom,
R⁷ represents a hydrogen atom, a fluorine atom, or a chlorine atom; and the like.

Active ingredients of the acaricides include, for example, acequinocyl, amitraz, benzoximate, bifenazate, bromopropylate, chinomethionat, chlorobenzilate, CPCBS (chlorfenson), clofentezine, cyflumetofen, kelthane (dicofol), etoxazole, fenbutatin oxide, fenothiocarb, fenpyroximate, fluacrypyrim, fluproxyfen, hexythiazox, propargite (BPPS), polynactins, pyridaben, Pyrimidifen, tebufenpyrad, tetradifon, spirodiclofen, amidoflumet and the like.

Active ingredients of the nematocides include, for example, DCIP, fosthiazate, levamisol, methylsothiocyanate, morantel tartarate and the like.

Active ingredients of the fungicides include, for example, acibenzolar-S-methyl, amobam, ampropylfos, anilazine, azoxystrobin, benalaxyl, benodanil, benomyl, benthiavalicarb, benthiazole, bethoxazin, bitertanol, blasticidin-S, Bordeaux mixture, boscalid, bromuconazole, buthiobate, Calcium hypochlorite, Calcium polysulfide, captan, carbendazol, carboxin, carpropamid, chlobenthiazone, chloroneb, chloropicrin, chlorothalonil (TPN), chlorthiophos, Cinnamaldehyde, clozylacon, CNA (2,6-Dichloro-4-nitroaniline), Copper hydroxide, Copper sulfate, cyazofamid, cyfluphenamid, cymoxanil, cyproconazole, cyprodinil, cyprofuram, dazomet, debacarb, dichlofluanid, D-D (1,3-Dichloropropene), diclocymet, diclomezine, diethofencarb, difenoconazole, diflumetorim, dimefluazole, dimethirimol, dimethomorph, diniconazole-M, dinocap, edifenphos, epoxiconazole, nickel dimethyldithiocarbamate, etaconazole, ethaboxam, ethirimol, etridiazole, famoxadone, fenamidone, fenarimol, fenbuconazole, Fendazosulam, fenhexamid, fenoxanil, fenpiclonil, fenpropidin, fenpropimorph, fentiazon, fentin hydroxide, ferimzone, fluazinam, fludioxonil, flumetover, flumorph, fluoroimide, fluotrimazole, fluoxastrobin, fluquinconazole, flusilazole, flusulfamide, flutolanil, flutriafol, fosetyl-Al, fthalide, fuberidazole, furalaxyl, furametpyr, furcarbanil, furconazole-cis, hexaconazole, hymexazol, IBP, imazalil, imibenconazole, iminoctadine-albesilate, iminoctadine-triacetate, iodocarb, ipconazole, iprodione, iprovalicarb, isoprothiolane, kasugamycin, kresoxim-methyl, mancozeb, maneb, mepanipyrim, mepronil, metalaxyl, metalaxyl-M, metam-sodium, methasulfocarb, Methyl bromide, metconazole, methfuroxam, metominostrobin, metrafenone, metsulfovax, mildiomycin, milneb, myclobutanil, myclozolin, nabam, orysastrobin, ofurace, oxadixyl, oxolinic acid, oxpoconazole, oxycarboxin, oxytetracycline, pefurazoate, penconazole, pencycuron, picoxystrobin, polycarbamate, polyoxin, Potassium hydrogen carbonate, probenazole, prochloraz, procymidone, propamocarb-hydrochloride, propiconaole, propineb, proquinazid, prothiocarb, prothioconazole, pyracarbolid, pyraclostrobin, pyrazophos, pyributicarb, pyrifenox, pyrimethanil, pyroquilon, quinoxyfen, quintozene (PCNB), silthiopham, simeconazole, sipconazole, Sodium bibarbonate, sodium hypochlorite, spiroxamine, ((E)-2[2-(2,5-dimethylphenoxymethyl)phenyl]-2-methoxyimino-N-methylacetamide), streptomycin, Sulfur, tebuconazole, tecloftalam, tetraconazole, thiabendazole, thiadinil, thiram (TMTD), thifluzamide, thiophanate-methyl, tolclofos-methyl, TPN, triadimefon, triadimenol, triazoxide, triclamide, tricyclazole, tridemorph, triflumizole, trifloxystrobin, triforine, triticonazole, validamycin, vinclozolin, viniconazole, zineb, ziram and zoxamide.

Active ingredients of the herbicides and plant growth regulators include, for example, Abscisic acid, acetochlor, acifluorfen-sodium, alachlor, alloxydim, ametryn, amicarbazone, amidosulfuron, aminoethoxyvinylglycine, aminopyralid, AC94, 377, amiprofos-methyl, ancymidol, asulam, atrazine, aviglycine, azimsulfuron, beflubutamid, benfluralin, benfuresate, bensulfuron-methyl, bensulide (SAP), bentazone, benthiocarb, benzamizole, benzfendizone, benzobicyclon, benzofenap, benzyl adenine, benzylaminopurine, bialaphos, bifenox, Brassinolide, bromacil, bromobutide, butachlor, butafenacil, butamifos, butylate, cafenstrole, Calcium carbonate, Calcium peroxide, carbaryl, chlomethoxynil, chloridazon, chlorimuron-ethyl, chlorphthalim, chlorpropham, chlorsulfuron, chlorthal-dimethyl, chlorthiamid (DCBN), choline chloride, cinidon-ethyl, cinmethylin, cinosulfuron, clethodim, clomeprop, cloxyfonac-sodium, chlormequat chloride, 4-CPA (4-chlorophenoxyacetic acid), cliprop, clofencet, cumyluron, cyanazine, cyclanilide, cyclosulfamron, cyhalofop-butyl, 2,4-Dichlorophenoxyacetic acid salts, dichlorprop (2,4-DP), daimuron, dalapon (DPA), dimethenamid-P, daminozide, dazomet, n-Decyl alcohol, dicamba-sodium (MDBA), dichlobenil (DBN), diflufenican, dikegulac, dimepiperate, dimethametryn, dimethenamid, diquat, dithiopyr, diuron, endothal, epocholeone, esprocarb, ethephon, ethidimuron, ethoxysulfuron, ethychlozate, etobenzanid, fenarimol, fenoxaprop-ethyl, fentrazamide, flazasulfuron, florasulam, fluazifop-butyl, fluazolate, flucarbazone, flufenacet, flufenpyr, flumetralin), flumioxazin, flupropanate-sodium, flupyrsulfuron-methyl-sodium, flurprimidol, fluthiacet-methyl, foramsulfuron, forchlorfenuron, formesafen, gibberellin, glufosinate, glyphosate, halosulfuron-methyl, hexazinone, imazamox, imazapic, imazapyr, imazaquin, imazosulfuron, inabenfide, Indole acetic acid (IAA), Indole butyric acid, iodosulfuron, ioxynil-octanoate, isouron, isoxachlortole, isoxadifen, karbutilate, lactofen, lenacil, linuron, LGC-42153, Maleic hydrazide, mecoprop (MCPP), 2-Methyl-4-chlorophenoxyacetic acid salts, MCPA-thioethyl, 2-Methyl-4-chlorophenoxybutanoic acid ethyl ester, mefenacet, mefluidide, mepiquat, mesosulfuron, mesotrione, methyl daimuron, metamifop, metolachlor, metribuzin, metsulfuron-methyl, molinate, naphthylacetic acid, 1-naphthaleneacetamide, naproanilide, napropamide, n-decyl alcohol, nicosulfuron, n-phenylphthalamic acid, orbencarb, oxadiazon, oxaziclomefone, oxine-sulfate, paclobutrazol, paraquat, Pelargonic acid, pendimethalin, penoxsulam, pentoxazone, pethoxamide, phenmedipham, picloram, picolinafen, piperonyl butoxide, piperophos, pretilachlor, primisulfuron-methyl, procarbazone, prodiamine, profluazol, profoxydim, prohexadione-calcium, prohydrojasmon, prometryn, propanil, propoxycarbazone, propyzamide, pyraclonil, pyraflufen-ethyl, pyrazolate, pyrazosulfuron-ethyl, pyrazoxyfen, pyribenzoxim, pyributicarb, pyridafol, pyridate, pyriftalid, pyriminobac-methyl, pyrithiobac, quiclorac, quinoclamine, quizalofop-ethyl, rimsulfuron, sethoxydim, siduron, simazine, simetryn, Sodium chlorate, sulfosulfuron, swep (MCC), tebuthiuron, tepraloxydim, terbacil, terbucarb (MBPMC), thenylchlor, thiazafluoron, thidiazuron, thifensulfuron-methyl, triaziflam, tribufos, triclopyr, tridiphane, trifloxysulfuron, trifluralin, trinexapac-ethyl, tritosulfuron, uniconazole-P and vemolate (PPTC).

The pesticide of the present invention can also be used further in admixture with a synergist such as piperonyl butoxide, sesamex, N-(2-ethylhexyl)-8,9,10-trinorborn-5-en-2,3-dicarboxylmide (MGK 264), WARF-antiresistant and diethylmaleate, and furthermore, may be used in admixture with a safener such as benoxacor, cloquintocet-mexyl, cyometrinil, daimuron, dichlormid, fenchlorazole-ethyl, fenclorim, flurazole, fluxofenim, furilazole, mefenpyr-diethyl, MG191, naphthalic anhydride and oxabetrinil.

Examples of the pest against which compound (I) or a salt thereof has an activity include arthropods such as insect pests, acarine pests and the like, and nematode pests. Specific examples are listed below:

Hemiptera: Delphacidae such as Laodelphax striatellus, Nilaparvata lugens, Sogatella furcifera and the like; Deltocephalidae such as Nephotettix cincticeps, Nephotettix virescens and the like; Aphididae such as Aphis gossypii, Myzus persicae, Brevicoryne brassicae, Macrosiphum euphorbiae, Aulacorthum solani, Rhopalosiphum padi, Toxoptera citricidus and the like; Pentatomidae such as Nezara antennata, Riptortus clavetus, Leptocorisa chinensis, Eysarcoris parvus, Halyomorpha mista and the like; Aleyrodidae such as Trialeurodes vaporariorum, Bemisia argentifolii and the like; Coccidae such as Aonidiella aurantii, Comstockaspis perniciosa, Unaspis citri, Ceroplastes rubens, Icerya purchasi and the like; Tingidae, Psyllidae, and the like.

Lepidoptera: Pyralidae such as Chilo suppressalis, Tryporyza incertulas, Cnaphalocrocis medinalis, Notarcha derogata, Plodia interpunctella, Ostrinia furnacalis, Hellula undalis, Pediasia teterrellus and the like; Noctuidae such as Spodoptera litura, Spodoptera exigua, Pseudaletia separata, Mamestra brassicae, Agrotis ipsilon, Plusia nigrisigna, Thoricoplusia spp., Heliothis spp., Helicoverpa spp., and the like; Pieridae such as Pieris rapae and the like; Tortricidae such as Adoxophyes spp., Grapholita molesta, Leguminivora glycinivorella, Matsumuraeses azukivora, Adoxophyes orana fasciata, Adoxophyes sp., Homona magnanima, Archips fuscocupreanus, Cydia pomonella and the like; Gracillariidae such as Caloptilia theivora, Phyllonorycter ringoneella and the like; Carposinidae such as Carposina niponensis and the like; Lyonetiidae such as Lyonetia spp. and the like; Lymantriidae such as Lymantria spp., Euproctis spp., and the like; Yponomeutidae such as Plutella xylostella and the like; Gelechiidae such as Pectinophora gossypiella, Phthorimaea operculella and the like; Arctiidae such as Hyphantria cunea and the like; Tineidae such as Tinea translucens, Tineola bisselliella and the like.

Thysanoptera: Thripidae such as Frankliniella occidentalis, Thrips parmi, Scirtothrips dorsalis, Thrips tabaci, Frankliniella intonsa and the like.

Diptera: Musca domestica, Culex popiens pallens, Tabanus trigonus, Hylemya antiqua, Hylemya platura, Anopheles sinensis, Agromyza oryzae, Hydrellia griseola, Chlorops oryzae, Dacus cucurbitae, Ceratitis capitata, Liriomyza trifolii and the like.

Coleoptera: Epilachna vigintioctopunctata, Aulacophora femoralis, Phyllotreta striolata, Oulema oryzae, Echinocnemus squameus, Lissorhoptrus oryzophilus, Anthonomus grandis, Callosobruchus chinensis, Sphenophorus venatus, Popillia japonica, Anomala cuprea, Diabrotica spp., Leptinotarsa decemlineata, Agriotes spp., Lasioderma serricorne, Anthrenus verbasci, Tribolium castaneum, Lyctus brunneus, Anoplophora malasiaca, Tomicus piniperda and the like.

Orthoptera: Locusta migratoria, Gryllotalpa africana, Oxya yezoensis, Oxya japonica and the like.

Hymenoptera: Athalia rosae, Acromyrmex spp., Solenopsis spp. and the like.

Nematode: Aphelenchoides besseyi, Nothotylenchus acris and the like.

Blattodea: Blattella germanica, Periplaneta fuliginosa, Periplaneta americana, Periplaneta brunnea, Blatta orientalis and the like.

Acarina: Tetranychidae such as Tetranychus urticae, Panonychus citri, Oligonychus spp., and the like; Eriophyidae such as Aculops pelekassi and the like; Tarsonemidae such as Polyphagotarsonemus latus and the like; Tenuipalpidae; Tuckerellidae; Ixodidae such as Haemaphysalis longicornis, Haemaphysalis flava, Dermacentor taiwanicus, Ixodes ovatus, Ixodes persulcatus, Boophilus microplus, Rhipicephalus sanguineus and the like; Acaridae such as Tyrophagus putrescentiae and the like; Epidermoptidae such as Dermatophagoides farinae, Dermatophagoides ptrenyssnus and the like; Cheyletidae such as Cheyletus eruditus, Cheyletus malaccensis, Cheyletus moorei and the like; and Dermanyssidae and the like.

Isoptera: Mastotermitidae, Termopsidae [Zootermopsis, Archotermopsis, Hodotermopsis, Porotermes, Stolotermes], Kalotermitidae [Kalotermes, Neotermes, Cryptotermes, Incistermes, Glyptotermes], Hodotermitidae [Hodotermes, Microhodotermes, Anacanthotermes], Rhinotermitidae [Reticulitermes, Heterotermes, Coptotermes, Schedolinotermes], Serritermitidae, Termitidae (Amitermes, Drepanotermes, Hopitalitermes, Trinervitermes, Macrotermes, Odontotermes, Microtermes, Nasutitermes, Pericapritermes, Anoplotermes);

Specifically, for example, Reticulitermes speratus, Coptotermes formosanus, Incisitermes minor, Cryptotermes domesticus, Odontotermes formosanus, Neotermes koshunensis, Glyptotermes satsumensis, Glyptotermes nakajimai, Glyptotermes fuscus, Glyptotermes kodamai, Glyptotermes kushimensis, Hodotermopsis japonica, Coptotermes guangzhoensis, Reticulitermes miyatakei, Reticulitermes flaviceps amamianus, Reticulitermes sp., Nasutitermes takasagoensis, Pericapritermes nitobei, Sinocapritermes mushae, Reticuliterumes flavipes, Reticulitermes hesperus, Reticulitermes virginicus, Reticulitermes tibialis, Heterotermes aureus, Zootermopsis nevadensis and the like;

Beetles: Lyctidae, Bostrychidae, Anobiidae, Cerambycidae and the like.

The method for controlling pests of the present invention is carried out by applying compound (I) or a salt thereof to pests directly, or habitats of pests.

In the method for controlling pests of the present invention, compound (I) or a salt thereof can be used as it is, but usually, a preparation of compound (I) or a salt thereof, or an aqueous dilution of the preparation is used.

Examples of the habitat of pests in the present invention include paddy fields, dry rice fields, fields, tea plantations, orchards, uncultivated fields, houses, seedling growing trays, nursery boxes, seedling growing medias, seedling growing mats, water culture mediums for hydroponic farm, and the like.

As a method for application, for example, a spray treatment, a soil treatment, a seed treatment and a hydroponic solution treatment are exemplified.

The spray treatment in the present invention is a method of treatment for expressing a controlling effect against pests by treating plant surface or pest itself with an active ingredient (compound (I) or a salt thereof), specifically for example, foliage application, spraying to tree trunk and the like. The soil treatment is a method of treatment for protecting crops from damages by pests, by treating soils, grown medias, irrigation solutions or the like with an active ingredient in order to penetrate and translocate from the root portion and the like into the plant interior of a crop to be protected from damages such as feeding and the like by pests, and specifically, for example, a planting hole treatment (planting hole spraying, soil-incorporation after planting hole treatment), a plant foot treatment (plant foot spraying, plant foot soil-incorporation, plant foot irrigation, plant foot treatment at latter half of raising seeding period), planting furrow treatment (planting furrow spraying, planting furrow soil-incorporation), planting row treatment (planting row spraying, planting row soil-incorporation, planting row spraying at growing period), planting row treatment at sowing (planting row spraying at sowing, planting row soil-incorporation at sowing), overall treatment (overall spraying, overall soil-incorporation), other spray treatment (foliar granule spraying at growing period, spraying under tree crown or around main stem, soil surface spraying, soil surface incorporation, sowing hole spraying, spraying on the ribbing ground, inter-plant spraying), other irrigation treatment (irrigation into soil, irrigation during raising seeding, injection treatment of pesticide solution, irrigation on plant foot, pesticide solution drip irrigation, chemigation), nursery box treatment (nursery box spraying, nursery box irrigation), nursery tray treatment (nursery tray spraying, nursery tray irrigation), nursery bed treatment (nursery bed spraying, nursery bed irrigation, nursery bed spraying in paddy field, immersion of nursery plant), seed bed soil-incorporation treatment (seed bed soil-incorporation, seed bed soil-incorporation before sowing), other treatment (growing media incorporation, plowing, surface soil-incorporation, soil incorporation into rain dropping, planting spot treatment, flower cluster granule spraying, paste fertilizer mixing), and the like are exemplified. The seed treatment is a method of treatment for expressing a controlling effect against pests by treating seeds, seed tubers, bulbs or the like of a crop to be protected from damages such as feeding and the like by pests directly, or neighborhood thereof, with an active ingredient, and specifically, for example, blowing treatment, painting treatment, immersion treatment, impregnation treatment, application treatment, film coating and a pellet coating treatment are exemplified. The hydroponic solution treatment is a method of treatment for protecting crops from damages by pests, by treating hydroponic solution or the like with an active ingredient in order to penetrate and translocate from the root portion and the like into the plant interior of a crop to be protected from damages such as feeding and the like by pests, and specifically, for example, hydroponic solution incorporation, hydroponic solution mixing, and the like are exemplified.

The amount of application of compound (I) or a salt thereof in the method for controlling pests in the present invention can be changed depending on the application time, application site, application method and the like, but in general, it is at a rate of 0.3 to 3000 g, preferably at a rate of 50 to 3000 g as an amount of the active ingredient (compound (I) or a salt thereof) per hectare. In addition, when the pesticide of the present invention is a wettable powder or the like, it may be diluted with water to use so that the final concentration of active ingredient comes to the range of about 0.1 to 1,000 ppm, preferably about 10 to 500 ppm.

Hereinafter, the present invention will be further illustrated by the following Production Examples, Examples, Preparation Examples, Test Examples and the Like, However, the present invention is not limited to these examples.

The elution in the column chromatography for Production Examples, Examples and Reference Production Examples was carried out under the observation by TLC (Thin Layer Chromatography). In the TLC observation, kieselgel 60F₂₅₄ manufactured by Merck & Co., Inc. was used as TLC plate; the solvent used as an elution solvent in column chromatography was used as developing solvent; and a UV detector was used for detection. Kieselgel 60 (70 to 230 meshes) manufactured by Merck & Co., Inc. was used as silica gel for column chromatography. As a medium pressure preparative high performance liquid chromatography, Ultrapack manufactured by Yamazen, Co., Ltd. (filler: silica gel) has been used. When a mixed solvent was used as developing solvent, the numeric value in parentheses shows a mixing ratio of solvents by volume. NMR spectra were proton NMR, and were determined with JEOL AL-400 (400 MHz) spectrometer and AVANCE 400 (400 MHz) spectrometer using tetramethylsilane as internal standard. All delta values were shown in ppm. The measurement-temperature is 25° C. unless otherwise mentioned, and the measurement temperature has been indicated for the rest.

Furthermore, the abbreviations used in the following Production Examples and Examples have the following meanings:

s: singlet, br: broad, brs; broad singlet, d: doublet, t: triplet, q: quartet, Me: methyl group, Et: ethyl group, Ph: phenyl group, Pr-n (or n-Pr): n-propyl, Pr-i (or i-Pr or ⁱPr): isopropyl, Pr-cyclo (or cyclo-Pr): cyclopropyl, Bu-n (or n-Bu): n-butyl, Bu-i (or i-Bu): isobutyl, Bu-s (or s-Bu): sec-butyl, Bu-t (or t-Bu): tert-butyl. In addition, room temperature means about 15 to 25° C.

### Production Example 1

2-Fluoro-4-(trifluoromethylthio)aniline (20.0 g), a 28% sodium methylate-methanol solution (91.0 g) and methanol (50 mL) were mixed, and methanol suspension (100 mL) of paraformaldehyde (4.0 g) (content; 90% by weight) was added thereto, and stirred for 6 hours at room temperature. The reaction mixture was poured into ice-cold water (300 mL), and filtered under reduced pressure. The obtained white solid was dried under reduced pressure to give 21.1 g of 2-fluoro-N-methoxymethyl-4-(trifluoromethylthio)aniline.

### 2-Fluoro-N-methoxymethyl-4-(trifluoromethylthio)aniline

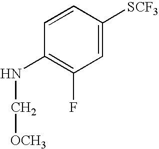

¹H-NMR (CDCl₃) δ [ppm]: 3.33 (3H, s), 4.69-4.71 (2H, m), 5.10-5.25 (1H, br), 6.94-6.96 (1H, m), 7.26-7.32 (2H, m)

### Production Example 2

2-Fluoro-N-methoxymethyl-4-(trifluoromethylthio)aniline (2.00 g) was dissolved in ethanol (35 mL), and sodium borohydride (0.70 g) (content; 90% by weight) was added thereto, and heated under reflux for 30 minutes. The reaction mixture cooled to room temperature was concentrated under reduced pressure, and water (50 mL) and hexane (50 mL) were added thereto and separated the layers. The organic layer was washed with water (50 mL), dried over anhydrous magnesium sulfate, and concentrated under reduced pressure to give 1.58 g of 2-fluoro-N-methyl-4-(trifluoromethylthio)aniline.

### 2-Fluoro-N-methyl-4-(trifluoromethylthio)aniline

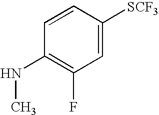

¹H-NMR (CDCl₃) δ (ppm): 2.91 (3H, m), 4.27 (1H, br), 6.62-6.67 (1H, m), 7.23-7.33 (2H, m).

### Example 1

A solution in which 0.83 g of 2,6-difluorobenzoylisocyanate was dissolved in 1.0 mL of diethyl ether under ice cooling was added to a solution of 1.02 g of 2-fluoro-N-methyl-4-(trifluoromethylthio)aniline in 4.0 mL of diethyl ether at 3° C., and stirred for 2 hours at room temperature. To the reaction mixture was added hexane (10 mL), filtered, and the filter cake was dried to give 1.58 g of 3-(2,6-difluorobenzoyl)-1-[2-fluoro-4-(trifluoromethylthio)phenyl]-1-methylurea (hereinafter, referred to as the present compound (1)).

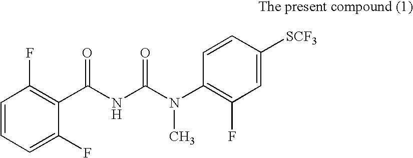

¹H-NMR (DMSO-d₆) δ (ppm): 3.23 (3H, s), 7.10-7.14 (2H, m), 7.48-7.62 (3H, m), 7.75-7.77 (1H, m), 10.90 (1H, brs)

### Examples 2 and 3

In the same way as in Example 1, the following compounds were produced.

### Example 2

3-(2,6-Difluorobenzoyl)-1-[2-fluoro-4-(1,1,2,2-tetrafluoroethylthio)phenyl]-1-methylurea (hereinafter, referred to as the present compound (2)).

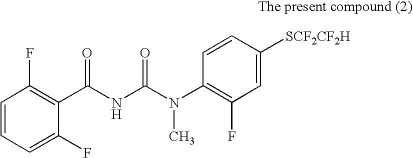

¹H-NMR (DMSO-d₆) δ (ppm): 3.23 (3H, s), 6.57-6.85 (1H, m), 7.09-7.15 (2H, m), 7.46-7.56 (3H, m), 7.65-7.68 (1H, m), 10.86 (1H, brs).

### Example 3

3-(2-Chloro-6-fluorobenzoyl)-1-[2-fluoro-4-(trifluoromethylthio)phenyl]-1-methylurea (hereinafter, referred to as the compound (3) of the present invention).

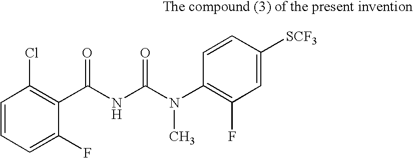

¹H-NMR (CDCl₃, TMS) δ (ppm): 3.23 (3H, s), 7.26-7.28 (1H, m), 7.32-7.34 (1H, m), 7.42-7.46 (1H, m), 7.57-7.61 (2H, m), 7.76-7.78 (1H, m), 10.92 (1H, brs).

### Production Example 3

To a solution of 2-fluoro-4-(trifluoromethylthio)aniline (5.00 g) in pyridine (20 mL) was added dropwise acetyl chloride (2.0 mL) under ice-cooling, and stirred at 3° C. for 30 minutes. To the reaction mixture were added water (50 mL) and ethyl acetate (50 mL), and layers separated. The organic layer was washed sequentially with 7% hydrochloric acid (50 mL), water (50 mL) and a saturated saline solution (50 mL), dried over anhydrous magnesium sulfate, and then concentrated under reduced pressure. The obtained residue was recrystallized from ethyl acetate-hexane to give 3.27 g of 2-fluoro-4-(trifluoromethylthio)acetanilide.

### 2-Fluoro-4-(trifluoromethylthio)acetanilide

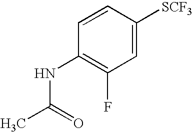

¹H-NMR (CDCl₃) δ (ppm): 2.14 (3H, s), 7.51-7.53 (1H, m), 7.66-7.69 (1H, m), 8.17-8.22 (1H, m), 10.02 (1H, brs)

### Production Example 4

To a solution of 2-fluoro-4-(trifluoromethylthio)acetanilide (1.50 g) in dimethylsulfoxide (30 mL) was added 0.35 g of sodium hydride (content; 60% by weight in oil), and stirred at room temperature for 30 minutes. Thereto 0.98 mL of iodoethane was added, and the mixture was stirred for 30 minutes. The reaction mixture was poured into 50 mL of ice-water and then extracted with 50 mL of ethyl acetate. The organic layer was washed with 50 mL of saturated saline solution, dried over anhydrous magnesium sulfate, and then concentrated under reduced pressure. The obtained residue was dissolved in 40 mL of methanol, and thereto 20 mL of 35% hydrochloric acid was added. The mixture was heated to reflux for 8 hours. The reaction mixture was allowed to cool to room temperature, poured into a mixture of a 20 wt % sodium hydroxide aqueous solution and 50 g of ice, and then extracted with 100 mL of chloroform. The organic layer was dried over anhydrous magnesium sulfate, and concentrated under reduced pressure. The obtained residue was purified by silica gel chromatography (ethyl acetate:hexane=1:5) to give 1.13 g of N-ethyl-2-fluoro-4-(trifluoromethylthio)aniline.

### N-ethyl-2-fluoro-4-(trifluoromethylthio)aniline

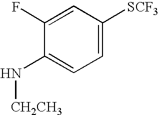

¹H-NMR (DMSO-d₆) δ (ppm): 1.27 (3H, t, J=8.0 Hz), 1.60 (1H, br), 3.20 (2H, q, J=8.0 Hz), 6.61-6.66 (1H, m), 7.20-7.28 (2H, m).

### Example 4

A solution of 0.86 g of 2,6-difluorobenzoyl isocyanate in 1.0 mL of diethyl ether prepared under ice-cooling was added at 3° C. to a solution of 1.13 g of N-ethyl-2-fluoro-4-(trifluoromethylthio)aniline in 9.0 mL of diethyl ether, and then stirred at room temperature for two hours. The reaction mixture was filtered, and the filter cake was dried to give 1.67 g of 3-(2,6-difluorobenzoyl)-1-ethyl-1-[2-fluoro-4-(trifluoromethylthio)phenyl]urea (hereinafter, referred to as the present compound (4)).

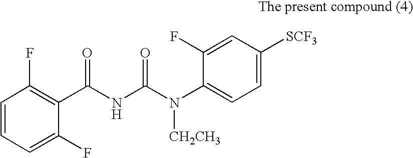

¹H-NMR (DMSO-d₆) δ (ppm): 1.04 (3H, t, J=6.8 Hz), 3.67 (2H q, J=6.8 Hz), 7.08-7.13 (2H, m), 7.45-7.55 (2H, m), 7.60-7.62 (1H, m), 7.75-7.77 (1H, m), 10.81 (1H, brs).

### Examples 5-9

In the same way as in Example 4, the following compounds were produced.

### Example 5

3-(2,6-Difluorobenzoyl)-1-(2-fluoro-4-methylthiophenyl)-1-methylurea (hereinafter, referred to as the present compound (5)).

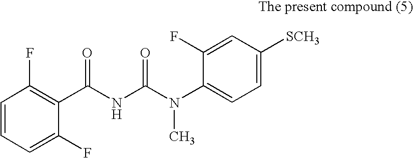

¹H-NMR (CDCl₃) δ (ppm): 2.52 (3H, s), 3.20 (3H, s), 6.91-6.98 (2H, m), 7.07-7.12 (2H, m), 7.19-7.26 (1H, m), 7.34-7.43 (1H, m), 7.59 (1H, brs).

### Example 6

3-(2-Chloro-6-fluorobenzoyl)-1-(2-fluoro-4-methylthiophenyl)-1-methylurea (hereinafter, referred to as the present compound (6)).

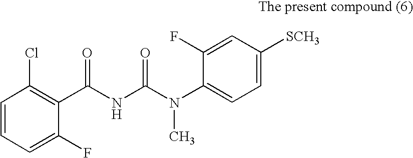

¹H-NMR (CDCl₃) δ (ppm): 2.53 (3H, s), 3.19 (3H, s), 7.02-7.13 (3H, m), 7.19-7.25 (2H, m), 7.29-7.37 (1H, m), 7.60 (1H, brs).

### Example 7

3-(2,6-Difluorobenzoyl)-1-[4-(trifluoromethylthio)phenyl]-1-methylurea (hereinafter, referred to as the present compound (7)).

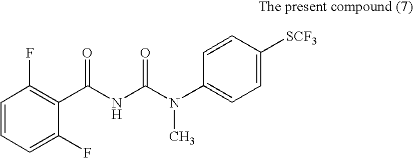

¹H-NMR (DMSO-d₆) δ (ppm): 3.22 (3H, s), 7.13-7.18 (2H, m), 7.43-7.55 (3H, m), 7.73-7.76 (2H, m), 10.84 (1H, brs).

### Example 8

3-(2,6-Difluorobenzoyl)-1-(2-fluoro-4-ethylthiophenyl)-1-methylurea (hereinafter, referred to as the present compound (8)).

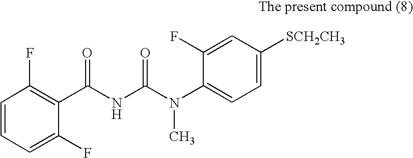

¹H-NMR (DMSO-d₆, Measurement temperature: 80° C.) δ (ppm): 1.27 (3H, t, J=7.2 Hz), 3.01 (2H, q, J=7.2 Hz), 3.17 (3H, s), 7.03-7.11 (2H, m), 7.12-7.16 (1H, m), 7.19-7.23 (1H, m), 7.24-7.30 (1H, m), 7.42-7.51 (1H, m), 10.35 (1H, brs).

### Example 9

3-(2-Chloro-6-fluorobenzoyl)-1-(2-fluoro-4-ethylthiophenyl)-1-methylurea (hereinafter, referred to as the present compound (9)).

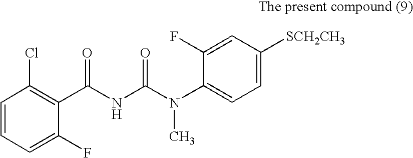

¹H-NMR (DMSO-d₆, Measurement temperature: 80° C.) δ (ppm): 1.27 (3H, t, J=7.3 Hz), 3.01 (2H, q, J=7.3 Hz), 3.16 (3H, s), 7.11-7.16 (1H, m), 7.16-7.23 (2H, m), 7.25-7.30 (2H, m), 7.38-7.45 (2H, m), 10.37 (1H, brs).

### Example 10

To a solution of 0.50 g of 3-(2,6-difluorobenzoyl)-1-[2-fluoro-4-(trifluoromethylthio)phenyl]-1-methylurea in 5.0 mL of 1-methyl-2-pyrrolidone was added 59 mg of sodium hydride (content; 60% by weight in oil) at 3° C. The mixture was stirred at 3° C. for 30 minutes, and thereto 0.18 mL of methyl iodide was added at 4° C. The reaction mixture was stirred at room temperature for four hours, and thereto a mixture of 5 mL of a saturated ammonium chloride aqueous solution and 5 mL of water was added under ice-cooling. The mixture was then extracted with 10 mL of ethyl acetate three times. Organic layers were combined, washed with a saturated saline solution three times, dried over anhydrous magnesium sulfate, and then concentrated under reduced pressure. The obtained residue was purified by medium pressure preparative high performance liquid chromatography (ethyl acetate:chloroform:hexane=15:15:70) to give 0.41 g of 1-(2,6-difluorobenzoyl)-3-[2-fluoro-4-(trifluoromethylthio)phenyl]-1,3-dimethylurea (hereinafter, referred to as the present compound (10)).

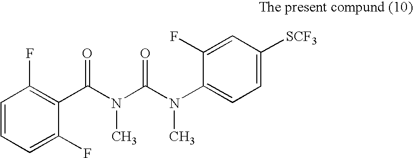

¹H-NMR (DMSO-d₆) δ (ppm): 3.07 (3H, brs), 3.26 (3H, brs), 7.12-7.16 (2H, m), 7.30-7.78 (4H, m).

### Examples 11-15

In the same way as in Example 10, the following compounds were produced.

### Example 11

1-(2,6-Difluorobenzoyl)-3-[2-fluoro-4-(trifluoromethylthio)phenyl]-1-(methoxymethyl)-3-methylurea (hereinafter, referred to as the present compound (11)).

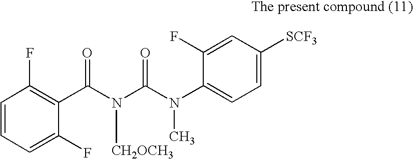

¹H-NMR (CDCl₃) δ (ppm): 3.38 (6H, m), 4.87 (2H, br), 6.84-6.88 (2H, m), 7.31-7.52 (4H, m).

### Example 12

1-(2,6-Difluorobenzoyl)-3-[2-fluoro-4-(1,1,2,2-tetrafluoroethylthio)phenyl]-1,3-dimethylurea (hereinafter, referred to as the present compound (12)).

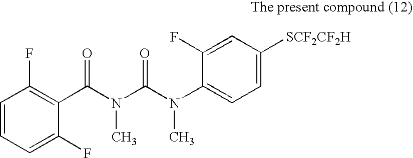

¹H-NMR (CDCl₃) δ (ppm): 3.09 (3H, s), 3.34 (3H, br), 5.67-5.94 (1H, m), 6.8-6.9 (2H, m), 7.34-7.37 (1H, m), 7.42-7.48 (3H, m).

### Example 13

1-(2-Chloro-6-difluorobenzoyl)-3-[2-fluoro-4-(trifluoromethylthio)phenyl]-1,3-dimethylurea (hereinafter, referred to as the present compound (13)).

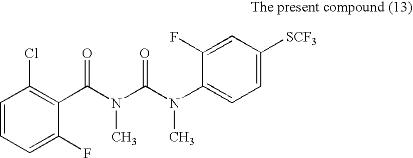

¹H-NMR (DMSO-d₆) δ (ppm): 3.00 (3H, br), 3.05 (3H, s), 7.20-7.24 (1H, m), 7.31-7.34 (1H, m), 7.45-7.58 (3H, m), 7.67-7.70 (1H, m).

### Example 14

1-(2,6-Difluorobenzoyl)-3-(2-fluoro-4-methylthiophenyl)-1,3-dimethylurea (hereinafter, referred to as the present compound (14)).

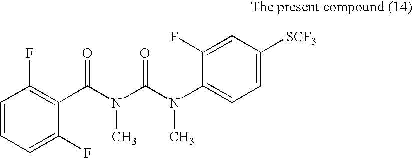

¹H-NMR (CDCl₃, TMS) δ (ppm): 2.48 (3H, s), 3.04 (3H, s), 3.26 (3H, brs), 6.77-7.24 (5H, m), 7.29-7.41 (1H, m).

### Example 15

1-(2-Chloro-6-fluorobenzoyl)-3-(2-fluoro-4-methylthiophenyl)-1,3-dimethylurea (hereinafter, referred to as the present compound (15)).

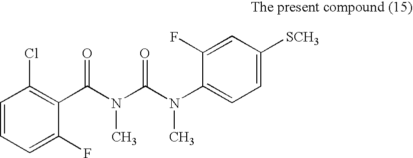

¹H-NMR (DMSO-d₆, Measurement temperature: 80° C.) δ (ppm): 2.49 (3H, s), 2.95 (3H, brs), 3.23 (3H, s), 7.08-7.13 (1H, m), 7.17-7.35 (4H, m), 7.43-7.51 (1H, m).

### Example 16

1-(2,6-Difluorobenzoyl)-3-(2-fluoro-4-ethylthiophenyl)-1,3-dimethylurea (hereinafter, referred to as the present compound (16)).

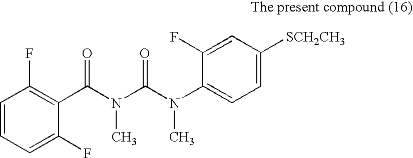

¹H-NMR (DMSO-d₆, Measurement temperature: 80° C.) δ (ppm): 1.26 (3H, t, J=7.4 Hz), 3.01 (2H, q, J=7.4 Hz), 3.02 (3H, s), 3.20 (3H, s), 7.06-7.17 (4H, m), 7.19-7.25 (1H, m), 7.47-7.56 (1H, m).

### Example 17

1-(2-Chloro-6-fluorobenzoyl)-3-(2-fluoro-4-ethylthiophenyl)-1,3-dimethylurea (hereinafter, referred to as the present compound (17)).

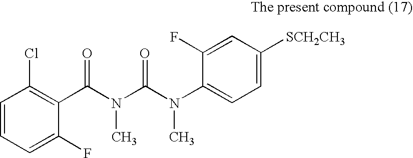

¹H-NMR (DMSO-d₆, Measurement temperature: 80° C.) δ (ppm): 1.27 (3H, t, J=7.3 Hz), 2.96 (3H, brs), 3.01 (2H, q, J=7.3 Hz), 3.24 (3H, s), 7.11-7.16 (1H, m), 7.19-7.29 (3H, m), 7.31 (1H, d, J=8.2 Hz), 7.43-7.50 (1H, m).

### Example 18

To a solution of 1.0 g of 3-(2,6-difluorobenzoyl)-1-[2-fluoro-4-(trifluoromethylthio)phenyl]-1-methylurea in 10 mL of chloroform was added 0.65 g of meta-chloroperbenzoic acid (content; 65% by weight) under ice-cooling, stirred for an hour under ice-cooling, and then left at room temperature for 72 hours. To the reaction mixture was added 10 mL of chloroform, and the mixture was washed three times with 20 mL of a saturated sodium hydrogen carbonate. Organic layers were combined, dried over anhydrous magnesium sulfate, and concentrated under reduced pressure. The obtained residue was purified by medium pressure preparative high performance liquid chromatography (ethyl acetate:hexane=33:67) to give 0.44 g of 3-(2,6-difluorobenzoyl)-1-[2-fluoro-4-(trifluoromethylsulfinyl)phenyl]-1-methylurea (hereinafter, referred to as the present compound (18)).

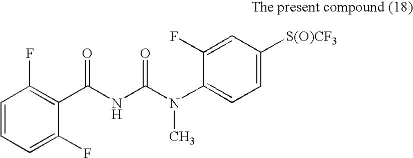

¹H-NMR (CDCl₃) δ (ppm): 3.31 (3H, s), 6.92-6.96 (2H, m), 7.38-7.41 (1H, m), 7.61-7.71 (3H, m), 8.00-8.30 (1H, m).

### Example 19

In the same way as in Example 18, the following compound was produced.

1-(2,6-Difluorobenzoyl)-3-[2-fluoro-4-(trifluoromethylsulfinyl)phenyl]-1,3-dimethylurea (hereinafter, referred to as the present compound (19)).

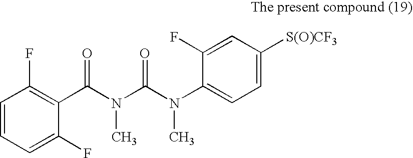

¹H-NMR (CDCl₃) δ (ppm): 3.12 (3H, s), 3.40 (3H, br), 6.80-6.90 (2H, br), 7.33-7.37 (1H, m), 7.53-7.54 (2H, m), 7.65-7.67 (1H, m).

### Example 20

To a solution of 1.0 g of 3-(2,6-difluorobenzoyl)-1-[2-fluoro-4-(trifluoromethylsulfinyl)phenyl]-1-methylurea in 20 mL of chloroform was added 1.6 g of meta-chloroperbenzoic acid (content; 65% by weight) under ice-cooling, stirred for one hour under ice-cooling, and then left at room temperature for 72 hours. To the reaction mixture was added 20 mL of chloroform, and the mixture washed three times with 40 mL of a saturated sodium hydrogen carbonate aqueous solution. Organic layers were combined, dried over anhydrous magnesium sulfate, and then concentrated under reduced pressure. The obtained residue was purified by medium pressure preparative high performance liquid chromatography (ethyl acetate:hexane=33:67) to give 0.55 g of 3-(2,6-difluorobenzoyl)-1-[2-fluoro-4-(trifluoromethylsulfonyl)phenyl]-1-methylurea (hereinafter, referred to as the present compound (20)).

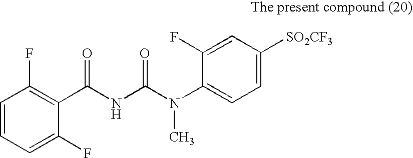

¹H-NMR (CDCl₃) δ (ppm): 3.35 (3H, s), 6.92-6.96 (2H, m), 7.37-7.44 (1H, m), 7.64-7.68 (3H, m), 8.51 (1H, br).

### Example 21

In the same way as in Example 20, the following compound was produced.

1-(2,6-Difluorobenzoyl)-3-[2-fluoro-4-(trifluoromethylsulfonyl)phenyl]-1,3-dimethylurea (hereinafter, referred to as the present compound (21)).

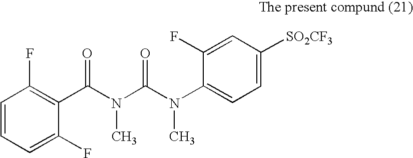

¹H-NMR (CDCl₃) δ (ppm): 3.14 (3H, s), 3.42 (3H, s), 6.85-6.89 (2H, m), 7.32-7.40 (1H, m), 7.50-7.70 (1H, br), 7.82-7.84 (2H, m).

### Example 22

To a solution of 1.5 g of 1-(2,6-difluorobenzoyl)-3-[2-fluoro-4-(1,1,2,2,3,3,3-heptafluoro-1-propylthio)phenylurea] in 15 mL of 1,3-dimethyl-2-imidazolizinone was added methyl iodide at 0° C., and then was added 331 mg of sodium hydride (content; 55% by weight in oil), and the mixture was stirred at 4° C. for 3 hours. To the reaction mixture was added 20 mL of a saturated ammonium chloride aqueous solution and then stirred for 30 minutes. To the mixture was added 50 mL of ethyl acetate, and layers separated. The organic layer was sequentially washed with water and a saturated saline solution, dried over anhydrous magnesium sulfate, and then concentrated under reduced pressure. The obtained residue was purified by silica gel chromatography (hexane:ethyl acetate=3:1) to give 400 mg of 1-(2,6-difluorobenzoyl)-3-[2-fluoro-4-(1,1,2,2,3,3,3-heptafluoro-1-propylthio)phenyl]-1,3-dimethylurea (hereinafter, referred to as the present compound (22)).

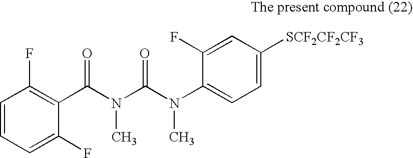

¹H-NMR (CDCl₃ δ (ppm): 3.09 (3H, s), 3.35 (3H, brs), 6.80-6.93 (2H, m), 7.20-7.52 (4H, m).

### Example 23

To a solution of 1.92 g of 2-fluoro-N-methyl-4-(1,1,2,2-tetrafluoroethylthio)aniline in 8.0 mL of diethyl ether was added a solution of 1.49 g of 2-chloro-6-fluorobenzoyl isocyanate in 2.0 mL of diethyl ether under ice-cooling, and stirred at room temperature for two hours. Hexane was added little by little to the reaction solution placed under ice-cooling, and then a white powder deposited. The powder was collected by filtration to give 3.09 g of 3-(2-chloro-6-fluorobenzoyl)-1-[2-fluoro-4-(1,1,2,2-tetrafluoroethylthio)phenyl]-1-methylurea (hereinafter, referred to as the present compound (23)).

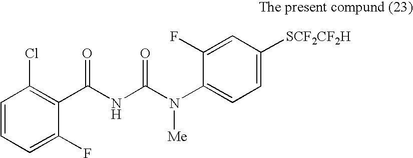

¹H-NMR (DMSO-d₆) δ (ppm): 3.23 (3H, s), 6.60-6.86 (1H, m), 7.26-7.28 (1H, m), 7.32-7.34 (1H, m), 7.44-7.46 (1H, m), 7.54-7.55 (2H, m), 7.66-7.68 (1H, m), 10.88 (1H, brs).

### Example 24

To a solution of 1.0 g of 3-(2,6-difluorobenzoyl)-1-[2-fluoro-4-(1,1,2,2-tetrafluoroethylthio)phenyl]-1-methylurea in 10.0 mL of chloroform was added 0.60 g of meta-chloroperbenzoic acid (content; 65% by weight) under ice-cooling, and stirred at room temperature for 65 hours. To the reaction mixture was added 10 mL of chloroform. The mixture was washed three times with 20 mL of a sodium hydrogen carbonate aqueous solution, dried over anhydrous magnesium sulfate, and then concentrated under reduced pressure. The obtained residue was purified by medium pressure preparative high performance liquid chromatography (ethyl acetate:hexane=34:66) to give 0.58 g of 3-(2,6-difluorobenzoyl)-1-[2-fluoro-4-(1,1,2,2-tetrafluoroethylsulfinyl)phenyl]-1-methylurea (hereinafter, referred to as the present compound (24)).

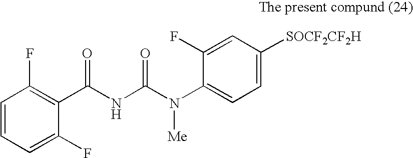

¹H-NMR (CDCl₃) δ (ppm): 3.31 (3H, s), 6.05-6.33 (1H, m), 6.92-6.96 (2H, m), 7.37-7.41 (1H, m), 7.59-7.66 (3H, m), 8.17 (1H, brs).

### Example 25

To a solution of 1.0 g of 3-(2,6-difluorobenzoyl)-1-[2-fluoro-4-(1,1,2,2-tetrafluoroethylthio)phenyl]-1-methylurea in 20.0 mL of chloroform was added 1.33 g of meta-chloroperbenzoic acid (content; 65% by weight) under ice-cooling, and stirred at room temperature for 65 hours. To the reaction mixture was added 20 mL of chloroform. The mixture was washed three times with 40 mL of a sodium hydrogen carbonate aqueous solution, dried over anhydrous magnesium sulfate, and then concentrated under reduced pressure. The obtained residue was purified by medium pressure preparative high performance liquid chromatography (ethyl acetate:hexane=34:66) to give 0.50 g of 3-(2,6-difluorobenzoyl)-1-[2-fluoro-4-(1,1,2,2-tetrafluoroethanesulfonyl)phenyl]-1-methylurea (hereinafter, referred to as the present compound (25)).

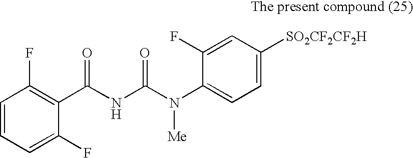

¹H-NMR (CDCl₃) δ (ppm): 3.31 (3H, s), 6.05-6.33 (1H, m), 6.92-6.96 (2H, m), 7.37-7.41 (1H, m), 7.59-7.66 (3H, m), 8.17 (1H, brs).

### Example 26

To a solution of 0.32 g of 2-fluoro-N-methyl-4-(2,2,2-trifluoroethylthio)aniline in 1.2 mL of diethyl ether was added a solution of 0.25 g of 2,6-difluorobenzoyl isocyanate in 0.3 mL of diethyl ether under ice-cooling, and stirred at room temperature for two hours. Hexane was added portionwise to the reaction solution under ice-cooling, and then a white powder deposited. The powder was collected by suction filtration to give 0.53 g of 3-(2,6-difluorobenzoyl)-1-[2-fluoro-4-(2,2,2-trifluoroethylthio)phenyl]-1-methylurea (hereinafter, referred to as the present compound (26)).

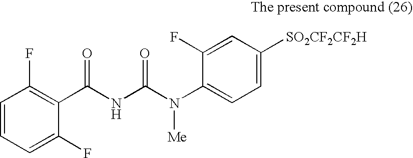

¹H-NMR (DMSO-d₆) δ (ppm): 3.17 (3H, s), 4.10-4.17 (2H, m), 7.11-7.15 (2H, m), 7.33-7.35 (2H, m), 7.48-7.54 (2H, m), 10.75 (1H, brs).

### Example 27

To a solution of 1.02 g of 2-chloro-N-methyl-4-(trifluoromethylthio)aniline in 4.1 mL of diethyl ether was added a solution of 0.77 g of 2,6-difluorobenzoyl isocyanate in 1.0 mL of diethyl ether under ice-cooling, and stirred at room temperature for 0.5 hours. Shortly after stirring, a white powder deposited. The powder was collected by filtration to give 1.50 g of 1-[2-chloro-4-(trifluoromethylthio)phenyl]-3-(2,6-difluorobenzoyl)-1-methylurea (hereinafter, referred to as the present compound (27)).

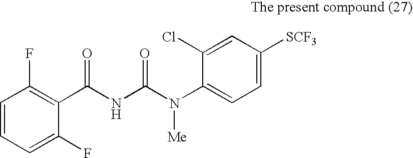

¹H-NMR (DMSO-d₆) δ (ppm): 3.19 (3H, brs), 7.10-7.14 (2H, m), 7.46-7.53 (1H, m), 7.59-7.61 (1H, m), 7.75-7.78 (1H, m), 7.96-7.97 (1H, m), 10.78 (1H, brs).

### Example 28

A solution of 173 mg of 2,6-difluorobenzoyl isocyanate in 1.0 mL of ethyl acetate was added at room temperature to a solution of 308 mg of 2-fluoro-4-(1,1,2,2,3,3,3-heptafluoro-1-propylthio)-N-methylaniline in 10 mL of ethyl acetate, and stirred for an hour. The reaction mixture was sequentially washed with water and a saturated saline solution, dried over anhydrous magnesium sulfate, and then concentrated under reduced pressure. The obtained residue (solid) was washed with a mixture solvent of hexane:tert-butylmethyl ether=3:1, and dried under reduced pressure to give 320 mg of 3-(2,6-difluorobenzoyl)-1-[2-fluoro-4-(1,1,2,2,3,3,3-heptafluoro-1-propylthio)phenyl]-1-methylurea (hereinafter, referred to as the present compound (28)).

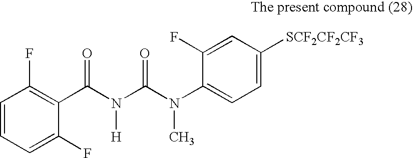

¹H-NMR (CDCl₃) δ (ppm): 3.25 (3H, s), 7.05 (1H, t, J=8.6 Hz), 7.22 (1H, d, J=8.2 Hz), 7.30-7.37 (1H, m), 7.38-7.44 (1H, m), 7.50-7.57 (2H, m), 8.03 (1H, br s).

### Example 29

A solution of 462 mg of 2-chloro-6-fluorobenzoyl isocyanate in 2.0 mL of ethyl acetate was added at room temperature to a solution of 752 mg of 2-fluoro-4-(1,1,2,2,3,3,3-heptafluoro-1-propylthio)-N-methylaniline in 10 mL of ethyl acetate, and stirred for an hour. The reaction mixture was concentrated under reduced pressure. The obtained solid was washed with a mixture solvent of hexane:tert-butylmethyl ether=3:1, and dried under reduced pressure to give 990 mg of 3-(2-chloro-6-fluorobenzoyl)-1-[2-fluoro-4-(1,1,2,2,3,3,3-heptafluoro-1-propylthio)phenyl]-1-methylurea (hereinafter, referred to as the present compound (29)).

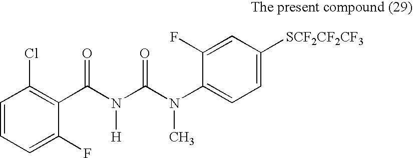

¹H-NMR (CDCl₃) δ (ppm): 3.25 (3H, s), 7.05 (1H, t, J=8.6 Hz), 7.22 (1H, d, J=8.2 Hz), 7.30-7.37 (1H, m), 7.38-7.44 (1H, m), 7.50-7.57 (2H, m), 8.03 (1H, br s).

### Example 30

A solution of 732 mg of 2,6-fluorobenzoyl isocyanate in 2.0 mL of ethyl acetate was added at room temperature to a solution of 1.1 g of 2-fluoro-N-methyl-4-(1,1,2,2,2-pentafluoroethylthio)aniline in 20 mL of ethyl acetate, and stirred for five minutes. The reaction mixture was concentrated under reduced pressure. The obtained residue was purified by medium pressure preparative high performance liquid chromatography (ethyl acetate:hexane=25:75) to give 1.6 g of 3-(2,6-difluorobenzoyl)-1-[2-fluoro-4-(1,1,2,2,2-pentafluoroethylthio)phenyl]-1-methylurea (hereinafter, referred to as the present compound (30)).

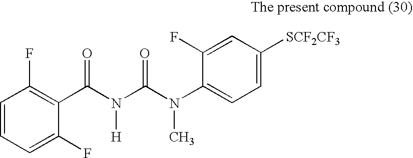

¹H-NMR (CDCl₃) δ (ppm): 3.27 (3H, s), 6.88-7.00 (2H, m), 7.35-7.45 (2H, m), 7.50-7.60 (2H, m), 7.80 (1H, br s).

### Example 31

A solution of 628 mg of 2,6-fluorobenzoyl isocyanate in 2.0 mL of tert-butylmethyl ether was added at room temperature to a solution of 1.04 g of 2-fluoro-4-(1,1,2,3,3,3-hexafluoro-1-propylthio)-N-methylaniline in 8.0 mL of tert-butylmethyl ether, and stirred for 30 minutes. To the reaction mixture was added 20 mL of hexane, the mixture was filtered. The filter cake was dried to give 1.63 g of 3-(2,6-difluorobenzoyl)-1-[2-fluoro-4-(1,1,2,3,3,3-hexafluoro-1-propylthio)phenyl]-1-methylurea (hereinafter, referred to as the present compound (31)).

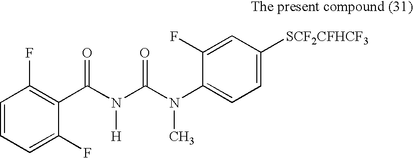

¹H-NMR (DMSO-d₆) δ (ppm): 3.24 (3H, s), 6.14-6.38 (1H, m), 7.08-7.17 (2H, m), 7.46-7.58 (3H, m), 7.64-7.70 (1H, m), 10.86 (1H, br s).

### Example 32

A solution of 638 mg of 2,6-difluorobenzoyl isocyanate in 2.0 mL of tert-butylmethyl ether was added at room temperature to a solution of 820 mg of 2,3-dimethyl-N-methyl-4-(trifluoromethylthio)aniline in 10 mL of tert-butylmethyl ether, and stirred for 30 minutes. To the reaction mixture was added 20 mL of hexane, and the mixture was filtered. The filter cake was dried to give 1.37 g of 3-(2,6-difluorobenzoyl)-1-[2,3-dimethyl-4-(trifluoromethylthio)phenyl]-1-methylurea (hereinafter, referred to as the present compound (32).

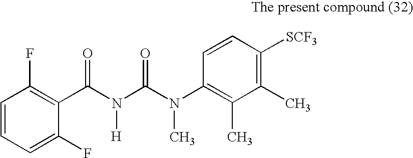

¹H-NMR (CDCl₃) δ (ppm): 2.27 (3H, s), 2.58 (3H, s), 3.17 (3H, s), 6.94 (2H, t, J=8.3 Hz), 7.16 (1H, d, J=8.3 Hz), 7.33-7.50 (2H, m), 7.69 (1H, d, J=8.3 Hz).

### Example 33

A solution of 487 mg of 2,6-difluorobenzoyl isocyanate in 1.0 mL of tert-butylmethyl ether was added at room temperature to a solution of 820 mg of 2,3-dimethyl-N-methyl-4-(1,1,2,2,2-pentafluoroethylthio)aniline in 10 mL of tert-butylmethyl ether, and stirred for 30 minutes. The reaction mixture was concentrated under reduced pressure. The obtained residue was purified by medium pressure preparative high performance liquid chromatography (ethyl acetate:hexane=25:75) to give 1.37 g of 3-(2,6-difluorobenzoyl)-1-[2,3-dimethyl-4-(1,1,2,2,2-pentafluoroethylthio)phenyl]-1-methylurea (hereinafter, referred to as the present compound (33)).

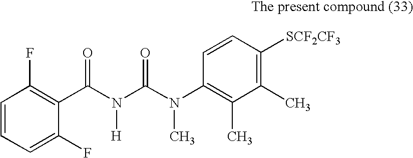

¹H-NMR (CDCl₃) δ (ppm): 2.28 (3H, s), 2.59 (3H, s), 3.18 (3H, s), 6.94 (2H, t, J=8.2 Hz), 7.17 (1H, d, J=8.2 Hz), 7.32-7.43 (2H, m), 7.68 (1H, d, J=8.2 Hz).

### Example 34

A solution of 819 mg of 2,6-difluorobenzoyl isocyanate in 2.0 mL of tert-butylmethyl ether was added at room temperature to a solution of 1.00 g of 2-chloro-4-(difluoromethylthio)-N-methylaniline in 10 mL of tert-butylmethyl ether, and stirred for 30 minutes. The reaction mixture was concentrated under reduced pressure The obtained residue was purified by medium pressure preparative high performance liquid chromatography (ethyl acetate:hexane=25:75) to give 1.74 g of 1-[2-chloro-4-(difluoromethylthio)phenyl]-3-(2,6-difluorobenzoyl)-1-methylurea (hereinafter, referred to as the present compound (34)).

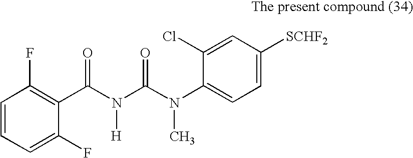

¹H-NMR (CDCl₃) δ (ppm): 3.21 (3H, s), 6.91 (1H, t, J=56.1 Hz), 6.94 (2H, t, J=8.2 Hz), 7.34-7.45 (2H, m), 7.52 (1H, br s), 7.60 (1H, dd, J=8.2, 2.0 Hz), 7.78 (1H, d, J=2.0 Hz).

### Example 35

A solution of 964 mg of 2,6-difluorobenzoyl isocyanate in 2.0 mL of tert-butylmethyl ether was added at room temperature to a solution of 1.07 g of 4-(difluoromethylthio)-N-methyl-2-methylaniline in 10 mL of tert-butylmethyl ether, and stirred for 30 minutes. The reaction mixture was concentrated under reduced pressure to give 2.14 g of 3-(2,6-difluorobenzoyl)-1-[4-(difluoromethylthio)-2-methylphenyl]-1-methylurea (hereinafter, referred to as the present compound (35)).

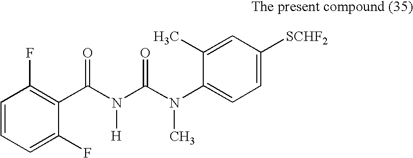

¹H-NMR (CDCl₃) δ (ppm): 2.33 (3H, s), 3.18 (3H, s), 6.90 (1H, t, J=56.3 Hz), 6.94 (2H, t, J=8.2 Hz), 7.26 (1H, d, J=8.0 Hz), 7.34-7.46 (2H, m), 7.54 (1H, d, J=8.0 Hz), 7.59 (1H, br s).

### Example 36

A solution of 0.53 g of 2,6-difluorobenzoyl isocyanate in 0.5 mL of diethyl ether prepared under ice-cooling was added at 3° C. to a solution of 0.64 g of N-methyl-2-methyl-4-(trifluoromethylthio)aniline in 2.5 mL of diethyl ether, stirred at room temperature for two hours. To the reaction mixture was added 6 mL of hexane, and the mixture was filtered. The filter cake was dried to give 1.58 g of 3-(2,6-difluorobenzoyl)-1-methyl-1-[2-methyl-4-(trifluoromethylthio)phenyl]urea (hereinafter, referred to as the present compound (36)).

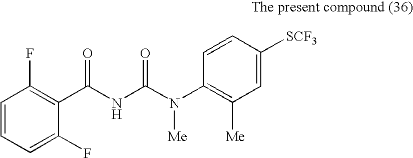

¹H-NMR (CDCl₃) δ (ppm): 2.35 (3H, s), 3.19 (3H, s), 6.93-6.97 (2H, m), 7.30-7.43 (3H, m), 7.62-7.67 (2H, m).

### Production Example 5

To a mixture of 1.00 g of t-butyl (4-amino-3-fluoro)benzoate and 0.21 g of paraformaldehyde (content; 90% by weight) in 5 mL of methanol was added a mixture of 4.50 g of a 28% sodium methylate-methanol solution and 2 mL of methanol, and stirred at room temperature for 18 hours. The reaction mixture was poured into 15 mL of ice water, and then extracted with 20 mL of chloroform. The organic layer was dried over anhydrous magnesium sulfate, and concentrated under reduced pressure to obtain a residue. The residue was dissolved in 20 mL of ethanol, and thereto 0.40 g of sodium borohydride (content; 90% by weight) was added. The mixture was heated to reflux for 30 minutes. The reaction mixture was allowed to cool to room temperature, and then concentrated under reduced pressure. To the residue were added 20 mL of water and 20 mL of chloroform, and then layers separated. The organic layer was dried over anhydrous magnesium sulfate, and concentrated under reduced pressure. The obtained residue was purified by silica gel chromatography (ethyl acetate:hexane=1:5) to give 0.51 g of t-butyl (3-fluoro-4-methylamino)benzoate.

### t-Butyl (3-fluoro-4-methylamino)benzoate

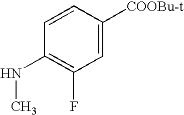

¹H-NMR (CDCl₃) δ (ppm): 1.57 (9H, s), 2.92 (3H, d, J=5.3 Hz), 4.33 (1H, br), 6.59-6.64 (1H, m), 7.54-7.58 (1H, m), 7.69-7.72 (1H, m).

### Example 37

A solution of 0.42 g of 2,6-difluorobenzoyl isocyanate in 0.5 mL of diethyl ether prepared under ice-cooling was added at 3° C. to a solution of 0.51 g of t-butyl (3-fluoro-4-methylamino)benzoate in 2.5 mL of diethyl ether, and stirred at room temperature for two hours. The reaction mixture was filtered, and the filter cake was dried to give 0.76 g of 3-(2,6-difluorobenzoyl)-1-[2-fluoro-4-(t-butoxycarbonyl)phenyl]-1-methylurea (hereinafter, referred to as the present compound (37)).

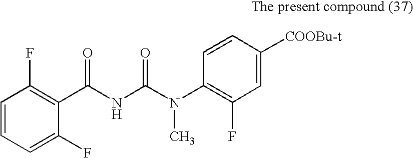

¹H-NMR (DMSO-d₆) δ (ppm): 1.55 (9H, s), 3.22 (3H, s), 7.12-7.16 (2H, m), 7.49-7.54 (2H, m), 7.69-7.77 (2H, m), 10.82 (1H, brs).

### Example 38

To a solution of 1.01 g of 3-(2-chloro-6-fluorobenzoyl)-1-[2-fluoro-4-(1,1,2,2-tetrafluoroethylthio)phenyl]-1-methylurea in 10.0 mL of 1-methyl-2-pyrrolidone was added 105 mg of sodium hydride (content; 55% by weight in oil) at 2° C., and stirred for 30 minutes. Then 0.33 mL of methyl iodide was added thereto at 2° C., and the mixture was stirred at 2-3° C. for three hours. To the reaction mixture was added a mixture of 10.0 mL of a saturated ammonium chloride aqueous solution and 10.0 mL of water under ice-cooling, and the mixture was extracted with 20 mL of ethyl acetate three times. Organic layers were combined, washed with a saturated saline solution three times, dried over anhydrous magnesium sulfate, and concentrated under reduced pressure. The obtained residue was purified by silica gel chromatography (ethyl acetate:chloroform:hexane=1:1:4) to give 0.48 g of 1-(2-chloro-6-fluorobenzoyl)-3-[2-fluoro-4-(1,1,2,2-tetrafluoroethylthio)phenyl]-1,3-dimethylurea (hereinafter, referred to as the present compound (38)).

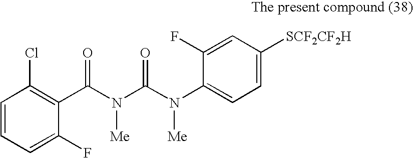

¹H-NMR (CDCl₃) δ (ppm): 3.03 (3H, s), 3.42 (3H, brs), 5.69-5.96 (1H, m), 6.9-7.2 (2H, m), 7.27 (1H, m), 7.46-7.48 (3H, m).

### Example 39

To a solution of 1.01 g of 1-(2,6-difluorobenzoyl)-3-[2-fluoro-4-(1,1,2,2-tetrafluoroethylthio)phenyl]-1,3-dimethylurea in 10.0 mL of chloroform was added 0.58 g of meta-chloroperbenzoic acid (content; 65% by weight) under ice-cooling, and stirred at room temperature for 72 hours. To the reaction mixture was added 10 mL of chloroform. The mixture was washed three times with 20 mL of a sodium hydrogen carbonate aqueous solution, dried over anhydrous magnesium sulfate, and then concentrated under reduced pressure. The obtained residue was purified by medium pressure preparative high performance liquid chromatography (ethyl acetate:hexane=34:66) to give 0.71 g of 1-(2,6-difluorobenzoyl)-3-[2-fluoro-4-(1,1,2,2-tetrafluoroethylsulfinyl)phenyl]-1,3-dimethylurea (hereinafter, referred to as the present compound (39)).

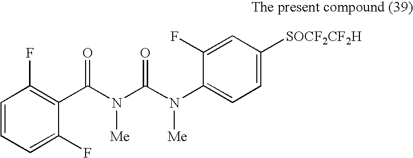

¹H-NMR (CDCl₃) δ (ppm): 3.12 (3H, s), 3.39 (3H, brs), 6.04-6.31 (1H, m), 6.88 (2H, m), 7.34-7.37 (1H, m), 7.50-7.51 (2H, m), 7.61-7.63 (1H, m).

### Example 40

To a solution of 1.01 g of 1-(2,6-difluorobenzoyl)-3-[2-fluoro-4-(1,1,2,2-tetrafluoroethylthio)phenyl]-1,3-dimethylurea in 20.0 mL of chloroform was added 1.28 g of meta-chloroperbenzoic acid (content; 65% by weight) under ice-cooling, and stirred at room temperature for 72 hours. To the reaction mixture was added 20 mL of chloroform. The mixture was washed three times with 40 mL of a sodium hydrogen carbonate aqueous solution, dried over anhydrous magnesium sulfate, and then concentrated under reduced pressure. The obtained residue was purified by medium pressure preparative high performance liquid chromatography (ethyl acetate:hexane=34:66) to give 0.97 g of 1-(2,6-difluorobenzoyl)-3-[2-fluoro-4-(1,1,2,2-tetrafluoroethanesulfonyl)phenyl]-1,3-dimethylurea (hereinafter, referred to as the present compound (40)).

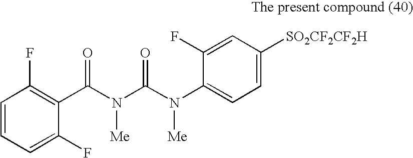

¹H-NMR (CDCl₃) δ (ppm): 3.14 (3H, s), 3.42 (3H, s), 6.14-6.40 (1H, m), 6.86-6.90 (2H, m), 7.35-7.37 (1H, m), 7.52-7.54 (1H, m), 7.78-7.81 (2H, m).

### Example 41

To a solution of 1.01 g of 3-(2,6-difluorobenzoyl)-1-ethyl-1-[2-fluoro-4-(trifluoromethylthio)phenyl]urea in 10.0 mL of 1-methyl-2-pyrrolidone was added 114 mg of sodium hydride (content; 55% by weight in oil) at 2° C. under ice-cooling, and stirred for 30 minutes. Then 0.35 mL of methyl iodide was added thereto, and the obtained mixture was stirred for 4 hours under ice-cooling. To the reaction mixture was added a mixture of 10 mL of a saturated ammonium chloride aqueous solution and 10 mL of water under ice-cooling, and the mixture was extracted with 20 mL of ethyl acetate three times. Organic layers were combined, washed with a saturated saline solution three times, dried over anhydrous magnesium sulfate, and then concentrated under reduced pressure. The obtained residue was purified by silica gel chromatography (ethyl acetate:chloroform:hexane=1:1:4) to give 0.37 g of 1-(2,6-difluorobenzoyl)-3-ethyl-3-[2-fluoro-4-(trifluoromethylthio)phenyl]-1-methylurea (hereinafter, referred to as the present compound (41)).

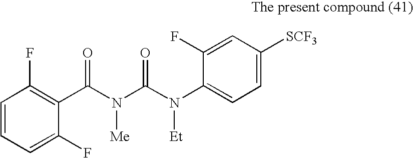

¹H-NMR (DMSO-d₆, Measurement temperature: 80° C.) δ (ppm): 1.07 (3H, t, J=7.2 Hz), 3.00 (3H, s), 3.69 (2H, q, J=7.2 Hz), 7.05-7.10 (2H, m), 7.37-7.39 (1H, m), 7.50-7.56 (2H, m), 7.67-7.70 (1H, m).

### Example 42

To a solution of 0.75 g of 3-(2,6-difluorobenzoyl)-1-methyl-1-[2-methyl-4-(trifluoromethylthio)phenyl]urea in 7.5 mL of 1-methyl-2-pyrrolidone was added 89 mg of sodium hydride (content; 55% by weight in oil) at 2° C., and stirred for 30 minutes. Then 0.28 mL of methyl iodide was added thereto at 1° C., and the mixture was stirred at room temperature for three hours. To the reaction mixture was added a mixture of 7.5 mL of a saturated ammonium chloride aqueous solution and 7.5 mL of water under ice-cooling. The mixture was extracted with 15 mL of ethyl acetate three times. Organic layers were combined, washed with a saturated saline solution three times, dried over anhydrous magnesium sulfate, and then concentrated under reduced pressure. The obtained residue was purified by silica gel chromatography (ethyl acetate:chloroform:hexane=1:1:4) to give 0.53 g of 1-(2,6-difluorobenzoyl)-1,3-dimethyl-3-[2-methyl-4-(trifluoromethylthio)phenyl]urea (hereinafter, referred to as the present compound (42)).

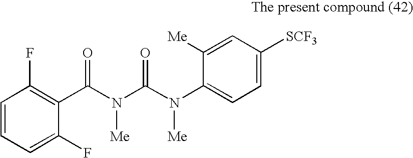

¹H-NMR (DMSO-d₆, Measurement temperature: 80° C.) δ (ppm): 2.18 (3H, s), 3.05 (3H, s), 3.23 (3H, s), 7.10-7.14 (2H, m), 7.29-7.31 (1H, m), 7.51-7.56 (2H, m), 7.62 (1H, m).

### Example 43

To a solution of 1.01 g of 3-(2,6-difluorobenzoyl)-1-[2-fluoro-4-(t-butoxycarbonyl)phenyl]-1-methylurea in 10.0 mL of 1-methyl-2-pyrrolidone was added 118 mg of sodium hydride (content; 55% by weight in oil) at 2° C., and stirred for 30 minutes. Then 0.37 mL of methyl iodide was added thereto at 1° C., and the mixture was stirred at room temperature for 3 hours. To the reaction mixture was added a mixture of 10.0 mL of a saturated ammonium chloride aqueous solution and 10.0 mL of water under ice-cooling, and the mixture was extracted with 20 mL of ethyl acetate three times. Organic layers were combined, washed three times with a saturated saline solution, dried over anhydrous magnesium sulfate, and then concentrated under reduced pressure. The obtained residue was purified by silica gel chromatography (ethyl acetate:chloroform:hexane=1:1:4) to give 0.67 g of 1-(2,6-difluorobenzoyl)-3-[2-fluoro-4-(t-butoxycarbonyl)phenyl]-1,3-dimethylurea (hereinafter, referred to as the present compound (43)).

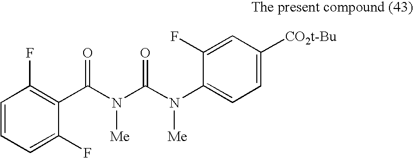

¹H-NMR (DMSO-d₆, Measurement temperature: 80° C.) δ (ppm): 1.55 (9H, s), 3.05 (3H, s), 3.24 (3H, s), 7.09-7.14 (2H, m), 7.32-7.37 (1H, m), 7.49-7.57 (1H, m), 7.66-7.73 (2H, m).

### Example 44

To a solution of 1.01 g of 3-(2,6-difluorobenzoyl)-1-[2-fluoro-4-(2,2,2-trifluoroethylthio)phenyl]-1-methylurea in 10.0 mL of 1-methyl-2-pyrrolidone was added 114 mg of sodium hydride (content; 55% by weight in oil) at 2° C., and stirred for 30 minutes. Then 0.35 mL of methyl iodide was added thereto at 1.5° C., and the mixture was stirred at 2-3° C. for 3 hours. To the reaction mixture was added a mixture of 10.0 mL of a saturated ammonium chloride aqueous solution and 10.0 mL of water under ice-cooling, and the mixture was extracted with 20 mL of ethyl acetate three times. Organic layers were combined, washed three times with a saturated saline solution, dried over anhydrous magnesium sulfate, and then concentrated under reduced pressure. The obtained residue was purified by silica gel chromatography (ethyl acetate:chloroform:hexane=1:1:4) to give 0.86 g of 1-(2,6-difluorobenzoyl)-3-[2-fluoro-4-(2,2,2-trifluoroethylthio)phenyl]-1,3-dimethylurea (hereinafter, referred to as the present compound (44)).

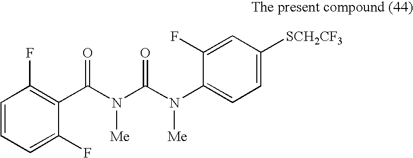

¹H-NMR (DMSO-d₆, Measurement temperature: 80° C.) δ (ppm): 3.02 (3H, s), 3.21 (3H, s), 3.97-4.07 (2H, m), 7.08-7.12 (2H, m), 7.20-7.22 (1H, m), 7.29-7.32 (1H, m), 7.44-7.54 (2H, m).

### Example 45

To a solution of 740 mg of 3-(2-chloro-6-fluorobenzoyl)-1-[2-fluoro-4-(1,1,2,2,3,3,3-heptafluoro-1-propylthio)phenyl]-1-methylurea in 10.0 mL of 1,3-dimethyl-2-imidazolizinone were added 200 mg of methyl iodide at 0° C., and then 74 mg of sodium hydride (content; 55% by weight in oil), and stirred at 4° C. for two hours. To the reaction mixture was added 20 mL of a saturated ammonium chloride aqueous solution, and stirred for 30 minutes. To the mixture was added 50 mL of ethyl acetate, and then layers separated. The organic layer was sequentially washed with water and a saturated saline solution, dried over anhydrous magnesium sulfate, and concentrated under reduced pressure. The obtained residue was purified by medium pressure preparative high performance liquid chromatography (ethyl acetate:hexane=25:75) to give 550 mg of 1-(2-chloro-6-fluorobenzoyl)-3-[2-fluoro-4-(1,1,2,2,3,3,3-heptafluoro-1-propylthio)phenyl]-1,3-dimethylurea (hereinafter, referred to as the present compound (45)).

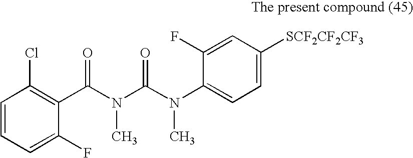

¹H-NMR ((DMSO-d₆, Measurement temperature: 80° C.) δ (ppm): 3.01 (3H, br s), 3.31 (3H, s), 7.20 (1H, t, J=8.8 Hz), 7.32 (1H, d, J=8.0 Hz), 7.44-7.61 (3H, m), 7.68 (1H, d, J=10.9 Hz).

### Example 46

To a solution of 1.1 g of 3-(2,6-difluorobenzoyl)-1-[2-fluoro-4-(1,1,2,2,2-pentafluoroethylthio)phenyl]-1-methylurea in 7 mL of 1,3-dimethyl-2-imidazolizinone were added 700 mg of methyl iodide and then 118 mg of sodium hydride (content; 55% by weight in oil), and stirred at room temperature for an hour. To the reaction mixture was added 20 mL of a saturated ammonium chloride aqueous solution, and stirred for 30 minutes. To the mixture was added 50 mL of ethyl acetate, and then layers separated. The organic layer was sequentially washed with water and a saturated saline solution, dried over anhydrous magnesium sulfate, and concentrated under reduced pressure. The obtained residue was purified by medium pressure preparative high performance liquid chromatography (ethyl acetate:hexane=25:75) to give 900 mg of 1-(2,6-difluorobenzoyl)-3-[2-fluoro-4-(1,1,2,2,2-pentafluoroethylthio)phenyl]-1,3-dimethylurea (hereinafter, referred to as the present compound (46)).

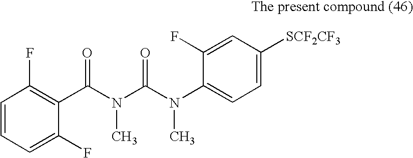

¹H-NMR ((DMSO-d₆, Measurement temperature: 80° C.) δ (ppm): 3.07 (3H, s), 3.27 (3H, s), 7.02-7.18 (2H, m), 7.39-7.60 (3H, m), 7.66 (1H, d, J=10.0 Hz).

### Example 47

To a solution of 1.12 g of 3-(2,6-difluorobenzoyl)-1-[2-fluoro-4-(1,1,2,3,3,3-hexafluoro-1-propylthio)phenyl]-1-methylurea in 10 mL of 1,3-dimethyl-2-imidazolizinone were added 300 mg of methyl iodide and then 101 mg of sodium hydride (content; 60% by weight in oil), and stirred at room temperature for an hour. To the reaction mixture were added 20 mL of water and 50 mL of ethyl acetate, and then layers separated. The organic layer was sequentially washed with water and a saturated saline solution, dried over anhydrous magnesium sulfate, and concentrated under reduced pressure. The obtained residue was purified by medium pressure preparative high performance liquid chromatography (ethyl acetate:hexane=25:75) to give 900 mg of 1-(2,6-difluorobenzoyl)-3-[2-fluoro-4-(1,1,2,3,3,3-hexafluoro-1-propylthio)phenyl]-1,3-dimethylurea (hereinafter, referred to as the present compound (47)).

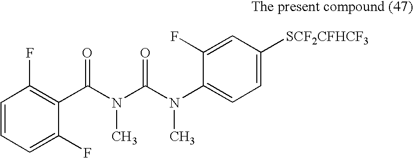

¹H-NMR (DMSO-d₆, Measurement temperature: 80° C.) δ (ppm): 3.05 (3H, s), 3.26 (3H, s), 6.03-6.25 (1H, m), 7.09 (2H, t, J=8.3 Hz), 7.36-7.43 (1H, m), 7.47-7.62 (3H, m).

### Example 48

To a solution of 860 mg of 3-(2,6-difluorobenzoyl)-1-[2,3-dimethyl-4-(trifluoromethylthio)phenyl]-1-methylurea in 10 mL of 1,3-dimethyl-2-imidazolidinone were added 581 mg of methyl iodide and then 90 mg of sodium hydride (content; 60% by weight in oil), and stirred at room temperature for 10 hours. To the reaction mixture were added 20 mL of water and 50 mL of ethyl acetate, and then layers separated. The organic layer was sequentially washed with water and a saturated saline solution, dried over anhydrous magnesium sulfate, and concentrated under reduced pressure. The obtained residue was purified by medium pressure preparative high performance liquid chromatography (ethyl acetate:hexane=20:80) to give 820 mg of 1-(2,6-difluorobenzoyl)-3-[2,3-dimethyl-4-(trifluoromethylthio)phenyl]-1,3-dimethylurea (hereinafter, referred to as the present compound (48)).

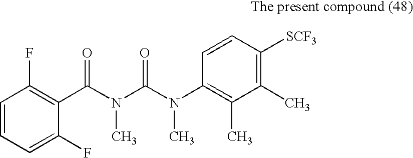

¹H-NMR (DMSO-d₆, Measurement temperature: 80° C.) δ (ppm): 2.12 (3H, s), 2.49 (3H, s), 3.04 (3H, s), 3.22 (3H, s), 7.06-7.20 (3H, m), 7.47-7.64 (2H, m).

### Example 49

To a solution of 870 mg of 3-(2,6-difluorobenzoyl)-1-[2,3-dimethyl-4-(1,1,2,2,2-pentafluoroethylthio)phenyl]-1-methylurea in 7 mL of 1,3-dimethyl-2-imidazolidinone were added 394 mg of methyl iodide and then 82 mg of sodium hydride (content; 60% by weight in oil), and stirred at room temperature overnight. To the reaction mixture were added 10 mL of a saturated ammonium chloride aqueous solution and 10 mL of ethyl acetate, and stirred for 10 minutes. To the mixture was further added 50 mL of ethyl acetate, and then layers separated. The organic layer was sequentially washed with water and a saturated saline solution, dried over anhydrous magnesium sulfate, and concentrated under reduced pressure. The obtained residue was purified by medium pressure preparative high performance liquid chromatography (ethyl acetate:hexane=25:75) to give 850 mg of 1-(2,6-difluorobenzoyl)-3-[2,3-dimethyl-4-(1,1,2,2,2-pentafluoroethylthio)phenyl]-1,3-dimethylurea (hereinafter, referred to as the present compound (49)).

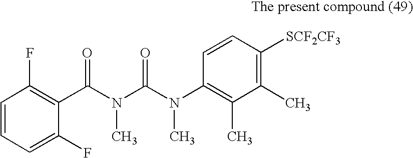

¹H-NMR (DMSO-d₆, Measurement temperature: 80° C.) δ (ppm): 2.12 (3H, s), 2.48 (3H, s), 3.04 (3H, s), 3.21 (3H, s), 7.06-7.21 (3H, m), 7.48-7.61-(2H, m).

### Example 50

To a solution of 1.16 g of 1-[2-chloro-4-(difluoromethylthio)phenyl]-3-(2,6-difluorobenzoyl)-1-methylurea in 10 mL of 1,3-dimethyl-2-imidazolidinone were added 608 mg of methyl iodide and then 125 mg of sodium hydride (content; 60% by weight in oil), and stirred at room temperature overnight. To the reaction mixture were added 20 mL of a saturated ammonium chloride aqueous solution and 50 mL of tert-butylmethyl ether, and then layers separated. The organic layer was sequentially washed with water and a saturated saline solution, dried over anhydrous magnesium sulfate, and concentrated under reduced pressure. The obtained residue was purified by medium pressure preparative high performance liquid chromatography (ethyl acetate:hexane=25:75) to give 1.17 g of 1-[2-chloro-4-(difluoromethylthio)phenyl]-3-(2,6-difluorobenzoyl)-1,3-dimethylurea (hereinafter, referred to as the present compound (50)).

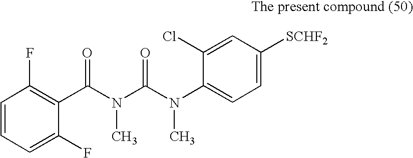

¹H-NMR (DMSO-d₆, Measurement temperature: 80° C.) δ (ppm): 3.06 (3H, s), 3.25 (3H, s), 7.11-(2H, t, J=8.5 Hz), 7.33-7.66 (4H, m), 7.74 (1H, d, J=2.0 Hz).

### Example 51

To a solution of 1.40 g of 3-(2,6-difluorobenzoyl)-1-[4-(difluoromethylthio)-2-methylphenyl]-1-methylurea in 15 mL of 1,3-dimethyl-2-imidazolidinone were added 773 mg of methyl iodide and then 159 mg of sodium hydride (content; 60% by weight in oil), and stirred at room temperature for two hours. To the reaction mixture were added 20 mL of a saturated ammonium chloride aqueous solution and 50 mL of tert-butylmethyl ether, and then layers separated. The organic layer was sequentially washed with water and a saturated saline solution, dried over anhydrous magnesium sulfate, and concentrated under reduced pressure. The obtained solid was washed with a mixture solvent of hexane:tert-butyl methyl ether=1:1, and dried under reduced pressure to give 1.21 g of 1-(2,6-difluorobenzoyl)-3-[4-(difluoromethylthio)-2-dimethylphenyl]-1,3-dimethylurea (hereinafter, referred to as the present compound (51)).

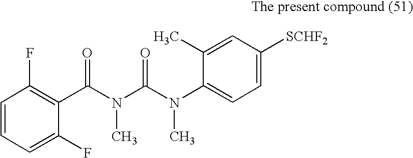

¹H-NMR (DMSO-d₆, Measurement temperature: 80° C.) δ (ppm): 2.15 (3H, s), 3.05 (3H, s), 3.22 (3H, s), 7.13 (2H, t, J=8.6 Hz), 7.21 (1H, d, J=8.2 Hz), 7.39-7.45 (1H, m), 7.41 (1H, t, J=56.3 Hz), 7.47-7.58 (2H, m).

### Example 52

To a solution of 1.01 g of 3-(2,6-difluorobenzoyl)-1-[2-fluoro-4-(trifluoromethylthio)phenyl]-1-methylurea in 10.0 mL of 1-methyl-2-pyrrolidone was added 12 mg of sodium hydride (content; 55% by weight in oil) at 2° C., and stirred for 30 minutes. Then 0.21 mL of acetyl chloride was added at 1° C. thereto. The obtained mixture was stirred at room temperature for 3 hours, poured into 10 mL of ice water, and then extracted with 20 mL of ethyl acetate three times. Organic layers were combined, washed three times with a saturated saline, dried over anhydrous magnesium sulfate, and concentrated under reduced pressure. The obtained residue was purified by silica gel chromatography (ethyl acetate:chloroform:hexane=1:1:4) to give a colorless oil. The oil was further purified by median pressure preparative high performance liquid chromatography (ethyl acetate:hexane=15:85) to give 0.62 g of 1-acetyl-(2,6-difluorobenzoyl)-3-[2-fluoro-4-(trifluormethylthio)phenyl]-3-methylurea (hereinafter, referred to as the present compound (52)).

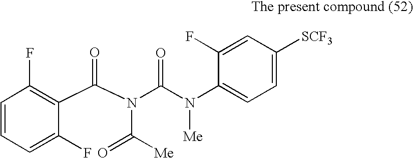

¹H-NMR (CDCl₃) δ (ppm): 2.37 (3H, brs), 3.45 (3H, brs), 6.70-6.90 (2H, brm), 7.32-7.45 (3H, m), 7.51-7.53 (1H, m).

### Example 53

To a solution of 1.01 g of 3-(2,6-difluorobenzoyl)-1-[2-fluoro-4-(trifluoromethylthio)phenyl]-1-methylurea in 10.0 mL of 1-methyl-2-pyrrolidone was added 12 mg of sodium hydride (content; 55% by weight in oil) at 2° C., and stirred for 30 minutes. Then 0.23 mL of methyl chlorocarbonate was added at 2° C. thereto. The obtained mixture was stirred at room temperature for 3 hours, poured into 10 mL of ice water, and then extracted with 20 mL of ethyl acetate three times. Organic layers were combined, washed with three times a saturated saline, dried over anhydrous magnesium sulfate, and concentrated under reduced pressure. The obtained residue was purified by silica gel chromatography (ethyl acetate:chloroform:hexane=1:1:4) to give a colorless oil. The oil was further purified by median pressure preparative high performance liquid chromatography (ethyl acetate:hexane=20:80) to give 0.62 g of 1-(2,6-difluorobenzoyl)-3-[2-fluoro-4-(trifluormethylthio)phenyl]-1-methoxycarbonyl-3-methylurea (hereinafter, referred to as the present compound (53)).

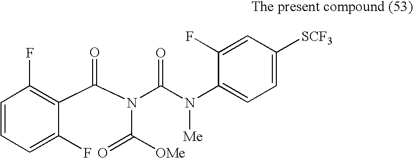

¹H-NMR (CDCl₃) δ (ppm): 3.46 (3H, brs), 3.76 (3H, brs), 6.78-6.83 (2H, m), 7.31-7.33 (1H, m), 7.43-7.51 (3H, m).

### Example 54

To a solution of 1.01 g of 3-(2,6-difluorobenzoyl)-1-[2-fluoro-4-(trifluoromethylthio)phenyl]-1-methylurea in 10.0 mL of 1-methyl-2-pyrrolidone was added 12 mg of sodium hydride (content; 55% by weight in oil) at 2° C., and stirred for 30 minutes. Then 0.23 mL of methanesulfonyl chloride was added at 2° C. thereto. The obtained mixture was stirred at room temperature for 5 hours, poured into 10 mL of ice water, and then extracted with 20 mL of ethyl acetate three times. Organic layers were combined, washed three times with a saturated saline, dried over anhydrous magnesium sulfate, and concentrated under reduced pressure. The obtained residue was purified by silica gel chromatography (ethyl acetate:chloroform:hexane=1:1:4) to give 0.17 g of 1-(2,6-difluorobenzoyl)-3-[2-fluoro-4-(trifluormethylthio)phenyl]-1-methanesulfonyl-3-methylurea (hereinafter, referred to as the present compound (54)).

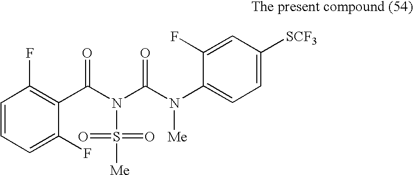

¹H-NMR (DMSO-d₆, Measurement temperature: 80° C.) δ (ppm): 3.40 (3H, brs), 3.51 (3H, brs), 7.16-7.20 (2H, m), 7.45-7.47 (1H, m), 7.61-7.63 (2H, m), 7.71-7.73 (1H, m).

### Example 55

To a solution of 1.01 g of 3-(2,6-difluorobenzoyl)-1-(2-fluoro-4-(trifluoromethylthio)phenyl]-1-methylurea in 10.0 mL of 1-methyl-2-pyrrolidone was added 118 mg of sodium hydride (content; 55% by weight in oil) at 2° C., and stirred for 30 minutes. Then 0.27 mL of dimethylcarbamoyl chloride was added at 2° C. thereto. The obtained mixture was stirred at room temperature for 21.5 hours and then at 80° C. for 5 hours. The reaction mixture was poured into 10 mL of ice water, and was extracted with 20 mL of ethyl acetate three times. Organic layers were combined, washed with a saturated saline three times, dried over anhydrous magnesium sulfate, and concentrated under reduced pressure. The obtained residue was purified by silica gel chromatography (ethyl acetate:chloroform:hexane=1:1:4) to give 0.10 g of 1-(2,6-difluorobenzoyl)-1-(N,N-dimethylcarbamoyl)-3-[2-fluoro-4-(trifluormethylthio)phenyl]-3-methylurea (hereinafter, referred to as the present compound (55)).

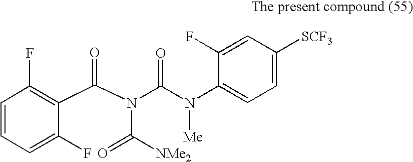

¹H-NMR (CDCl₃) δ (ppm): 2.71 (6H, brs), 3.28 (3H, s), 6.93-6.98 (2H, m), 7.39-7.43 (1H, m), 7.48-7.58 (3H, m).

### Production Example 6

To a solution of 1.00 g of 2-fluoro-N-methyl-4-(trifluoromethylthio)aniline in 10 mL of toluene was added 0.60 mL of triethylamine. Thereto was added dropwise a solution of 1.35 g of bis(trichloromethyl)carbonate in 4 mL of toluene at 1° C. to 8° C. The obtained mixture was stirred for an hour, and then concentrated under reduced pressure. To the residue were added 20 mL of water and 20 mL of chloroform, and then layers separated. The organic layer was washed with 20 mL of a saturated sodium hydrogen carbonate aqueous solution, dried over anhydrous magnesium sulfate, and concentrated under reduced pressure to give 1.26 g of N-[2-fluoro-4-(trifluoromethylthio)phenyl]-N-methylcarbamoyl chloride (purity 91%: as determined by ¹H-NMR).

### N-[2-fluoro-4-(trifluoromethylthio)phenyl]-N-methylcarbamoyl chloride

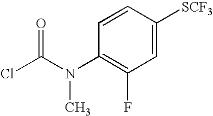

¹H-NMR (CDCl₃) δ (ppm): 3.36-3.49 (3H, m), 7.35-7.39 (1H, m), 7.51-7.53 (2H, m).

### Production Example 7

To a solution of 1.00 g of N-[2-fluoro-4-(trifluoromethylthio)phenyl]-N-methylcarbamoyl chloride in 10 mL of acetonitrile was added 1.00 mL of a 70% ethylamine aqueous solution. The obtained mixture was stirred at room temperature for 20 minutes and then concentrated under reduced pressure. To the residue were added 20 mL of water and 20 mL of chloroform, and then layers separated. The organic layer was dried over anhydrous magnesium sulfate, and concentrated under reduced pressure to give 0.80 g of 3-ethyl-1-[2-fluoro-4-(trifluoromethylthio)phenyl]-1-methylurea.

### 3-Ethyl-1-[2-fluoro-4-(trifluoromethylthio)phenyl]-1-methylurea

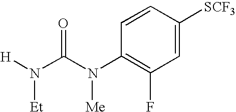

¹H-NMR (CDCl₃) δ (ppm): 1.09 (3H, t, J=7.1 Hz), 3.23 (3H, s), 3.26 (2H, q, J=7.1 Hz), 4.28 (1H, br), 7.35-7.39 (1H, m), 7.47-7.51 (2H, m).

### Example 56

To a solution of 0.80 g of 3-ethyl-1-[2-fluoro-4-(trifluoromethylthio)phenyl]-1-methylurea in 4.0 mL of pyridine was added 0.37 mg of 2,6-difluorobenzoyl chloride. The obtained mixture was stirred at room temperature for 6 days. The reaction mixture was added 20 mL of water and 20 mL of ethyl acetate, and then layers separated. The organic layer was sequentially washed with 20 mL of 7% hydrochloric acid, 20 mL of water and 20 mL of a saturated saline solution, dried over anhydrous magnesium sulfate, and concentrated under reduced pressure. The obtained residue was purified by silica gel chromatography (ethyl acetate:chloroform:hexane=1:1:5) to give 0.90 g of 1-(2,6-difluorobenzoyl)-1-ethyl-3-[2-fluoro-4-(trifluormethylthio)phenyl]-3-methylurea (hereinafter, referred to as the present compound (56)).

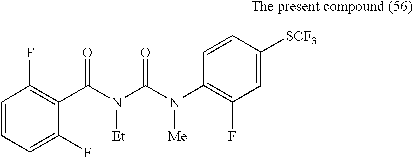

¹H-NMR (DMSO-d₆, Measurement temperature: 80° C.) δ (ppm): 3.18 (3H, t, J=7.1 Hz), 3.26 (3H, s), 3.54 (2H, q, J=7.1 Hz), 7.09-7.13 (2H, m), 7.38-7.42 (1H, m), 7.50-7.58 (2H, m), 7.65-7.68 (1H, m).

### Production Example 8

To a solution of 1.00 g of 2-fluoro-N-methyl-4-(trifluoromethylthio)aniline in 10 mL of toluene was added 0.60 mL of triethylamine. Thereto was added dropwise a solution of 1.30 g of bis(trichloromethyl)carbonate in 4 mL of toluene at 1° C. to 8° C. The obtained mixture was stirred for an hour and then concentrated under reduced pressure to obtain a residue. The residue was dissolved in 10 mL of acetonitrile, and thereto 2.00 mL of a 40% methylamine aqueous solution was added. The mixture was stirred at room temperature for 20 minutes, and then concentrated under reduced pressure. To the residue were added 20 mL of water and 20 mL of chloroform, and then layers separated. The organic layer was dried over anhydrous magnesium sulfate, and concentrated under reduced pressure to give 0.61 g of 1-[2-fluoro-4-(trifluoromethylthio)phenyl]-1,3-dimethylurea.

### 1-[2-Fluoro-4-(trifluoromethylthio)phenyl]-1,3-dimethylurea

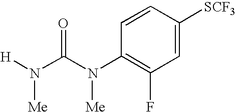

¹H-NMR (CDCl₃) δ (ppm): 2.79 (3H, s), 3.24 (3H, s), 4.23 (1H, br), 7.35-7.39 (1H, m), 7.47-7.51 (2H, m).

### Example 10-(1)

To a solution of 1.00 g of 1-[2-fluoro-4-(trifluoromethylthio)phenyl]-1,3-dimethylurea in 5.0 mL of pyridine was added 0.50 mL of 2,6-difluorobenzoyl chloride. The obtained mixture was stirred at room temperature for three days. The reaction mixture was added to 20 mL of water and 20 mL of ethyl acetate, and then layers separated. The organic layer was washed with 20 mL of 7% hydrochloric acid, washed sequentially with 20 mL of water and 20 mL of a saturated saline solution, dried over anhydrous magnesium sulfate, and concentrated under reduced pressure. The obtained residue was purified by silica gel chromatography (ethyl acetate:hexane=1:1) to give 0.90 g of 1-(2,6-difluorobenzoyl)-3-[2-fluoro-4-(trifluoromethylthio)phenyl]-1,3-dimethylurea (the present compound (10)).

### Example 57

A solution of 1.85 g of 2,6-difluorobenzoyl isocyanate in 2.0 mL of tert-butylmethyl ether was added to a solution of 2.09 g of 4-(difluoromethylthio)-2-fluoro-N-methylaniline in 10 mL of tert-butylmethyl ether at room temperature, and stirred for 30 minutes. A produced solid was collected by filtration, and dried under reduced pressure to give 3.36 g of 3-(2,6-difluorobenzoyl)-1-[4-(difluormethylthio)-2-fluorophenyl]-1-methylurea (hereinafter, referred to as the present compound (57)).

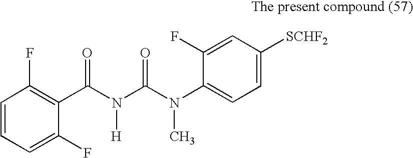

¹H-NMR (CDCl₃) δ (ppm): 3.24 (3H, s), 6.89 (1H, t, J=56.3 Hz), 6.94 (2H, t, J=8.5 Hz), 7.32-7.51 (4H, m), 7.86 (1H, br s).

### Example 58

To a of 2.22 g of 3-(2,6-difluorobenzoyl)-1-[4-(difluoromethylthio)-2-fluorophenyl]-1-methylurea in 15 mL of 1,3-dimethyl-2-imidazolidinone were added 1.60 g of methyl iodide and then 250 mg of sodium hydride (content; 60% by weight in oil), and stirred at room temperature for an hour. To the reaction mixture were added 20 mL of a saturated ammonium chloride aqueous solution and 50 mL of tert-butyl methyl ether, and then layers separated. The organic layer was sequentially washed with water and a saturated saline solution, dried over anhydrous magnesium sulfate, and concentrated under reduced pressure. The obtained residue was purified by medium pressure preparative high performance liquid chromatography (ethyl acetate:hexane=25:75) to give 1.15 g of 1-(2,6-difluorobenzoyl)-3-[4-(difluoromethylthio)]-2-fluorophenyl]-1,3-dimethylurea (hereinafter, referred to as the present compound (58)).

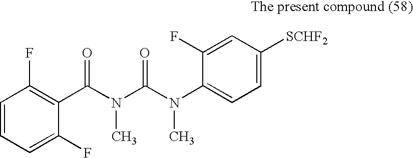

¹H-NMR (DMSO-d₆, Measurement temperature: 80° C.) δ (ppm): 3.05 (3H, s), 3.25 (3H, s), 7.10 (2H, t, J=8.3 Hz), 7.30-7.37 (1H, m), 7.39-7.43 (1H, m), 7.47-7.57 (2H, m), 7.48 (1H, t, J=56.0 Hz).

### Production Example 9

To a solution of 150 mg of allylamine in 10 mL of tert-butylmethyl ether were added 0.36 mL of triethylamine and then 500 mg of N-[2-fluoro-4-(trifluoromethylthio)phenyl]-N-methylcarbamoyl chloride, and stirred at room temperature for 20 minutes. The reaction solution was filtered through Celite, and the filtrate was concentrated under reduced pressure to give 536 mg of 3-allyl-1-[2-fluoro-4-(trifluoromethylthio)phenyl]-1-methylurea.

### 3-Allyl-1-[2-fluoro-4-(trifluoromethylthio)phenyl]-1-methylurea

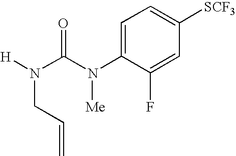

¹H-NMR (CDCl₃) δ (ppm): 3.25 (3H, s), 3.80-3.91-(2H, m), 4.34 (1H, br), 5.04-5.17 (2H, m), 5.77-5.89 (1H, m), 7.35-7.42 (1H, m), 7.46-7.54 (2H, m).

### Example 59

To a solution of 536 mg of 3-allyl-1-[2-fluoro-4-(trifluoromethylthio)phenyl]-1-methylurea and 270 mg of diisopropylethylamine in 7 mL of toluene was added 338 mg of 2,6-difluorobenzoyl chloride, and stirred for 3 hours with heating to reflux. The reaction solution was cooled to room temperature, and thereto 30 mL of tert-butylmethyl ether was added. The mixture was washed sequentially with a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=66:34) to give 0.59 g of 1-allyl-1-(2,6-difluorobenzoyl)-3-[2-fluoro-4-(trifluoromethylthio)phenyl]-3-methylurea (hereinafter, referred to as the present compound (59)).

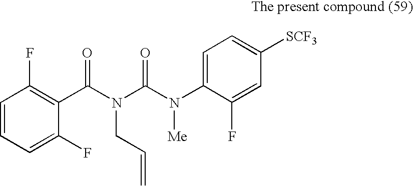

¹H-NMR (DMSO-d₆, Measurement temperature: 80° C.) δ (ppm): 3.26 (3H, s), 4.12 (2H, d, J=5.8 Hz), 5.01-5.18 (2H, m), 5.76-5.89 (1H, m), 7.09 (2H, t, J=8.5 Hz), 7.41 (1H, t, J=8.2 Hz), 7.49-7.59 (2H, m), 7.66 (1H, dd, J=10.0, 1.8 Hz).

### Production Example 10

To a solution of 300 mg of propargylamine in 10 mL of tert-butylmethyl ether were added 0.36 mL of triethylamine and then 500 mg of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure to give 440 mg of 1-[2-fluoro-4-(trifluoromethylthio)phenyl]-1-methyl-3-propargylurea.

### 1-[2-Fluoro-4-(trifluoromethylthio)phenyl]-1-methyl-3-propargylurea

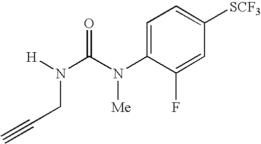

¹H-NMR (CDCl₃) δ (ppm): 2.20 (1H, t, J=2.6 Hz), 3.26 (3H, s), 4.02 (2H, dd, J=5.4, 2.4 Hz), 4.48 (1H, br), 7.38 (1H, t, J=8.2 Hz), 7.47-7.56 (2H, m).

### Example 60

To a solution of 440 mg of 1-[2-fluoro-4-(trifluoromethylthio)phenyl]-1-methyl-3-propargylurea and 223 mg of diisopropylethylamine in 7 mL of toluene was added 279 mg of 2,6-difluorobenzoyl chloride, and stirred for 3 hours with heating to reflux. The reaction solution was cooled to room temperature, and thereto 30 mL of tert-butylmethyl ether was added. The mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=66:34) to give 0.21 g of 1-(2,6-difluorobenzoyl)-3-[2-fluoro-4-(trifluoromethylthio)phenyl]-3-methyl-1-propargylurea (hereinafter, referred to as the present compound (60)).

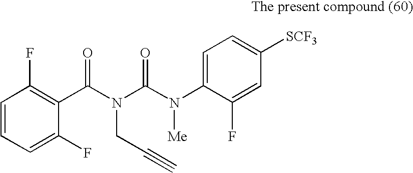

¹H-NMR (DMSO-d₆, Measurement temperature 80° C.) δ (ppm): 3.12 (1H, t, J=2.4 Hz), 3.28 (3H, s), 4.36 (2H, d, J=2.4 Hz), 7.11-(2H, t, J=8.3 Hz), 7.43 (1H, t, J=8.2 Hz), 7.52-7.61-(2H, m), 7.64 (1H, dd, J=10.1, 1.9 Hz).

### Production Example 11

To a solution of 559 mg of benzylamine in 15 mL of tert-butylmethyl ether were added 0.36 mL of triethylamine and then 500 mg of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure to give 690 mg of 3-benzyl-1-[2-fluoro-4-(trifluoromethylthio)phenyl]-1-methylurea.

### 3-Benzyl-1-[2-fluoro-4-(trifluoromethylthio)phenyl]-1-methylurea

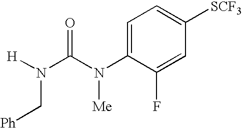

¹H-NMR (CDCl₃) δ (ppm): 3.27 (3H, s), 4.42 (2H, d, J=5.6 Hz), 4.61 (1H, br), 7.21-7.41 (6H, m), 7.43-7.53 (2H, m).

### Example 61

To a solution of 690 mg of 3-benzyl-1-[2-fluoro-4-(trifluoromethylthio)phenyl]-1-methylurea and 0.4 mL of diisopropylethylamine in 10 mL of toluene was added 374 mg of 2,6-difluorobenzoyl chloride, and stirred for 3 hours with heating to reflux. The reaction solution was cooled to room temperature, and thereto 30 mL of tert-butylmethyl ether was added. The mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 0.69 g of 1-benzyl-1-(2,6-difluorobenzoyl)-3-[2-fluoro-4-(trifluoromethylthio)phenyl]-3-methylurea (hereinafter, referred to as the present compound (61)).

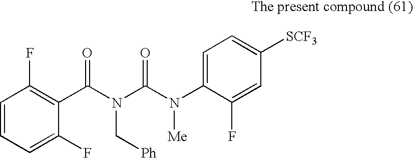

¹H-NMR (DMSO-d₆, Measurement temperature 80° C.) δ (ppm): 3.23 (3H, s), 4.76 (2H, s), 7.06 (2H, t, J=8.5 Hz), 7.18-7.34 (6H, m), 7.48-7.57 (2H, m), 7.64 (1H, dd, J=10.2, 2.0 Hz).

### Production Example 12

To a solution of 1.03 g of 2-phenoxyethylamine in 15 mL of tert-butylmethyl ether were added 0.4 mL of triethylamine and then 650 mg of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=40:60) to give 700 mg of 1-[2-fluoro-4-(trifluoromethylthio)phenyl]-1-methyl-3-(2-phenoxyethyl)urea.

### 1-[2-Fluoro-4-(trifluoromethylthio)phenyl]-1-methyl-3-(2-phenoxyethyl)urea

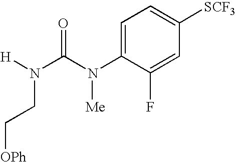

¹H-NMR (CDCl₃) δ (ppm): 3.24 (3H, s), 3.62 (2H, q, J=5.2 Hz), 4.03 (2H, t, J=5.2 Hz), 4.80 (1H, br), 6.80 (2H, d, J=7.8 Hz), 6.96 (1H, t, J=7.4 Hz), 7.22-7.36 (3H, m), 7.41-7.51-(2H, m).

### Example 62

To a solution of 690 mg of 1-[2-fluoro-4-(trifluoromethylthio)phenyl]-1-methyl-3-(2-phenoxyethyl)urea and 0.4 mL of diisopropylethylamine in 10 mL of toluene was added 376 mg of 2,6-difluorobenzoyl chloride, and stirred for 3 hours with heating to reflux. The reaction solution was cooled to room temperature, and thereto 30 mL of tert-butylmethyl ether was added. The mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=66:34) to give 0.67 g of 1-(2,6-difluorobenzoyl)-3-[2-fluoro-4-(trifluoromethylthio)phenyl]-3-methyl-1-(2-phenoxyethyl)urea (hereinafter, referred to as the present compound (62)).

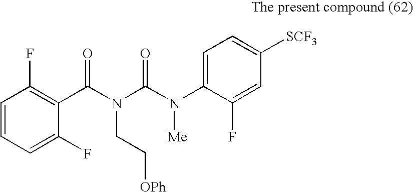

¹H-NMR (DMSO-d₆, Measurement temperature 80° C.) δ (ppm): 3.27 (3H, s), 3.93 (2H, t, J=5.3 Hz), 4.19 (2H, t, J=5.3 Hz), 6.87 (2H, d, J=8.4 Hz), 6.94 (1H, t, J=8.4 Hz), 7.12 (2H, t, J=8.7 Hz), 7.27 (2H, t, J=8.4 Hz), 7.40 (1H, t, J=8.3 Hz), 7.48-7.60 (2H, m), 7.64 (1H, dd, J=10.0, 1.7 Hz).

### Production Example 13

To a solution of 738 mg of tetrahydrofurfurylamine in 15 mL of tert-butylmethyl ether were added 0.4 mL of triethylamine and then 700 mg of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=50:50) to give 760 mg of 1-[2-fluoro-4-(trifluoromethylthio)phenyl]-1-methyl-3-(2-tetrahydrofurylmethyl)urea.

### 1-[2-Fluoro-4-(trifluoromethylthio)phenyl]-1-methyl-3-(2-tetrahydrofurylmethyl)urea

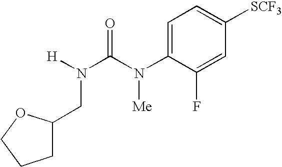

¹H-NMR (CDCl₃) δ (ppm): 1.49-1.62 (1H, m), 1.77-2.00 (3H, m), 3.10-3.21 (1H, m), 3.24 (3H, s), 3.44-3.55 (1H, m), 3.63-3.76 (2H, m), 3.88-3.99 (1H, m), 4.67 (1H, br), 7.37 (1H, t, J=8.2 Hz), 7.44-7.53 (2H, m).

### Example 63

To a solution of 750 mg of 1-[2-fluoro-4-(trifluoromethylthio)phenyl]-1-methyl-3-(2-tetrahydrofurylmethyl)urea and 0.44 mL of diisopropylethylamine in 10 mL of toluene was added 413 mg of 2,6-difluorobenzoyl chloride, and stirred for 3 hours with heating to reflux. The reaction solution was cooled to room temperature, and thereto 30 mL of tert-butylmethyl ether was added. The mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=66:34) to give 0.77 g of 1-(2,6-difluorobenzoyl)-3-[2-fluoro-4-(trifluoromethylthio)phenyl]-3-methyl-1-(2-tetrahydrofurylmethyl)urea (hereinafter, referred to as the present compound (63)).

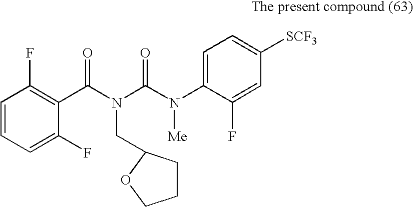

¹H-NMR (DMSO-d₆, Measurement temperature 80° C.) δ (ppm): 1.45-1.58 (1H, m), 1.72-1.85 (2H, m), 1.87-1.99 (1H, m), 3.27 (3H, s), 3.52-3.71 (4H, m), 4.07-4.20 (1H, m), 7.12 (2H, t, J=8.5 Hz), 7.43-7.60 (3H, m), 7.64 (1H, dd, J=10.2, 1.9 Hz).

### Production Example 14

To a solution of 1.0 g of furfurylamine in 25 mL of tert-butylmethyl ether were added 0.53 mL of triethylamine and then 1.0 g of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (ethyl acetate) to give 830 mg of 1-[2-fluoro-4-(trifluoromethylthio)phenyl]-3-(2-furylmethyl)-1-methylurea.

### 1-[2-Fluoro-4-(trifluoromethylthio)phenyl]-3-(2-furylmethyl)-1-methylurea

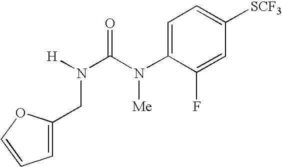

¹H-NMR (CDCl₃) δ (ppm): 3.25 (3H, s), 4.40 (2H, d, J=5.6 Hz), 4.63 (1H, br), 6.17-6.22 (1H, m), 6.27-6.32 (1H, m), 7.30-7.39 (2H, m), 7.43-7.53 (2H, m).

### Example 64

To a solution of 700 mg of 1-[2-fluoro-4-(trifluoromethylthio)phenyl]-3-(2-furylmethyl)-1-methylurea and 0.42 mL of diisopropylethylamine in 10 mL of toluene was added 390 mg of 2,6-difluorobenzoyl chloride, and stirred for 3 hours with heating to reflux. The reaction solution was cooled to room temperature, and thereto 30 mL of tert-butylmethyl ether was added. The mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 0.36 g of 1-(2,6-difluorobenzoyl)-3-[2-fluoro-4-(trifluoromethylthio)phenyl]-1-(2-furylmethyl)-3-methylurea (hereinafter, referred to as the present compound (64)).

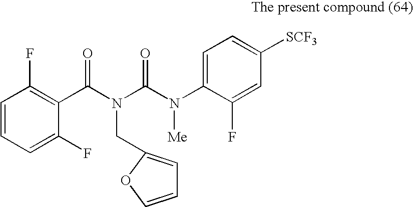

¹H-NMR (DMSO-d₆, Measurement temperature 80° C.) δ (ppm): 3.21 (3H, s), 4.76 (2H, s), 6.24-6.31 (1H, m), 6.37-6.43 (1H, m), 7.05-7.22 (3H, m), 7.45-7.69 (4H, m).

### Production Example 15

To a solution of 1.38 g of N,N-dimethylethylenediamine in 35 mL of tert-butylmethyl ether were added 0.87 mL of triethylamine and then 1.5 g of N-[2-fluoro-4-(trifluoromethylthio)phenyl]-N-methylcarbamoyl chloride, and stirred at room temperature for an hour. The reaction solution was washed sequentially with water and a saturated saline solution, dried over anhydrous magnesium sulfate, and concentrated under reduced pressure. The obtained residue was purified by medium pressure preparative high performance liquid chromatography (ethyl acetate:methanol=90:10) to give 1.46 g of 3-(2-dimethylaminoethyl)-1-[2-fluoro-4-(trifluoromethylthio)phenyl]-1-methylurea.

### 3-(2-Dimethylaminoethyl)-1-[2-fluoro-4-(trifluoromethylthio)phenyl]-1-methylurea

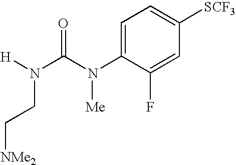

¹H-NMR (CDCl₃) δ (ppm): 2.13 (6H, s), 2.34 (2H, t, J=6.0 Hz), 3.22-3.32 (5H, m), 5.01 (1H, br), 7.37 (1H, t, J=8.2 Hz), 7.44-7.51-(2H, m).

### Example 65

To a solution of 1.2 g of 3-(2-dimethylaminoethyl)-1-[2-fluoro-4-(trifluoromethylthio)phenyl]-1-methylurea and 0.74 mL of diisopropylethylamine in 15 mL of toluene was added 687 mg of 2,6-difluorobenzoyl chloride, and stirred for 3 hours with heating to reflux. The reaction solution was cooled to room temperature, and thereto 80 mL of tert-butylmethyl ether was added. The mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (ethyl acetate) to give 1.46 g of 1-(2,6-difluorobenzoyl)-2-(2-dimethylaminoethyl)-3-[2-fluoro-4-(trifluoromethylthio)phenyl]-3-methylurea (hereinafter, referred to as the present compound (65)).

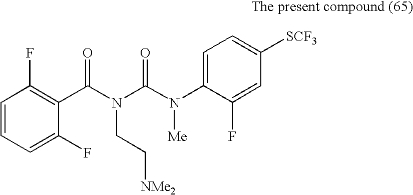

¹H-NMR (DMSO-d₆, Measurement temperature 80° C.) δ (ppm): 2.10 (6H, s), 2.48 (2H, t, J=6.4 Hz), 3.26 (3H, s), 3.61-(2H, t, J=6.4 Hz), 7.12 (2H, t, J=8.5 Hz), 7.46 (1H, t, J=8.0 Hz), 7.51-7.60 (2H, m), 7.65 (1H, dd, J=10.1, 1.7 Hz).

### Production Example 16

To a solution of 2.74 g of aminoacetoaldehyde dimethylacetal in 80 mL of tert-butylmethyl ether were added 3.6 mL of triethylamine and then 5.0 g of N-[2-fluoro-4-(trifluoromethylthio)phenyl]-N-methylcarbamoyl chloride, and stirred at room temperature for an hour. The reaction solution was washed sequentially with water and a saturated saline solution, dried over anhydrous magnesium sulfate, and concentrated under reduced pressure. The obtained residue was purified by medium pressure preparative high performance liquid chromatography (hexane:ethyl acetate=50:50) to give 6.18 g of 3-(2,2-dimethoxyethyl)-1-[2-fluoro-4-(trifluoromethylthio)phenyl]-1-methylurea.

### 3-(2,2-Dimethoxyethyl)-1-[2-fluoro-4-(trifluoromethylthio)phenyl]-1-methylurea

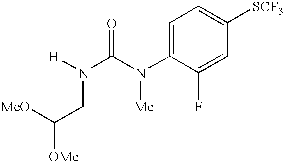

¹H-NMR (CDCl₃) δ (ppm): 3.24 (3H, s), 3.34 (2H, t, J=5.5 Hz), 3.36 (6H, s), 4.35 (1H, t, J=5.5 Hz), 4.52 (1H, br), 7.37 (1H, t, J=8.3 Hz), 7.45-7.53 (2H, m).

### Example 66

To a solution of 6.18 g of 3-(2,2-dimethoxylethyl)-1-[2-fluoro-4-(trifluoromethylthio)phenyl]-1-methylurea and 5.4 mL of diisopropylethylamine in 50 mL of toluene was added 3.98 g of 2,6-difluorobenzoyl chloride, and stirred for 3 hours with heating to reflux. The reaction solution was cooled to room temperature, and thereto 150 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 7.78 g of 1-(2,6-difluorobenzoyl)-2-(2,2-dimethoxyethyl)-3-[2-fluoro-4-(trifluoromethylthio)phenyl]-3-methylurea (hereinafter, referred to as the present compound (66)).

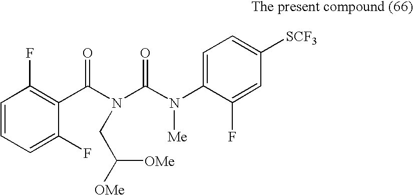

¹H-NMR (DMSO-d₆, Measurement temperature: 80° C.) δ (ppm): 3.27 (3H, s), 3.30 (6H, s), 3.61-(2H, d, J=5.0 Hz), 4.63 (1H, t, J=5.0 Hz), 7.12 (2H, t, J=8.6 Hz), 7.44 (1H, t, J=8.1 Hz), 7.51-7.60 (2H, m), 7.65 (1H, dd, J=10.1, 1.9 Hz).

### Production Example 17

To a solution of 660 mg of 2-aminomethyl-1,3-dioxolan in 20 mL of tert-butylmethyl ether were added 0.33 mL of triethylamine and then 614 mg of N-[2-fluoro-4-(trifluoromethylthio)phenyl]-N-methylcarbamoyl chloride, and stirred at room temperature for an hour. The reaction solution was washed sequentially with water and a saturated saline solution, dried over anhydrous magnesium sulfate, and concentrated under reduced pressure. The obtained residue was purified by medium pressure preparative high performance liquid chromatography (hexane:ethyl acetate=50:50) to give 600 mg of 3-[(1,3-dioxolan-2-yl)methyl]-1-[2-fluoro-4-(trifluoromethylthio)phenyl]-1-methylurea.

### 3-(1,3-Dioxolan-2-yl)methyl]-1-[2-fluoro-4-(trifluoromethylthio)phenyl]-1-methylurea

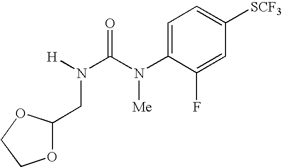

¹H-NMR (CDCl₃) δ (ppm): 3.25 (3H, s), 3.45 (2H, dd, J=5.9, 3.7 Hz), 3.82-3.92 (4H, m), 4.54 (1H, br), 4.93 (1H, t, J=3.7 Hz), 7.38 (1H, t, J=8.2 Hz), 7.45-7.53 (2H, m).

### Example 67

To a solution of 450 mg of 3-[(1,3-dioxolan-2-yl)methyl]-1-[2-fluoro-4-(trifluoromethylthio)phenyl]-1-methylurea and 0.2 mL of diisopropylethylamine in 10 mL of toluene was added 247 mg of 2,6-difluorobenzoyl chloride, and stirred for 3 hours with heating to reflux. The reaction solution was cooled to room temperature, and thereto 30 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=66:34) to give 390 mg of 1-(2,6-difluorobenzoyl)-1-[(1,3-dioxolan-2-yl)methyl-3-[2-fluoro-4-(trifluoromethylthio)phenyl]-3-methylurea (hereinafter, referred to as the present compound (67)).

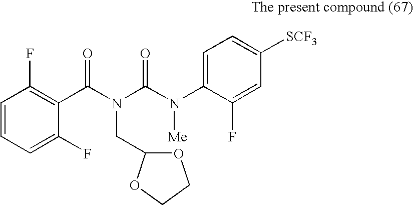

¹H-NMR (DMSO-d₆, Measurement temperature 80° C.) δ (ppm): 3.26 (3H, s), 3.68 (2H, d, J=4.2 Hz), 3.81 (4H, s), 5.14 (1H, t, J=4.2 Hz), 7.13 (2H, t, J=8.6 Hz), 7.45 (1H, t, J=8.0 Hz), 7.51-7.61-(2H, m), 7.66 (1H, dd, J=10.4, 1.9 Hz).

### Production Example 18

To a solution of 261 mg of 2-methoxyethylamine in 25 mL of tert-butylmethyl ether were added 0.73 mL of triethylamine and then 1.0 g of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure to give 1.06 g of 1-[(2-fluoro-4-(trifluoromethylthio)phenyl]-3-(2-methoxyethyl)-1-methylurea.

### 1-[(2-Fluoro-4-(trifluoromethylthio)phenyl]-3-(2-methoxyethyl)-1-methylurea

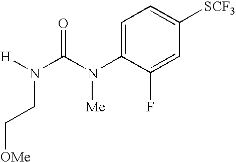

¹H-NMR (CDCl₃) δ (ppm): 3.24 (3H, s), 3.29 (3H, s), 3.36-3.46 (4H, m), 4.70 (1H, br), 7.37 (1H, t, J=8.2 Hz), 7.45-7.53 (2H, m).

### Example 68

To a solution of 840 mg of 1-[(2-fluoro-4-(trifluoromethylthio)phenyl]-3-(2-methoxyethyl)-1-methylurea and 0.54 mL of diisopropylethylamine in 15 mL of toluene was added 500 mg of 2,6-difluorobenzoyl chloride, and stirred for 3 hours with heating to reflux. The reaction solution was cooled to room temperature, and thereto 50 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 1.04 g of 1-(2,6-difluorobenzoyl)-3-[2-fluoro-4-(trifluoromethylthio)phenyl-1-(2-methoxyethyl)-3-methylurea (hereinafter, referred to as the present compound (68)).

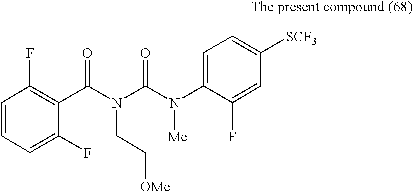

¹H-NMR (DMSO-d₆, Measurement temperature 80° C.) δ (ppm): 3.22 (3H, s), 3.25 (3H, s), 3.53 (2H, t, J=5.6 Hz), 3.70 (2H, t, J=5.6 Hz), 7.11 (2H, t, J=8.3 Hz), 7.42 (1H, t, J=8.1 Hz), 7.50-7.59 (2H, m), 7.65 (1H, dd, J=10.1, 1.9 Hz).

### Production Example 19

To a solution of 600 mg of cyclopropylamine in 30 mL of tert-butylmethyl ether were added 0.73 mL of triethylamine and then 1.0 g of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure to give 950 mg of 3-cyclopropyl-1-[2-fluoro-4-(trifluoromethylthio)phenyl]-1-methylurea.

### 3-Cyclopropyl-1-[2-fluoro-4-(trifluoromethylthio)phenyl]-1-methylurea

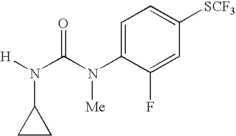

¹H-NMR (CDCl₃) δ (ppm): 0.39-0.46 (2H, m), 0.67-0.74 (2H, m), 2.57-2.66 (1H, m), 3.23 (3H, s), 4.49 (1H, br), 7.33 (1H, t, J=8.2 Hz), 7.44-7.52 (2H, m).

### Example 69

To a solution of 750 mg of 3-cyclopropyl-1-[2-fluoro-4-(trifluoromethylthio)phenyl]-1-methylurea and 0.5 mL of diisopropylethylamine in 15 mL of toluene was added 472 mg of 2,6-difluorobenzoyl chloride, and stirred for 3 hours with heating to reflux. The reaction solution was cooled to room temperature, and thereto 50 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 930 mg of 1-cyclopropyl-1-(2,6-difluorobenzoyl)-3-[2-fluoro-4-(trifluoromethylthio)phenyl-3-methylurea (hereinafter, referred to as the present compound (69)).

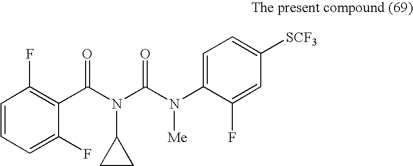

¹H-NMR (DMSO-d₆, Measurement temperature 80° C.) δ (ppm): 0.60-0.73 (4H, m), 2.55-2.65 (1H, m), 3.30 (3H, s), 7.11-(2H, t, J=8.3 Hz), 7.46-7.63 (3H, m), 7.71 (1H, dd, J=10.1, 1.4 Hz).

### Production Example 20

To a solution of 1.03 mg of 2,2,2-trifluoroethylamine in 30 mL of tert-butylmethyl ether were added 0.73 mL of triethylamine and then 1.0 g of N-[2-fluoro-4-(trifluoromethylthio)phenyl]-N-methylcarbamoyl chloride, and stirred at room temperature for 24 hours. The reaction solution was washed sequentially with 2N hydrochloric acid, a saturated sodium hydrogen carbonate aqueous solution and a saturated saline solution, dried over anhydrous magnesium sulfate, and concentrated under reduced pressure to give 1.1 g of 1-[2-fluoro-4-(trifluoromethylthio)phenyl]-1-methyl-3-(2,2,2-trifluoroethyl)urea.

### 1-[2-Fluoro-4-(trifluoromethylthio)phenyl]-1-methyl-3-(2,2,2-trifluoroethyl)urea

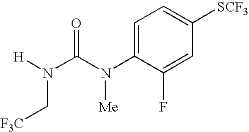

¹H-NMR (CDCl₃) δ (ppm): 3.27 (3H, s), 3.81-3.95 (2H, m), 4.56 (1H, br), 7.38 (1H, t, J=8.2 Hz), 7.48-7.59 (2H, m).

### Example 70

To a solution of 893 mg of 1-[2-fluoro-4-(trifluoromethylthio)phenyl]-1-methyl-3-(2,2,2-trifluoroethyl)urea and 0.58 mL of diisopropylethylamine in 15 mL of toluene was added 1.5 g of 2,6-difluorobenzoyl chloride, and stirred for 26 hours with heating to reflux. The reaction solution was cooled to room temperature, and thereto 50 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 1.0 g of 1-(2,6-difluorobenzoyl)-3-[2-fluoro-4-(trifluoromethylthio)phenyl-3-methyl-1-(2,2,2-trifluoroethyl)urea (hereinafter, referred to as the present compound (70)).

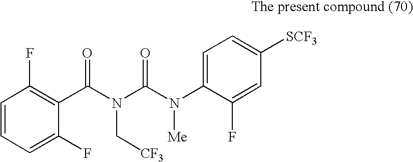

¹H-NMR (DMSO-d₆, Measurement temperature 80° C.) δ (ppm): 3.25 (3H, s), 4.54 (2H, q, J=9.1 Hz), 7.18 (2H, t, J=8.7 Hz), 7.30 (1H, t, J=8.1 Hz), 7.56 (1H, d, J=8.2 Hz), 7.59-7.69 (2H, m).

### Production Example 21

To a solution of 383 mg of cyclopropylmethylamine in 30 mL of tert-butylmethyl ether were added 0.73 mL of triethylamine and then 1.0 g of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure to give 613 mg of 3-cyclopropylmethyl-1-[2-fluoro-4-(trifluoromethylthio)phenyl]-1-methylurea.

### 3-Cyclopropylmethyl-1-[2-fluoro-4-(trifluoromethylthio)phenyl]-1-methylurea

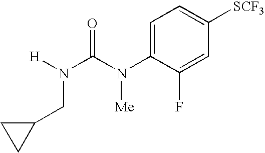

¹H-NMR (CDCl₃) δ (ppm): 0.09-0.16 (2H, m), 0.39-0.48 (2H, m) 0.84-0.98 (1H, m), 3.09 (2H, dd, J=6.8, 5.8 Hz), 3.24 (3H, s), 4.34 (1H, br), 7.37 (1H, t, J=8.2 Hz), 7.44-7.53 (2H, m).

### Example 71

To a solution of 500 mg of 3-cyclopropylmethyl-1-[2-fluoro-4-(trifluoromethylthio)phenyl]-1-methylurea and 0.32 mL of diisopropylethylamine in 10 mL of toluene was added 301 mg of 2,6-difluorobenzoyl chloride, and stirred for 3 hours with heating to reflux. The reaction solution was cooled to room temperature, and thereto 50 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 665 mg of 1-cyclopropylmethyl-1-(2,6-difluorobenzoyl)-3-[2-fluoro-4-(trifluoromethylthio)phenyl]-3-methylurea (hereinafter, referred to as the present compound (71))

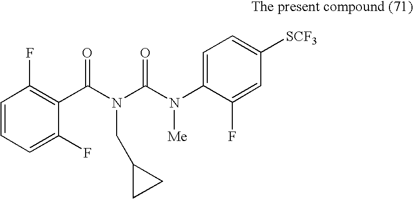

¹H-NMR (DMSO-d₆, Measurement temperature 80° C.) δ (ppm): 0.11-0.29 (2H, m), 0.43-0.59 (2H, m), 1.01-1.19 (1H, m), 3.29 (3H, s), 3.43 (2H, d, J=6.8 Hz), 7.13 (2H, t, J=8.4 Hz), 7.44 (1H, t, J=8.0 Hz), 7.50-7.61-(2H, m), 7.67 (1H, dd, J=10.2, 2.0 Hz).

### Production Example 22

To a solution of 590 mg of cyclohexylmethylamine in 30 mL of tert-butylmethyl ether were added 0.73 mL of triethylamine and then 1.0 g of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure to give 910 mg of 3-cyclohexylmethyl-1-[2-fluoro-4-(trifluoromethylthio)phenyl]-1-methylurea.

### 3-Cyclohexylmethyl-1-[2-fluoro-4-(trifluoromethylthio)phenyl]-1-methylurea

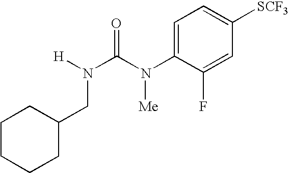

¹H-NMR (CDCl₃) δ (ppm): 0.78-0.91-(2H, m), 1.04-1.27 (3H, m), 1.36-1.48 (1H, m), 1.59-1.75 (5H, m), 3.04 (2H, t, J=6.4 Hz), 3.24 (3H, s), 4.31 (1H, br), 7.37 (1H, t, J=8.3 Hz), 7.46-7.54 (2H, m).

### Example 72

To a solution of 700 mg of 3-cyclohexylmethyl-1-[2-fluoro-4-(trifluoromethylthio)phenyl]-1-methylurea and 0.40 mL of diisopropylethylamine in 15 mL of toluene was added 373 mg of 2,6-difluorobenzoyl chloride, and stirred for 3 hours with heating to reflux. The reaction solution was cooled to room temperature, and thereto 50 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 690 mg of 1-cyclohexylmethyl-1-(2,6-difluorobenzoyl)-3-[2-fluoro-4-(trifluoromethylthio)phenyl]-3-methylurea (hereinafter, referred to as the present compound (72)).

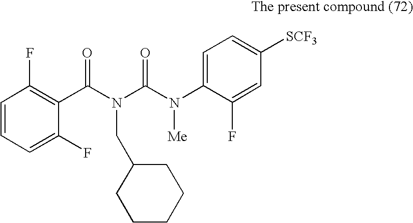

¹H-NMR (DMSO-d₆, Measurement temperature 80° C.) δ (ppm): 0.81-0.98 (2H, m), 1.05-1.26 (3H, m), 1.54-1.80 (6H, m), 3.26 (3H, s), 3.40 (2H, d, J=6.3 Hz), 7.12 (2H, t, J=8.3 Hz), 7.41 (1H, t, J=8.2 Hz), 7.49-7.62 (2H, m), 7.69 (1H, dd, J=10.1, 1.9 Hz).

### Production Example 23

To a solution of 2.0 g of 2-methylthioethylamine in 80 mL of tert-butylmethyl ether were added 3.1 mL of triethylamine and then 4.2 g of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure to give 4.5 g of 1-[2-fluoro-4-(trifluoromethylthio)phenyl]-1-methyl-3-(2-methylthioethyl)urea.

### 1-[2-Fluoro-4-(trifluoromethylthio)phenyl]-1-methyl-3-(2-methylthioethyl)urea

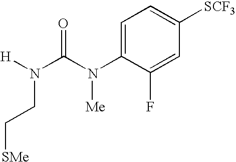

¹H-NMR (CDCl₃) δ (ppm): 2.05 (3H, s), 2.62 (2H, t, J=6.3 Hz), 3.25 (3H, s), 3.41 (2H, q, J=6.3 Hz), 4.76 (1H, br), 7.39 (1H, t, J=8.2 Hz), 7.46-7.54 (2H, m)

### Example 73

To a solution of 4.33 g of 1-[2-fluoro-4-(trifluoromethylthio)phenyl]-1-methyl-3-(2-methylthioethyl)urea and 2.6 mL of diisopropylethylamine in 50 mL of toluene was added 2.45 g of 2,6-difluorobenzoyl chloride, and stirred for 6 hours with heating to reflux. The reaction solution was cooled to room temperature, and thereto 100 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 5.58 g of 1-(2,6-difluorobenzoyl)-3-[2-fluoro-4-(trifluoromethylthio)phenyl]-3-methyl-3-(2-methylthioethyl)urea (hereinafter, referred to as the present compound (73)).

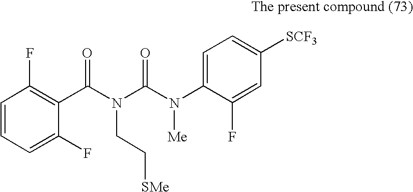

¹H-NMR (DMSO-d₆, Measurement temperature 80° C.) δ (ppm): 1.98 (3H, s), 2.72 (2H, t, J=7.4 Hz), 3.26 (3H, s), 3.71-(2H, t, J=7.4 Hz), 7.13 (2H, t, J=8.5 Hz), 7.40 (1H, t, J=8.2 Hz), 7.52-7.62 (2H, m), 7.67 (1H, dd, J=10.1, 1.9 Hz).

### Examples 74 and 75

To a solution of 2.00 g of 1-(2,6-difluorobenzoyl)-3-[2-fluoro-4-(trifluoromethylthio)phenyl]-3-methyl-3-(2-methylthioethyl)urea in 35 mL of chloroform which was ice-cooled to 5° C. was added 1.63 g of meta-chloroperbenzoic acid (content; 65% by weight), and stirred at room temperature for an hour. The reaction solution was washed sequentially with a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=50:50) to give 794 mg of 1-(2,6-difluorobenzoyl)-3-[2-fluoro-4-(trifluoromethylthio)phenyl]-3-methyl-3-(2-methylsulfinylethyl)urea (hereinafter, referred to as the present compound (74)) and 1.29 g of 1-(2,6-difluorobenzoyl)-3-[2-fluoro-4-(trifluoromethylthio)phenyl]-3-methyl-3-(2-methylsulfonylethyl)urea (hereinafter, referred to as the present compound (75)).

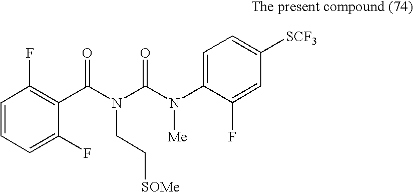

¹H-NMR (DMSO-d₆, Measurement temperature 80° C.) δ (ppm): 2.57 (3H, s), 2.96 (1H, dt, J=13.4, 7.2 Hz), 3.13 (1H, dt, J=13.4, 7.2 Hz), 3.25 (3H, s), 3.96 (2H, t, J=7.2 Hz), 7.13 (2H, t, J=8.3 Hz), 7.38 (1H, t, J=8.2 Hz), 7.53-7.62 (2H, m), 7.66 (1H, dd, J=10.1, 1.9 Hz).

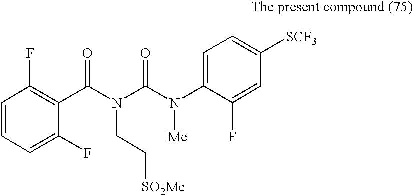

¹H-NMR (DMSO-d₆, Measurement temperature 80° C.) δ (ppm): 3.03 (3H, s), 3.24 (3H, s), 3.48 (2H, t, J=7.5 Hz), 4.02 (2H, t, J=7.5 Hz), 7.13 (2H, t, J=8.6 Hz), 7.33 (1H, t, J=8.2 Hz), 7.51-7.69 (3H, m).

### Production Example 24

To a solution of 1.2 g of 2-pyridylmethylamine in 30 mL of tert-butylmethyl ether were added 1.2 mL of triethylamine and then 1.5 g of N-[2-fluoro-4-(trifluoromethylthio)phenyl]-N-methylcarbamoyl chloride, and stirred at room temperature for an hour. The reaction solution was washed sequentially with water and a saturated saline solution, dried over anhydrous magnesium sulfate, and concentrated under reduced pressure. The obtained residue was purified by medium pressure preparative high performance liquid chromatography (ethyl acetate) to give 1.55 g of 1-[2-fluoro-4-(trifluoromethylthio)phenyl]-1-methyl-3-(2-pyridylmethyl)urea.

### 1-[2-Fluoro-4-(trifluoromethylthio)phenyl]-1-methyl-3-(2-pyridylmethyl)urea

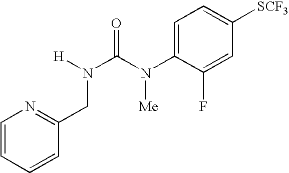

¹H-NMR (CDCl₃) δ (ppm): 3.29 (3H, s), 4.52 (2H, d, J=5.1 Hz), 5.67 (1H, br), 7.15 (1H, dd, J=7.7, 5.1 Hz), 7.27 (1H, d, J=7.7 Hz), 7.43 (1H, t, J=8.1 Hz), 7.47-7.53 (2H, m), 7.64 (1H, td, J=7.7, 1.8 Hz), 8.41 (1H, dd, J=5.1, 1.8 Hz).

### Example 76

To a solution of 1.46 g of 1-[2-fluoro-4-(trifluoromethylthio)phenyl]-1-methyl-3-(2-pyridylmethyl)urea and 0.85 mL of diisopropylethylamine in 20 mL of toluene was added 789 mg of 2,6-difluorobenzoyl chloride, and stirred for 3 hours with heating to reflux. The reaction solution was cooled to room temperature, and thereto 70 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=66:34) to give 1.55 g of 1-(2,6-difluorobenzoyl)-3-[2-fluoro-4-(trifluoromethylthio)phenyl]-3-methyl-1-(2-pyridylmethyl)urea (hereinafter, referred to as the present compound (76)).

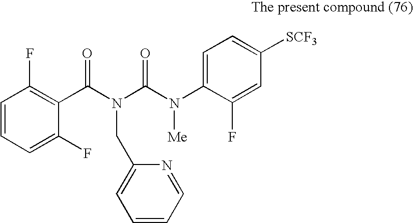

¹H-NMR (DMSO-d₆, Measurement temperature 80° C.) δ (ppm): 3.28 (3H, s), 4.87 (2H, s), 7.06 (2H, t, J=8.7 Hz), 7.25 (1H, dd, J=7.7, 4.9 Hz), 7.33 (1H, d, J=7.7 Hz), 7.44 (1H, t, J=8.1 Hz), 7.47-7.57 (2H, m), 7.63 (1H, dd, J=10.1, 1.9 Hz), 7.73 (1H, td, J=7.7, 1.5 Hz), 8.46 (1H, dd, J=4.9, 1.5 Hz).

### Production Example 25

To a solution of 1.13 g of 3-pyridylmethylamine in 30 mL of tert-butylmethyl ether were added 1.1 mL of triethylamine and then 1.5 g of N-[2-fluoro-4-(trifluoromethylthio)phenyl]-N-methylcarbamoyl chloride, and stirred at room temperature for an hour. The reaction solution was washed sequentially with water and a saturated saline solution, dried over anhydrous magnesium sulfate, and concentrated under reduced pressure. The obtained residue was purified by medium pressure preparative high performance liquid chromatography (ethyl acetate) to give 1.55 g of 1-[2-fluoro-4-(trifluoromethylthio)phenyl]-1-methyl-3-(3-pyridylmethyl)urea.

### 1-[2-Fluoro-4-(trifluoromethylthio)phenyl]-1-methyl-3-(3-pyridylmethyl)urea

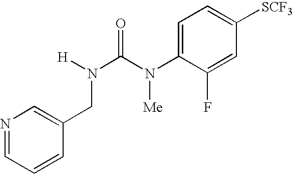

¹H-NMR (CDCl₃) δ (ppm): 3.27 (3H, s), 4.43 (2H, d, J=6.0 Hz), 4.73 (1H, br), 7.22-7.28 (1H, m), 7.37 (1H, t, J=8.2 Hz), 7.45-7.53 (2H, m), 7.60-7.66 (1H, m), 8.46-8.52 (2H, m).

### Example 77

To a solution of 1.32 g of 1-[2-fluoro-4-(trifluoromethylthio)phenyl]-1-methyl-3-(3-pyridylmethyl)urea and 0.77 mL of diisopropylethylamine in 20 mL of toluene was added 713 mg of 2,6-difluorobenzoyl chloride, and stirred for 3 hours with heating to reflux. The reaction solution was cooled to room temperature, and thereto 70 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=50:50) to give 1.55 g of 1-(2,6-difluorobenzoyl)-3-[2-fluoro-4-(trifluoromethylthio)phenyl]-3-methyl-1-(3-pyridylmethyl)urea (hereinafter, referred to as the present compound (77)).

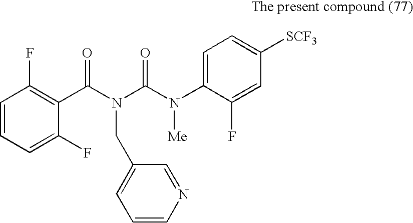

¹H-NMR (DMSO-d₆, Measurement temperature 80° C.) δ (ppm): 3.26 (3H, s), 4.82 (2H, s), 7.07 (2H, t, J=8.5 Hz), 7.24-7.36 (2H, m), 7.49-7.59 (2H, m), 7.65 (1H, dd, J=10.1, 1.9 Hz), 7.71 (1H, d, J=8.0 Hz), 8.42-8.50 (2H, m).

### Production Example 26

To a solution of 775 mg of 5-aminomethyl-2-chlorothiazole in 30 mL of tert-butylmethyl ether were added 0.7 mL of triethylamine and then 1.0 g of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure to give 1.20 g of 3-[2-chlorothizol-5-yl)methyl]-1-[2-fluoro-4-(trifluoromethylthio)phenyl]-1-methylurea.

### 3-[2-Chlorothizol-5-yl)methyl]-1-[2-fluoro-4-(trifluoromethylthio)phenyl]-1-methylurea

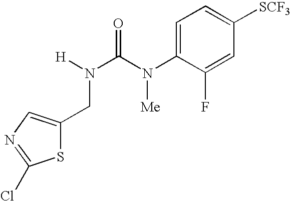

¹H-NMR (CDCl₃) δ (ppm): 3.26 (3H, s), 4.47 (2H, d, J=5.7 Hz), 4.79 (1H, br), 7.32 (1H, s), 7.35 (1H, t, J=8.2 Hz), 7.47-7.54 (2H, m).

### Example 78

To a solution of 994 mg of 3-[2-chlorothizol-5-yl)methyl]-1-[2-fluoro-4-(trifluoromethylthio)phenyl]-1-methylurea and 1.0 mL of diisopropylethylamine in 20 mL of toluene was added 983 mg of 2,6-difluorobenzoyl chloride, and stirred for 3 hours with heating to reflux. The reaction solution was cooled to room temperature, and thereto 80 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 1.30 g of 1-[(2-chlorothiazol-5-yl)methyl]-1-(2,6-difluorobenzoyl)-3-[2-fluoro-4-(trifluoromethylthio)phenyl]-3-methylurea (hereinafter, referred to as the present compound (78)).

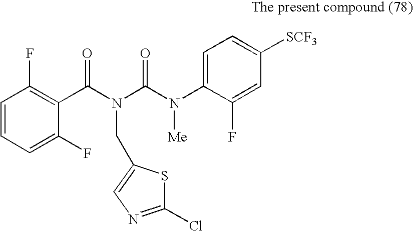

¹H-NMR (DMSO-d₆, Measurement temperature 80° C.) δ (ppm): 3.25 (3H, s), 4.96 (2H, s), 7.10 (2H, t, J=8.5 Hz), 7.26 (1H, t, J=8.3 Hz), 7.39 (1H, s), 7.48-7.68 (3H, m).

### Production Example 27

To a solution of 533 mg of 1-aminomorpholine in 30 mL of tert-butylmethyl ether were added 1.5 mL of triethylamine and then 1.0 g of N-[2-fluoro-4-(trifluoromethylthio)phenyl]-N-methylcarbamoyl chloride, and stirred at room temperature for 16 hours. The reaction solution was washed sequentially with water and a saturated sodium bicarbonate aqueous solution, dried over anhydrous magnesium sulfate, and concentrated under reduced pressure. The obtained residue was purified by medium pressure preparative high performance liquid chromatography (ethyl acetate) to give 1.02 g of 1-[2-fluoro-4-(trifluoromethylthio)phenyl]-1-methyl-3-morpholinourea.

### 1-[2-Fluoro-4-(trifluoromethylthio)phenyl]-1-methyl-3-morpholinourea

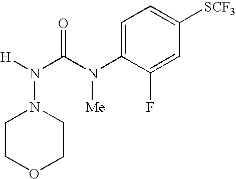

¹H-NMR (CDCl₃) δ (ppm): 2.57 (4H, t, J=4.6 Hz), 3.22 (3H, s), 3.46 (4H, t, J=4.6 Hz), 5.15 (1H, br), 7.30 (1H, t, J=8.1 Hz), 7.43-7.52 (2H, m).

### Example 79

To a solution of 800 mg of 1-[2-fluoro-4-(trifluoromethylthio)phenyl]-1-methyl-3-morpholinourea and 1.0 mL of diisopropylethylamine in 20 mL of toluene was added 800 mg of 2,6-difluorobenzoyl chloride, and stirred for 3 hours with heating to reflux. The reaction solution was cooled to room temperature, and thereto 80 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 1.02 g of 1-(2,6-difluorobenzoyl)-3-[2-fluoro-4-(trifluoromethylthio)phenyl]-3-methyl-1-morpholinourea (hereinafter, referred to as the present compound (79)).

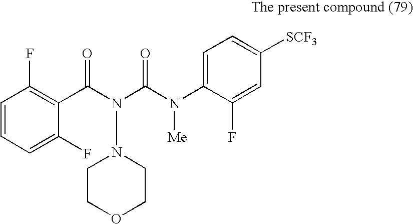

¹H-NMR (DMSO-d₆, Measurement temperature 80° C.) δ (ppm): 2.94-3.11 (4H, br), 3.22-3.43 (4H, br), 3.30 (3H, s), 7.11-(2H, t, J=8.6 Hz), 7.47-7.63 (3H, m), 7.74 (1H, dd, J=10.1, 1.9 Hz).

### Production Example 28

To a solution of 1.03 g of 1-(aminoethyl)morpholine in 30 mL of tert-butylmethyl ether were added 1.5 mL of triethylamine and then 1.0 g of N-[2-fluoro-4-(trifluoromethylthio)phenyl]-N-methylcarbamoyl chloride, and stirred at room temperature for 3 hours. The reaction solution was washed sequentially with water and a saturated saline solution, dried over anhydrous magnesium sulfate, and concentrated under reduced pressure. The obtained residue was purified by medium pressure preparative high performance liquid chromatography (ethyl acetate) to give 1.23 g of 1-[2-fluoro-4-(trifluoromethylthio)phenyl]-1-methyl-3-(2-morpholinoethyl)urea.

### 1-[2-Fluoro-4-(trifluoromethylthio)phenyl]-1-methyl-3-(2-morpholinoethyl)urea

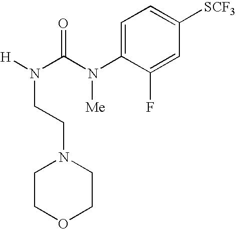

¹H-NMR (CDCl₃) δ (ppm): 2.32-2.38 (4H, br), 2.42 (2H, t, J=6.0 Hz), 3.25 (3H, s), 3.27-3.33 (2H, m), 3.47-3.59 (4H, br), 5.02 (1H, br), 7.38 (1H, t, J=8.3 Hz), 7.48-7.54 (2H, m).

### Example 80

To a solution of 1.0 g of 1-[2-fluoro-4-(trifluoromethylthio)phenyl]-1-methyl-3-(2-morpholinoethyl)urea and 1.0 mL of diisopropylethylamine in 15 mL of toluene was added 694 mg of 2,6-difluorobenzoyl chloride, and stirred for 3 hours with heating to reflux. The reaction solution was cooled to room temperature, and thereto 50 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=66:34) to give 1.30 g of 1-(2,6-difluorobenzoyl)-3-[2-fluoro-4-(trifluoromethylthio)phenyl]-3-methyl-1-(2-morpholinoethyl)urea (hereinafter, referred to as the present compound (80)).

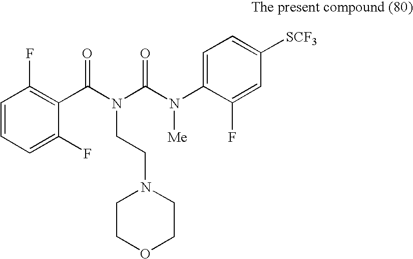

¹H-NMR (DMSO-d₆, Measurement temperature 80° C.) δ (ppm): 2.28-2.40 (4H, br), 2.53 (2H, t, J=6.2 Hz), 3.28 (3H, s), 3.53 (4H, t, J=4.6 Hz), 3.62-3.74 (2H, br), 7.13 (2H, t, J=8.5 Hz), 7.48 (1H, t, J=8.0 Hz), 7.52-7.61-(2H, m), 7.66 (1H, dd, J=10.1, 1.9 Hz).

### Production Example 29

To a mixture of 655 mg of glycine methyl ester hydrochloride, 30 mL of tert-butylmethyl ether and 1.5 mL of triethylamine was added 1.0 g of N-[2-fluoro-4-(trifluoromethylthio)phenyl]-N-methylcarbamoyl chloride, and stirred at room temperature for an hour. The reaction solution was washed sequentially with 2N hydrochloric acid, a saturated sodium hydrogen carbonate aqueous solution and a saturated saline solution, dried over anhydrous magnesium sulfate, and concentrated under reduced pressure to give 1.20 g of 1-[2-fluoro-4-(trifluoromethylthio)phenyl]-3-methoxycarbonylmethyl-1-methylurea.

### 1-[2-Fluoro-4-(trifluoromethylthio)phenyl]-3-methoxycarbonylmethyl-1-methylurea

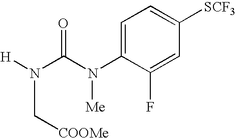

¹H-NMR (CDCl₃) δ (ppm): 3.26 (3H, s), 3.73 (3H, s), 4.00 (2H, d, J=5.4 Hz), 4.80 (1H, br), 7.43-7.55 (3H, m).

### Example 81

To a solution of 904 mg of 1-[2-fluoro-4-(trifluoromethylthio)phenyl]-3-methoxycarbonylmethyl-1-methylurea and 0.9 mL of diisopropylethylamine in 20 mL of toluene was added 704 mg of 2,6-difluorobenzoyl chloride, and stirred for 3 hours with heating to reflux. The reaction solution was cooled to room temperature, and thereto 50 mL of tert-butylmethyl ether was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 817 mg of 1-(2,6-difluorobenzoyl)-3-[2-fluoro-4-(trifluoromethylthio)phenyl]-1-methoxycarbonylmethyl-3-methylurea (hereinafter, referred to as the present compound (81)).

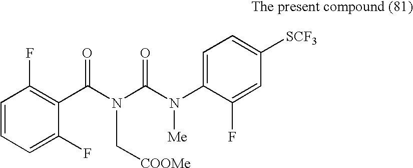

¹H-NMR (DMSO-d₆, Measurement temperature 80° C.) δ (ppm): 3.24 (3H, s), 3.65 (3H, s), 4.37 (2H, s), 7.10 (2H, t, J=8.5 Hz), 7.42 (1H, t, J=8.2 Hz), 7.49-7.59 (2H, m), 7.62 (1H, dd, J=10.1, 1.9 Hz).

### Production Example 30

To a mixture of 1.00 g of t-butyl 4-aminobenzoate and 0.23 g of paraformaldehyde (content; 90% by weight) in 5 mL of methanol was added a mixture of 4.91 g of a 28% sodium methylate-methanol solution and 2 mL of methanol, and stirred at room temperature for 18 hours. The reaction mixture was poured into 15 mL of ice water, and then extracted with 20 mL of chloroform. The organic layer was dried over anhydrous magnesium sulfate, and concentrated under reduced pressure to obtain a residue. The residue was dissolved in 20 mL of ethanol, and thereto 0.43 g of sodium borohyride (content; 90% by weight) was added. The mixture was heated to reflux for 30 minutes. The reaction mixture was allowed to cool to room temperature and then concentrated under reduced pressure. To the residue were added 20 mL of water and 20 mL of chloroform, and then layers separated. The organic layer was dried over anhydrous magnesium sulfate, and concentrated under reduced pressure. The obtained residue was purified by silica gel chromatography (ethyl acetate:hexane=1:5) to give 0.73 g of t-butyl 4-methylaminobenzoate.

### t-Butyl 4-methylaminobenzoate

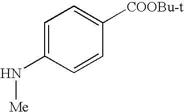

¹H-NMR (CDCl₃) δ (ppm): 1.58 (9H, s), 2.88 (3H, brs), 4.12 (1H, br), 6.52-6.55 (2H, m), 7.81-7.84 (2H, m).

### Example 82

A solution of 0.64 g of 2,6-difluorobenzoyl isocyanate in 0.5 mL of diethyl ether was added at room temperature to a solution of 0.73 of t-butyl 4-methylaminobenzoate in 3.5 mL of diethyl ether, and stirred for an hour. Then 3.5 mL of hexane was added thereto, and a produced solid was collected by filtration and dried to give 1.20 g of 1-[4-(t-butoxycarbonyl)phenyl]-3-(2,6-difluorobenzoyl)-1-methylurea (hereinafter, referred to as the present compound (82)).

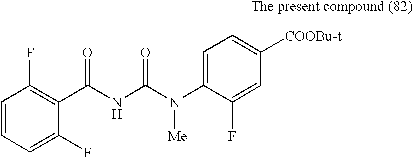

¹H-NMR (DMSO-d₆) δ (ppm): 1.57 (9H, s), 3.28 (3H, s), 7.13-7.19 (2H, m), 7.38-7.41-(2H, m), 7.48-7.56 (1H, m), 7.89-7.92 (2H, m), 10.74 (1H, brs).

### Example 83

A solution of 3.78 g of 2,6-difluorobenzoyl isocyanate in 3.0 mL of diethyl ether was added at room temperature to a solution of 3.79 g of 2-fluoro-N-methyl-4-(2-propenylthio)aniline in 18 mL of diethyl ether, and stirred for an hour. A produced solid was collected by filtration, and dried to give 5.83 g of 3-(2,6-difluorobenzoyl)-1-[2-fluoro-4-(2-propenylthio)phenyl]-1-methylurea (hereinafter, referred to as the present compound (83)).

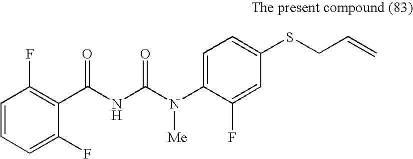

¹H-NMR (DMSO-d₆) δ (ppm): 3.15 (3H, s), 3.72-3.74 (2H, s), 5.10-5.13 (1H, m), 5.27-5.32 (1H, m), 5.79-5.88 (1H, m), 7.11-7.17 (3H, m), 7.26-7.30 (2H, m), 7.46-7.52 (1H, m), 10.70 (1H, br).

### Example 84

To a solution of 1.01 g of 3-(2,6-difluorobenzoyl)-1-[2-fluoro-4-(2-propenylthio)phenyl]-1-methylurea in 10.0 mL of 1-methyl-2-pyrrolidone was added 126 mg of sodium hydride (content; 55% by weight in oil) at 2° C., and stirred for 30 minutes. Thereto 0.39 mL of methyl iodide was added at 1° C., and the mixture was stirred for 3 hours at 2-3° C. To the reaction mixture was added a mixture of 10 mL of a saturated ammonium chloride aqueous solution and 10 mL of water under ice-cooling, and the mixture was extracted with 20 mL of ethyl acetate three times. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) to give 0.66 g of 1-(2,6-difluorobenzoyl)-3-[2-fluoro-4-(2-propenylthio)phenyl]-1,3-dimethylurea (hereinafter, referred to as the present compound (84)).

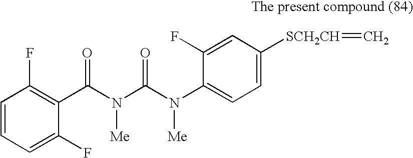

¹H-NMR (CDCl₃) δ (ppm): 3.04 (3H, s), 3.26 (3H, brs), 3.56-3.58 (2H, m), 5.12-5.25 (2H, m), 5.82-5.92 (1H, m), 6.87-6.92 (2H, m), 7.06-7.11 (3H, m), 7.31-7.38 (1H, m).

### Example 85

To a solution of 0.50 g of 3-(2,6-difluorobenzoyl)-1-[2-fluoro-4-(2-propenylthio)phenyl]-1-methylurea in 5.0 mL of chloroform was added 0.35 g of meta-chloroperbenzoic acid (content; 65% by weight) under ice-cooling, and stirred for an hour. To the reaction mixture was added 5 mL of chloroform. The reaction mixture was washed three times with 10 mL of a sodium hydrogen carbonate aqueous solution, dried over anhydrous magnesium sulfate, and concentrated under reduced pressure. The obtained residue was purified by silica gel chromatography (ethyl acetate:hexane=2:1) to give 0.45 g of 3-(2,6-difluorobenzoyl)-1-[2-fluoro-4-(2-propenylsulfinyl)phenyl]-1-methylurea (hereinafter, referred to as the present compound (85)).

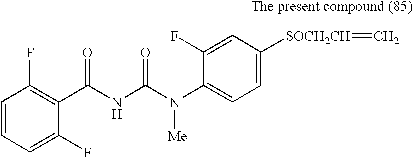

¹H-NMR (CDCl₃) δ (ppm): 3.28 (3H, s), 3.49-3.64 (2H, m), 5.24-5.41 (2H, m), 5.61-5.72 (1H, m), 6.93-6.97 (2H, m), 7.38-7.43 (2H, m), 7.49-7.53 (2H, m), 8.03 (1H, brs).

### Example 86

To a solution of 0.50 g of 3-(2,6-difluorobenzoyl)-1-[2-fluoro-4-(2-propenylsulfinyl)phenyl]-1-methylurea in 10.0 mL of chloroform was added 0.77 g of meta-chloroperbenzoic acid (content; 65% by weight) under ice-cooling, and stirred at room temperature for two hours. To the reaction solution was added 10 mL of chloroform. The mixture was washed three times with 20 mL of a sodium hydrogen carbonate aqueous solution, dried over anhydrous magnesium sulfate, and concentrated under reduced pressure. The obtained residue was purified by silica gel chromatography (ethyl acetate:hexane=1:1) to give 0.41 g of 3-(2,6-difluorobenzoyl)-1-[2-fluoro-4-(2-propenylsulfonyl)phenyl]-1-methylurea (hereinafter, referred to as the present compound (86)).

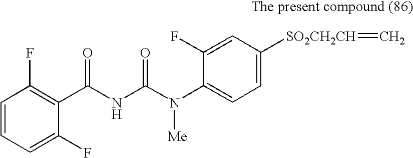

¹H-NMR (CDCl₃) δ (ppm): 3.29 (3H, s), 3.80-3.81 (2H, d), 5.20-5.39 (2H, m), 5.72-5.83 (1H, m), 6.92-6.97 (2H, m), 7.37-7.44 (1H, m), 7.53-7.57 (1H, m), 7.68-7.72 (2H, m), 8.28 (1H, brs).

### Production Example 31

To a mixture of 15 g of 2-fluoro-N-methylaniline and 31.0 g of sodium thiocyanate in 90 mL of methanol was added dropwise a mixture of 6.8 mL of bromine and 60 mL of a saturated sodium bromide-methanol solution at −18° C. The obtained mixture was stirred at −5° C. for two hours, poured into 240 mL of ice water, and then adjusted to pH 8 by an addition of 45 g of sodium carbonate. The reaction solution was extracted twice with 90 mL of chloroform. The organic layers were combined, and dried over anhydrous magnesium sulfate and concentrated under reduced pressure. The obtained residue was added to 40 mL of water, and 30.0 g of sodium sulfide nonahydrate was added thereto. The mixture was heated to reflux for two hours, and then allowed to cool to room temperature. The reaction mixture was adjusted to pH 5 by an addition of 8.0 mL of acetic acid, and extracted three times with 80 mL of chloroform. The organic layers were combined, and dried over anhydrous magnesium sulfate, and concentrated under reduced pressure to give 21.7 g of 3-fluoro-4-methylaminobenzenethiol.

### 3-Fluoro-4-methylaminobenzenethiol

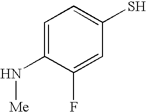

¹H-NMR (CDCl₃) δ (ppm): 2.86 (1H, brs), 2.88 (3H, s), 4.19 (1H, br), 6.51-6.61 (1H, m), 7.10-7.18 (2H, m).

### Production Example 32

To a solution of 5.00 g of 3-fluoro-4-methylaminobenzenethiol in 50 mL of N,N-dimethylformamide were added 3.6 mL of 1,1,3-trichloro-1-propene and 4.80 g of potassium carbonate, and stirred at room temperature for two days. The reaction mixture was filtered, and the filtrate was concentrated under reduced pressure to obtain a residue. The residue was purified by silica gel chromatography (ethyl acetate:hexane=1:10) to give 1.76 g of 4-(3,3-dichloro-2-propenylthio)-2-fluoro-N-methylaniline.

### 4-(3,3-dichloro-2-propenylthio)-2-fluoro-N-methylaniline

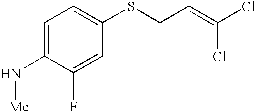

¹H-NMR (CDCl₃) δ (ppm): 2.89 (3H, s), 3.46 (2H, d, J=8.0 Hz), 4.06 (1H, br), 5.92 (1H, t, J=8.0 Hz), 6.58-6.62 (1H, m), 7.08-7.17 (2H, m).

### Example 87

A solution of 1.21 g of 2,6-difluorobenzoyl isocyanate in 1.0 mL of diethyl ether was added at room temperature to a solution of 1.76 g of 4-(3,3-dichloro-2-propenylthio)-2-fluoro-N-methylaniline in 8.0 mL of diethyl ether, and stirred for two hours. Then 9.0 mL of hexane was added thereto, and a produced solid was collected by filtration and dried to give 1.96 g of 1-[4-(3,3-dichloro-2-propenylthio)-2-fluorophenyl]-3-(2,6-difluorobenzoyl)-1-methylurea (hereinafter, referred to as the present compound (87)).

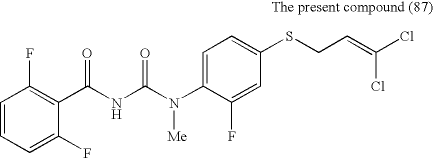

¹H-NMR (DMSO-d₆) δ (ppm): 3.16 (3H, s), 3.82 (2H, d, J=7.7 Hz), 6.29 (1H, t, J=7.7 Hz), 7.11-7.15 (2H, m), 7.20-7.23 (1H, m), 7.31-7.50 (3H, m), 10.71 (1H, brs).

### Example 88

To a solution of 1.01 g of 1-[4-(3,3-dichloro-2-propenylthio)-2-fluorophenyl]-3-(2,6-difluorobenzoyl)-1-methylurea in 10.0 mL of 1-methyl-2-pyrrolidone was added 112 mg of sodium hydroxide at 1° C., and stirred for 30 minutes. Then 0.33 mL of methyl iodide was added at 1° C. thereto. The obtained mixture was stirred at 2-3° C. for three hours. To the reaction mixture was added a mixture of 10 mL of a saturated ammonium chloride aqueous solution and 10 mL of water under ice-cooling, and extracted three times with 20 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) to give 0.65 g of 1-[4-(3,3-dichloro-2-propenylthio)-2-fluorophenyl]-3-(2,6-difluorobenzoyl)-1,3-dimethylurea (hereinafter referred to as the present compound (88)).

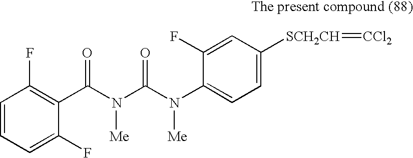

¹H-NMR (CDCl₃) δ (ppm): 3.06 (3H, s), 3.29 (3H, brs), 3.66 (2H, d), 5.97 (1H, t), 6.88-6.92 (2H, m), 7.10-7.15 (3H, m), 7.31-7.39 (1H, m).

### Production Example 33

To a solution of 6.09 g of 3-fluoro-4-methylaminobenzenethiol in 60 mL of acetone were added 3.2 mL of propargyl bromide and 10.7 g of potassium carbonate, and heated at 50° C. for 6 hours. The reaction mixture was allowed to cool to room temperature and then filtered. The filtrate was concentrated under reduced pressure to obtain a residue. The residue was purified by silica gel chromatography (ethyl acetate:hexane=1:10) to give, 1.28 g of 2-fluoro-N-methyl-4-(2-propynylthio)aniline.

### 2-Fluoro-N-methyl-4-(2-propynylthio)aniline

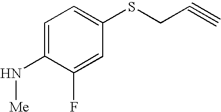

¹H-NMR (CDCl₃) δ (ppm): 2.23 (1H, t, J=4.0 Hz), 2.89 (3H, s), 3.45 (2H, d, J=4.0 Hz), 4.11 (1H, br), 6.60-6.64 (1H, m), 7.19-7.26 (2H, m).

### Example 89

A solution of 1.20 g of 2,6-difluorobenzoyl isocyanate in 1.0 mL of diethyl ether was added at room temperature to a solution of 1.28 g of 2-fluoro-N-methyl-4-(2-propynylthio)aniline in 6.0 mL of diethyl ether, and stirred for 17 hours. The reaction mixture was concentrated under reduced pressure to obtain a residue. The residue was purified by silica gel chromatography (ethyl acetate:hexane=1:10) to give 1.75 g of 3-(2,6-difluorobenzoyl)-1-[2-fluoro-4-(2-propynylthio)phenyl]-1-methylurea (hereinafter referred to as the present compound (89)).

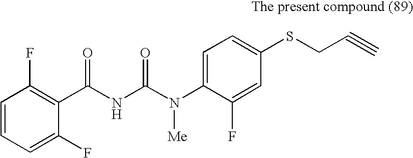

¹H-NMR (DMSO-d₆) δ (ppm): 3.16 (3H, s), 3.20 (1H, t, J=2.5 Hz), 3.94 (2H, d, J=2.5 Hz), 7.11-7.16 (2H, m), 7.21-7.24 (1H, m), 7.31-7.38 (2H, m), 7.48-7.52 (1H, m), 10.74 (1H, brs).

### Example 90

To a solution of 0.50 g of 3-(2,6-difluorobenzoyl)-1-[2-fluoro-4-(2-propynylthio)phenyl]-1-methylurea in 5.0 mL of 1-methyl-2-pyrrolidone was added 70 mg of sodium hydroxide at 2° C., and then stirred for 30 minutes. Then 0.20 mL of methyl iodide was added at 1° C. thereto. The obtained mixture was stirred at 2-3° C. for two hours. To the reaction mixture was added a mixture of 5 mL of a saturated ammonium chloride aqueous solution and 5 mL of water was under ice-cooling, and extracted three times with 10 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) to give 0.43 g of 1-(2,6-difluorobenzoyl)-3-[2-fluoro-4-(2-propynylthio)phenyl]-1,3-dimethylurea (hereinafter referred to as the present compound (90)).

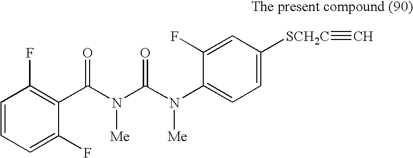

¹H-NMR (DMSO-d₆) δ (ppm): 3.02 (3H, brs), 3.03 (1H, brs), 3.20 (3H, s), 3.86 (2H, m), 7.09-7.16 (2H, m), 7.18-7.23 (2H, m), 7.31-7.34 (1H, m), 7.48-7.56 (1H, m).

### Example 91

To a solution of 0.50 g of 3-(2,6-difluorobenzoyl)-1-[2-fluoro-4-(2-propynylthio)phenyl]-1-methylurea in 5.0 mL of chloroform was added 0.35 g of meta-chloroperbenzoic acid (content; 65% by weight) under ice-cooling, and stirred at room temperature for 0.5 hours. To the reaction solution was added 5 mL of chloroform. The reaction mixture was washed three times with 10 mL of a sodium hydrogen carbonate aqueous solution, dried over anhydrous magnesium sulfate, and concentrated under reduced pressure. The obtained residue was purified by silica gel chromatography (ethyl acetate:hexane=1:1) to give 0.38 g of 3-(2,6-difluorobenzoyl)-1-[2-fluoro-4-(2-propynylsulfinyl)phenyl]-1-methylurea (hereinafter referred to as the present compound (91)).

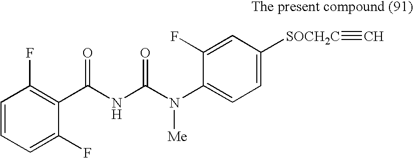

¹H-NMR (CDCl₃) δ (ppm): 2.42 (1H, t, J=2.7 Hz), 3.28 (3H, s), 3.64-3.76 (2H, m), 6.93-6.97 (2H, m), 7.36-7.43 (1H, m), 7.51-7.57 (2H, m), 7.61-7.64 (1H, m), 8.07 (1H, brs).

### Example 92

To a solution of 0.50 g of 3-(2,6-difluorobenzoyl)-1-[2-fluoro-4-(2-propynylthio)phenyl]-1-methylurea in 10.0 mL of chloroform was added 0.77 g of meta-chloroperbenzoic acid (content; 65% by weight) under ice-cooling, and stirred at room temperature for two hours. To the reaction solution was added 10 mL of chloroform. The mixture was washed three times with 20 mL of a sodium hydrogen carbonate aqueous solution, dried over anhydrous magnesium sulfate, and concentrated under reduced pressure. The obtained residue was purified by silica gel chromatography (ethyl acetate:hexane=1:1) to give 0.42 g of 3-(2,6-difluorobenzoyl)-1-[2-fluoro-4-(2-propynylsulfonyl)phenyl]-1-methylurea (hereinafter referred to as the present compound (92)).

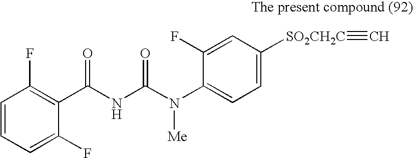

¹H-NMR (CDCl₃) δ (ppm): 2.42 (1H, t, J=2.7 Hz), 3.31 (3H, s), 3.99 (2H, d, J=2.7 Hz), 6.93-6.97 (2H, m), 7.37-7.44 (1H, m), 7.57-7.61 (1H, m), 7.82-7.86 (2H, m), 8.09 (1H, brs).

### Example 93

A solution of 1.97 g of 2,6-difluorobenzoyl isocyanate in 1.0 mL of diethyl ether was added to a solution of 3.32 g of 3,5-dichloro-N-methyl-4-(1,1,2,2-tetrafluoroethylthio)aniline in 10 mL of diethyl ether at room temperature, and stirred for one hour. To the reaction mixture was added 22 mL of hexane, and a produced solid was collected by filtration and then dried to give 4.17 g of 1-[3,5-dichloro-4-(1,1,2,2-tetrafluoroethylthio)phenyl]-3-(2,6-difluorobenzoyl)-1-methylurea (hereinafter referred to as the present compound (93)).

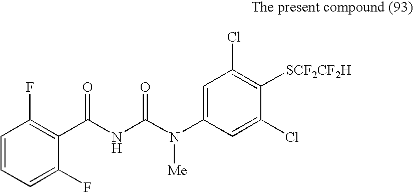

¹H-NMR (DMSO-d₆) δ (ppm): 3.32 (3H, s), 6.66-6.93 (1H, m), 7.15-7.19 (2H, m), 7.52-7.57 (1H, m), 7.70-7.72 (2H, m), 11.05 (1H, brs).

### Example 94

A solution of 0.53 g of 2,6-difluorobenzoyl isocyanate in 0.5 mL of diethyl ether was added to a solution of 0.70 g of N-methyl-4-(1,1,2,2-tetrafluoroethylthio)aniline in 3.5 mL of diethyl ether at room temperature, and stirred for one hour. To the reaction mixture was added 4.0 mL of hexane, and a produced solid was collected by filtration and then dried to give 1.15 g of 3-(2,6-difluorobenzoyl)-1-methyl-1-[4-(1,1,2,2-tetrafluoroethylthio)phenyl]urea (hereinafter referred to as the present compound (94)).

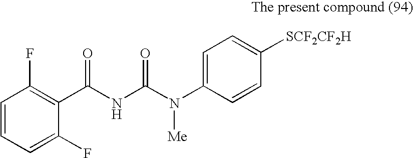

¹H-NMR (DMSO-d₆) δ (ppm): 3.29 (3H, s), 6.56-6.83 (1H, m), 7.13-7.17 (2H, m), 7.41-7.44 (2H, m), 7.48-7.55 (1H, m), 7.67-7.69 (2H, m), 10.79 (1H, brs).

### Example 95

To a solution of 0.8 g of 2-fluoro-N-methyl-4-(trifluoromethylthio)aniline in 3.2 mL of diethyl ether was added a solution of 0.77 g of 2,6-difluorobenzoyl isocyanate in 0.8 mL of diethyl ether under ice-cooling, and stirred at room temperature for two hours. The reaction mixture was concentrated to obtain a residue. The residue was purified by silica gel chromatography (ethyl acetate:chloroform:hexane=1:1:4) to give 1.43 g of 3-(2,6-dichlorobenzoyl)-1-[2-fluoro-4-(trifluoromethylthio)phenyl]-1-methylurea (hereinafter referred to as the present compound (95)).

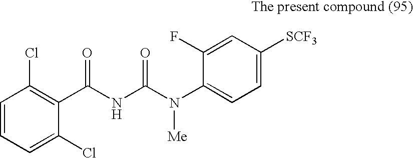

¹H-NMR (DMSO-d₆) δ (ppm): 3.22 (3H, brs), 7.38-7.47 (3H, m), 7.56-7.63 (2H, m), 7.76-7.78 (1H, m), 10.80 (1H, brs).

### Example 96

To a solution of 1.24 g of 3-(2,6-dichlorobenzoyl)-1-[2-fluoro-4-(trifluoromethylthio)phenyl]-1-methylurea in 12.0 mL of 1-methyl-2-pyrrolidone was added 135 mg of sodium hydride (content; 55% by weight in oil) at 2° C., and stirred for 30 minutes. Then 0.42 mL of methyl iodide was added at 1.5° C. thereto. The mixture was stirred at 2-3° C. for three hours, and a mixture of 12.0 mL of a saturated ammonium chloride aqueous solution and 12.0 mL of water was added to the reaction mixture under ice-cooling. The mixture was extracted three times with 24 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) and then purified by medium pressure preparative high performance liquid chromatography (ethyl acetate:hexane=15:85) to give 0.22 g of 1-(2,6-dichlorobenzoyl)-3-[2-fluoro-4-(trifluoromethylthio)phenyl]-1,3-dimethylurea (hereinafter, referred to as the present compound (96)).

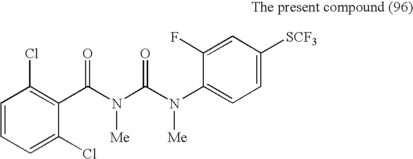

¹H-NMR (DMSO-d₆, Measurement temperature 80° C.) δ (ppm): 2.87 (3H, brs), 3.33 (3H, brs), 7.45 (3H, m), 7.57-7.59 (1H, m), 7.64-7.71 (2H, m).

### Example 97

To a solution of 1.01 g of 3-(2,6-difluorobenzoyl)-1-[2-fluoro-4-(trifluoromethylthio)phenyl]-1-methylurea in 10.0 mL of 1-methyl-2-pyrrolidone was added 118 mg of sodium hydride (content; 55% by weight in oil) at 2° C., and stirred for 30 minutes. Then 1.68 mL of 50% benzyl chlorocarbonate was added at 2° C. thereto and stirred at room temperature for 17 hours. The reaction mixture was poured into 10 mL of ice water and then extracted three times with 20 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) to give 0.52 g of 1-benzyloxycarbonyl-1-(2,6-difluorobenzoyl)-3-[2-fluoro-4-(trifluormethylthio)phenyl]-3-methylurea (hereinafter, referred to as the present compound (97)).

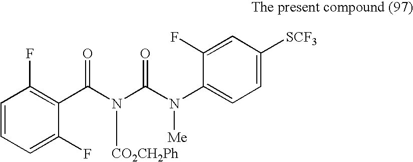

¹H-NMR (DMSO-d₆, Measurement temperature 80° C.) δ (ppm): 3.33 (3H, s), 5.17 (2H, s), 7.00-7.04 (2H, m), 7.23-7.25 (2H, m), 7.35-7.36 (3H, m), 7.41-7.45 (1H, m), 7.49-7.55 (2H, m), 7.69-7.72 (1H, m).

### Example 98

To a solution of 1.01 g of 3-(2,6-difluorobenzoyl)-1-[2-fluoro-4-(trifluoromethylthio)phenyl]-1-methylurea in 10.0 mL of 1-methyl-2-pyrrolidone was added 118 mg of sodium hydride (content; 55% by weight in oil) at 1° C., and stirred for 30 minutes. Then 0.74 mL of phenyl chlorocarbonate was added at 2° C. thereto and stirred at room temperature for 4 hours. The reaction mixture was poured into 10 mL of ice water and then extracted three times with 20 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) and then purified by medium pressure preparative high performance liquid chromatography (ethyl acetate:hexane=15:85) to give 0.45 g of 1-(2,6-difluorobenzoyl)-3-[2-fluoro-4-(trifluormethylthio)phenyl]-3-methyl-1-phenoxycarbonylurea (hereinafter, referred to as the present compound (98)).

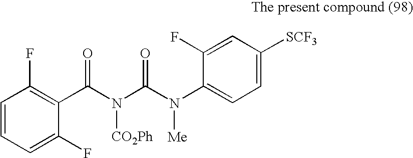

¹H-NMR (DMSO-d₆, Measurement temperature 80° C.) δ (ppm): 3.45 (3H, s), 7.04-7.12 (4H, m), 7.29-7.32 (1H, m), 7.40-7.44 (2H, m), 7.51-7.57 (1H, m), 7.60-7.69 (2H, m), 7.81-7.83 (1H, m).

### Example 99

To a solution of 3.01 g of 3-(2,6-difluorobenzoyl)-1-[2-fluoro-4-(trifluoromethylthio)phenyl]-1-methylurea in 30.0 mL of 1-methyl-2-pyrrolidone was added 353 mg of sodium hydride (content; 55% by weight in oil) at 1.5° C., and stirred for 30 minutes. Then 2.25 mL of benzenesulfonyl chloride was added at 1.5° C. thereto and stirred at room temperature for 22 hours. The reaction mixture was poured into 30 mL of ice water and then extracted three times with 60 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) and then purified by medium pressure preparative high performance liquid chromatography (ethyl acetate:hexane=15:85) to give 0.28 g of 1-(2,6-difluorobenzoyl)-3-[2-fluoro-4-(trifluormethylthio)phenyl]-3-methyl-1-phenoxysulfonylurea (hereinafter, referred to as the present compound (99)).

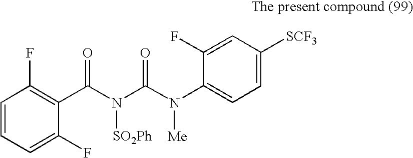

¹H-NMR (DMSO-d₆, Measurement temperature 80° C.) δ (ppm): 3.49 (3H, s), 7.01-7.06 (2H, m), 7.52-7.65 (5H, m), 7.74-7.83 (4H, m).

### Example 100

To a solution of 2.33 g of 2,5-difluoro-N-methyl-4-(trifluoromethylthio)aniline in 8.0 mL of diethyl ether was added a solution of 1.75 g of 2,6-difluorobenzoyl isocyanate in 2.0 mL of diethyl ether under ice-cooling, and stirred at room temperature for two hours. The reaction mixture was placed under ice-cooling and thereto hexane was added portionwise. A deposited white powder was collected by filtration to give 3.54 g of 3-(2,6-difluorobenzoyl)-1-[2,5-difluoro-4-(trifluormethylthio)phenyl]-1-methylurea (hereinafter, referred to as the present compound (100)).

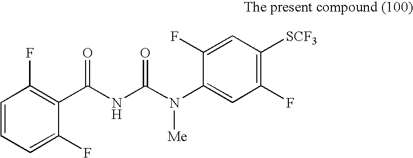

¹H-NMR (DMSO-d₆) δ (ppm): 3.26 (3H, s), 7.12-7.16 (2H, m), 7.47-7.55 (1H, m), 7.68-7.72 (1H, m), 7.89-7.93 (1H, m), 10.98 (1H, brs).

### Example 101

To a solution of 1.01 g of 3-(2,6-difluorobenzoyl)-1-[2,5-difluoro-4-(trifluoromethylthio)phenyl]-1-methylurea in 10.0 mL of 1-methyl-2-pyrrolidone was added 113 mg of sodium hydride (content; 55% by weight in oil) at 3° C., and stirred for 30 minutes. Then 0.35 mL of methyl iodide was added at 1° C. thereto. The obtained mixture was stirred at 2-3° C. for three hours, and a mixture of 10 mL of a saturated ammonium chloride aqueous solution and 10 mL of water was added to the reaction mixture under ice-cooling. The mixture was extracted three times with 20 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) to give 0.63 g of 1-(2,6-difluorobenzoyl)-3-[2,5-difluoro-4-(trifluoromethylthio)phenyl]-1,3-dimethylurea (hereinafter, referred to as the present compound (101)).

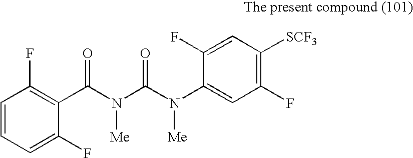

¹H-NMR (DMSO-d₆, Measurement temperature 80° C.) δ (ppm): 3.08 (3H, s), 3.28 (3H, s), 7.08-7.13 (2H, m), 7.44-7.58 (2H, m), 7.77-7.81 (1H, m).

### Example 102

To a solution of 1.20 g of 2,6-difluoro-N-methyl-4-(trifluoromethylthio)aniline in 4.8 mL of diethyl ether was added a solution of 0.90 g of 2,6-difluorobenzoyl isocyanate in 1.2 mL of diethyl ether under ice-cooling, and stirred at room temperature for 0.5 hours. A deposited white powder was collected by filtration to give 1.76 g of 3-(2,6-difluorobenzoyl)-1-[2,6-difluoro-4-(trifluoromethylthio)phenyl]-1-methylurea (hereinafter referred to as the present compound (102)).

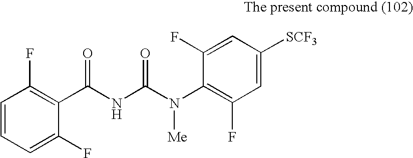

¹H-NMR (DMSO-d₆, Measurement temperature 80° C.) δ (ppm): 3.20 (3H, s), 7.05-7.09 (2H, m), 7.44-7.51 (1H, m), 7.58-7.60 (2H, m), 10.79 (1H, brs).

### Example 103

To a solution of 1.01 g of 3-(2,6-difluorobenzoyl)-1-[2,6-difluoro-4-(trifluoromethylthio)phenyl]-1-methylurea in 10.0 mL of 1-methyl-2-pyrrolidone was added 113 mg of sodium hydride (content; 55% by weight in oil) at 1° C., and stirred for 30 minutes. Then 0.35 mL of methyl iodide was added at 1° C. thereto. The obtained mixture was stirred at 2-3° C. for four hours, and a mixture of 10 mL of a saturated ammonium chloride aqueous solution and 10 mL of water was added to the reaction mixture under ice-cooling. The mixture was extracted three times with 20 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) to give 0.49 g of 1-(2,6-difluorobenzoyl)-3-[2,6-difluoro-4-(trifluoromethylthio)phenyl]-1,3-dimethylurea (hereinafter, referred to as the present compound (103)).

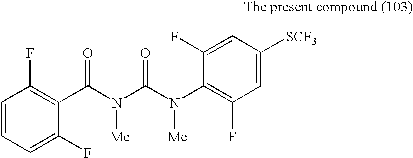

¹H-NMR (DMSO-d₆, Measurement temperature 80° C.) δ (ppm): 3.08 (3H, s), 3.27 (3H, s), 7.09-7.13 (2H, m), 7.50-7.56 (1H, m), 7.57-7.61 (2H, m).

### Example 104

To a solution of 1.01 g of 1-(2-chloro-4-(trifluoromethylthio)phenyl]-3-(2,6-difluorobenzoyl)-1-methylurea in 10.0 mL of 1-methyl-2-pyrrolidone was added 113 mg of sodium hydride (content; 55% by weight in oil) at 2° C., and stirred for 30 minutes. Then 0.35 mL of methyl iodide was added at 1° C. thereto. The obtained mixture was stirred at 2-3° C. for three hours, and a mixture of 10 mL of a saturated ammonium chloride aqueous solution and 10 mL of water was added to the reaction mixture under ice-cooling. The mixture was extracted three times with 20 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) to give 1.03 g of 1-[2-chloro-4-(trifluoromethylthio)phenyl]-3-(2,6-difluorobenzoyl)-1,3-dimethylurea (hereinafter, referred to as the present compound (104)).

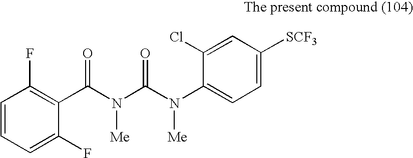

¹H-NMR (DMSO-d₆) δ (ppm): 3.05 (3H, s), 3.27 (3H, s), 7.08-7.12 (2H, m), 7.49-7.54 (2H, m), 7.70-7.72 (1H, m), 7.89-7.90 (1H, m).

### Example 105

To a solution of 0.79 g of 2-methyl-N-methyl-4-(pentafluoroethylthio)aniline in 3.2 mL of diethyl ether was added a solution of 0.53 g of 2,6-difluorobenzoyl isocyanate in 0.8 mL of diethyl ether under ice-cooling, and stirred at room temperature for two hours. The reaction solution was placed under ice-cooling and hexane was added portionwise thereto. A deposited white powder was collected by filtration to give 1.18 g of 3-(2,6-difluorobenzoyl)-1-methyl-1-[2-methyl-4-(pentafluoroethylthio)phenyl]urea (hereinafter, referred to as the present compound (105)).

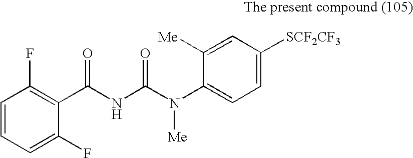

¹H-NMR (DMSO-d₆) δ (ppm): 2.24 (3H, brs), 3.16 (3H, brs), 7.11-7.13 (2H, m), 7.35-7.37 (1H, m), 7.49-7.51 (1H, m), 7.59-7.61 (1H, m), 7.68-7.69 (1H, m), 10.42 (1H, brs).

### Example 106

To a solution of 0.85 g of 3-(2,6-difluorobenzoyl)-1-methyl-1-[2-methyl-4-(pentafluoroethylthio)phenyl]urea in 8.5 mL of 1-methyl-2-pyrrolidone was added 90 mg of sodium hydride (content; 55% by weight in oil) at 2° C., and stirred for 30 minutes. Then 0.28 mL of methyl iodide was added at 1° C. thereto. The obtained mixture was stirred at 2-3° C. for three hours, and a mixture of 8.5 mL of a saturated ammonium chloride aqueous solution and 8.5 mL of water was added to the reaction mixture under ice-cooling. The mixture was extracted three times with 20 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) to give 0.64 g of 1-(2,6-difluorobenzoyl)-1,3-dimethyl-3-[2-methyl-4-(pentafluoroethylthio)phenyl]urea (hereinafter, referred to as the present compound (106)).

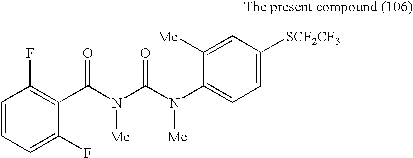

¹H-NMR (DMSO-d₆) δ (ppm): 2.18 (3H, brs), 3.05 (3H, brs), 3.23 (3H, brs), 7.11-7.14 (2H, m), 7.30-7.32 (1H, m), 7.53-7.56 (2H, m), 7.62 (1H, m).

### Example 107

To a solution of 1.02 g of 2-chloro-N-methyl-4-(pentafluoroethylthio)aniline in 4.0 mL of diethyl ether was added a solution of 0.64 g of 2,6-difluorobenzoyl isocyanate in 1.0 mL of diethyl ether under ice-cooling, and stirred at room temperature for two hours. The reaction mixture was placed under ice-cooling and hexane was added portionwise thereto. A deposited white powder was collected by filtration to give 1.44 g of 1-[2-chloro-4-(pentafluoroethylthio)phenyl]-3-(2,6-difluorobenzoyl)-1-methylurea (hereinafter, referred to as the present compound (107)).

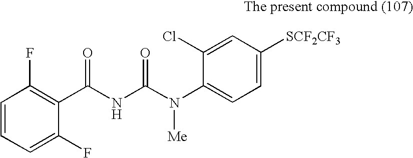

¹H-NMR (DMSO-d₆) δ (ppm): 3.19 (3H, brs), 7.09-7.14 (2H, m), 7.47-7.52 (1H, m), 7.59-7.61 (1H, m), 7.75-7.78 (1H, m), 7.96-7.97 (1H, m), 10.80 (1H, brs).

### Example 108

To a solution of 1.01 g of 1-[2-chloro-4-(pentafluoroethylthio)phenyl]-3-(2,6-difluorobenzoyl)-1-methylurea in 10.0 mL of 1-methyl-2-pyrrolidone was added 101 mg of sodium hydride (content; 55% by weight in oil) at 2° C., and stirred for 30 minutes. Then 0.32 mL of methyl iodide was added at 1° C. thereto. The obtained mixture was stirred at 2-3° C. for three hours, and a mixture of 10 mL of a saturated ammonium chloride aqueous solution and 10 mL of water was added to the reaction mixture under ice-cooling. The mixture was extracted three times with 20 mL of ethyl acetate. Organic layers were combined, washed with saturated saline solution three times, dried over anhydrous magnesium sulfate, and concentrated under reduced pressure. The obtained residue was purified by silica gel chromatography (ethyl acetate:chloroform:hexane=1:1:4) to give 0.92 g of 1-[2-chloro-4-(pentafluoroethylthio)phenyl]-3-(2,6-difluorobenzoyl)-1,3-dimethylurea (hereinafter, referred to as the present compound (108)).

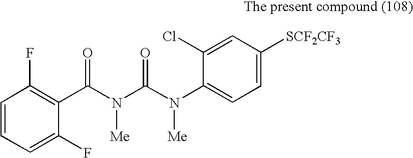

¹H-NMR (DMSO-d₆) δ (ppm): 3.05 (3H, s), 3.27 (3H, s), 7.08-7.12 (2H, m), 7.50-7.54 (2H, m), 7.70-7.73 (1H, m), 7.89-7.90 (1H, m).

### Example 109

A solution of 0.59 g of 2,6-difluorobenzoyl isocyanate in 1.0 mL of diethyl ether was added at room temperature to a solution of 2.09 g of 2-fluoro-N-methyl-4-(1,1,2-trifluoro-2-trifluoromethoxyethylthio)aniline in 5.0 mL of diethyl ether and stirred for an hour. A produced solid was collected by filtration and then dried to give 1.27 g of 3-(2,6-difluorobenzoyl)-1-[2-fluoro-4-(1,1,2-trifluoro-2-trifluoromethoxyethylthio)phenyl]-1-methylurea (hereinafter, referred to as the present compound (109)).

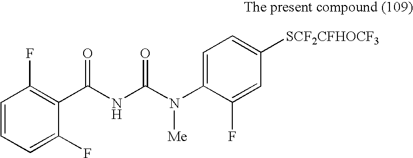

¹H-NMR (DMSO-d₆) δ (ppm): 3.23 (3H, s), 7.11-7.25 (3H, m) 7.47-7.55 (3H, m), 7.65-7.68 (1H, m), 10.89 (1H, brs).

### Example 110

To a solution of 1.01 g of 3-(2,6-difluorobenzoyl)-1-[2-fluoro-4-(1,1,2-trifluoro-2-trifluoromethoxyethylthio)phenyl]-1-methylurea in 10.0 mL of 1-methyl-2-pyrrolidone was added 95 mg of sodium hydride (content; 55% by weight in oil) at 2° C., and stirred for 30 minutes. Then 0.29 mL of methyl iodide was added at 1° C. thereto. The obtained mixture was stirred at 2-3° C. for three hours, and a mixture of 10 mL of a saturated ammonium chloride aqueous solution and 10 mL of water was added to the reaction mixture under ice-cooling. The mixture was extracted three times with 20 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) to give 0.57 g of 1-(2,6-difluorobenzoyl)-3-[2-fluoro-4-(1,1,2-trifluoro-2-trifluoromethoxyethylthio)phenyl]-1,3-dimethylurea (hereinafter, referred to as the present compound (110)).

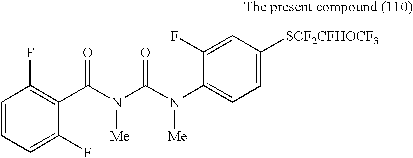

¹H-NMR (DMSO-d₆) δ (ppm): 3.05 (3H, s), 3.25 (3H, s), 6.91-7.06 (1H, m), 7.06-7.11 (2H, m), 7.36-7.40 (1H, m), 7.49-7.60 (3H, m).

### Example 111

To a solution of 160 mg of 4-(difluoromethylthio)-2,3-dimethyl-N-methylaniline in 3 mL of tert-butyl methyl ether was added 135 mg of 2,6-difluorobenzoyl isocyanate, and stirred at room temperature for 30 minutes. The reaction mixture was concentrated under reduced pressure. The obtained residue was purified by medium pressure preparative high performance liquid chromatography (hexane:ethyl acetate=66:34) to give 0.29 g of 3-(2,6-difluorobenzoyl)-1-[4-(difluoromethylthio)-2,3-dimethylphenyl]-1-methylurea (hereinafter, referred to as the present compound (111)).

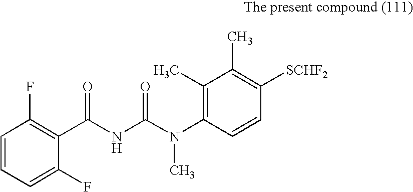

¹H-NMR (CDCl₃) δ (ppm): 2.26 (3H, s), 2.55 (3H, s), 3.17 (3H, s), 6.85 (1H, t, J=56.5 Hz), 6.95 (2H, t, J=8.2 Hz), 7.13 (1H, d, J=8.2 Hz), 7.33-7.46 (2H, m), 7.61 (1H, d, J=8.5 Hz).

### Example 112

To a solution of 190 mg of 3-(2,6-difluorobenzoyl)-1-[4-(difluoromethylthio)-2,3-dimethylphenyl]-1-methylurea in 3 mL of 1,3-dimethyl-2-imidazolidinone were added 81 mg of iodomethane and then 21 mg of sodium hydride (content; 60% by weight in oil), and stirred at room temperature over night. To the reaction mixture was added 30 mL of tert-butyl methyl ether. The mixture was washed sequentially with water and a saturated saline solution, dried over anhydrous magnesium sulfate, and concentrated under reduced pressure. The obtained residue was purified by medium pressure preparative high performance liquid chromatography (hexane:ethyl acetate=66:34) to give 0.19 g of 1-(2,6-difluorobenzoyl)-3-[4-(difluoromethylthio)-2,3-dimethylphenyl]-1,3-dimethylurea (hereinafter, referred to as the present compound (112)).

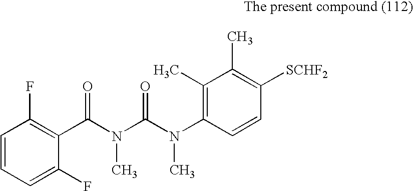

¹H-NMR (DMSO-d₆, Measurement temperature 80° C.) δ (ppm): 2.09 (3H, s), 2.43 (3H, s), 3.04 (3H, s), 3.20 (3H, s), 7.00-7.22 (3H, m), 7.32 (1H, t, J=56.6 Hz), 7.42-7.63 (2H, m).

### Example 10-(2)

To a solution of 1.00 g of 1-[2-fluoro-4-(trifluoromethylthio)phenyl]-1,3-dimethylurea in 10.0 mL of toluene were added 0.49 mL of 2,6-difluorobenzoyl chloride and 0.55 g of diisopropylethylamine, and stirred for three hours in an oil bath at 120° C. The reaction mixture was washed with 10 mL of water, dried over anhydrous magnesium sulfate, and concentrated under reduced pressure. The obtained residue was purified by silica gel chromatography (ethyl acetate:chloroform:hexane=1:1:4) to give 1.14 g of the present compound (10).

### Example 10-(3)

To a solution of 1.00 g of 1-[2-fluoro-4-(trifluoromethylthio)phenyl]-1,3-dimethylurea in 10.0 mL of toluene were added 0.49 mL of 2,6-difluorobenzoyl chloride and 0.59 mL of triethylamine, and stirred for six hours in an oil bath at 120° C. The reaction mixture was washed with 10 mL of water, dried over anhydrous magnesium sulfate, and concentrated under reduced pressure. The obtained residue was purified by silica gel chromatography (ethyl acetate:chloroform:hexane=1:1:4) to give 1.05 g of the present compound (10).

### Example 10-(4)

To a solution of 1.00 g of 1-[2-fluoro-4-(trifluoromethylthio)phenyl]-1,3-dimethylurea in 10.0 mL of toluene were added 0.49 mL of 2,6-difluorobenzoyl chloride and 0.34 mL of pyridine, and stirred for six hours in an oil bath at 120° C. The reaction mixture was washed with 10 mL of water, dried over anhydrous magnesium sulfate, and concentrated under reduced pressure. The obtained residue was purified by silica gel chromatography (ethyl acetate:chloroform:hexane=1:1:4) to give 0.85 g of the present compound (10).

### Example 10-(5)

To a solution of 1.00 g of 1-[2-fluoro-4-(trifluoromethylthio)phenyl]-1,3-dimethylurea in 10.0 mL of toluene were added 0.49 mL of 2,6-difluorobenzoyl chloride and 0.64 mL of 1,8-diazabicyclo[5.4.0]undec-7-ene, and stirred for five hours in an oil bath at 120° C. The reaction mixture was washed with 10 mL of water, dried over anhydrous magnesium sulfate, and concentrated under reduced pressure. The obtained residue was purified by silica gel chromatography (ethyl acetate:chloroform:hexane=1:1:4) to give 0.14 g of the present compound (10).

### Example 10-(6)

To a solution of 1.00 g of 1-[2-fluoro-4-(trifluoromethylthio)phenyl]-1,3-dimethylurea in 10.0 mL of xylene were added 0.49 mL of 2,6-difluorobenzoyl chloride and 0.55 g of diisopropylethylamine, and stirred for six hours in an oil bath at 120° C. The reaction mixture was washed with 10 mL of water, dried over anhydrous magnesium sulfate, and concentrated under reduced pressure. The obtained residue was purified by silica gel chromatography (ethyl acetate:chloroform:hexane=1:1:4) to give 1.19 g of the present compound (10).

### Example 10-(7)

To a solution of 1.00 g of 1-[2-fluoro-4-(trifluoromethylthio)phenyl]-1,3-dimethylurea in 10.0 mL of chlorobenzene were added 0.49 mL of 2,6-difluorobenzoyl chloride and 0.55 g of diisopropylethylamine, and stirred for three hours in an oil bath at 120° C. The reaction mixture was washed with 10 mL of water, dried over anhydrous magnesium sulfate, and concentrated under reduced pressure. The obtained residue was purified by silica gel chromatography (ethyl acetate:chloroform:hexane=1:1:4) to give 1.17 g of the present compound (10).

### Example 10-(8)

To a solution of 1.00 g of 1-[2-fluoro-4-(trifluoromethylthio)phenyl]-1,3-dimethylurea in 10.0 mL of N,N-dimethylformamide were added 0.49 mL of 2,6-difluorobenzoyl chloride and 0.55 g of diisopropylethylamine, and stirred for three hours in an oil bath at 120° C. The reaction mixture was washed with 10 mL of water, dried over anhydrous magnesium sulfate, and concentrated under reduced pressure. The obtained residue was purified by silica gel chromatography (ethyl acetate:chloroform:hexane=1:1:4) to give 0.82 g of the present compound (10).

### Example 10-(9)

To a solution of 1.00 g of 1-[2-fluoro-4-(trifluoromethylthio)phenyl]-1,3-dimethylurea in 10.0 mL of toluene were added 0.49 mL of 2,6-difluorobenzoyl chloride and 0.98 g of potassium carbonate, and stirred for five hours in an oil bath at 120° C. The reaction mixture was added to 20 mL of water. The mixture was extracted with 20 mL of ethyl acetate. The organic layer was washed with 20 mL of saturated saline solution, dried over anhydrous magnesium sulfate, and concentrated under reduced pressure. The obtained residue was purified by silica gel chromatography (ethyl acetate:chloroform:hexane=1:1:4) to give 0.67 g of the present compound (10).

### Example 10-(10)

To a solution of 1.00 g of 2,6-difluoro-N-methylbenzamide and 0.75 mL of chlorotrimethylsilane in 10.0 mL of chloroform was added dropwise a solution of 0.82 mL of triethylamine in 5.0 mL of chloroform at room temperature, and stirred at 40° C. for 40 minutes. Then a solution of 0.78 g of bis(trichloromethyl)carbonate in 10 mL of chloroform was added dropwise at 3° C. thereto, and the mixture was stirred at room temperature for two hours. The reaction mixture was concentrated under reduced pressure. The obtained residue was dissolved in 20 mL of toluene, and thereto 1.30 g of 2-fluoro-N-methyl-4-(trifluoromethylthio)aniline and 1.2 mL of diisopropylethylamine were added, and the mixture was heated at 110° C. for an hour. The reaction mixture was washed with 20 mL of water. The organic layer was dried over anhydrous magnesium sulfate and concentrated under reduced pressure. The obtained residue was purified by silica gel chromatography (ethyl acetate:chloroform:hexane=1:1:4) to give 1.36 g of the present compound (10).

### Production Example 34

To a solution of 3.00 g of 2-fluoro-4-(1,1,2,2-tetrafluoroethylthio)aniline and 0.53 g of paraformaldehyde (content; 90% by weight) in 15 mL of methanol was added a mixture of 11.6 g of a 28% sodium methylate-methanol solution and 7 mL of methanol, and stirred at room temperature for 18 hours. The reaction mixture was poured into 50 mL of ice water and then extracted with 70 mL of chloroform. The organic layer was dried over anhydrous magnesium sulfate and concentrated under reduced pressure. The obtained residue was dissolved in 60 mL of ethanol, and thereto 1.10 g of sodium borohydride (content; 90% by weight) was added. The mixture was heated to reflux for 30 minutes. The reaction mixture was allowed to cool to room temperature and then concentrated under reduced pressure. To the residue were added 50 mL of water and 50 mL of chloroform, and layers separated. The organic layer was dried over anhydrous magnesium sulfate and concentrated under reduced pressure. The obtained residue was purified by silica gel chromatography (ethyl acetate:hexane=1:5) to give 3.18 g of 2-fluoro-N-methyl-4-(1,1,2,2-tetrafluoroethylthio)aniline.

### 2-Fluoro-N-methyl-4-(1,1,2,2-tetrafluoroethylthio)aniline

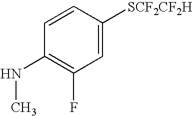

¹H-NMR (CDCl₃) δ (ppm): 2.91 (3H, d, J=5.1 Hz), 4.26 (1H, br), 5.60-5.89 (1H, m), 6.62-6.66 (1H, m), 7.21-7.32 (2H, m).

### Production Example 35

To a solution of 3.18 g of 2-fluoro-N-methyl-4-(1,1,2,2-tetrafluoroethylthio)aniline in 30 mL of toluene was added 1.90 mL of triethylamine. Thereto a solution of 1.65 g of bis(trichloromethyl)carbonate in 10 mL of toluene was added dropwise at 1-8° C. The obtained reaction mixture was stirred at 3° C. for an hour and then concentrated under reduced pressure. To the residue were added 60 mL of water and 60 mL of chloroform, and layers separated. The organic layer was washed with 60 mL of a saturated sodium hydrogen carbonate aqueous solution, dried over anhydrous magnesium sulfate, and concentrated under reduced pressure. The obtained residue was dissolved in 30 mL of acetonitrile, and thereto 5.00 mL of a 40% methylamine-methanol solution was added. The obtained mixture was stirred at room temperature for two hours and then concentrated under reduced pressure. To the residue were added 60 mL of water and 60 mL of chloroform, and layers separated. The organic layer was dried over anhydrous magnesium sulfate, and then concentrated under reduced pressure to give 3.72 g of 1,3-dimethyl-1-[2-fluoro-4-(1,1,2,2-tetrafluoroethylthio)phenyl]urea.

### 1-[2-Fluoro-4-(1,1,2,2-tetrafluoroethylthio)phenyl]-1,3-dimethylurea

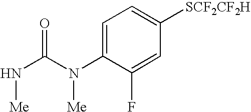

¹H-NMR (CDCl₃) δ (ppm): 2.79 (3H, d, J=4.8 Hz), 3.24 (3H, s), 4.24 (1H, br), 5.72-6.01 (1H, m), 7.33-7.37 (1H, m), 7.46-7.51-(2H, m).

### Example 12-(1)

To a solution of 1.00 g of 1-[2-fluoro-4-(1,1,2,2-tetrafluoroethylthio)phenyl]-1,3-dimethylurea in 10.0 mL of toluene were added 0.44 mL of 2,6-difluorobenzoyl chloride and 0.66 mL of diisopropylethylamine, and stirred for four hours in an oil bath at 120° C. The reaction mixture was washed with 10 mL of water, dried over anhydrous magnesium sulfate, and concentrated under reduced pressure. The obtained residue was purified by silica gel chromatography (ethyl acetate:chloroform:hexane=1:1:4) to give 1.39 g of the present compound (12).

### Example 96-(1)

To a solution of 1.00 g of 1-[2-fluoro-4-(trifluoromethylthio)phenyl]-1,3-dimethylurea in 10.0 mL of toluene were added 0.56 mL of 2,6-difluorobenzoyl chloride and 0.55 g of diisopropylethylamine, and stirred for 19 hours in an oil bath at 120° C. The reaction mixture was washed with 10 mL of water, dried over anhydrous magnesium sulfate, and concentrated under reduced pressure. The obtained residue was purified by silica gel chromatography (ethyl acetate:chloroform:hexane=1:1:4) and then purified by medium pressure preparative high performance liquid chromatography (ethyl acetate:hexane=13:87) to give 0.34 g of the present compound (96).

### Example 113

To a solution of 2.26 g of 1-allyl-1-(2,6-difluorobenzoyl)-3-[2-fluoro-4-(trifluoromethylthio)phenyl]-3-methylurea in chloroform was added 1.2 g of meta-chloroperbenzoic acid (content; 65% by weight) under ice-cooling, and stirred at room temperature for 24 hours. To the reaction mixture was added tert-butylmethyl ether, and the mixture was washed sequentially with a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (ethyl acetate:hexane=75:25) to give 1.55 g of 1-allyl-1-(2,6-difluorobenzoyl)-3-[2-fluoro-4-(trifluoromethylsulfinyl)phenyl]-3-methylurea (hereinafter, referred to as the present compound (113)).

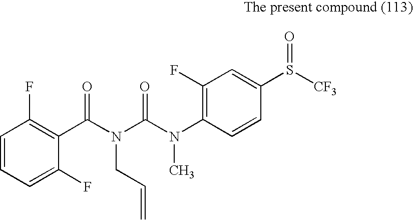

¹H-NMR (DMSO-d₆, Measurement temperature 80° C.) δ: 3.30 (3H, s), 4.13 (2H, d, J=5.8 Hz), 5.03-5.14 (2H, m), 5.77-5.89 (1H, m), 7.09 (2H, t, J=8.3 Hz), 7.50-7.58 (1H, m), 7.61 (1H, t, J=8.1 Hz), 7.74 (1H, d, J=8.1 Hz), 7.82 (1H, d, J=9.7 Hz).

### Production Example 36

To a solution of 776 mg of cyclohexylamine in tert-butylmethyl ether were added 1.1 mL of triethylamine and then 1.5 g of N-[2-fluoro-4-(trifluoromethylthio)phenyl]-N-methylcarbamoyl chloride, and stirred at room temperature for an hour. The reaction mixture was washed sequentially with 2N hydrochloric acid, a saturated sodium hydrogen carbonate aqueous solution and a saturated saline solution, dried over anhydrous magnesium sulfate, and concentrated under reduced pressure to give 1.65 g of 3-cyclohexyl-1-[2-fluoro-4-(trifluoromethylthio)phenyl]-1-methylurea.

### 3-Cyclohexyl-1-[2-fluoro-4-(trifluoromethylthio)phenyl]-1-methylurea

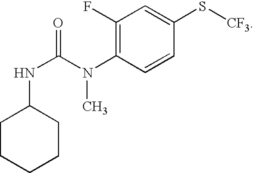

¹H-NMR (CDCl₃) δ: 0.95-1.17 (3H, m), 1.26-1.41-(2H, m), 1.52-1.68 (3H, m), 1.84-1.95 (2H, m), 3.23 (3H, s), 3.60-3.71 (1H, m), 4.11 (1H, d, J=7.5 Hz), 7.36 (1H, t, J=8.3 Hz), 7.45-7.52 (2H, m).

### Example 114

To a solution of 1.52 g of 3-cyclohexyl-1-[2-fluoro-4-(trifluoromethylthio)phenyl]-1-methylurea in 15 mL of toluene were added 0.9 mL of diisopropylethylamine and 919 mg of 2,6-difluorobenzoyl chloride, and stirred for three hours with heating to reflux. The reaction mixture was cooled to room temperature, and thereto ethyl acetate was added. The mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (ethyl acetate:hexane=75:25) to give 2.17 g of 1-cyclohexyl-1-(2,6-difluorobenzoyl)-3-[2-fluoro-4-(trifluoromethylthio)phenyl]-3-methylurea (hereinafter, referred to as the present compound (114)).

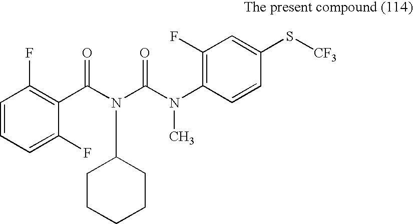

¹H-NMR (DMSO-d₆, Measurement temperature 80° C.) δ: 0.99-1.24 (3H, m), 1.51-1.60 (1H, m), 1.67-1.91 (6H, m), 3.27 (3H, s), 3.54-3.74 (1H, m), 7.16 (2H, t, J=8.4 Hz), 7.31-7.43 (1H, m), 7.52-7.62 (2H, m), 7.67 (1H, dd, J=9.9, 1.8 Hz).

### Production Example 37

To a solution of 470 mg of 1,1-dimethylhydrazine in 25 mL of tert-butylmethyl ether were added 1.1 mL of triethylamine and then 1.5 g of N-[2-fluoro-4-(trifluoromethylthio)phenyl]-N-methylcarbamoyl chloride, and stirred at room temperature for an hour. The reaction mixture was washed sequentially with water and a saturated saline solution, dried over anhydrous magnesium sulfate, and concentrated under reduced pressure. The obtained solid was washed with hexane, and dried under reduced pressure to give 930 g of 3-dimethylamino-1-[2-fluoro-4-(trifluoromethylthio)phenyl]-1-methylurea.

### 3-Dimethylamino-1-[2-fluoro-4-(trifluoromethylthio)phenyl]-1-methylurea

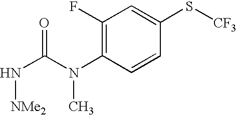

¹H-NMR (CDCl₃) δ: 2.44 (6H, s), 3.23 (3H, s), 5.06 (1H, br), 7.32 (1H, t, J=8.2 Hz), 7.44-7.51-(2H, m).

### Example 115

To a solution of 804 mg of 3-dimethylamino-1-[2-fluoro-4-(trifluoromethylthio)phenyl]-1-methylurea in 8 mL of toluene were added 1.1 mL of diisopropylethylamine and 912 mg of 2,6-difluorobenzoyl chloride, and stirred for six hours with heating to reflux. The reaction mixture was cooled to room temperature, and thereto ethyl acetate was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=90:10) to give 1.10 g of 1-dimethylamino-1-(2,6-difluorobenzoyl)-3-[2-fluoro-4-(trifluoromethylthio)phenyl]-3-methylurea (hereinafter, referred to as the present compound (115)).

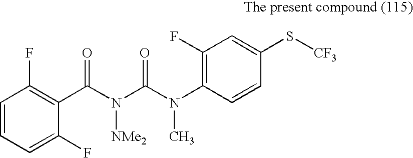

¹H-NMR (DMSO-d₆, Measurement temperature 80° C.) δ: 2.60 (6H, s), 3.27 (3H, s), 7.09 (2H, t, J=8.4 Hz), 7.43-7.62 (3H, m), 7.71 (1H, dd, J=10.0, 1.7 Hz).

### Production Example 38

To a solution of 1.02 g of O-methylhydroxylamine hydrochloride in 30 mL of tetrahydrofuran, were added 2 mL of triethylamine, 3 mL of water and 1.5 g of N-[2-fluoro-4-(trifluoromethylthio)phenyl]-N-methylcarbamoyl chloride, and stirred at room temperature for 24 hours. The reaction mixture was washed sequentially with 2N hydrochloric acid, a saturated sodium hydrogen carbonate aqueous solution and a saturated saline solution, dried over anhydrous magnesium sulfate, and concentrated under reduced pressure to give 1.51 g of 1-[2-fluoro-4-(trifluoromethylthio)phenyl]-3-methoxy-1-methylurea.

### 1-[2-Fluoro-4-(trifluoromethylthio)phenyl]-3-methoxy-1-ethylurea

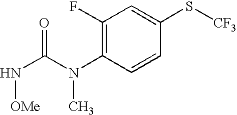

¹H-NMR (CDCl₃) δ: 3.25 (3H, s), 3.61 (3H, s), 7.07 (1H, br), 7.37 (1H, t, J=8.0 Hz), 7.46-7.55 (2H, m).

### Example 116

To a solution of 1.3 g of 1-[2-fluoro-4-(trifluoromethylthio)phenyl]-3-methoxy-1-methylurea in 10 mL of toluene were added 1.1 mL of diisopropylethylamine and 923 mg of 2,6-difluorobenzoyl chloride, and stirred for three hours with heating to refluxing. The reaction mixture was cooled to room temperature, and thereto ethyl acetate was added. The reaction mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=80:20) to give 1.12 g of 1-(2,6-difluorobenzoyl)-3-[2-fluoro-4-(trifluoromethylthio)phenyl]-1-methoxy-3-methylurea (hereinafter, referred to as the present compound (116)).

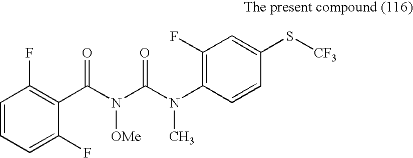

¹H-NMR (DMSO-d₆, Measurement temperature 80° C.) δ: 3.30 (3H, s), 3.63 (3H, s), 7.14 (2H, t, J=8.2 Hz), 7.50-7.63 (3H, m), 7.71 (1H, dd, J=10.1, 1.8 Hz).

### Production Examples 39-52

In the same way as in Production Example 1, 2, 3 or 4, the following compounds were produced.

### Production Example 39

### N-methyl-4-(trifluoromethylthio)aniline

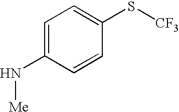

¹H-NMR (CDCl₃) δ (ppm): 2.86 (3H, s), 4.50 (1H, br), 6.61 (2H, d, J=8.0 Hz), 7.44 (2H, d, J=8.0 Hz).

### Production Example 40

### N-methyl-2-methyl-4-(trifluoromethylthio)aniline

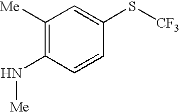

¹H-NMR (CDCl₃) δ (ppm): 2.12 (3H, s), 2.92 (3H, d, J=5.3 Hz), 3.87 (1H, br), 6.56-6.58 (1H, m), 7.30-7.43 (2H, m).

### Production Example 41

### 2-Chloro-N-methyl-4-(trifluoromethylthio)aniline

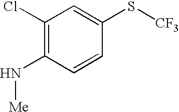

¹H-NMR (DMSO-d₆) δ (ppm): 2.80 (3H, d, J=4.9 Hz), 6.23-6.24 (1H, m), 6.71-6.74 (1H, m), 7.45-7.48 (1H, m), 7.53-7.56 (1H, m).

### Production Example 42

### 2,5-Difluoro-N-methyl-4-(trifluoromethylthio)aniline

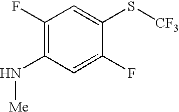

¹H-NMR (DMSO-d₆) δ (ppm): 2.77-2.78 (3H, m), 6.63-6.68 (1H, m), 6.70 (1H, br), 7.34-7.38 (1H, m).

### Production Example 43

### 2,6-Difluoro-N-methyl-4-(trifluoromethylthio)aniline

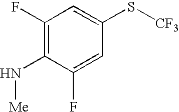

¹H-NMR (DMSO-d₆) δ (ppm): 2.96-2.99 (3H, m), 6.00 (1H, m) 7.30-7.32 (2H, m).

### Production Example 44

### N-methyl-4-(1,1,2,2-tetrafluoroethylthio)aniline

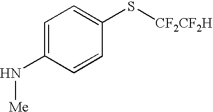

¹H-NMR (CDCl₃) δ (ppm): 2.86 (3H, s), 4.03 (1H, br), 5.59-5.86 (1H, m), 6.57 (2H, d, J=8.7 Hz), 7.42 (2H, d, J=8.7 Hz).

### Production Example 45

### 3,5-Dichloro-N-methyl-4-(1,1,2,2-tetrafluoroethylthio)aniline

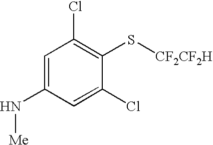

¹H-NMR (CDCl₃) δ (ppm): 2.85 (3H, d, J=5.3 Hz), 4.22 (1H, br), 5.73-6.02 (1H, m), 6.72 (2H, s).

### Production Example 46

### 2-Fluoro-N-methyl-4-(1,1,2-trifluoro-2-trifluoromethoxyethylthio)aniline

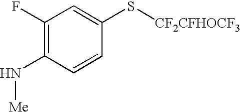

¹H-NMR (CDCl₃) δ (ppm): 2.91 (3H, s), 4.27 (1H, br), 5.68-5.84 (1H, m), 6.62-6.67 (1H, m), 7.20-7.30 (2H, m).

### Production Example 47

### 2-Chloro-N-methyl-4-(1,1,2,2,2-pentafluoroethylthio)aniline

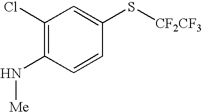

¹H-NMR (DMSO-d₆) δ (ppm): 2.80 (3H, d, J=4.8 Hz), 6.27-6.28 (1H, m), 6.72-6.74 (1H, m), 7.43-7.46 (1H, m), 7.52-7.53 (1H, m).

### Production Example 48

### 2-Methyl-N-methyl-4-(1,1,2,2,2-pentafluoroethylthio)aniline

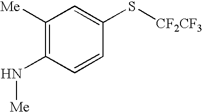

¹H-NMR (DMSO-d₆) δ (ppm): 2.03 (3H, s), 2.76 (1H, d, J=4.6 Hz), 5.74-5.75 (1H, m), 6.53-6.55 (1H, m), 7.21-7.24 (1H, m), 7.32-7.34 (1H, m).

### Production Example 49

### 4-(Difluoromethylthio)-2-fluoro-N-methylaniline

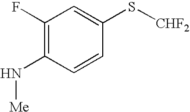

¹H-NMR (CDCl₃) δ: 2.90 (3H, d, J=5.1 Hz), 4.19 (1H, br), 6.64 (1H, t, J=8.7 Hz), 6.71 (1H, t, J=57.5 Hz), 7.19 (1H, dd, J=11.3, 2.2 Hz), 7.24-7.29 (1H, m).

### Production Example 50

### 2-Fluoro-4-(1,1,2,3,3,3-hexafluoro-1-propylthio)-N-methylaniline

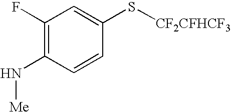

¹H-NMR (CDCl₃) δ: 2.92 (3H, d, J=5.1 Hz), 4.29 (1H, br), 4.60-4.83 (1H, m), 6.65 (1H, t, J=8.7 Hz), 7.23 (1H, d, J=11.3 Hz), 7.31 (1H, d, J=8.7 Hz).

### Production Example 51

### 2-Chloro-4-(difluoromethylthio)-N-methylaniline

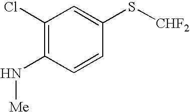

¹H-NMR (CDCl₃) δ: 2.93 (3H, d, J=5.1 Hz), 4.61 (1H, br), 6.61 (1H, d, J=8.4 Hz), 6.70 (1H, t, J=57.2 Hz), 7.38 (1H, dd, J=8.4, 2.0 Hz), 7.48 (1H, d, J=2.0 Hz).

### Production Example 52

### 4-(Difluoromethylthio)-2-methyl-N-methylaniline

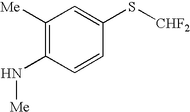

¹H-NMR (CDCl₃) δ: 2.12 (3H, s), 2.91 (3H, d, J=5.1 Hz), 3.80 (1H, br), 6.57 (1H, d, J=8.4 Hz), 6.70 (1H, t, J=57.5 Hz), 7.26 (1H, d, J=2.1 Hz), 7.37 (1H, dd, J=8.4, 2.1 Hz).

### Production Examples 53-60

The following compounds were produced by a method described in Journal of American Chemical Society Vol. 115, No. 6, 2156-2164 (1993), Journal of Fluorine Chemistry 69, 207-212 (1994) or the like.

### Production Example 53

To a solution of 101.0 g of 2-fluoro-4-mercaptoaniline in 1400 mL of acetonitrile was added 109 mL of triethylamine. Thereto 303 g of iodotrifluoromethane was added at an inner temperature of 27-53° C. over 10 minutes. The mixture was stirred at room temperature for 25 minutes, cooled under ice-cooling, and then added to 2000 mL of water. The mixture was extracted twice with 1000 mL of diethyl ether. The organic layer was washed with 1000 mL of 3.5% hydrochloric acid, dried over anhydrous magnesium sulfate, and concentrated by the reduced pressure. To 94.2 g of the obtained residue was added 200 mL of diethyl ether, and insoluble substances were removed by filtration. The filtrate was concentrated under reduced pressure, and 90.2 g of the obtained residue was distilled under reduced pressure to give 66.8 g of 2-fluoro-4-(trifluoromethylthio)aniline.

### 2-Fluoro-4-(trifluoromethylthio)aniline

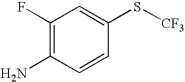

¹H-NMR (CDCl₃) δ (ppm): 4.01 (2H, br), 6.73-7.78 (1H, m), 7.22-7.30 (2H, m).

### Production Example 54

### 2-Methyl-4-(trifluoromethylthio)aniline

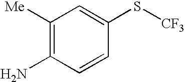

¹H-NMR (CDCl₃) δ (ppm): 2.15 (3H, s), 3.36 (2H, br), 6.64-6.66 (1H, m), 7.25-7.31 (2H, m).

### Production Example 55

### 2-Chloro-4-(trifluoromethylthio)aniline

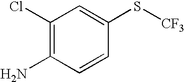

¹H-NMR (CDCl₃) δ (ppm): 4.35 (2H, br), 6.73-6.75 (1H, d, J=8.4 Hz), 7.32-7.34 (1H, dd, J=8.4, 2.1 Hz), 7.54 (1H, d, J=2.1 Hz).

### Production Example 56

### 2,6-Difluoro-4-(trifluoromethylthio)aniline

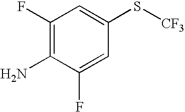

¹H-NMR (CDCl₃) δ (ppm): 4.05 (2H, br), 7.14-7.19 (2H, m).

### Production Example 57

### 2,3-Dimethyl-4-(trifluoromethylthio)aniline

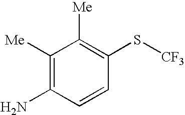

¹H-NMR (CDCl₃) δ (ppm): 2.12 (3H, s), 2.50 (3H, s), 4.10 (2H, br), 6.57 (1H, d J=8.3 Hz), 7.36 (1H, d J=8.3 Hz).

### Production Example 58

### 2,5-Difluoro-4-(trifluoromethylthio)aniline

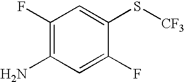

¹H-NMR (CDCl₃) δ (ppm): 4.17 (2H, br), 6.53-6.57 (1H, m), 7.20-7.26 (1H, m).

### Production Example 59

### 2-Fluoro-4-(1,1,2,2,2-pentafluoroethylthio)aniline

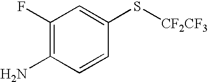

¹H-NMR (CDCl₃) δ (ppm): 3.60-4.40 (2H, br), 6.73-6.77 (1H, m), 7.20-7.28 (2H, m).

### Production Example 60

### 2-Fluoro-4-(1,1,2,2,3,3,3-heptafluoro-1-propylthio)aniline

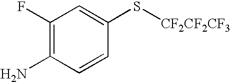

¹H-NMR (CDCl₃) δ (ppm): 4.04 (2H, br, NH₂), 6.73-6.78 (1H, m, Ph), 7.21-7.29 (2H, m, Ph).

### Example 117

To a solution of 1.01 g of 3-(2,6-difluorobenzoyl)-1-methyl-1-[4-(trifluoromethylthio)phenyl]urea in 10.0 mL of 1-methyl-2-pyrrolidone was added 123 mg of sodium hydride (content; 60% by weight in oil) at 2° C., and stirred for 30 minutes. Then 0.38 mL of methyl iodide was added at 2° C. thereto. The obtained mixture was stirred at 2-3° C. for three hours, and a mixture of 10 mL of a saturated ammonium chloride aqueous solution and 10 mL of water was added to the reaction mixture. The mixture was extracted three times with 20 mL of ethyl acetate. Organic layers were combined, washed three times with a saturated saline solution, dried over anhydrous magnesium sulfate, and concentrated under reduced pressure. The obtained residue was purified by silica gel chromatography (ethyl acetate:chloroform:hexane=1:1:4) to give 0.92 g of 1-(2,6-difluorobenzoyl)-1,3-dimethyl-3-[(4-trifluoromethylthio)phenyl]urea (hereinafter, referred to as the present compound (117)).

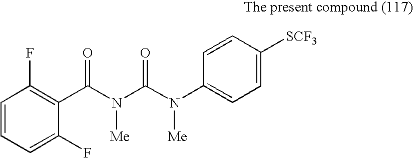

¹H-NMR (DMSO-d₆, 80° C.) δ (ppm): 3.03 (3H, s), 3.27 (3H, s), 7.07-7.11 (2H, m), 7.27-7.29 (2H, m), 7.48-7.56 (1H, m), 7.67-7.70 (2H, m).

### Production Example 61

To a solution of 358 mg of 2-methoxybenzylamine in 20 mL of tert-butylmethyl ether were added 0.36 mL of triethylamine and then 500 mg of N-[2-fluoro-4-(trifluoromethylthio)phenyl]-N-methylcarbamoyl chloride, and stirred at room temperature for an hour. The reaction mixture was washed sequentially with 2N hydrochloric acid, a saturated sodium hydrogen carbonate aqueous solution and a saturated saline solution, dried over anhydrous magnesium sulfate, and concentrated under reduced pressure to give 670 mg of 1-[2-fluoro-4-(trifluoromethylthio)phenyl]-3-(2-methoxybenzyl)-1-methylurea.

### 1-2-Fluoro-4-(trifluoromethylthio)phenyl]-3-(2-methoxybenzyl)-1-methylurea

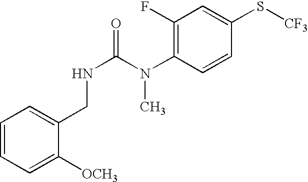

¹H-NMR (CDCl₃) δ: 3.23 (3H, s), 3.68 (3H, s), 4.38 (2H, d, J=6.1 Hz), 5.05 (1H, br), 6.82 (1H, d, J=8.0 Hz), 6.91 (1H, t, J=7.1 Hz), 7.21-7.28 (2H, m), 7.31 (1H, t, J=8.0 Hz), 7.42-7.51-(2H, m).

### Example 118

To a solution of 670 mg of 1-[2-fluoro-4-(trifluoromethylthio)phenyl]-3-(2-methoxybenzyl)-1-methylurea in 7 mL of toluene were added 0.45 mL of diisopropylethylamine and 500 mg of 2,6-difluorobenzoyl chloride, and stirred for three hours with heating to reflux. The reaction mixture was cooled to room temperature, and ethyl acetate was added to the reaction mixture. The mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 897 mg of 1-(2,6-difluorobenzoyl)-3-[2-fluoro-4-(trifluoromethylthio)phenyl]-1-(2-methoxybenzyl)-3-methylurea (hereinafter, referred to as the present compound (118)).

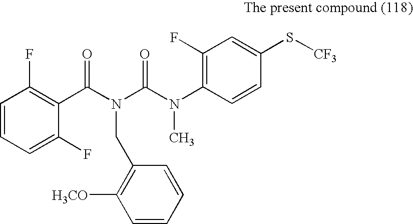

¹H-NMR (DMSO-d₆, Measurement temperature 80° C.) δ: 3.19 (3H, s), 3.68 (3H, s), 4.69 (2H, s), 6.88 (1H, t, J=7.5 Hz), 6.93 (1H, d, J=8.0 Hz), 7.08 (2H, t, J=8.5 Hz), 7.17 (1H, t, J=8.2 Hz), 7.21-7.30 (2H, m), 7.47-7.57 (2H, m), 7.63 (1H, dd, J=10.1, 1.9 Hz).

### Production Example 62

To a solution of 358 mg of 3-methoxybenzylamine in 20 mL of tert-butylmethyl ether were added 0.36 mL of triethylamine and then 500 mg of N-[2-fluoro-4-(trifluoromethylthio)phenyl]-N-methylcarbamoyl chloride, and stirred at room temperature for an hour. The reaction mixture was washed sequentially with 2N hydrochloric acid, a saturated sodium hydrogen carbonate aqueous solution and a saturated saline solution, dried over anhydrous magnesium sulfate, and concentrated under reduced pressure to give 670 mg of 1-[2-fluoro-4-(trifluoromethylthio)phenyl]-3-(3-methoxybenzyl)-1-methylurea.

### 1-[2-Fluoro-4-(trifluoromethylthio)phenyl]-3-(3-methoxybenzyl)-1-methylurea

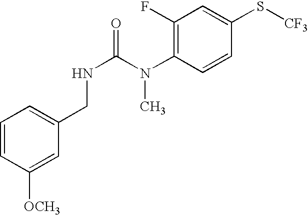

¹H-NMR (CDCl₃) δ: 3.27 (3H, s), 3.79 (3H, s), 4.40 (2H, d, J=5.6 Hz), 4.62 (1H, br), 6.76-6.87 (3H, m), 7.18-7.25 (1H, m), 7.38 (1H, t, J=7.9 Hz), 7.44-7.52 (2H, m).

### Example 119

To a solution of 670 mg of 1-(2-fluoro-4-(trifluoromethylthio)phenyl]-3-(3-methoxybenzyl)-1-methylurea in 7 mL of toluene were added 0.45 mL of diisopropylethylamine and 500 mg of 2,6-difluorobenzoyl chloride, and stirred for three hours with heating to reflux. The reaction mixture was cooled to room temperature, and ethyl acetate was added to the reaction mixture. The mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 923 mg of 1-(2,6-difluorobenzoyl)-3-[2-fluoro-4-(trifluoromethylthio)phenyl]-1-(3-methoxybenzyl)-3-methylurea (hereinafter, referred to as the present compound (119)).

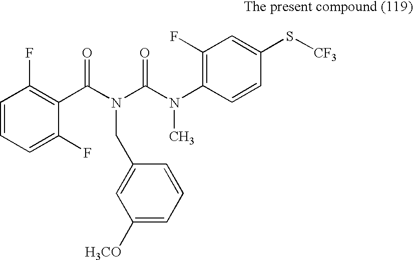

¹H-NMR (DMSO-d₆, Measurement temperature; 80° C.) δ: 3.24 (3H, s), 3.71 (3H, s), 4.72 (2H, s), 6.79-6.86 (3H, m), 7.06 (2H, t, J=8.5 Hz), 7.16-7.27 (2H, m), 7.47-7.57 (2H, m), 7.63 (1H, dd, J=10.1, 1.9 Hz).

### Production Example 63

To a solution of 358 mg of 4-methoxybenzylamine in 20 mL of tert-butylmethyl ether were added 0.36 mL of triethylamine and then 500 mg of N-[2-fluoro-4-(trifluoromethylthio)phenyl]-N-methylcarbamoyl chloride, and stirred at room temperature for an hour. The reaction mixture was washed sequentially with 2N hydrochloric acid, a saturated sodium hydrogen carbonate aqueous solution and a saturated saline solution, dried over anhydrous magnesium sulfate, and concentrated under reduced pressure to give 670 mg of 1-[2-fluoro-4-(trifluoromethylthio)phenyl]-3-(4-methoxybenzyl)-1-methylurea.

### 1-[2-Fluoro-4-(trifluoromethylthio)phenyl]-3-(4-methoxybenzyl)-1-methylurea

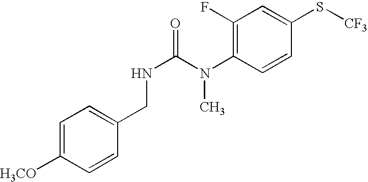

¹H-NMR (CDCl₃) δ: 3.26 (3H, s), 3.78 (3H, s), 4.35 (2H, d, J=5.6 Hz), 4.55 (1H, br s), 6.84 (2H, d, J=8.8 Hz), 7.18 (2H, d, J=8.8 Hz), 7.36 (1H, t, J=8.0 Hz), 7.42-7.51-(2H, m).

### Example 120

To a solution of 670 mg of 1-[2-fluoro-4-(trifluoromethylthio)phenyl]-3-(4-methoxybenzyl)-1-methylurea in 7 mL of toluene were added 0.45 mL of diisopropylethylamine and 500 mg of 2,6-difluorobenzoyl chloride, and stirred for three hours with heating to reflux. The reaction mixture was cooled to room temperature, and ethyl acetate was added to the reaction mixture. The mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=75:25) to give 897 mg of 1-(2,6-difluorobenzoyl)-3-[2-fluoro-4-(trifluoromethylthio)phenyl]-1-(4-methoxybenzyl)-3-methylurea (hereinafter, referred to as the present compound (120)).

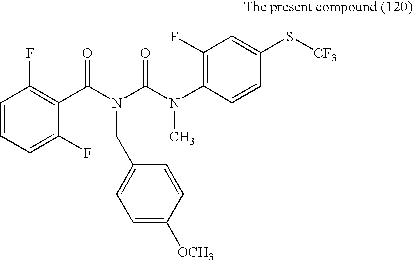

¹H-NMR (DMSO-d₆, Measurement temperature 80° C.) δ: 3.21 (3H, s), 3.74 (3H, s), 4.68 (2H, s), 6.85 (2H, d, J=8.7 Hz), 7.07 (2H, t, J=8.5 Hz), 7.14-7.24 (3H, m), 7.47-7.57 (2H, m), 7.63 (1H, dd, J=10.1, 1.9 Hz).

### Production Example 64

To a solution of 6.43 g of 4-mercapto-2-trifluoromethylaniline in 20 mL of tetrahydrofuran was added 3.1 mL of methyl iodide. The solution was adjusted to 0° C., and 1.46 g of sodium hydride (content; 60% by weight in oil) was added thereto. The mixture was stirred at 0° C. for two hours. Water was added to the reaction mixture, and extracted with tert-butylmethyl ether. The obtained organic layer was washed with a saturated saline solution, dried over anhydrous magnesium sulfate, and concentrated under reduced pressure. The obtained residue was purified by medium pressure preparative high performance liquid chromatography (hexane:ethyl acetate=90:10) to give 402 mg of N-methyl-4-methylthio-2-trifluoromethylaniline.

### N-methyl-4-methylthio-2-trifluoromethylaniline

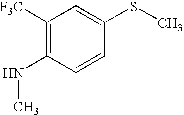

¹H-NMR (CDCl₃) δ: 2.42 (3H, s), 2.89 (3H, d, J=4.8 Hz), 4.42 (1H, br), 6.67 (1H, d, J=8.7 Hz), 7.43 (1H, dd, J=8.7, 2.2 Hz), 7.48 (1H, d, J=2.2 Hz).

### Example 121

To a solution of 400 mg of N-methyl-4-methylthio-2-trifluoromethylaniline in 4 mL of tert-butylmethyl ether was added 331 mg of 2,6-difluorobenzoyl isocyanate, and stirred at room temperature for five minutes. The reaction mixture was concentrated under reduced pressure. The obtained solid was washed with hexane, and dried under reduced pressure to give 675 mg of 1-(2,6-difluorobenzoyl)-3-methyl-3-(2-trifluoromethyl-4-methylthiophenyl)urea (hereinafter, referred to as the present compound (121)).

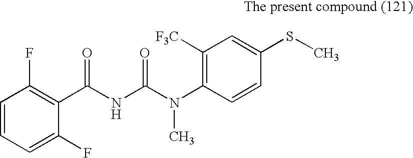

¹H-NMR (CDCl₃) δ: 2.58 (3H, s), 3.18 (3H, s), 6.94 (2H, t, J=8.4 Hz), 7.27 (1H, br), 7.31 (1H, d, J=8.3 Hz), 7.38 (1H, tt, J=8.4, 6.5 Hz), 7.51 (1H, dd, J=8.3, 2.1 Hz), 7.61 (1H, d, J=2.1 Hz).

### Example 122

To a solution of 500 mg of 1-(2,6-difluorobenzoyl)-3-methyl-3-(2-trifluoromethyl-4-methylthiophenyl)urea in 3 mL of 1,3-dimethyl-2-imidazolidinone were added 0.1 mL of iodomethane and then 59 mg of sodium hydride (content; 60% by weight in oil), and stirred at room temperature for 16 hours. To the reaction mixture was added water. The mixture was extracted with ethyl acetate, washed with a saturated saline solution, dried over anhydrous magnesium sulfate, and concentrated under reduced pressure. The obtained solid was washed with hexane and dried under reduced pressure to give 466 mg of 1-(2,6-difluorobenzoyl)-1,3-dimethyl-3-(2-trifluoromethyl-4-methylthiophenyl)urea (hereinafter, referred to as the present compound (122)).

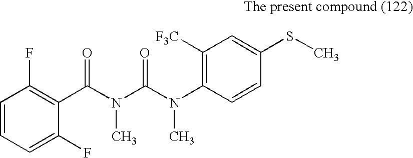

¹H-NMR (DMSO-d₆, Measurement temperature 80° C.) δ: 2.54 (3H, s), 3.10 (3H, s), 3.26 (3H, s), 7.14 (2H, t, J=8.5 Hz), 7.29 (1H, d, J=7.8 Hz), 7.48-7.64 (3H, m).

### Production Example 65

To a solution of 264 mg of 4-tetrahydropyranylamine in 16 mL of tetrahydrofuran were added 0.36 mL of triethylamine and then 500 mg of N-[2-fluoro-4-(trifluoromethylthio)phenyl]-N-methylcarbamoyl chloride, and stirred at room temperature for three hours. The reaction mixture was washed sequentially with 2N hydrochloric acid, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=50:50) to give 520 mg of 1-[2-fluoro-4-(trifluoromethylthio)phenyl]-1-methyl-3-(4-tetrahydropyranyl)urea.

### 1-[2-Fluoro-4-(trifluoromethylthio)phenyl]-1-methyl-3-(4-tetrahydropyranyl)urea

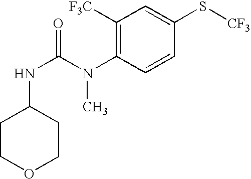

¹H-NMR (CDCl₃) δ: 1.28-1.40 (2H, m), 1.84-1.93 (2H, m), 3.24 (3H, s), 3.45 (2H, td, J=11.7, 2.1 Hz), 3.82-3.95 (3H, m), 4.13 (1H, d, J=7.5 Hz), 7.36 (1H, t, J=8.2 Hz), 7.47-7.54 (2H, m).

### Example 123

To a solution of 510 mg of 1-[2-fluoro-4-(trifluoromethylthio)phenyl]-1-methyl-3-(4-tetrahydropyranyl)urea in 8 mL of toluene were added 0.45 mL of diisopropylethylamine and 600 mg of 2,6-difluorobenzoyl chloride, and stirred for three hours with heating to reflux. The reaction mixture was cooled to room temperature, and ethyl acetate was added to the reaction mixture. The mixture was washed sequentially with water, a saturated sodium hydrogen carbonate aqueous solution and a saturated saline solution, dried over anhydrous magnesium sulfate, and concentrated under reduced pressure. The obtained residue was purified by medium pressure preparative high performance liquid chromatography (hexane:ethyl acetate=66:34) to give 654 mg of 1-(2,6-difluorobenzoyl)-3-[2-fluoro-4-(trifluoromethylthio)phenyl]-3-methyl-1-(4-tetrahydropyranyl)urea (hereinafter, referred to as the present compound (123)).

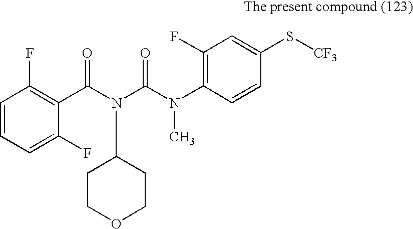

¹H-NMR (DMSO-d₆, Measurement temperature 80° C.) δ: 1.72-1.83 (2H, m), 1.99-2.13 (2H, m), 3.21-3.35 (5H, m), 3.88 (2H, dd, J=11.3, 4.3 Hz), 3.98 (1H, br), 7.16 (2H, t, J=8.5 Hz), 7.35 (1H, t, J=7.4 Hz), 7.53-7.63 (2H, m), 7.67 (1H, dd, J=10.1, 1.9 Hz).

### Production Example 66

To a solution of 500 mg of 1-(2,6-difluorobenzoyl)-3-[2-fluoro-4-(trifluoromethylthio)phenyl]urea in 5 mL of 1,3-dimethyl-2-imidazolidinone were added 200 mg of chloromethyl ethyl ether and then 50 mg of sodium hydride (content; 60% by weight in oil), and stirred at room temperature for 30 minutes. To the reaction mixture were added water and ethyl acetate, and layers separated. The organic layer was washed sequentially with water and a saturated saline solution, dried over anhydrous magnesium sulfate, and concentrated under reduced pressure. The obtained residue was purified by medium pressure preparative high performance liquid chromatography (hexane:ethyl acetate=85:15) to give 390 mg of 1-(2,6-difluorobenzoyl)-1-ethoxymethyl-3-[2-fluoro-4-(trifluoromethylthio)phenyl]urea.

### 1-(2,6-Difluorobenzoyl)-1-ethoxymethyl-3-[2-fluoro-4-(trifluoromethylthio)phenyl]urea

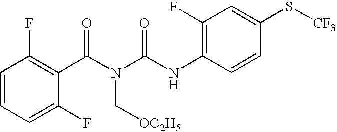

¹H-NMR (CDCl₃) δ: 1.15 (3H, t, J=7.0 Hz), 3.55 (2H, q, J=7.0 Hz), 5.26 (2H, s), 7.01-(2H, t, J=8.0 Hz), 7.39-7.53 (3H, m), 8.36 (1H, t, J=8.0 Hz), 11.38 (1H, br).

### Example 124

To a solution of 380 mg of 1-(2,6-difluorobenzoyl)-1-ethoxymethyl-3-[2-fluoro-4-(trifluoromethylthio)phenyl]urea in 5 mL of 1,3-dimethyl-2-imidazolidinone were added 0.11 mL of methyl iodide and then 50 mg of sodium hydride (content; 60% by weight in oil), and stirred at room temperature for an hour. To the reaction mixture was added ethyl acetate. The mixture was washed sequentially with water and a saturated saline solution, dried over anhydrous magnesium sulfate, and concentrated under reduced pressure. The obtained residue was purified by medium pressure preparative high performance liquid chromatography (hexane:ethyl acetate=85:15) to give 123 mg of 1-(2,6-difluorobenzoyl)-1-ethoxymethyl-3-[2-fluoro-4-(trifluoromethylthio)phenyl]-3-methylurea (hereinafter, referred to as the present compound (124)).

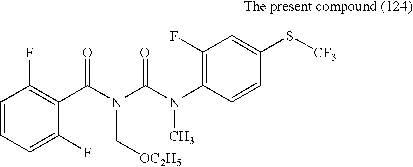

¹H-NMR (DMSO-d₆, Measurement temperature 80° C.) δ: 1.04 (3H, t, J=7.0 Hz), 3.30 (3H, s), 3.48 (2H, q, J=7.0 Hz), 4.86 (2H, s), 7.10 (2H, t, J=8.3 Hz), 7.45-7.59 (3H, m), 7.67 (1H, dd, J=10.1, 1.9 Hz).

### Production Example 67

To a solution of 1.0 g of 1-(2,6-difluorobenzoyl)-3-[2-fluoro-4-(trifluoromethylthio)phenyl]urea in 8 mL of 1,3-dimethyl-2-imidazolidinone were added 654 mg of 2-chloroethyl chloromethyl ether and then 110 mg of sodium hydride (content; 60% by weight in oil), and stirred at room temperature for 3 hours. To the reaction mixture were added water and ethyl acetate, and layers separated. The organic layer was washed sequentially with water and a saturated saline solution, dried over anhydrous magnesium sulfate, and concentrated under reduced pressure. The obtained residue was purified by medium pressure preparative high performance liquid chromatography (hexane:ethyl acetate=85:15) to give 876 mg of 1-(2-chloroethoxymethyl)-1-(2,6-difluorobenzoyl)-3-[2-fluoro-4-(trifluoromethylthio)phenyl]urea.

### 1-(2-Chloroethoxymethyl)-1-(2,6-difluorobenzoyl)-3-[2-fluoro-4-(trifluoromethylthio)phenyl]urea

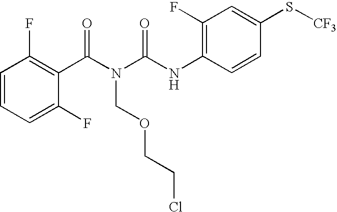

¹H-NMR (CDCl₃) δ: 3.57 (2H, t, J=5.6 Hz), 3.82 (2H, t, J=5.6 Hz), 5.32 (2H, s), 7.02 (2H, t, J=8.0 Hz), 7.40-7.54 (3H, m), 8.35 (1H, t, J=8.1 Hz), 11.39 (1H, br).

### Example 125

To a solution of 876 mg of 1-(2-chloroethoxymethyl)-1-(2,6-difluorobenzoyl)-3-[2-fluoro-4-(trifluoromethylthio)phenyl]urea in 8 mL of 1,3-dimethyl-2-imidazolidinone were added 0.2 mL of methyl iodide and then 100 mg of sodium hydride (content; 60% by weight in oil), and stirred at room temperature for 16 hours. To the reaction mixture was added ethyl acetate, washed sequentially with water and a saturated saline solution, dried over anhydrous magnesium sulfate, and concentrated under reduced pressure. The obtained residue was purified by medium pressure preparative high performance liquid chromatography (hexane:ethyl acetate=85:15) to give 235 mg of 1-(2-chloroethoxymethyl)-1-(2,6-difluorobenzoyl)-3-[2-fluoro-4-(trifluoromethylthio)phenyl]-3-methylurea (hereinafter, referred to as the present compound (125)).

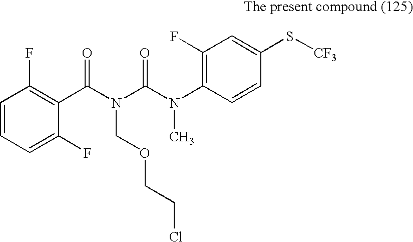

¹H-NMR (DMSO-d₆, Measurement temperature; 80° C.) δ: 3.30 (3H, s), 3.64 (2H, t, J=5.5 Hz), 3.76 (2H, t, J=5.5 Hz), 4.97 (2H, s), 7.10 (2H, t, J=8.4 Hz), 7.46-7.59 (3H, m), 7.67 (1H, dd, J=10.0, 2.0 Hz).

### Production Example 68

To a solution of 1.0 g of 1-(2,6-difluorobenzoyl)-3-[2-fluoro-4-(trifluoromethylthio)phenyl]urea in 8 mL of 1,3-dimethyl-2-imidazolidinone were added 440 mg of benzyl chloromethyl ether and then 110 mg of sodium hydride (content; 60% by weight in oil), and stirred at room temperature for three hours. To the reaction mixture were added water and ethyl acetate, washed sequentially with water and a saturated saline solution, dried over anhydrous magnesium sulfate, and concentrated under reduced pressure. The obtained residue was purified by medium pressure preparative high performance liquid chromatography (hexane:ethyl acetate=85:15) to give 762 mg of 1-benzyloxymethyl-1-(2,6-difluorobenzoyl)-3-[2-fluoro-4-(trifluoromethylthio)phenyl]urea.

### 1-Benzyloxymethyl-1-(2,6-difluorobenzoyl)-3-[2-fluoro-4-(trifluoromethylthio)phenyl]urea

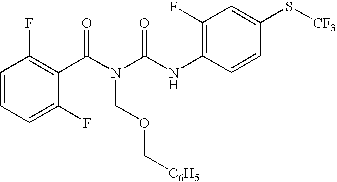

¹H-NMR (CDCl₃) δ: 4.58 (2H, s), 5.32 (2H, s), 6.99 (2H, t, J=8.0 Hz), 7.19-7.35 (5H, m), 7.39-7.51 (3H, m), 8.37 (1H, t, J=8.3 Hz), 11.35 (1H, br).

### Example 126

To a solution of 762 mg of 1-benzyloxymethyl-1-(2,6-difluorobenzoyl)-3-[2-fluoro-4-(trifluoromethylthio)phenyl]urea in 8 mL of 1,3-dimethyl-2-imidazolidinone were added 0.2 mL of methyl iodide and then 100 mg of sodium hydride (content; 60% by weight in oil), and stirred at room temperature for 24 hours. To the reaction mixture was added ethyl acetate, washed sequentially with water and a saturated saline solution, dried over anhydrous magnesium sulfate, and concentrated under reduced pressure. The obtained residue was purified by medium pressure preparative high performance liquid chromatography (hexane:ethyl acetate=85:15) to give 149 mg of 1-benzyloxymethyl-1-(2,6-difluorobenzoyl)-3-[2-fluoro-4-(trifluoromethylthio)phenyl]-3-methylurea (hereinafter, referred to as the present compound (126)).

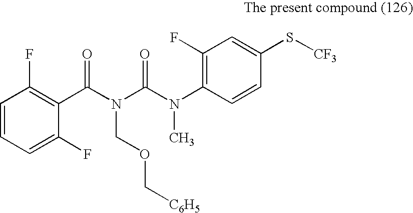

¹H-NMR (DMSO-d₆, Measurement temperature; 80° C.) δ: 3.31 (3H, s), 4.54 (2H, s), 4.96 (2H, s), 7.09 (2H, t, J=8.5 Hz), 7.19-7.24 (2H, m), 7.26-7.34 (3H, m), 7.48 (1H, t, J=8.1 Hz), 7.51-7.58 (2H, m), 7.66 (1H, dd, J=10.0, 2.0 Hz).

### Production Example 69

1-(2,6-Difluorobenzoyl)-3-[2-fluoro-4-(trifluoromethylthio)phenyl]urea (2.0 g) was dissolved in 1,3-dimethyl-2-imidazolidinone (10 ml), and chloromethyl 2-methoxyethyl ether (758 mg) and sodium hydride (60% in oil; 200 mg) were added thereto, followed by stirring at room temperature for 3 hrs. To the resulting reaction mixture was added water, and extracted with ethyl acetate. The organic layer was washed sequentially with water and saturated saline, dried over anhydrous magnesium sulfate, and concentrated under reduced pressure. The obtained residue was purified by medium pressure preparative high performance liquid chromatography (hexane:ethyl acetate=75:25). To the resulting residue was added hexane, filtered, and the filtrate was concentrated under reduced pressure to give 1.57 g of 1-(2,6-difluorobenzoyl)-3-[2-fluoro-4-(trifluoromethylthio)phenyl]-1-(2-methoxyethoxymethyl)urea.

### 1-(2,6-difluorobenzoyl)-3-[2-fluoro-4-(trifluoromethylthio)phenyl]-1-(2-methoxyethoxymethyl)urea

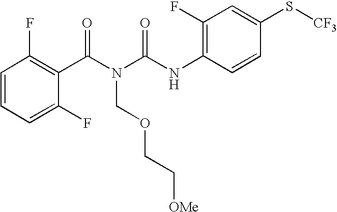

¹H-NMR (CDCl₃) δ: 3.31 (3H, s), 3.47 (2H, t, J=4.5 Hz), 3.69 (2H, t, J=4.5 Hz), 5.33 (2H, s), 7.00 (2H, t, J=8.0 Hz), 7.39-7.52 (3H, m), 8.35 (1H, t, J=8.0 Hz), 11.36 (1H, br).

### Example 127

1-(2,6-Difluorobenzoyl)-3-[2-fluoro-4-(trifluoromethylthio)phenyl]-1-(2-methoxyethoxymethyl)urea (1.57 g) was dissolved in 1,3-dimethyl-2-imidazolidinone (10 ml), and methyl iodide (0.4 ml) and sodium hydride (60% in oil; 156 mg) were added thereto, followed by stirring at room temperature for 16 hrs. To the reaction mixture was added ethyl acetate. The solution was washed sequentially with water and saturated saline, dried over anhydrous magnesium sulfate, and concentrated under reduced pressure. The obtained residue was purified by medium pressure preparative high performance liquid chromatography (hexane:ethyl acetate=75:25) to give 300 mg of 1-(2,6-difluorobenzoyl)-3-[2-fluoro-4-(trifluoromethylthio)phenyl]-1-(2-methoxyethoxymethyl)-3-methylurea (hereinafter, referred to as the present compound (127)).

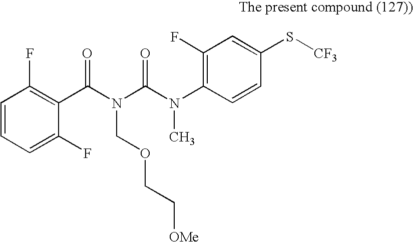

¹H-NMR (DMSO-d₆, Measurement temperature; 80° C.) δ: 3.20 (3H, s), 3.30 (3H, s), 3.38 (2H, t, J=4.9 Hz), 3.58 (2H, t, J=4.9 Hz), 4.92 (2H, s), 7.10 (2H, t, J=8.5 Hz), 7.45-7.59 (3H, m), 7.66 (1H, dd, J=10.1, 1.9 Hz).

### Example 128

To a mixture of 2-chloro-N-methyl-4-(1,1,2,2-tetrafluoroethylthio)aniline (1.0 g) and diethyl ether (4.0 ml) was added a solution of 2,6-difluorobenzoyl isocyanate (0.67 g) in diethyl ether (1.0 ml) under ice-cooling, and the resulting mixture was stirred at room temperature for 2 hrs. To the reaction mixture was added hexane little by little under ice-cooling, and white solids were deposited. By collecting the solids with filtration, 1.40 g of 1-[2-chloro-4-(1,1,2,2-tetrafluoroethylthio)phenyl]-3-(2,6-difluorobenzoyl)-1-methylurea (hereinafter, referred to as the present compound (128)) was obtained.

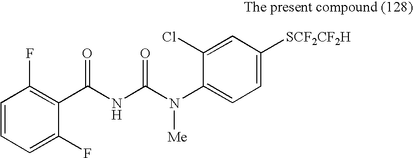

¹H-NMR (CDCl₃) δ (ppm): 3.22 (3H, brs), 5.75-6.02 (1H, m), 6.92-6.99 (2H, m), 7.36-7.45 (2H, m), 7.51 (1H, br), 7.67-7.69 (1H, m), 7.86 (1H, brs)

### Example 129

1-[2-Chloro-4-(1,1,2,2-tetrafluoroethylthio)phenyl]-3-(2,6-difluorobenzoyl)-1-methylurea (1.01 g) was dissolved in 1-methyl-2-pyrrolidone (10 ml), and sodium hydride (105 mg) was added thereto at 2° C., followed by stirring for 30 minutes. To the reaction mixture was added methyl iodide (0.33 ml) at 2° C., and stirred at 2 to 3° C. for 3 hrs. To the reaction mixture was added a mixed solution of saturated aqueous ammonium chloride solution (10 ml) and water (10 ml) under ice-cooling, and extracted three times with ethyl acetate (20 ml). The combined organic layer was washed three times with saturated saline, dried over anhydrous magnesium sulfate, and concentrated under reduced pressure. The obtained residue was purified by silica gel chromatography (ethyl acetate:chloroform:hexane=1:1:4) to give 0.85 g of 1-[2-chloro-4-(1,1,2,2-tetrafluoroethylthio)phenyl]-3-(2,6-difluorobenzoyl)-1,3-dimethylurea (hereinafter, referred to as the present compound (129)).

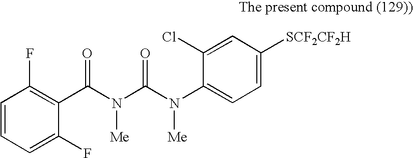

¹H-NMR (DMSO-d₆, Measurement temperature 80° C.) δ (ppm): 3.07 (3H, brs), 3.26 (3H, brs), 6.50-6.78 (1H, m), 7.08-7.12 (2H, m), 7.46-7.56 (2H, m), 7.64-7.67 (1H, m), 7.82 (1H, s).

### Example 130

To a mixture of 2-methyl-N-methyl-4-(1,1,2,2-tetrafluoroethylthio)aniline (1.0 g) and diethyl ether (4.0 ml) was added a solution of 2,6-difluorobenzoyl isocyanate (0.72 g) in diethyl ether (1.0 ml) under ice-cooling, and the resulting mixture was stirred at room temperature for 2 hrs. The reaction mixture was concentrated. The residue was purified by silica gel chromatography (ethyl acetate:chloroform:hexane=1:1:4) to give 1.32 g of 3-(2,6-difluorobenzoyl)-1-methyl-1-[2-methyl-4-(1,1,2,2-tetrafluoroethylthio)phenyl]urea (hereinafter, referred to as the present compound (130)).

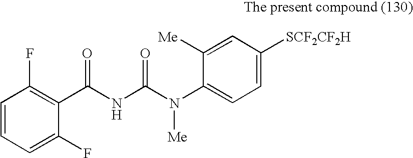

¹H-NMR (CDCl₃) δ (ppm): 2.34 (3H, brs), 3.19 (3H, brs), 5.72-6.00 (1H, m), 6.92-6.96 (2H, m), 7.26-7.30 (1H, m), 7.35-7.43 (1H, m), 7.47 (1H, br), 7.60-7.62 (1H, m), 7.66 (1H, brs).

### Example 131

3-(2,6-Difluorobenzoyl)-1-methyl-1-[2-methyl-4-(1,1,2,2-tetrafluoroethylthio)phenyl]urea (1.01 g) was dissolved in 1-methyl-2-pyrrolidone (10.0 ml), and sodium hydride (110 mg) was added thereto at 2° C., followed by stirring for 30 minutes. To the resulting mixture was added methyl iodide (0.34 ml) at 2° C., and stirred at 2 to 3° C. for 3 hrs. To the reaction mixture was added a mixed solution of saturated aqueous ammonium chloride solution (10 ml) and water (10 ml) under ice-cooling, and extracted three times with ethyl acetate (20 ml). The combined organic layer was washed three times with saturated saline, dried over anhydrous magnesium sulfate, and concentrated under reduced pressure. The obtained residue was purified by silica gel chromatography (ethyl acetate:chloroform:hexane=1:1:4) to give 0.85 g of 1-(2,6-difluorobenzoyl)-1,3-dimethyl-3-[2-methyl-4-(1,1,2,2-tetrafluoroethylthio)phenyl]urea (hereinafter, referred to as the present compound (131)).

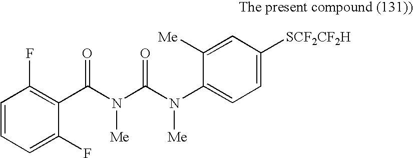

¹H-NMR (DMSO-d₆, Measurement temperature 80° C.) δ (ppm): 2.17 (3H, brs), 3.05 (3H, brs), 3.23 (3H, s), 6.44-6.72 (1H, m), 7.10-7.14 (2H, m), 7.26-7.28 (1H, m), 7.49-7.54 (3H, m)

### Example 132

To a mixture of 2,3-dimethyl-N-methyl-4-(1,1,2,2-tetrafluoroethylthio)aniline (1.0 g) and diethyl ether (4.0 ml) was added a solution of 2,6-difluorobenzoyl isocyanate (0.69 g) in diethyl ether (1.0 ml) under ice-cooling, and the resulting mixture was stirred at room temperature for 1 hr. By collecting white solids deposited in the reaction mixture with filtration, 1.63 g of 1-(2,6-difluorobenzoyl)-3-[2,3-dimethyl-4-(1,1,2,2-tetrafluoroethylthio)phenyl]-3-methylurea (hereinafter, referred to as the present compound (132)) was obtained.

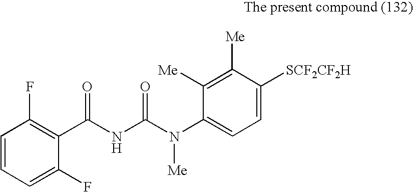

¹H-NMR (CDCl₃) δ (ppm): 2.27 (3H, s), 2.58 (3H, s), 3.18 (3H, s), 5.72-5.99 (1H, m), 6.92-6.96 (2H, m), 7.14-7.16 (1H, m), 7.35-7.43 (2H, m), 7.67-7.69 (1H, m)

### Example 133

1-(2,6-Difluorobenzoyl)-3-[2,3-dimethyl-4-(1,1,2,2-tetrafluoroethylthio)phenyl]-3-methylurea (1.01 g) was dissolved in 1-methyl-2-pyrrolidone (10.0 ml), and sodium hydride (107 mg) was added thereto at 2° C., followed by stirring for 30 minutes. To the resulting mixture was added methyl iodide (0.33 ml) at 2° C., and stirred at 2 to 3° C. for 3 hrs. To the reaction mixture was added a mixed solution of saturated aqueous ammonium chloride solution (10 ml) and water (10 ml) under ice-cooling, and extracted three times with ethyl acetate (20 ml). The combined organic layer was washed three times with saturated saline, dried over anhydrous magnesium sulfate, and concentrated under reduced pressure. The obtained residue was purified by silica gel chromatography (ethyl acetate:chloroform:hexane=1:1:4) to give 0.98 g of 1-(2,6-difluorobenzoyl)-3-[2,3-dimethyl-4-(1,1,2,2-tetrafluoroethylthio)phenyl]-1,3-dimethylurea (hereinafter, referred to as the present compound (133)).

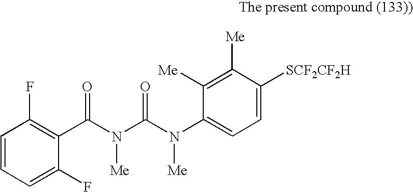

¹H-NMR (DMSO-d₆, Measurement temperature 80° C.) δ (ppm): 2.12 (3H, brs), 2.48 (3H, brs), 3.07 (3H, brs), 3.22 (3H, brs), 6.42-6.70 (1H, m), 7.10-7.14 (3H, m), 7.49-7.55 (2H, m)

### Production Example 70

To a mixture of bis(4-aminophenyl)disulfide (5.0 g), chloroform (100 ml) and triethylamine (8.4 ml) was added dropwise trifluoroacetic anhydride (8.4 ml) at 2 to 7° C. The resulting mixture was stirred at 20° C. for 1 hr. The reaction mixture was poured to ice-water (100 ml), and extracted with ethyl acetate (200 ml). The organic layer was dried over anhydrous magnesium sulfate, and concentrated under reduced pressure to give 9.55 g of bis[4-(N-trifluoroacetylamino)phenyl]disulfide.

### Bis[4-(N-trifluoroacetylamino)phenyl]disulfide

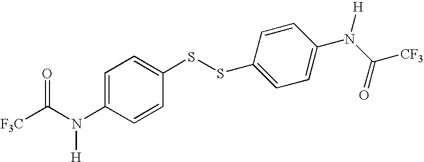

¹H-NMR (DMSO-d₆) δ: 7.55-7.59 (4H, m), 7.68-7.72 (4H, m), 11.37 (2H, brs).

### Production Example 71

To a mixture of bis[4-(N-trifluoroacetylamino)phenyl]disulfide (9.21 g) and dimethylsulfoxide (90 ml) was added sodium hydride (60% in oil; 2.40 g), and stirred at room temperature for 30 minutes. Methyl iodide (7.5 ml) was added dropwise thereto, and stirred at room temperature for 1 hr. To the reaction mixture were added ice-water (150 ml) and ethyl acetate (150 ml), and separated the layers. The organic layer was washed twice with saturated saline (150 ml), dried over anhydrous magnesium sulfate, and concentrated under reduced pressure. The obtained residue was purified by silica gel chromatography (hexane:ethyl acetate=1:5 to 1:2) to give 7.56 g of bis[4-(N-methyl-N-trifluoroacetylamino)phenyl]disulfide.

### Bis[4-(N-methyl-N-trifluoroacetylamino)phenyl]disulfide

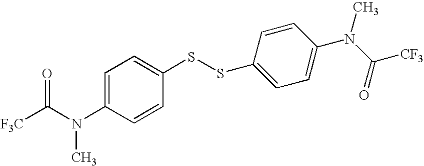

¹H-NMR (CDCl₃) δ: 3.33 (6H, s), 7.16-7.21 (4H, m), 7.51-7.59 (4H, m).

### Production Example 72

To a mixture of bis[4-(N-methyl-N-trifluoroacetylamino)phenyl]disulfide (6.87 g) and methanol (60 ml) was added potassium carbonate (4.10 g), and stirred at room temperature for 4 hrs. Sodium hydride (60% in oil; 200 mg) was added thereto, and stirred at room temperature for 3 hrs. The reaction mixture was filtered, and the filtrate was concentrated under reduced pressure. To the resulting residue were added water (100 ml) and chloroform (100 ml), and separated the layers. The organic layer was dried over anhydrous magnesium sulfate, and concentrated under reduced pressure. The obtained residue was purified by silica gel chromatography (chloroform) to give 3.39 g of bis[4-(N-methylamino)phenyl]disulfide.

### Bis[4-(N-methylamino)phenyl]disulfide

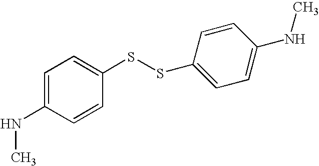

¹H-NMR (CDCl₃) δ: 2.83 (6H, s), 3.86 (2H, brs), 6.49-6.53 (4H, m), 7.25-7.30 (4H, m).

### Production Example 73

To a mixture of bis[4-(N-methylamino)phenyl]disulfide (1.00 g) and diethyl ether (10 ml) was added a mixture of 2,6-difluorobenzoyl isocyanate (1.46 g) and diethyl ether (2.0 ml) under ice-cooling. The resulting mixture was stirred at room temperature for 6 hrs. The solids deposited in the reaction mixture were collected by filtration, and 2.30 g of bis[4-[N-[(2,6-difluorobenzoyl)aminocarbonyl]-N-methylamino]phenyl]disulfide was obtained.

### Bis[4-[N-[(2,6-difluorobenzoyl)aminocarbonyl]-N-methylamino]phenyl]disulfide

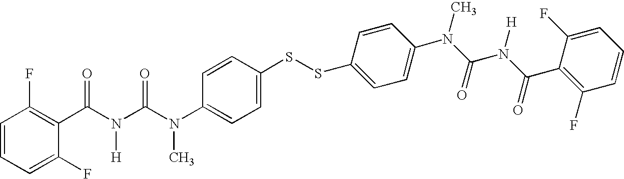

¹H-NMR (DMSO-d₆) δ: 3.22 (6H, s), 7.11-7.15 (4H, m), 7.29-7.31 (4H, m), 7.47-7.52 (2H, m), 7.58-7.59 (4H, m), 7.39-7.52 (3H, m), 10.66 (2H, brs).

### Production Example 74

To a mixture of bis[4-[N-[(2,6-difluorobenzoyl)aminocarbonyl]-N-methylamino]phenyl]disulfide (2.00 g) and 1-methyl-2-pyrrolidone (20.0 ml) was added sodium hydride (0.31 g) at 2° C., and stirred for 30 minutes. To the resulting mixture was added methyl iodide (0.93 ml) at 2° C., and stirred at 2 to 3° C. for 3 hrs. To the reaction mixture was added a mixed solution of saturated aqueous ammonium chloride solution (20 ml) and water (20 ml) under ice-cooling, and extracted three times with ethyl acetate (40 ml). The combined organic layer was washed three times with saturated saline, dried over anhydrous magnesium sulfate, and concentrated under reduced pressure. The obtained residue was purified by silica gel chromatography (ethyl acetate:hexane=1:1) to give 1.67 g of bis[4-[N—[N′-(2,6-difluorobenzoyl)-N′-methylaminocarbonyl]-N-methylamino]phenyl]disulfide.

### Bis[4-[N—[N′-(2,6-difluorobenzoyl)-N′-methylaminocarbonyl]-N-methylamino]phenyl]disulfide

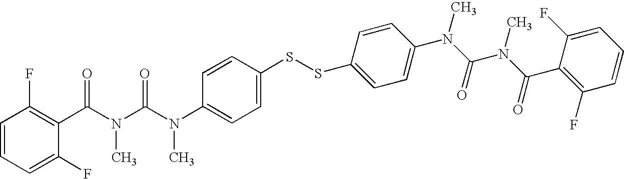

¹H-NMR (CDCl₃) δ: 3.03 (6H, s), 3.29 (6H, s), 6.85-7.15 (8H, m), 7.32-7.36 (2H, m), 7.46-7.48 (4H, m).

### Example 134

To a mixture of bis[4-[N—[N′-(2,6-difluorobenzoyl)-N′-methylaminocarbonyl]-N-methylamino]phenyl]disulfide (1.67 g) and N,N-dimethylformamide (16 ml) was added sodium trichloroacetate (1.60 g), and heated at 100° C. for 10 minutes. The reaction mixture was filtered through Celite. The filtrate was concentrated under reduced pressure. The residue was purified by silica gel chromatography (ethyl acetate:hexane=1:1), then purified by medium pressure preparative high performance liquid chromatography (hexane:ethyl acetate=15:85 to 20:80) to give 0.49 g of 1-(2,6-difluorobenzoyl)-1,3-dimethyl-3-[4-(trichloromethylthio)phenyl]urea (hereinafter, referred to as the present compound (134)).

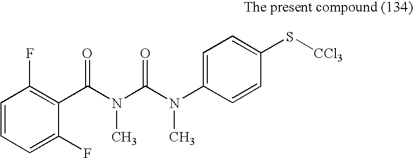

¹H-NMR (DMSO-d₆) δ: 3.05 (3H, s), 3.29 (3H, s), 7.13-7.50 (4H, m), 7.51-7.59 (1H, m), 7.79-7.81 (2H, m).

In the following, Preparation Examples will be shown. In addition, parts represent parts by weight.

### Preparation Example 1

Into a mixture of 35 parts of xylene and 35 parts of N,N-dimethylformamide, 10 parts of any one of the present compounds (1) to (134) is dissolved, and then 14 parts of polyoxyethylene styrylphenyl ether and 6 parts of calcium dodecylbenzenesulfonate are added. The mixture is stirred thoroughly to obtain a 10% emulsion.

### Preparation Example 2

To a mixture of 4 parts of sodium laurylsulfate, 2 parts of calcium ligninsulfonate, 20 parts of synthetic hydrous silicon oxide fine powder and 54 parts of diatomaceous earth, 20 parts of any one of the present compounds (1) to (134) is added. The mixture is stirred thoroughly to obtain a 20% wettable powder.

### Preparation Example 3

To 2 parts of any one of the present compounds (1) to (134), 1 part of synthetic hydrous silicon oxide fine powder, 2 parts of calcium ligninsulfonate, 30 parts of bentonite and 65 parts of kaolin clay are added, and then stirred thoroughly. Then, an appropriate amount of water is added to the mixture. The mixture is further stirred, granulated with a granulator, and forced-air dried to obtain a 2% granule.

### Preparation Example 4

Into an appropriate amount of acetone, 1 part of any one of the present compounds (1) to (134) is dissolved, and then 5 parts of synthetic hydrous silicon oxide fine powder, 0.3 part of PAP and 93.7 parts of fubasami clay are added. The mixture is stirred thoroughly. Then, acetone is removed from the mixture by evaporation to obtain a 1% powder.

### Preparation Example 5

A mixture of 10 parts of any one of the present compounds (1) to (134); 35 parts of white carbon containing 50 parts of polyoxyethylene alkyl ether sulfate ammonium salt; and 55 parts of water is finely ground by a wet grinding method to obtain a 10% flowable agent.

### Preparation Example 6

In 5 parts of xylene and 5 parts of trichloroethane, 0.1 part of any one of the present compounds (1) to (134) is dissolved. The solution is mixed with 89.9 parts of deodorized kerosene to obtain a 0.1% oil.

### Preparation Example 7

In 0.5 ml of acetone, 10 mg of any one of the present compounds (1) to (134) is dissolved. The solution is mixed uniformly with 5 g of a solid feed powder for animals (solid feed powder for rearing and breeding CE-2, manufactured by CLEA Japan, Inc.), and then dried by evaporation of acetone to obtain poison feed.

Then, it will be shown by Test Examples that the present compound is effective for controlling pests.

### Test Example 1

Ten parts of the present compound (1), (2), (5) to (19), (21) to (34), (36) to (61), (63), (64), (68) to (71), (74), (76) to (78), (82) to (83), (89), (93), (94), (97), (99), (102) to (110), (111), (115), (116) (117) to (120) or (123) to (134); 35 parts of white carbon containing 50 parts of polyoxyethylene alkyl ether sulfate ammonium salt; and 55 parts of water were mixed, and finely ground by a wet grinding method to obtain a 10% flowable agent. The obtained flowable agent was diluted with water so that the active ingredient concentration became to 500 ppm to prepare a spray solution for test.

Cabbages were planted in polyethylene cups, and grown until the third true leaf or the fourth true leaf was developed. The spray solution for test prepared above was sprayed at a rate of 20 ml/cup on the cabbages.

After the pesticidal solution sprayed onto the cabbages was dried, 5 second-instar larvae of Plutella xylostella were put on the cabbages. After 5 days, the number of dead Plutella xylostella was examined, and a dead pest rate was calculated by the following equation:

Dead pest rate(%)=(the number of dead pests/the number of tested pests)×100

As a result, in the treated-area with each of the spray solutions for test of the present compounds (1), (2), (5) to (19), (21) to (34), (36) to (61), (63), (64), (68) to (71), (74), (76) to (78), (82), (83), (89), (93), (94), (97), (99), (102) to (110), (111), (115), (116), (117) to (120) and (123) to (134), a control value of 100% was exhibited.

### Test Example 2

In 0.25 mL of a mixture solution of Sorgen TW-20 (manufactured by Daiichi Industries Pharmaceuticals, Co., Ltd.) and acetone (mixture volume ratio; Sorgen TW-20:acetone=1:19), 2.5 mg of the present compound (1), (2), (3), (4), (7), (10), (11), (12), (16), (18), (19), (20), (21), (22), (25), (30), (36), (37), (41), (42), (52), (54), (56), (57), (60), (62), (105) or (109) was dissolved. The solution was diluted with ion-exchanged water so that the active ingredient concentration became to a predetermined concentration to prepare a pesticidal solution for test of the test compound. The root part of cabbages in the fourth leaf period were washed with tap water to remove soils, and then immersed in the pesticidal solution for test. After 5 days from immersion of the root part, the root part was removed, and the leaves and stems were put in a cup (volume; 180 mL). In the cup 10 second-instar larvae of Plutella xylostella were released, and the cup was stored at 24° C. After 5 days, the number of dead pests was counted, and the dead pest rate was calculated by the following equation:

Dead pest rate(%)=(the number of dead pests/the number of tested pests)×100

As a result, the present compounds (1), (2), (3), (4), (7)**, (10), (11), (12), (16), (18)*, (19)*, (20)**, (21)*, (22)**, (25)**, (30)**, (36)**, (37), (41), (42), (52)**, (54), (56), (57)**, (60), (62), (105) and (109) each exhibited a dead pest rate of 100%. *: test concentration of 1 ppm**: test concentration of 5 ppm

The test concentrations of the other present compounds were 25 ppm.

### Test Example 3

Ten parts of the present compound (1) to (5), (7) to (13), (18) to (33), (35) to (49), (52) to (56), (58) to (61), (71), (73) to (77), (79), (80), (82), (83), (93), (94), (95), (96), (97), (100) to (109), (111), (112), (115), (117), (119), (121), or (123) to (133), 35 parts of white carbon containing 50 parts of polyoxyethylene alkyl ether sulfate ammonium salt, and 55 parts of water were mixed, and finely ground by a wet grinding method to obtain a preparation. The obtained preparation was diluted with water so that the active ingredient concentration became to 500 ppm to prepare a pesticidal solution for test. On the bottom of a polyethylene cup having a diameter of 5.5 cm, a filter paper having a diameter of 5.5 cm was placed, on which Insecta LF (Nippon Agriculture Industries, Co., Ltd.) sliced in a thickness of 6 mm and further cut half was laid, and to which 2 mL of the above-described pesticidal solution for test was added. After air-dried, 5 fourth-instar larvae of Spondoptela litura were released in the cup, and the cup was capped. After 6 days, the number of dead pests was counted, and the dead pest rate was calculated by the following equation:

Dead pest rate(%)=(the number of dead pests/the number of tested pests)×100

As a result, the present compounds (1) to (5), (7) to (13), (18) to (33), (35) to (49), (52) to (56), (58) to (61), (71), (73) to (77), (79), (80), (82), (83), (93), (94), (95), (96), (97), (100) to (109), (111), (112), (115), (117), (119), (121), and (123) to (133) each exhibited a dead pest rate of 100%.

### Test Example 4

Ten parts of the present compound (1), (2), (4), (7), (10) to (13), (18) to (20), (22), (23), (24), (25), (28), (29), (30), (31), (34), (36), (40), (41), (45), (47), (49), (52) to (54), (56), (62), (69), (71), (73), (76), (81), (97), (101), (104), (105), (106), (107), (108), (109), (110), (117) or (131); 35 parts of white carbon containing 50 parts of polyoxyethylene alkyl ether sulfate ammonium salt; and 55 parts of water were mixed, and finely ground by a wet grinding method to obtain a preparation. The obtained preparation was diluted with water so that the active ingredient concentration became to a predetermined concentration to prepare a spray solution for test. Cucumbers were planted in polyethylene cups, and were grown until the first true leaf was developed. The spray solution for test prepared above was sprayed at the rate of 20 ml/cup on the cucumber. After the pesticidal solution sprayed onto the cucumber was dried, the first true leaf was cut off and then placed on a filter paper (diameter: 70 mm) containing water in a polyethylene cup (diameter: 110 mm). On the cucumber leaf, 30 larvae of Frankliniella occidentalis were released, and the polyethylene cup was capped. Seven days after spraying, the number of pests surviving on the cucumber leaf was counted, and a control value was calculated by the following equation:

Control value(%)={1−(Cb×Tai)/(Cai×Tb)}×100

wherein, each symbol has following meaning:

Cb: the number of pests before treatment in a non-treated area

Cai: the number of pests at the time of observation in a non-treated area

Tb: the number of pests before treatment in a treated-area

Tai: the number of pests at the time of observation in a treated-area.

As a result, in the treated-area with each of the test spray solutions of the present compounds (1), (2), (4), (7)**, (10) to (13), (18) to (20), (22)*, (23)**, (24)**, (25)**, (28), (29), (30), (31), (34), (36)**, (40)**, (41), (45), (47), (49), (52) to (54), (56), (62), (69), (71), (73), (76), (81), (97), (101), (104), (105), (106), (107), (108), (109), (110), (117) and (131), a control value of 100% was exhibited. *: test concentration 3.2 ppm**: test concentration 12.5 ppm

The test concentrations of the other present compounds were 50 ppm.

### Test Example 5

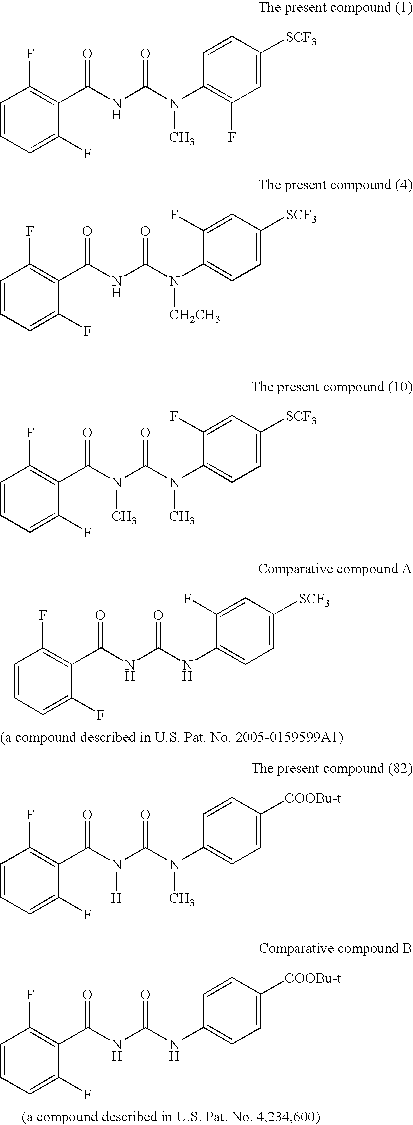

In 0.25 mL of a mixed solution of Tween-20 and acetone (mixture volume ratio: Tween-20:acetone=1:19), 2.5 mg of the present compound (1), (4), (10), (82), comparative compound A or B was dissolved. The solution was diluted with ion-exchanged water so that the active ingredient concentration became to the predetermined concentration (25 ppm) to prepare a pesticidal solution for test of the test compound. The root part of cabbages in the fourth leaf period was washed with tap water to remove soils, and then immersed in the pesticidal solution for test. After 5 days from immersion of the root part, the root part was removed, and the leaves and stems were put in a cup (volume; 180 mL). In the cup 10 second-instar larvae of Plutella xylostella were released, and the cup was stored at 24° C. After 5 days, the number of dead pests was countered, and the dead pest rate was calculated by the following equation:

Dead pest rate(%)=(the number of dead pests/the number of tested pests)×100

As a result, the present compounds (1), (4), (10) and (82) each exhibited a dead pest rate of 80 to 100%. On the other hand, the comparative compound A exhibited a dead pest rate of 5%, and the comparative compound B exhibited a dead pest rate of 0%.

### Test Example 6

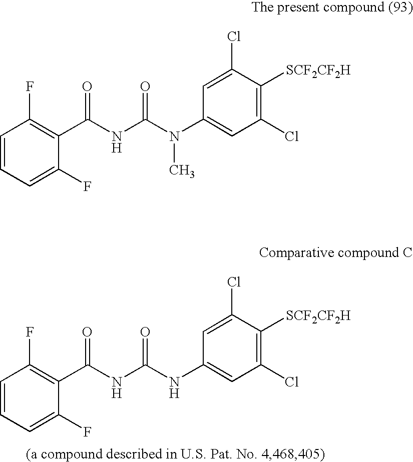

In 0.25 mL of a mixed solution of Tween-20 and acetone (mixture volume ratio: Tween-20:acetone=1:19), 2.5 mg of the present compound (93) or comparative compound C was dissolved. The solution was diluted with ion-exchanged water so that the active ingredient concentration became to the predetermined concentration (100 ppm) to prepare a pesticidal solution for test of the test compound. The root part of cabbages in the fourth leaf period was washed with tap water to remove soils, and then immersed in the pesticidal solution for test. After 5 days from immersion of the root part, the root part was removed, and the leaves and stems were put in a polyethylene cup (volume; 180 mL). In the cup 10 second-instar larvae of Plutella xylostella were released, and the cup was stored at 24° C. After 5 days, the number of dead pests was countered, and the dead pest rate was calculated by the following equation:

Dead pest rate(%)=(the number of dead pests/the number of tested pests)×100

As a result, the present compound (93) exhibited a dead pest rate of 90%. On the other hand, the comparative compound exhibited a dead pest rate of 20%.

### Test Example 7

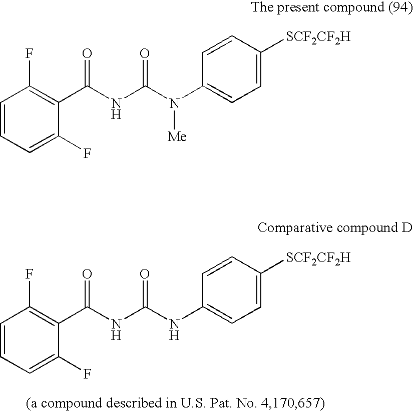

In 0.25 mL of a mixed solution of Tween-20 and acetone (mixture volume ratio: Tween-20:acetone=1:19), 2.5 mg of the present compound (94) or comparative compound D was dissolved. The solution was diluted with ion-exchanged water so that the active ingredient concentration became to the predetermined concentration (25 ppm) to prepare a pesticidal solution for test of the test compound. The root part of cabbages in the fourth leaf period was washed with tap water to remove soils, and then immersed in the pesticidal solution for test. After 5 days from immersion of the root part, the root part was removed, and the leaves and stems were put in a ice cream cup (volume; 180 mL). In the cup 10 second-instar larvae of Plutella xylostella were released, and the cup was stored at 24° C. After 5 days, the number of dead pests was countered, and the dead pest rate was calculated by the following equation:

Dead pest rate(%)=(the number of dead pests/the number of tested pests)×100

As a result, the present compound (94) exhibited a dead pest rate of 100%. On the other hand, the comparative compound exhibited a dead pest rate of 20%.

### Test Example 8

A filter paper having a diameter of 33 mm (No. 1026 manufactured by Toyo Filter Paper, Co., Ltd.) was treated with acetone solution (1 mL) of 10 mg/mL of the present compounds (1) or (12) using a pipette, and dried at room temperature. Hereinafter, the resulting filter paper is referred to as a filter paper bait. 4% agarose was poured into a plastic petri dish having a diameter of 9 cm to make the thickness about 5 mm, and solidified by leaving at rest at room temperature. One circular hole having a diameter of 35 mm (hereinafter, referred to as a well) was made in the solidified agarose. One filter paper bait was put into the well. Then, after 20 ergates of Coptotermes formosanus were released in the above petri dish, the petri dish was capped, and sealed with a parafilm. After storing in a dark place for 6 weeks, the petri dish was opened. The control rate was calculated by observing the life and death of the Coptotermes formosanus in the petri dish. As a result, the present compound (10) exhibited a control rate of 65%, and the present compound (12) exhibited a control rate of 60%.

### Test Example 9

In a mixed solution (0.1 mL) of xylene and N,N-dimethylformamide (mixture volume ratio; xylene:N,N-dimethylformamide=1:1), 30 mg of the present compounds (3), (4), (20), (35), (62), (81), (100), (101) or (112) was dissolved, and further a mixed solution (0.1 mL) of xylene and SORPOL 3005X (manufactured by Toho Chemcials, Co., Ltd.) (mixture volume ratio; xylene:SORPOL 3005X=1:9) was added thereto. The solution was diluted with ion-exchanged water so that the active ingredient became to the predetermined concentration to prepare a pesticidal solution for test of the test compound.

Cucumbers were planted in polyethylene cups, and grown until the third true leaf or the fourth true leaf was developed. The spray solution for test prepared above was sprayed at a rate of 20 ml/cup on the cabbages.

After the pesticidal solution for test was dried, the aerial part of the cabbage was cut, and put into a polyethylene cup having a volume of 100 ml together with 10 third-instar larvae of Plutella xylostella, and stored at 25° C. After 5 days, the number of dead pests was countered, and the dead pest rate was calculated by the following equation:

Dead pest rate(%)=(the number of dead pests/the number of tested pests)×100

As a result, the present compound (3)*, (4)**, (20)*, (35)**, (62), (81), (100), (101), and (112)** exhibited a dead pest rate of 100%, respectively. *: test concentration 12.5 ppm**: test concentration 50 ppm

Test concentrations of the others were 200 ppm.

### INDUSTRIAL APPLICABILITY

The compound (I) or a salt thereof is useful as an active ingredient for pesticides since it has an excellent controlling efficacy against pests.

1. A benzoylurea compound represented by formula (I): 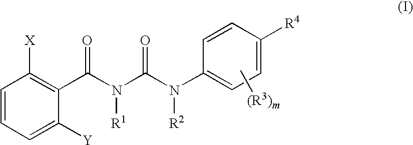
wherein, X and Y independently represent a fluorine atom or chlorine atom, respectively,
R¹ represents a hydrogen atom, a lower alkyl group optionally substituted with one or more of halogen atoms, a lower alkenyl group optionally substituted with one or more of halogen atoms, a lower alkynyl group, an aryl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, a lower alkoxycarbonyl group, an aryl lower alkyloxycarbonyl group, a N,N-di(lower alkyl)carbamoyl group, a lower alkanoyl group optionally substituted with one or more of halogen atoms, formyl group, a lower alkylsulfonyl group optionally substituted with one or more of halogen atoms, an arylsulfonyl group, an aryloxycarbonyl group, a lower cycloalkyl group, a lower cycloalkyl lower alkyl group, a di(lower alkyl)amino group, a lower alkoxy group, a lower alkanoyloxy lower alkyl group, an aryl lower alkoxy lower alkyl group, 6-membered saturated heterocyclic group, or a group represented by —(CH₂)ₗ-A wherein l represents an integer of 1 to 4 and A represents a di(lower alkoxy)methyl group, a lower alkoxycarbonyl group, or a 5- or 6-membered heterocyclic group optionally substituted with a halogen atom,
R² represents a lower alkyl group,
R³ represents a halogen atom or a lower alkyl group optionally substituted with one or more of halogen atoms,
R⁴ represents a lower alkoxycarbonyl group, or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, and
m represents an integer of 0 to 4; or a salt thereof.

2. The compound according to claim 1, wherein X and Y independently represents a fluorine atom or a chlorine atom, respectively,
R¹ represents a hydrogen atom, a lower alkyl group optionally substituted with one or more of halogen atoms, a lower alkenyl group optionally substituted with one or more of halogen atoms, a lower alkynyl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a N,N-di(lower alkyl)amino lower alkyl group, a lower alkylthio lower alkyl group, a lower alkylsulfinyl lower alkyl group, a lower alkylsulfonyl lower alkyl group, a lower alkoxycarbonyl group, an aryl lower alkoxycarbonyl group, a N,N-di(lower alkyl)carbamoyl group, a lower alkanoyl group optionally substituted with one or more of halogen atoms, a lower alkylsulfonyl group optionally substituted with one or more of halogen atoms, an arylsulfonyl group, an aryloxycarbonyl group, a lower cycloalkyl group, a lower cycloalkyl lower alkyl group, a di(lower alkyl)amino group, a lower alkoxy group, an aryl lower alkoxy lower alkyl group, a 6-membered saturated heterocyclic group, or a group represented by —(CH₂)ₗ-A wherein l represents an integer of 1 or 2, and A represents a di(lower alkoxy)methyl group, a lower alkoxycarbonyl group, or a 5- or 6-membered heterocyclic group optionally substituted with a halogen atom,
R² represents a lower alkyl group,
R³ represents a halogen atom or a lower alkyl group optionally substituted with one or more of halogen atoms,
R⁴ represents a lower alkoxycarbonyl group, or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2,
m represents an integer of 0 to 2.

3. The compound according to claim 1, wherein R¹ represents a hydrogen atom, a lower alkyl group optionally substituted with one or more of halogen atoms, a lower alkenyl group, a lower alkynyl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a lower alkylthio lower alkyl group, a lower alkylsulfinyl lower alkyl group, a lower alkylsulfonyl lower alkyl group, a lower alkoxycarbonyl group, an aryl lower alkyloxycarbonyl group, a N,N-di(lower alkyl)carbamoyl group, a lower alkanoyl group, a lower alkylsulfonyl group, an arylsulfonyl group, a lower cycloalkyl group, a lower cycloalkyl lower alkyl group, a di(lower alkyl)amino group, a lower alkoxy group, a 6-membered saturated heterocyclic group, or a group represented by —(CH₂)ₗ-A wherein l represents an integer of 1 or 2, and A represents a lower alkoxycarbonyl group, or a 5- or 6-membered heterocyclic group optionally substituted with a halogen atom,
R³ represents a halogen atom or a lower alkyl group,
R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms.

4. The compound according to claim 1, wherein n represents an integer of 1 or 2.

5. The compound according to claim 1, wherein R⁴ represents a lower alkoxycarbonyl group.

6. The compound according to claim 1, wherein R¹ represents a lower alkyl group substituted with one or more of halogen atoms.

7. A benzoylurea compound represented by formula (I-a): 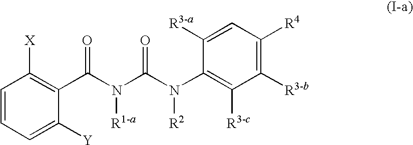
wherein, X and Y independently represent a fluorine atom or chlorine atom, respectively,
R^{1-a} represents a hydrogen atom or a lower alkyl group,
R² represents a lower alkyl group, and
(1) when R^{3-a} and R^{3-b} represent a halogen atom, R^{3-c} represents a hydrogen atom, or
(2) when R^{3-a} and R^{3-c} represent a halogen atom, R^{3-b} represents a hydrogen atom, or
(3) when R^{3-a} represents a halogen atom or a lower alkyl group, R^{3-b} and R^{3-c} represent a hydrogen atom, and
R⁴ represents a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, or a salt thereof.

8. The compound according to claim 7, wherein (1) when R^{3-a} and R^{3-b} represent a halogen atom, R^{3-c} represents a hydrogen atom, or
(2) when R^{3-a} and R^{3-c} represent a halogen atom, R^{3-b} represents a hydrogen atom, and
R⁵ represents a lower alkyl group optionally substituted with one or more of halogen atoms.

9. The compound according to claim 7, wherein R^{3-a} represents a halogen atom or a lower alkyl group, R^{3-b} and R^{3-c} represent a hydrogen atom,
R⁵ represents a lower alkyl group optionally substituted with one or more of halogen atoms.

10. The compound according to claim 1, wherein R³ represents a lower alkyl group substituted with a halogen atom.

11. A process for producing a compound represented by formula (I-7) 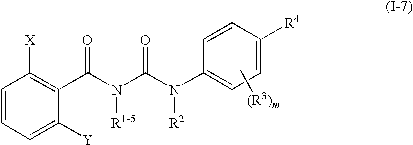
wherein, X and Y independently represent a fluorine atom or a chlorine atom, respectively,
R¹⁻⁵ represents a lower alkyl group optionally substituted with one or more of halogen atoms, a lower alkenyl group optionally substituted with one or more of halogen atoms, a lower alkynyl group, an aryl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, a lower alkoxycarbonyl group, an aryl lower alkoxycarbonyl group, a N,N-di(lower alkyl)carbamoyl group, a lower alkanoyl group optionally substituted with one or more of halogen atoms, formyl group, a lower alkylsulfonyl group optionally substituted with one or more of halogen atoms, an arylsulfonyl group, an aryloxycarbonyl group, a lower cycloalkyl group, a lower cycloalkyl lower alkyl group, a di(lower alkyl)amino group, a lower alkoxy group, a lower alkanoyloxy lower alkyl group, an aryl lower alkoxy lower alkyl group, 6-membered saturated heterocyclic group, or a group represented by —(CH₂)ₗ-A wherein l represents an integer of 1 to 4, and A represents a di(lower alkoxy)methyl group, a lower alkoxycarbonyl group, or a 5- or 6-membered heterocyclic group optionally substituted with a halogen atom,
R² represents a lower alkyl group,
R³ represents a halogen atom, or a lower alkyl group optionally substituted with one or more of halogen atoms,
R⁴ represents a lower alkoxycarbonyl group, or a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, and
m represents an integer of 0 to 4,
which comprises reacting a compound represented by formula (II) 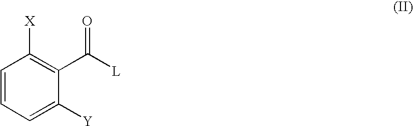
wherein X and Y are as defined above, and L represents a halogen atom, with a compound represented by formula (III) 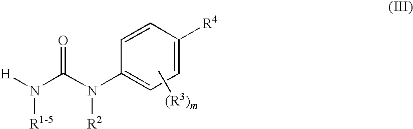
wherein each symbol is as defined above, in an organic solvent in the presence of an organic base or a metal carbonate, and isolating.

12. The process according to claim 11, wherein R¹⁻⁵ represents a lower alkyl group,
R³ represents a halogen atom, or a lower alkyl group,
R⁴ represents a group represented by S(O)ₙR⁵ wherein
R⁵ represents a lower alkyl group optionally substituted with one or more of halogen atoms, and n represents an integer of 0, and
m represents an integer of 1.

13. A pesticide comprising the compound or a salt thereof according to claim 1 as an active ingredient.

14. Use of the compound or a salt thereof according to claim 1 for pest control.

15. Use of the compound according to claim 1 for manufacturing a pesticide for controlling pests.

16. A method for controlling pests which comprises applying effective amount of the compound or a salt thereof according to claim 1 to pests directly or habitat of pests.

17. A compound represented by formula (III) 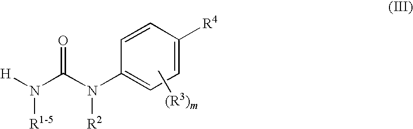
wherein, R¹⁻⁵ represents a lower alkyl group optionally substituted with one or more of halogen atoms, a lower alkenyl group optionally substituted with one or more of halogen atoms, a lower alkynyl group, an aryl group, an aryl lower alkyl group optionally substituted with one or more of lower alkoxy groups, a lower alkoxy lower alkyl group optionally substituted with one or more of halogen atoms, an aryloxy lower alkyl group optionally substituted with one or more of halogen atoms, a lower alkanoyloxy lower alkyl group, an aryl lower alkoxy lower alkyl group, a N,N-di(lower alkyl)amino lower alkyl group, a lower alkylthio lower alkyl group, a lower alkylsulfinyl lower alkyl group, a lower alkylsulfonyl lower alkyl group, a lower alkoxy lower alkoxy lower alkyl group, a lower alkoxycarbonyl group, an aryl lower alkyloxycarbonyl group, a N,N-di(lower alkyl)carbamoyl group, a lower alkanoyl group optionally substituted with one or more of halogen atoms, formyl group, a lower alkylsulfonyl group optionally substituted with one or more of halogen atom, an aryl sulfonyl group, an aryloxycarbonyl group, a lower cycloalkyl group, a lower cycloalkyl lower alkyl group, a di(lower alkyl)amino group, a lower alkoxy group, 6-membered saturated heterocyclic group, or a group represented by —(CH₂)ₗ-A wherein l represents an integer of 1 to 4, and A represents a di(lower alkoxy)methyl group, a lower alkoxycarbonyl group, or a 5- or 6-membered heterocyclic group optionally substituted with a halogen atom,
R² represents a lower alkyl group,
R³ represents a halogen atom, or a lower alkyl group optionally substituted with one or more of halogen atoms,
R⁴ represents a group represented by S(O)ₙR⁵ wherein R⁵ represents a lower alkyl group optionally substituted with one or more of halogen atoms, a lower alkenyl group optionally substituted with one or more of halogen atoms, a lower alkynyl group, or a lower alkoxy lower alkyl group optionally substituted with one or more of halogen atoms, and n represents an integer of 0 to 2, and
m represents an integer of 0 to 4.

18. The compound according to claim 17, wherein R¹⁻⁵ represents a lower alkyl group optionally substituted with one or more of halogen atoms, a lower alkenyl group optionally substituted with one or more of halogen atoms, a lower alkynyl group, an aryl lower alkyl group optionally substituted with one or more of lower alkyl groups, a lower alkoxy lower alkyl group optionally substituted with one or more of halogen atoms, an aryloxy lower alkyl group optionally substituted with one or more of halogen atoms, a N,N-di(lower alkyl)amino lower alkyl group, a lower alkylthio lower alkyl group, a lower alkylsulfinyl lower alkyl group, a lower alkylsulfonyl lower alkyl group, a lower cycloalkyl group, a lower cycloalkyl lower alkyl group, a di(lower alkyl)amino group, a lower alkoxy group, 6-membered saturated heterocyclic group, or a group represented by —(CH₂)ₗ-A wherein l represents an integer of 1 to 4, and A represents a di(lower alkoxy)methyl group, a lower alkoxycarbonyl group, or a 5- or 6-membered heterocyclic group optionally substituted with a halogen atom.
